(19) United States
(12) Patent Application Publication
(10) Pub. No.: US 2025/0257316 A1
(43) Pub. Date: Aug. 14, 2025

(54) CONTROL OF NITROGEN FIXATION IN RHIZOBIA THAT ASSOCIATE WITH CEREALS

(71) Applicant: Massachusetts Institute of Technology, Cambridge, MA (US)

(72) Inventors: Christopher A. Voigt, Belmont, MA (US); Min-Hyung Ryu, Cambridge, MA (US)

(73) Assignee: Massachusetts Institute of Technology, Cambridge, MA (US)

(21) Appl. No.: 19/174,691

(22) Filed: Apr. 9, 2025

Related U.S. Application Data
(63) Continuation of application No. 16/746,215, filed on Jan. 17, 2020, now Pat. No. 12,281,299.
(60) Provisional application No. 62/820,765, filed on Mar. 19, 2019.

(51) Int. Cl. C12N 1/20; C05F 11/08; C12N 15/87

(52) U.S. Cl. CPC C12N 1/20; C05F 11/08; C12N 15/87; C12N 2510/00

(57) ABSTRACT

Disclosed herein are engineered rhizobia having nif clusters that enable the fixation of nitrogen under free-living conditions, as well as ammonium and oxygen tolerant nitrogen fixation under free-living conditions. Also provided are methods for producing nitrogen for consumption by a cereal crop using these engineered rhizobia.

### RELATED APPLICATION

This application claims priority under 35 U.S.C. § 119 (e) to U.S. Provisional Application Ser. No. 62/820,765, filed Mar. 19, 2019, the entire contents of which are incorporated by reference herein.

### GOVERNMENT SUPPORT

This invention was made with government support under Grant No. IOS1331098, awarded by the National Science Foundation (NSF). The government has certain rights in this invention.

### BACKGROUND OF THE INVENTION

In agriculture, nitrogen is a limiting nutrient that needs to be added as fertilizer to those crops that cannot produce it on their own, including the cereals rice, corn, and wheat. In contrast, legumes are able to obtain nitrogen from the atmosphere using nitrogen-fixing bacteria that reside in root nodules. However, the majority of the world's calories are from cereals; thus, it has been a longstanding problem in genetic engineering to transfer this ability to these crops. This would reduce the need for nitrogenous fertilizer and the economic, environmental, and energy burdens that it brings.

### SUMMARY OF THE INVENTION

The present disclosure is based, at least in part, rhizobia and methods for making rhizobia that can fix nitrogen under aerobic free-living conditions. The present disclosure also provides refactored nif-clusters that confer the ability to fix nitrogen under aerobic free-living conditions.

Accordingly, one aspect of the present disclosure provides a rhizobium that can fix nitrogen under aerobic free-living conditions, comprising a symbiotic rhizobium having an exogenous nif cluster, wherein the exogenous nif cluster confers nitrogen fixation capability on the symbiotic rhizobium under aerobic free-living conditions, and wherein the rhizobium is not Azorhizobium caulinodans. In some embodiments, the exogenous nif cluster is from a free-living diazotroph. In some embodiments, the exogenous nif cluster is from a symbiotic diazotroph. In some embodiments, the exogenous nif cluster is from a photosynthetic Alphaproteobacteria. In some embodiments, the exogenous nif cluster is from a Gammaproteobacteria. In some embodiments, the exogenous nif cluster is from a cyanobacteria. In some embodiments, the exogenous nif cluster is from a firmicutes. In some embodiments, the exogenous nif cluster is from Rhodobacter sphaeroides. In some embodiments, the exogenous nif cluster is from Rhodopseudomonas palustris. In some embodiments, the exogenous nif cluster is an inducible refactored nif cluster. In some embodiments, the inducible refactored nif cluster is an inducible refactored Klebsiella nif cluster. In some embodiments, the rhizobium is IRBG74. In some embodiments, the exogenous nif cluster comprises 6 nif genes. In some embodiments, the 6 nif genes are nifHDK(T)Y, nifEN(X), nifJ, nifBQ, nifF, and nifUSVWZM. In some embodiments, each nif gene of the exogenous nif cluster is preceded by a T7 promoter. In some embodiments, the T7 promoter is a wild-type promoter. In some embodiments, the rhizobium further comprises an endogenous nif cluster. In some embodiments, the nif cluster has a nifV gene. In some embodiments, the nifV gene is endogenous. In some embodiments, the exogenous nif cluster further comprises a terminator. In some embodiments, the T7 promoter has a terminator and the terminator is downstream from the T7 promoter. In some embodiments, the exogenous nif cluster is a refactored v3.2 nif cluster as shown in FIG. 2H.

Another aspect of the present disclosure provides a plant growth promoting bacterium that can fix nitrogen under aerobic free-living conditions, comprising a bacterium having an exogenous nif cluster having at least one inducible promoter, wherein the exogenous nif cluster confers nitrogen fixation capability on the bacterium, under aerobic free-living conditions, and wherein the bacterium is not Azorhizobium caulinodans. In some embodiments, the bacterium is a symbiotic bacterium. In some embodiments, the bacterium is an endophyte. In some embodiments, the endophyte is rhizobium IRBG74. In some embodiments, the bacterium is an epiphyte. In some embodiments, the epiphyte is pseudomonas protogens PF-5. In some embodiments, the plant growth promoting bacterium is associated with a genetically modified cereal plant. In some embodiments, the genetically modified cereal plant includes an exogenous gene encoding a chemical signal. In some embodiments, the nitrogen fixation is under the control of the chemical signal. In some embodiments, the chemical signal is opine, phlorogluconol or rhizopene. In some embodiments, the exogenous nif cluster comprises 6 nif genes. In some embodiments, the 6 nif genes are nifHDK(T)Y, nifEN(X), nifJ, nifBQ, nifF, and nifUSVWZM. In some embodiments, the inducible promoter is a T7 promoter. In some embodiments, the inducible promoter is P_{A1lacO1} promoter. In some embodiments, the inducible promoter is activated by an agent selected from a group that includes IPTG, sodium salicylate, octapine, nopaline, the quorum signal 3OC6HSL, aTe, cuminic acid, DAPG, and salicylic acid. In some embodiments, the exogenous nif cluster further comprises a terminator. In some embodiments, the inducible promoter has a terminator and the terminator is downstream from the inducible promoter.

Another aspect of the present disclosure provides an Azorhizobium caulinodans capable of inducible ammonium-independent nitrogen fixation in a cereal crop, comprising:
(i) a modified nif cluster, wherein an endogenous nifA gene is deleted or altered; and
(ii) at least one operon comprising nifA and RNA polymerase sigma factor (RpoN), wherein the operon comprises a regulatory element including an inducible promoter. In some embodiments, the inducible promoter is P_{A1lacO1} promotor. In some embodiments, the inducible promoter is activated by an agent selected from the group consisting of IPTG, sodium salicylate, octapine, nopaline, the quorum signal 3OC6HSL, aTc, cuminic acid, DAPG, and salicylic acid. In some embodiments, the endogenous nifA gene is altered with at least one of the following substitutions: (i) L94Q, (ii) D95Q, and (iii) both L94Q and D95Q.

Another aspect of the present disclosure provides a method of engineering a rhizobium that can fix nitrogen under aerobic free-living conditions, comprising transferring an exogenous nif cluster to a symbiotic rhizobium, wherein the exogenous nif cluster confers nitrogen fixation capability on the symbiotic rhizobium, under aerobic free-living conditions, and wherein the rhizobium is not Azorhizobium caulinodans. In some embodiments, the exogenous nif cluster comprises 6 nif genes. In some embodiments, the 6 nif genes are nifHDK(T)Y, nifEN(X), nifJ, nifBQ, nifF and nifUSVWZM. In some embodiments, each of the nif genes is preceded by a wild-type T7 promoter. In some embodiments, the exogenous nif cluster is transferred to the rhizobium in a plasmid. In some embodiments, the exogenous nif cluster further comprises a terminator. In some embodiments, the wild-type T7 promoter has a terminator, and the terminator is downstream from the wild-type T7 promoter. In some embodiments, the endogenous NifL gene is deleted.

Another aspect of the present disclosure provides a method of producing nitrogen for consumption by a cereal plant, comprising providing a plant growth promoting bacterium that can fix nitrogen under aerobic free-living conditions in proximity of the cereal plant, wherein the plant growth promoting bacterium is a symbiotic bacterium having an exogenous nif cluster, wherein the exogenous nif cluster confers nitrogen fixation capability on the symbiotic bacterium, enabling nitrogen fixation under aerobic free-living conditions. In some embodiments, the plant growth promoting bacterium is a rhizobium. In some embodiments, the plant growth bacterium is a bacterium as described in the present disclosure. In some embodiments, the cereal plant is a genetically modified cereal plant. In some embodiments, the genetically modified cereal plant includes an exogenous gene encoding a chemical signal. In some embodiments, the nitrogen fixation is under the control of the chemical signal. In some embodiments, the chemical signal is opine, phlorogluconol or rhizopene. In some embodiments, the nitrogen fixation is under the control of a chemical signal. In some embodiments, the chemical signal is a root exudate, biocontrol agent or phytohormone. In some embodiments, the root exudate is selected from the group consisting of sugars, hormones, flavonoids, and antimicrobials. In some embodiments, the chemical signal is vanillate. In some embodiments, the chemical signal is IPTG, aTe, cuminic acid, DAPG, and salicylic acid, 3,4-dihydroxybenzoic acid, 3OC6HSL or 3OC14HSL.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIGS. 1A-1F include diagrams showing transfer of nif clusters across species. (FIG. 1A) Eight nif clusters from free-living nitrogen fixing bacteria are aligned based on phylogenetic relationships of 16S rRNA sequences. The genes and operons are based on K. oxytoca M5al. Dots in the DNA line indicate where multiple regions were cloned from genomic DNA and combined to form one large plasmid-borne nif cluster. A complete list of strain genotypes is provided in Table 8. Nitrogenase activity from transfer of the native nif clusters was measured in three species. The activities of the R. palustris and R. sphaeroides nif clusters were also measured in 12 Rhizobia strains. Asterisks indicate ethylene production below the detection limit (<10 a.u.). Error bars represent s.d. from three independent experiments. (FIG. 1B) Transcriptomic profile of the native K. oxytoca nif cluster in K. oxytoca, compared with those obtained from its transfer to the indicated species. (FIG. 1C) Transcription levels (FPKM) of the native K. oxytoca nif cluster across species. Transcriptional units are underlined. (FIG. 1D) Transcription levels (FPKM) of the K. oxytoca nif genes in K. oxytoca (→Klebsiella) compared to that obtained when transferred to a new host. (FIG. 1E) Same as in (FIG. 1C), except the translational efficiency is compared, as calculated using ribosome profiling. (FIG. 1F) Same as in (FIG. 1D), except the ribosome densities (RD) are compared, as calculated using ribosome profiling. R2 in log-log plots was calculated from the line (y=x+b), where b is an expression variable between hosts.

FIGS. 2A-2M include diagrams showing the transfer of the refactored K. oxytoca nif clusters to R. sp. IRBG74. (FIG. 2A) The genetic systems for the controller for E. coli MG1655 (left) and R. sp. IRBG74 (right) are shown. A variant of T7 RNAP (R6232S, N-terminal lon tag, GTG start codon) is used for the E. coli controller. Several genetic parts were substituted to build the R. sp. IRBG74 controller (red) (FIG. 16). The sequences for the genetic parts are provided in Table 10. (FIG. 2B) The response functions for the controllers with the reporter plasmid pMR-79 (Table 9 and Table 10). The IPTG concentrations used to induce nitrogenase were circled in red. (FIG. 2C) The genetic parts used to build the refactored v2.1 nif gene cluster are shown (Table 10). (FIG. 2D) The activity of the refactored nif gene cluster v2.1 in different hosts is shown. Asterisks indicate ethylene production below the detection limit (<10 a.u.). (FIG. 2E) The activities of the v2.1 promoters and terminators in E. coli MG1655 and R. sp. IRBG74 as calculated from RNA-seq data (see Materials and Methods). (FIG. 2F) The translation efficiency of the v2.1 nif genes in E. coli MG1655 and R. sp. IRBG74, as calculated using ribosome profiling and RNA-seq. Lines connect points that occur in the same operon. (FIG. 2G) The ribosome density (RD) is compared for the refactored v2.1 nif genes in a new host (E. coli MG155; R. sp. IRBG74) versus that measured for the nif genes from the native K. oxytoca cluster in K. oxytoca (→Klebsiella). The points corresponding to nifH is marked H. (FIGS. 2H-2L) The same as (FIGS. 2C-2G) but with the refactored nif cluster v3.2. Genetic parts are provided in Table 10. (FIG. 2M) Nitrogenase activity is shown as a function of T7 promoter strength. The refactored nif cluster v3.2 was expressed from three controller strains with varying strengths (FIG. 16). Error bars represent s.d. from three independent experiments.

FIGS. 3A-3F include diagrams showing the control of nitrogen fixation in A. caulinodans ORS571. (FIG. 3A) The controller is shown, carried on a pBBR1 origin plasmid (genetic parts are provided in Table 10). NifA and RpoN co-induce the expression of three sites in the genome (identified by consensus NifA binding sequences). (FIG. 3B) Expression from the nifH promoter was evaluated using a fluorescent reporter (see Materials and Methods). NifA and RpoN were complemented (+) individually or in combination in the A. caulinodans ΔnifA strain where the genomic rpoN remains intact. (FIG. 3C) The response function for the induction of the nifH promoter by the controller is shown. (FIG. 3D) The nitrogenase activity is shown for wild-type A. caulinodans ORS571 compared to the ΔnifA complemented with the controller plasmid (+) and the addition of 1 mM IPTG (+). (FIG. 3E) The effect of the absence or presence of 10 mM ammonium chloride is shown. The WT NifA from A. caulinodans ORS571 is compared to different combinations of amino acid substitutions with additional RpoN expression. NifA/RpoN expression is induced by 1 mM IPTG (+) for the ΔnifA strain containing the controller plasmid pMR-121, 122, 123, and 124 (+). Asterisks indicate ethylene production below the detection limit (<10 au). (FIG. 3F) The nitrogenase activity is shown as a function of the oxygen concentration in the headspace (see Materials and Methods). The native nif cluster (wild-type A. caulinodans ORS571) is compared to the inducible version including the controller plasmid and 1 mM IPTG. Error bars represent s.d. from three independent experiments.

FIGS. 4A-4F include diagrams showing Nitrogenase activity of the inducible nif clusters in Pseudomonas protegens Pf-5. (FIG. 4A) The controllers, based on P. stutzeri NifA, were used for all three clusters. Plasmids and genetic parts are provided in Table 9 and Table 10. (FIG. 4B) The nif clusters from K. oxytoca, P. stutzeri, and A. vinelandii are shown. The deleted regions corresponding the NifLA regulators are marked. The dotted lines indicate that multiple regions from the genome were cloned and combined for form the nif cluster. The clusters were carried the plasmids pMR-4, 6, 8 (Table 9). (FIG. 4C) The induction of the nifH promoters from each species by the controller are shown (0.5 mM IPTG) (see Materials and Methods). (FIG. 4D) The nitrogenase activities of the native cluster (intact nifLA) is compared to the inducible clusters in the presence and absence of 0.5 mM IPTG. The dashed lines indicate the activity of the native clusters in the wild-type context (top to bottom, K. oxytoca M5al, P. stutzeri A1501 and A. vinelandii DJ). (FIG. 4E) The sensitivity of the native and inducible (+0.5 mM IPTG) nif clusters to 17.1 mM ammonium acetate are compared. Asterisks indicate ethylene production below the detection limit (<10 au). (FIG. 4F) The nitrogenase activity is shown as a function of the oxygen concentration in the headspace (see Materials and Methods). The native nif cluster is compared to the inducible version including the controller plasmid and 0.5 mM IPTG. Error bars represent s.d. from three independent experiments.

FIGS. 5A-5D include diagrams showing the control of nitrogenase activity with sensors that respond to diverse chemicals in the rhizosphere. (FIG. 5A) Schematic showing the origins of the chemicals. “Introduced DNA” refers to the genetic modification of the plant to produce nopaline and octopine. (FIG. 5B) The genetic sensors built for A. caulinodans are shown. Sequences for the genetic parts are provided in Table 10. (FIG. 5C) The response functions for the sensors are shown. Either the sensor expresses T7 RNAP, which then activates PT7, or it expresses NifA (P. protegens Pf-5) or NifA/RpoN (A. caulinodans) and activates the nifH promoter (species origin in parentheses). (FIG. 5D) The nitrogenase activity is measured in the presence or absence of inducer (see Materials and Methods). The refactored Klebsiella nif clusters v2.1 and v3.2 were used in E. coli MG1655 and R. sp. IRBG74, respectively. The inducible A. vinelandii nif cluster was used in P. protegens Pf-5. The controller containing nifA/rpoN was used in A. caulinodans ΔnifA. The inducer concentrations are: 50 μM vanillic acid. 500 μM DHBA, 50 UM cuminic acid, 25 nM 3OC6HSL, 500 nM 3OC14HSL, 33μ M arabinose, 100μ M naringenin, 100 nM DAPG, 200 μM salicylic acid, 1 mM nopaline and 1 mM octopine. Error bars represent s.d. from three independent experiments.

FIG. 6 includes a plot of the growth curve of R. sp. IRBG74 in UMS minimal medium with varying carbon sources. Cultures grown overnight in 2 mL TY medium in 15 mL culture tubes at 30° C. and 250 rpm were diluted to an OD₆₀₀ of 0.02 into 1 mL of UMS minimal medium plus varying carbon sources in 96-deepwell plates and incubated for 16 hours at 30° C. and 900 rpm. Bacterial growth was spectrophotometrically monitored at OD₆₀₀ nm. Error bars represent s.d. from three independent experiments.

FIGS. 7A-7F include diagrams showing the nitrogenase activity when different inducible nif clusters are transferred to E. coli MG1655. (FIG. 7A) The same controller system based on K. oxytoca NifA was used for all three clusters. The controller plasmid pMR-99 and genetic parts are provided in Table 9 and Table 10. (FIG. 7B) The nif clusters from K. oxytoca, P. stutzeri, and A. vinelandii are shown. The deleted regions corresponding the NifLA regulators are marked. The dotted lines indicate that multiple regions from the genome were cloned and combined for form the nif cluster. The clusters were carried the plasmids pMR-3, 5, 7 (Table 9). (FIG. 7C) The induction of the nifH promoters from each species by the controller is shown (50 μM IPTG) (see Materials and Methods) (FIG. 7D) The nitrogenase activities of the native cluster (intact nifLA) is compared to the inducible clusters in the presence and absence of 50 μM IPTG. The dashed lines indicate the activity of the native clusters in the wild-type context (top to bottom, K. oxytoca MSal, P. stutzeri A1501 and A. vinelandii DJ). (FIG. 7E) Regulation of nitrogenase activity by ammonia. Ammonium tolerance of nitrogenase from the native (black bar) and inducible (gray bar) systems was tested in the presence of 17.1 mM ammonium acetate. Asterisks indicate ethylene production below the detection limit (<10 au). (FIG. 7F) Regulation of nitrogenase activity by oxygen. The native nif cluster is compared to the inducible version including the controller plasmid and 50 μM IPTG. Nitrogenase activities were measured after 3 h of incubation at constant oxygen concentrations (0 to 3%) in the headspace (see Materials and Methods). Error bars represent s.d. from three independent experiments.

FIGS. 8A-8B include plots showing ammonium repression of the transferred nif clusters. Nitrogenase sensitivity to ammonium was measured by nitrogenase assay in the absence (−) or presence (+) of 17.1 mM ammonium acetate. The sensitivity of the native and inducible nif clusters in E. coli MG1655 (FIG. 8A) and P. protegens Pf-5 (FIG. 8B). Note that the data are from FIGS. 4A-4F and FIGS. 7A-7F. The nif clusters were induced by 50 μM and 0.5 mM IPTG in E. coli MG1655 and P. protegens Pf-5, respectively. Asterisks indicate ethylene production below the detection limit (<10 au). Error bars represent s.d. from three independent experiments.

FIG. 9 includes a diagram showing the ribosome profiling data for the K. oxytoca native nif cluster in K. oxytoca M5al, E. coli MG1655, P. protegens Pf-5 and R. sp. IRBG74 (see Materials and Methods).

FIGS. 10A-10B include diagrams showing the effect of NifA overexpression on the nifH promoter activity in R. sp. IRBG74. (FIG. 10A) The reporter construct used to measure nifH promoter activity is shown. The nifH promoter activity was analyzed in the R. sp. IRBG74 wild-type background using flow cytometry. Additional copies of NifA of R. sp. IRBG74 increased activity of the R. sp. IRBG74 nifH promoter but failed to complement or enhance activity of the other nifH promoters including K. oxytoca, P. stutzeri and A. caulinodans. Error bars represent s.d. from three independent experiments. (FIG. 10B) Plasmid maps used to assess the effect of NifA overexpression in R. sp. IRBG74. WT, wild-type; Rsp, R. sp. IRBG74; Kox, K. oxytoca M5al; Pst, P. stutzeri A1501; Aca, A. caulinodans ORS571

FIGS. 11A-11C include diagrams showing Promoter characterization in R. sp. IRBG74 and P. protegens Pf-5. (FIG. 11A) Constitutive promoters are rank-ordered by their strength. Plasmids used to measure promoter activity are depicted on the top. (FIG. 11B) The strength of the T7 promoter wild-type and its variants was analyzed in the controller strains containing the IPTG-inducible T7 RNAP on the genome of R. sp. IRBG74 and P. protegens Pf-5 with 1 mM IPTG induction. A reporter plasmid used to measure T7 promoter activity is shown on the right. (FIG. 11C) Correlation of T7 promoter strength between species. Error bars represent s.d. from three independent experiments.

FIGS. 12A-12B include diagrams showing RBS characterization in R. sp. IRBG74 and P. protegens Pf-5. RBS library for GFP was designed using the RBS library calculator at the highest-resolution mode. (FIG. 12A) The strengths of the synthetic RBSs in R. sp. IRBG74 were analyzed in the plasmid pMR-40 containing the IPTG-inducible system with 1 mM IPTG induction. 33 of the RBSs spanning a range of 5,684-fold expression were selected and their sequences are provided in Table 11. (FIG. 12B) The strengths of the synthetic RBSs in P. protegens Pf-5 was analyzed in the plasmid pMR-65 containing the arabinose-inducible system with 7 μM arabinose induction. 33 of the RBSs spanning a range of 1,075-fold expression were selected and their sequences are provided in Table 11.

FIGS. 13A-13B include diagrams showing the characterization of terminators for T7 RNAP in R. sp. IRBG74 (FIG. 13A) and P. protegens Pf-5 (FIG. 13B). (FIG. 13A) The strength of terminators was analyzed in the controller R. sp. IRBG74 strains MR16 containing the IPTG-inducible T7 RNAP on the genome with 1 mM IPTG induction. (FIG. 13B) Plasmids used to measure terminator strength are shown on right. Genetic parts are provided in Table 10. Error bars represent s.d. from three independent experiments.

FIG. 14 includes diagrams showing the response functions for the sensors in R. sp. IRBG74. Plasmids used to characterize the sensors are shown on top of each panel and provided in Table 9. Genetic parts are provided in Table 10. Error bars represent s.d. from three independent experiments. Experimental details are provided in Methods.

FIGS. 15A-15C include diagrams showing the response functions for the sensors in P. protegens Pf-5. The output changes as a function of input inducer concentrations. Plasmids used to characterize the sensors are shown on top of each panel. (FIG. 15A) Inducible promoter characterization in P. protegens Pf-5. (FIG. 15B) Optimization of the arabinose-inducible systems. Constitutive expression of a plasmid-borne AraE transporter decreased a dissociation constant of arabinose (dark gray). A mutation in the −10 region (TACTGT to TATATT) of the P_{BAD} promoter increased promoter strength (black). (FIG. 15C) Optimization of IPTG-inducible systems. The IPTG-inducible promoters were induced by 1 mM IPTG. The combination of the P_{tac} promoter and the LacI (Q18M/A47V/F161Y) protein yielded an expression range of 110-fold. Plasmids and genetic parts are provided in Table 9 and Table 10. Error bars represent s.d. from three independent experiments.

FIG. 16 includes diagrams showing the tuning controller strength in R. sp. IRBG74. The controller containing the IPTG-inducible T7 RNAP is integrated into the genome of R. sp. IRBG74 (top). Controller strengths were adjusted by modulating the RBS of T7 RNAP in the plasmids pMR-81, 82, and 83. Response functions of the T7 promoter were measured with the reporter plasmid pMR-79 (right) in the R. sp. IRBG74 controller strains MR16, MR17, and MR18. Genetic parts and RBS sequences are provided in Table 10 and Table 5. Error bars represent s.d. from three independent experiments.

FIG. 17 includes a plot showing the nitrogenase activity of the refactored nif clusters across species. Error bars represent s.d. from three independent experiments.

FIG. 18 includes diagrams showing RNA-seq (top) and Ribosome profiling (bottom) data, respectively in E. coli MG1655 and R. sp. IRBG74. The nif genes were induced by 1 mM IPTG for 6 hours (see Materials and Methods).

FIG. 19 includes diagrams showing RNA-seq (top) and ribosome profiling (bottom) data, respectively, in E. coli MG1655 and P. protegens Pf-5 and R. sp. IRBG74. The nif genes were induced by 1 mM, 0.1 mM, and 0.5 mM IPTG for 6 h in E. coli MG1655, P. protegens Pf-5 and R. sp. IRBG74, respectively (see Materials and Methods).

FIGS. 20A-20F include diagrams showing the transfer of the refactored nif cluster v3.2 in P. protegens Pf-5. (FIG. 20A) Controllers whose output is T7 RNAP from the genome of P. protegens Pf-5 are described. Substituted genetic parts including a new RBS and IPTG-inducible promoter for the controller optimization compared to the controller module pKT249 in E. coli MG1655 highlighted in red. The response functions for the controllers with the reporter plasmid pMR-80 was measured in the P. protegens Pf-5 controller strain MR7. Controllers driving the expression of GFP by the T7 promoter achieved large dynamic to 96-fold activation by IPTG. Error bars represent s.d. from three independent experiments. (FIG. 20B) The genetic parts used to build the refactored v3.2 nif gene cluster are shown (Table 10). (FIG. 20C) The activity of the refactored nif cluster v3.2. Nitrogenase expression was induced by 1 mM IPTG. (FIG. 20D) Function of the transcriptional parts of the cluster v3.2 was analyzed by RNA-seq (FIG. 19). The performance of the promoters (left) and terminators (right) was calculated (see Materials and Methods). (FIG. 20E) The translation efficiency of the nif genes v3.2 as calculated using ribosome profiling and RNA-seq. Lines connect points that occur in the same operon. (FIG. 20F) The ribosome density (RD) is compared for the refactored v3.2 nif genes in P. protegens Pf-5 versus that measured for the nif genes from the native K. oxytoca cluster in K. oxytoca (→Klebsiella).

FIG. 21 includes diagrams showing the response function of inducible promoters in A. caulinodans ORS571. Plasmids used to characterize inducible promoters are shown on top of each panel and provided in Table 9. Genetic parts are provided in Table 10. Error bars represent s.d. from three independent experiments.

FIG. 22 includes a diagram showing the multiple sequence alignment of NifA of A. caulinodans ORS571 with R. spheroides 2.4.1 was generated using MUSCLE2. The corresponding residues for ammonium tolerance in R. sphaeroides are outlined in red. The A. caulinodans strand corresponds to SEQ ID NO: 293, and the R. sphaeroides strand corresponds to SEQ ID NO: 292.

FIGS. 23A-23B include diagrams showing functional testing of the NifA homologues that activate the nifH promoters. (FIG. 23A) The ability of the various NifA to activate the nifH promoters was tested with pairwise combinations of the nifH promoters and the NifA in E. coli MG1655 and P. protegens Pf-5. Error bars represent s.d. from three independent experiments. (FIG. 23B) Plasmids used to measure nifH promoter activity by NifA overexpression are shown and provided in Table 9. Genetic parts are provided in Table 10. Pst, P. stutzeri A1501; Avi, A. vinelandii DJ; Kox, K. oxytoca M5al

FIGS. 24A-24B include diagrams showing optimization of the controllers in P. protegens Pf-5 and E. coli MG1655 that induce the nifH promoters. (FIG. 24A) The controllers with different strengths were designed by RBS replacement and tested with the reporter plasmids (pMR 103-105) in which each of the three nifH promoter is fused to sfgfp (Methods). The nifH promoters were induced with 0.5 mM IPTG. Genetic parts and RBS sequences are provided in Table 10 and 11, respectively. (FIG. 24B) Activation of the nifH promoters in the E. coli MG1655 containing the controller plasmid pMR102 was tested with the reporter plasmids pMR106-108. The P. protegens Pf-5 controller strain MR10 was used to drive expression of the nifH promoter of K. oxytoca and the controller strain MR9 was used to drive expression of the nifH promoters of P. stutzeri and A. vinelandii. The nifH promoters were induced with 0.05 mM IPTG and 0.5 mM IPTG in E. coli MG1655 and P. protegens Pf-5, respectively. Error bars represent s.d. from three independent experiments.

FIG. 25 includes diagrams showing the effect of oxygen on the activity of the nifH promoters. Expression from the nifH promoters was analyzed in E. coli MG1655 containing the controller plasmid pMR 102, P. protegens Pf-5 MR 10 (for K. oxytoca) and MR9 (for P. stutzeri and A. vinelandii) at varying initial oxygen levels in the headspace. The three nifH promoters were induced with 0.05 mM IPTG and 0.5 mM IPTG in E. coli MG1655 and P. protegens Pf-5, respectively, and incubated at varying initial oxygen concentrations. Oxygen has no effects on nifH expression in both strains. Error bars represent s.d. from three independent experiments.

FIGS. 26A-26B include diagrams describing the nitrogenase activity assay. (FIG. 26A) Nitrogenase activity assay at constant oxygen levels in the headspace. Experimental setup used in this study to analyze oxygen tolerance of nitrogenase. Following the expression induction of nitrogenase with preincubation under low oxygen conditions, targeted oxygen concentrations in the headspace is maintained by oxygen spiking while monitoring with oxygen monitoring system (Methods). (FIG. 26B) Nitrogenase activity in E. Coli MG1655 and P. protegens Pf-5 over a course of three hours.

FIG. 27 includes diagrams showing the effect of the rnf and fix complex on nitrogenase activity. The modified nif clusters of A. vinelandii on the plasmids pMR25-28 were analyzed in the controller strain P. protegens Pf-5 MR9. The deleted regions from the clusters were provided in Table 9. Nitrogenase was induced with 0.5 mM IPTG. Removing the mf complex from the cluster abrogated activity. The cluster without the fixABCX complex showed identical oxygen tolerance to the cluster with the complex. Error bars represent s.d. from three independent experiments.

FIGS. 28A-28C include diagrams showing regulation of nitrogenase activity in E. coli MG1655 “Marionette” strain5. (FIG. 28A) Controller plasmids used to drive expression of T7 promoters. (FIG. 28B) Inducibility of the T7 promoter by the controller plasmids encoding T7 RNAP under the regulation of the 12 sensors was tested with a reporter plasmid pMR121 (right). (FIG. 28C) Inducible control of nitrogenase activity in response to 12 inducers was with the plasmid pMR 136 (right) carrying the refactored nif cluster v2.1 on pBBR1 origin. The choline-Cl inducible system was omitted for activity assay as the system was not inducible. For the DAPG-, DHBA-, and vanillic acid-inducible system, the refactored cluster was carried on a lower copy number plasmid pMR31 (right) as transformation of the plasmid pMR29 gave rise to no colony formation. The inducers concentrations are: 400 μM arabinose, 1 mM choline-C1, 500 nM 3OC14HSL, 50 UM cuminic acid, 25 nM 3OC6HSL, 25μ M DAPG, 500μ M DHBA, 1 mM IPTG, 100 nM aTc, 250μ M naringenin, 50μ M vanillic acid, and 250 M salicylic acid. Plasmid and genetic parts are provided in Table 9 and 10. Error bars represent s.d. from three independent experiments.

FIG. 29 includes schematic plasmid maps used to assess the effect of inducible expression of NifA/RpoN on the activity of the nifH promoter in A. caulinodans ORS571.

### DETAILED DESCRIPTION OF THE INVENTION

Nitrogen fixation in the root nodules of leguminous plants is a major contributor to world food production and therefore, the practical applications of this field are of major interest. Legumes obtain nitrogen from air through bacteria residing in root nodules, some species of which also associate with cereals but do not fix nitrogen under these conditions. Disabling native regulation can turn on expression, even in the presence of nitrogenous fertilizer and low O₂, but continuous nitrogenase production confers an energetic burden.

The present disclosure in some aspects describes the surprising discovery that bacteria can be genetically altered in a manner that will enable the bacteria to deliver fixed nitrogen to cereal crops. Several strategies to implement control over nitrogen fixation in bacteria that live on or inside the roots of cereals are described. At least two approaches can be taken. In one embodiment, the native regulation is replaced. In alternative embodiments, a nif cluster is transferred from another species and placed under inducible control. The Examples section below includes a description of the achievement of these two approaches in multiple species with multiple constructs. For example, A. caulinodans, ammonium-independent control was achieved using a sensor to drive the co-expression of a NifA mutant and RpoN in a ΔnifA strain. Rhizobium sp. IRBG74 can be engineered to express functional nitrogenase under free living conditions either by transferring a native nif cluster from Rhodobacter or a refactored cluster from Klebsiella. Multiple approaches enable P. protegens Pf-5 to express functional nitrogenase, of which the transfer of the nif cluster from Azotobacter vinelandii DJ yields the highest activity and O₂ tolerance.

To date, it has not been shown that a Rhizobium strain can be engineered to fix nitrogen under free-living conditions when it does not do so naturally. Some Rhizobia isolated from legume root nodules are also cereal endophytes, however most are unable tofix nitrogen under free-living conditions (outside of the nodule) (Ramachandran, V. K., East, A. K., Karunakaran, R., Downie, J. A. & Poole, P. S. Adaptation of Rhizobium leguminosarum to pea, alfalfa and sugar beet rhizospheres investigated by comparative transcriptomics. Genome biology 12, R106 (2011); Frans, J. et al. in Nitrogen Fixation 33-44 (Springer, 1990)). There have been reports of cereal yield improvements due to these bacteria, including a 20% increase for rice by Rhizobium sp. IRBG74, but this is likely due to other growth-promoting mechanisms, such as improved nutrient uptake or root formation (Ramachandran, V. K., East, A. K., Karunakaran, R., Downie, J. A. & Poole, P. S. Adaptation of Rhizobium leguminosarum to pea, alfalfa and sugar beet rhizospheres investigated by comparative transcriptomics. Genome biology 12, R106 (2011); Delmotte, N. et al. An integrated proteomics and transcriptomics reference data set provides new insights into the Bradyrhizobium japonicum bacteroid metabolism in soybean root nodules. Proteomics 10, 1391-1400 (2010); Hoover, T. R., Imperial, J., Ludden, P. W. & Shah, V. K. Homocitrate is a component of the iron-molybdenum cofactor of nitrogenase. Biochemistry 28, 2768-2771 (1989)). Azorhizobium caulinodans ORS571 is exceptional because it is able to fix nitrogen in both aerobic free-living and symbiotic states, has been shown to be a rice and wheat endophyte, and does not rely on plant metabolites to produce functional nitrogenase. However, when Rhizobia or Azorhizobium are living in cereal roots, there is low nitrogenase expression and 15N2 transfer rates suggest any reported uptake is due to bacterial death.

### Cereal Crops, Nitrogen Fixation, and Bacteria

Cereal crops are broadly defined as any grass cultivated for the edible components of its grain (also referred to as caryopsis), composed of the endosperm, germ, and bran. Cereal crops are considered staple crops in many parts of the world. They are grown in greater quantities and provide more food energy worldwide than any other type of crop. Non-limiting examples of cereal crops include maize, rye, barley, wheat, sorghum, oats, millet and rice. As used herein, the terms “cereal crop” and “cereal plant” are used interchangeably.

Nitrogen fixation is the process by which atmospheric nitrogen is assimilated into organic compounds as part of the nitrogen cycle. The fixation of atmospheric nitrogen associated with specific legumes is the result of a highly specific symbiotic relationship with rhizobial bacteria. These indigenous bacteria dwell in the soil and are responsible for the formation of nodules in the roots of leguminous plants as sites for the nitrogen fixation. Most Rhizobium symbioses are confined to leguminous plants. Furthermore, Rhizobium strains which fix nitrogen in association with the agriculturally-important temperate legumes are usually restricted in their host range to a single legume genus.

The nif genes are genes encoding enzymes involved in the fixation of atmospheric nitrogen into a form of nitrogen available to living organisms. The primary enzyme encoded by the nif genes is the nitrogenase complex which converts atmospheric nitrogen (N₂) to other nitrogen forms (e.g. ammonia) which the organism can process. As used herein, the term “nif cluster” refers to a gene cluster comprising nif genes. As used herein, the term “refactored” refers to an engineered gene clusture, i.e. its genes have reordered, deleted or altered in some way.

Rhizobia are diazotrophic bacteria. In general, they are gram negative, motile, non-sporulating rods. In terms of taxonomy, they fall into two classes: alphaproteobacteria and betaproteobacteria. Non-limiting examples of rhizobia include Azorhizobium caulinodans, Rhizobium (R.) sp. IRBG74, R. radiobacter, R. rhizogenes, R. rubi, R. vitis, Alfalfa Rhizobia (R. meliloti), Chickpea Rhizobia (Rhizobium sp.), Soybean Rhizobia (Bradyrhizobium japonicum), Leucaena Rhizobia (Rhizobium sp.), R. leguminosarum by trifolii, R. leguminosarum by phaseoli, and Rhizobium leguminosarum by viciae (see for example U.S. Pat. No. 7,888,552, herein incorporated by reference). In some embodiments, the rhizobia of the present invention are Azorhizobium caulinodans. In some embodiments, the rhizobia of the present invention are not Azorhizobium caulinodans.

As used herein, the term “free-living conditions” refers to a bacterium (e.g. rhizobium) that is not within a leguminous root nodule. It generally refers to something that has not formed a parasitic (or dependent) relationship with another organism or is not on a substrate. As used herein, the term “symbiotic” refers to the interaction between two organisms living in close proximity. Close proximity can be about 0.2 μm, 0.4 μm, 0.6 μm, 0.8 μm, 1 μm, 5 μm, 10 μm, 20 μm, 50 μm, 100 μm, 500 μm, 1 mm, 1 cm, 5 cm, 10 cm. Close proximity can also be less than 0.2 μm. In many cases, a symbiotic relationship refers to a mutually beneficial interaction.

As used herein, “aerobic free-living conditions” refer to conditions under which a bacterium is not within a leguminous root nodule and the bacterium is in the presence of oxygen. Aerobic free-living conditions can also be referred to as nonsymbiotic or non-parasitic conditions in the presence of oxygen. The bacterium can be in close proximity to a crop, as defined above.

As used herein, the term “endophyte” refers to a group of organisms, often fungi and bacteria, that live within living plant cells for at least part of its life cycle without having an apparent detrimental effect on the plant cell. This is contrasting with an epiphyte, which is a plant that grows on another plant, without being parasitic.

As used herein, the term “diazotroph” refers to microorganisms that are able to grow without external sources of fixed nitrogen. The group includes some bacteria and some archae. There are free-living and symbiotic diazotrophs. An example of a free-living diazotroph is Klebsiella pneumoniae. K. pneumoniae is a facultative anaerobes—these species can grow either with or without oxygen, but they only fix nitrogen anaerobically.

As used herein, the term “Alphaproteobacteria” refers to a diverse class of bacteria falling under the phylum Proteobacteria. Non-limiting examples of Alphaproteobacteria include species Rhodobacter sphaeroides and Rhodopseudomonas palustris. As used herein, the term “Gammaproteobacteria” refers to another class of bacteria falling under the phylum of Proteobacteria. All proteobacteria are gram negative. As used herein, the term “Cyanobacteria” refers to a phylum of bacteria that obtain their energy through photosynthesis. They are also referred to as Cyanophyta. They have characteristic internal membranes and thylakoids, the latter being for photosynthetic purposes. As used herein, the term “Firmicutes” refer to a phylum of bacteria. This phylum includes the classes Bacilli. Clostridia, and Thermolithobacteria.

### Nif Genes

Typically, the genes necessary for nitrogen fixation occur together in a gene cluster, including the nitrogenase subunits, the biosynthesis of metalloclusters cluster and, e-transport, and regulator proteins. Nif genes are genes that encode the enzyme involved in nitrogen fixation. In most cases nif genes occur as an operon. Some of these genes encode the subunits for the nitrogenase complex, which is the primary enzyme imparting the ability to convert atmospheric nitrogen (N₂) to forms of nitrogen accessible to living organisms. In most genes, the regulation of the nif gene transcription is conducted by NifA protein, which is responsive to nitrogen levels. When there are nitrogen deficits, NtrC activates NifA expression, which in turn leads to the activation of the remaining nif genes. When nitrogen levels are adequate or in excess, NifL protein, encoded by NifL. NifL inhibits NifA activity.

Nif gene pathways are generally sensitive to small changes in expression. The genes that form nitrogenase. Important genes include nifHDK, which form the subunits for nitrogenase. The chaperone NifY is required to achieve full activity and broadens the tolerance to changes in expression level. NifJ and nif regulate electron transport. The nifUSVWZM operon encodes proteins for early Fe—S cluster formation (NifUS) and proteins for component maturation (NifVWZ for Component I and NifM for Component II), whereas nifBQ encodes proteins for FeMo-co core synthesis (NifB) and molybdenum integration (NifQ). NifEN is tolerant to varied expression levels.

Exemplary sequences for various nif genes are provided in Table 10. Non-limiting examples of nif genes include nifH, nifD, nifK, nifE, nifN, nifU, nifS, nifV, nifW, nifX, nifB, nifQ, nifY, nifT, nifJ, nifF, nifX, nifU, and nifS

Nitrogen fixation and Regulatory Elements

The nitrogen fixation (nif) genes are organized as genomic clusters, ranging from a 10.5 kb single operon in Paenibacillus to 64 kb divided amongst three genomic locations in A. caulinodans. Conserved genes include those encoding the nitrogenase enzyme (nifHDK), FeMoCo biosynthesis, and chaperones. Species that can fix nitrogen under more conditions tend to have larger gene clusters that include environment-specific paralogues, alternative electron transport routes, and oxygen protective mechanisms. Often, the functions of many genes in the larger clusters are unknown.

There is evolutionary evidence for the lateral transfer of nif clusters between species (Pascuan, C., Fox, A. R., Soto, G. & Ayub. N. D. Exploring the ancestral mechanisms of regulation of horizontally acquired nitrogenases. Journal of molecular evolution 81, 84-89 (2015); Kechris, K. J., Lin, J. C., Bickel, P. J. & Glazer, A. N. Quantitative exploration of the occurrence of lateral gene transfer by using nitrogen fixation genes as a case study. Proceedings of the National Academy of Sciences 103, 9584-9589 (2006)). However, achieving such a transfer via genetic engineering poses a challenge as many things can go awry, including differences in regulation, missing genes, and the intracellular environment (Frans, J. et al. in Nitrogen Fixation 33-44 (Springer, 1990); Poudel, S. et al. Electron transfer to nitrogenase in different genomic and metabolic backgrounds. Journal of bacteriology 200, e00757-00717 (2018); Thony, B., Anthamatten, D. & Hennecke, H. Dual control of the Bradyrhizobium japonicum symbiotic nitrogen fixation regulatory operon fixR nifA: analysis of cis- and trans-acting elements. Journal of bacteriology 171, 4162-4169 (1989); Han, Y. et al. Interspecies Transfer and Regulation of Pseudomonas stutzeri A1501 Nitrogen Fixation Island in Escherichia coli. Journal of microbiology and biotechnology 25, 1339-1348 (2015)). Nitrogenase is under stringent control because it is oxygen sensitive and energetically expensive: it can make up 20% of the cell mass and each NH3 requires ˜40 ATP. It is also irreversibly deactivated by oxygen. Across species, transcription of nif genes is strongly repressed by fixed nitrogen (ammonia) and oxygen with these signals converging on the NifA regulatory protein that works in concert with the sigma factor RpoN. Diverse, species-specific, and often poorly understood signals control these regulators, including plant-produced chemicals, ATP, reducing power, temperature, and carbon sources. Those bacteria that can fix nitrogen in a wider range of environmental conditions tend to be controlled by more complex regulatory networks.

When a nif cluster is transferred from one species to another, it either preserves its regulation by environmental stimuli or has an unregulated constitutive phenotype. Maintaining the native regulation, notably ammonium repression, limits their use in agriculture because such levels are likely to fluctuate according to soil types, irrigation, and fertilization. Nitrogen-fixing diazotrophs have been engineered to reduce ammonia sensitivity by disrupting NifL or mutating NifA and placing the entire cluster under the control of T7 RNA polymerase (RNAP). Constitutive expression of nitrogenase is also undesirable as it imparts a fitness burden on the cells. For example, when the nif cluster from P. stutzeri A1501 was transferred to P. protegens Pf-5, this was reported to result in sufficient ammonia production to support maize and wheat growth, but the bacteria quickly declined after a month when competing with other species in soil. Constitutive activity is detrimental even before the bacteria are introduced to the soil, impacting production, formulation, and long-term storage. Therefore, uncontrolled nitrogenase production could lead to more expensive production, shorter shelf life, and more in-field variability.

An important aspect of the nif clusters or nif genes the present disclosure is that they can each be under the control of a regulatory element. In some embodiments 2 or more genes are under the control of a regulatory element. In some embodiments, all the genes are under the control of a regulatory element. The regulatory elements may also be activation elements or inhibitory elements. An activation element is a nucleic acid sequence that when presented in context with a nucleic acid to be expressed will cause expression of the nucleic acid in the presence of an activation signal. An inhibitory signal is a nucleic acid sequence that when presented in context with a nucleic acid to be expressed will cause expression of the nucleic acid unless an inhibitory signal is present. Each of the activation and inhibitory elements may be a promoter, such as a bacteriophage T7 promoter, sigma 70 promoter, sigma 54 promoter, lac promoter, etc. As used herein, the term “promoter” is intended to refer to those regulatory sequences which are sufficient to enable the transcription of an operably linked DNA molecule. Promoters may be constitutive or inducible. As used herein, the term “constitutive promoter” refers to a promoter that is always on (i.e. causing transcription at a constant level). Examples of constitutive promoters include, without limitation, sigma 70 promoter, bla promoter, lacL promoter, etc. Non-limiting examples of inducible promoters are shown in Table 6. The P_{A1lacO1} promoter is another example of an inducible promoter that can be used in the present invention.

TABLE 6
Examples of regulatory elements (e.g.
inducible promoters, repressors).

| | | Essential |
|---|---|---|
| | | regulatory |
| Name | Chemical inducer and/or repressor | gene(s) |
| ParaBAD (“PBAD”) | L-arabinose (ON) & glucose (OFF) | araC |
| PrhaBAD | L-rhamnose (ON) & glucose (OFF) | rhaR & rhaS |
| Plac | lactose or IPTG (ON) & glucose | lacI |
| | (OFF) | |
| Ptac | lactose or IPTG (ON) | lacI |
| Plux | acyl-homoserine lactone (ON) | luxR |
| Ptet | tetracycline or aTc (ON) | tetR |
| Psal | salycilate (ON) | nahR |
| Ptrp | tryptophan (OFF) | (NONE) |
| Ppho | phosphate (OFF) | phoB & phoR |

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Still other types of inducible promoters which may be useful in this context are those which are regulated by a specific physiological state, e.g., temperature, acute phase, a particular differentiation state of the cell, or in replicating cells only.

As used herein, the term “terminator” (as referred to as a transcription terminator) is a section of nucleic acid sequence that marks the end of a gene or operon in genomic DNA during transcription. They stop transcription of a polymerase. Terminators can be classified into several groups. At the first group of termination signals the core enzyme can terminate in vitro at certain sites in the absence of any other factors (as tested in vitro). These sites of termination are called intrinsic terminators or also class I terminators. Intrinsic terminators usually share one common structural feature, the so called hairpin or stem-loop structure. On the one hand the hairpin comprises a stem structure, encoded by a dG-dC rich sequence of dyad symmetrical structure. On the other hand the terminator also exhibits a dA-dT rich region at the 3′-end directly following the stem structure. The uridine rich region at the 3′ end is thought to facilitate transcript release when RNA polymerase pauses at hairpin structures. Two or more terminators can be operatively linked if they are positioned to each other to provide concerted termination of a preceding coding sequence. Particularly preferred, the terminator sequences are downstream of coding sequences, i.e. on the 3′ position of the coding sequence. The terminator can e.g. be at least 1, at least 10, at least 30, at least 50, at least 100, at least 150, at least 200, at least 250, at least 300, at least 400, at least 500 nucleotides downstream of the coding sequence or directly adjacent. Examples of terminators include, but are not limited to, T7 terminator, rrnBT1, L3S2P21, tonB, rrnA, rrnB, rrnD, RNAI, crp, his, ilv lambda, M13, rpoC, and up (see for example U.S. Pat. No. 9,745,588, incorporated herein by reference).

### RpoN

As used herein “RpoN” refers to a gene that encodes the sigma factor sigma-54 (σ54, sigma N, or RpoN), a protein in Escherichia coli and other species of bacteria. Sigma factors are initiation factors that promote attachment of RNA polymerase to specific initiation sites and are then released. Bacteria normally only have one functional copy of the alternative sigma factor, σ54 or RpoN, which regulates a complex genetic network that extends into various facets of bacterial physiology, including metabolism, survival in strenuous environments, production of virulence factors, and formation of biofilms. RpoN is one of seven RNA polymerase sigma subunits in E. coli required for promoter-initiated transcription and RpoN plays a major role in the response of E. coli to nitrogen-limiting conditions. Under such conditions, RpoN directs the transcription of at least 14 E. coli operons/regulators in the nitrogen regulatory (Ntr) response. RpoN also plays an important role in stress resistance (e.g. resistance to osmotic stress) and virulence of bacteria. RpoN is structurally and functionally distinct from the other E. coli σ factors. It is able to bind promoter DNA in the absence of core RNA polymerase and it recognizes promoter sequences with conserved GG and GC elements located −24 to −12 nucleotides upstream of the transcription start site. Additionally, Regulatory proteins like NtrB and NtrC can activate σ54 holoenzyme.

Without being bound by theory or mechanism, it is believed that RpoN works in concert with NifA to turn on the transcription of nif clusters. An exemplary sequence for RpoN is provided in Table 10.

### Gene Cluster Nucleic Acids

In some embodiments of the present disclosure a genetic cluster includes a nucleotide sequence that is at least about 85% or more homologous or identical to the entire length of a naturally occurring genetic cluster sequence, e.g., at least 5%, 10%, 15%, 20%, 25%, 30%, 35%, 40%, 50% or more of the full length naturally occurring genetic cluster sequence). In some embodiments, the nucleotide sequence is at least about 90%, 91%, 92%, 93%, 94%, 95%, 96%, 97%, 98%, 99% or 100% homologous or identical to a naturally occurring genetic cluster sequence. In some embodiments, the nucleotide sequence is at least about 85%, e.g., is at least about 90%, 91%, 92%, 93%, 94%, 95%, 96%, 97%, 98%, 99% or 100% homologous or identical to a genetic cluster sequence, in a fragment thereof or a region that is much more conserved, such as an essential, but has lower sequence identity outside that region.

Calculations of homology or sequence identity between sequences (the terms are used interchangeably herein) are performed as follows. To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

In some embodiments the gene clusters are native gene clusters. In some embodiments, the gene clusters are refactored gene clusters. In some instances, the nucleic acids may include non-naturally occurring nucleotides and/or substitutions, i.e. Sugar or base substitutions or modifications.

One or more substituted sugar moieties include, e.g., one of the following at the 2′ position: OH, SH, SCH3, F, OCN, OCH3OCH3, OCH3O(CH2)n CH3, O(CH2)n NH2 or O(CH2) n CH3 where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of a nucleic acid; or a group for improving the pharmacodynamic properties of a nucleic acid and other substituents having similar properties. Similar modifications may also be made at other positions on the nucleic acid, particularly the 3′ position of the sugar on the 3′ terminal nucleotide and the 5′ position of 5′ terminal nucleotide. Nucleic acids may also have sugar mimetics such as cyclobutyls in place of the pentofuranosyl group.

Nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl) adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-′ 7′ 7; and Gebeyehu, G., et al. Nucl. Acids Res., 15:4513 (1987)). A “universal” base known in the art, e.g., inosine, can also be included.

As used herein, the equivalent terms “expression” or “gene expression” are intended to refer to the transcription of a DNA molecule into RNA, and the translation of such RNA into a polypeptide.

As used herein, a “gene cluster” refers to a set of two or more genes that encode gene products. As used herein, a “nif gene cluster” refers to a set of two or more genes that encode nitrogen fixation genes.

“Exogenous” with respect to genes indicates that the nucleic acid or gene is not in its natural (native) environment. For example, an exogenous gene can refer to a gene that is from a different species. In contrast, “endogenous” with respect to genes indicates that the gene is in its native environment. As used herein, the terms “endogenous” and “native” are used interchangeably.

As used herein, the term “delete” or “deleted” refers to the removal of a gene (e.g. endogenous gene) from a sequence or cluster. As used herein, the term “alter” or “altered” refers to the modification of one or more nucleotides in a gene or the deletion of one or more base pairs in a gene. This alteration may render the gene dysfunctional. Herein, “ΔnifA” refers to a strain or cluster within which NifA was deleted or altered. Method of deletion and alteration, in the context of genes, are known in the art.

As used herein, the term “chemical signals” refers to chemical compounds. Any substance consisting of two or more different types of atoms (chemical elements) in a fixed stoichiometric proportion can be termed a chemical compound. Chemical signals can be synthetic or natural chemical compounds. In some embodiments of the present invention, a bacterium of the present disclosure or a sensor of the present disclosure is under the control of a chemical signal. In some embodiments, the signal is a native biological signal (e.g. root exudate, biological control agent, etc.). In some embodiments, the chemical signal is a quorum sensing signal from the bacterium. Non-limiting examples of chemical signals include root exudates (as defined below), biocontrol agents (as defined below), phytohormones, vanillate, IPTG, aTc, cuminic acid, DAPG, and salicylic acid, 3,4-dihydroxybenzoic acid, 3OC6HSL and 3OC14HSL.

As used herein, the term “root exudate” refers to chemicals secreted or emitted by plant roots in response to their environment. These allow plant to manipulate or alter their immediate environment, specifically their rhizosphere. Root exudates are a complex mixture of soluble organic substances, which may contain sugars, amino acids, organic acids, enzymes, and other substances. Root exudates include, but are not limited to, ions, carbon-based compounds, amino acids, sterols, sugars, hormones (phytohormones), flavonoids, antimicrobials, and many other chemical compounds. The exudates can serve as either positive regulators or negative regulators.

As used herein, the term “phytohormone” refers plant hormones and they are any of various hormones produced by plants that influence process such as germination, growth, and metabolism in the plant.

As used herein, the term “vanillate” refers to a methoxybenzoate that is the conjugate base of vanillic acid. It is a plant metabolite.

Biological control or biocontrol is a method of controlling pests such as insects, mites, weeds and plant diseases using other organisms. Natural enemies of insect pests, also known as biological control agents, include predators, parasitoids, pathogens, and competitors. Biological control agents of plant diseases are most often referred to as antagonists. Biological control agents of weeds include seed predators, herbivores and plant pathogens. The inducible clusters or promoters of the present invention may be modulated by a secretion of (or chemical otherwise associated with) a biological control agent. Herein, that is referred to as a “biocontrol agent”.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

Herein, inducible nitrogenase activity is engineered in two cereal endophytes (Azorhizobium caulinodans ORS571 and Rhizobium sp. IRBG74) and the epiphyte Pseudomonas protegens Pf-5, a maize seed inoculant. For each organism, different strategies are taken to eliminate ammonium repression and place nitrogenase expression under the control of agriculturally-relevant signals, including root exudates, biocontrol agents, and phytohormones. The present disclosure demonstrates that Rhizobium sp. (e.g., IRBG74) can be engineered to fix nitrogen under free living conditions, inter alia, by transferring either a nif cluster from Rhodobacter or Klebsiella. For P. protegens Pf-5, the transfer of an inducible cluster from Azotobacter vinelandii yields the highest ammonia and oxygen tolerance. Collectively, data from the transfer of 12 nif gene clusters between diverse species (including E. coli and 12 additional Rhizobia) help identify the barriers that must be overcome to engineer a bacterium to deliver a high nitrogen flux to a cereal crop and provide a solution such that Rhizobium can be engineered to fix nitrogen under free living conditions.

### Materials and Methods

Bacterial strains and growth media. All bacterial strains and their derivatives used in this study are listed in Table 7. E. coli DH10-beta (New England Biolabs, MA, Cat #C3019) was used for cloning. E. coli K-12 MG1655 was used for the nitrogenase assay. P. protegens Pf-5 was obtained from the ATCC (BAA-477). Strains used in this study are listed in Table 8. For rich media, LB medium (10 g/L tryptone, 5 g/L yeast extract, 10 g/L NaCl), LB-Lennox medium (10 g/L tryptone, 5 g/L yeast extract, 5 g/L NaCl), and TY medium (5 g/L tryptone, 3 g/L yeast extract, 0.87 g/L CaCl2·2H2O) were used. For minimal media, BB medium (0.25 g/L MgSO4·7H₂O, 1 g/L NaCl, 0.1 g/L. CaCl₂·2H₂O, 2.9 mg/L FeCl₃, 0.25 mg/L Na₂MoO₄·2H₂O, 1.32 g/L NH₄CH₃CO₂, 25 g/L Na₂HPO₄, 3 g/L KH₂PO₄ pH [7.4]), UMS medium (0.5 g/L MgSO₄·7H₂O, 0.2 g/L NaCl, 0.375 mg/L EDTA-Na₂, 0.16 ZnSO₄·7H₂O, 0.2 mg/L Na: MoO₄·2H₂O, 0.25 mg/L H₃BO₃, 0.2 mg/L MnSO₄·H₂O, 0.02 mg/L CuSO₄·5H₂O, 1 mg/L CoCl₂·6H₂O, 75 mg/L CaCl₂·2H₂O, 12 mg/L FeSO₄·7H₂O, 1 mg/L thiamine hydrochloride 2 mg/L D-pantothenic acid hemicalcium salt, 0.1 mg/L biotin, 87.4 mg/L K₂HPO, 4.19 g/L MOPS pH [7.0]), and Burk medium (0.2 g/L MgSO₄·7H₂O, 73 mg/L CaCl₂·H₂O, 5.4 mg/L FeCl₃·6H₂O, 4.2 mg/L Na₂MoO₄·2H₂O, 0.2 g/L KH₂PO₄, 0.8 g/L K₂HPO₄ pH [7.4]) were used. Antibiotics were used at the following concentrations (μg/mL): E. coli (kanamycin, 50; spectinomycin, 100; tetracycline, 15; gentamicin, 15). P. protegens Pf-5 (kanamycin, 30; tetracycline, 50; gentamicin, 15; carbenicillin, 50). R. sp. IRBG74 (neomycin, 150; gentamicin, 150; tetracycline, 10; nitrofurantoin, 10). A. caulinodans (kanamycin, 30; gentamicin, 15; tetracycline, 10; nitrofurantoin, 10). Chemicals including inducers used in this study are listed in Table 12.

Strain construction. In order to increase transformation efficiency in R. sp. IRBG74, a type-I restriction modification system was inactivated by deleting hsdR, which encodes a restriction enzyme for foreign DNA (this strain was the basis for all experiments) (Ferri, L., Gori, A., Biondi, E. G., Mengoni, A. & Bazzicalupo, M. J. P. Plasmid electroporation of Sinorhizobium strains: The role of the restriction gene hsdR in type strain Rm1021. 63, 128-135 (2010)). A sacB markerless insertion method was utilized to allow replacements of a native locus with synthetic parts by homologous recombination. Two homology arms of −500 bp flanking the hsdR gene were amplified by PCR, cloned and yielded a suicide plasmid pMR-44. The suicide plasmid was mobilized into R. sp. IRBG74 by triparental mating. Single-crossover recombinants were selected for resistance to gentamicin and subsequently grown and plated on LB plates supplemented with 15% sucrose to induce deletion of the vector DNA part containing the counter selective marker sacB which converts sucrose into a toxic product (levan). Two native nif gene clusters encompassing nifHDKENX (genomic location 219.579-227,127) and nifSW-fixABCX-nifAB-fdxN-nifTZ (genomic location 234,635-234,802) of R. sp. IRBG74 were sequentially deleted using pMR45-46. To increase genetic stability recA gene was deleted using the plasmid pMR47. The R. sp. IRBG74 Δnif, hsdR, recA strain was the basis for all experiments unless indicated otherwise. Two homology arms of ˜900 bp flanking the nifA gene were amplified by PCR, cloned and yielded a suicide plasmid pMR-47 to generate nifA deletion in A. caulinodans ORS571, The suicide plasmid pMR47 in E. coli was mobilized into A. caulinodans by triparental mating. Single-crossover recombinants were selected for resistance to gentamicin and subsequently grown and plated on plain TY plates supplemented with 15% sucrose to induce deletion of the vector DNA part. All markerless deletions were confirmed by gentamicin sensitivity and diagnostic PCR. A list of the mutant strains is provided in Table 8.

Plasmid system. Plasmids with the pBBR1 origin were derived from pMQ131 and pMQ132. Plasmids with the pRO1600 origin were derived from pMQ80. Plasmids with the RK2 origin were derived from pJP2. Plasmids with the RSF1010 origin were derived from pSEVA651. Plasmids with the IncW origin were derived from pKT249. Plasmids used in this study are provided in Table 9.

Phylogenetic analysis of nif clusters. Phylogenetic analysis was performed based on the full-length 16S rRNA gene sequences (K. oxytoca, BWI76_05380; A. vinelandii, Avin_55000; R. sphaeroides, DQL45_00005; Cyanothece ATCC51142, cce_RNA045; A. brasilense, AMK58_25190; R. palustris, RNA_55; P. protegens, PST_0759; Paenibacillus sp. WLY78, JQ003557). A multiple sequence alignment was generated using MUSCLE (Edgar, R. C. J. N. a. r. MUSCLE: multiple sequence alignment with high accuracy and high throughput. 32, 1792-1797 (2004)). A phylogenetic tree was constructed using the Geneious software (R9.0.5) with the Jukes-Cantor distance model and UPGMA as a tree build method, with bootstrap values from 1,000 replicates.

nif cluster construction. To obtain large nif clusters on mobilizable plasmids that carry origin of transfer (oriT) for conjugative transfer of the plasmids, the genomic DNAs from K. oxytoca, P. stutzeri, A. vinelandii, A. caulinodans and R. sphaeroides were purified using Wizard genomic DNA purification kit, following the isolation protocol for gram negative bacteria (Promega, Cat #A1120). The genomic DNAs of Cyanothece ATCC51142, A. brasilense ATCC29729. R. palustris ATCC BAA-98, and G. diazotrophicus ATCC49037 were obtained from ATCC. Each nif cluster was amplified into several fragments (4-10 kb) with upstream and downstream 45 bp linkers at the 5′ and 3′ most end of the cluster by PCR with primer sets (Table 7) and assembled onto linearized E. coli-yeast shuttle vectors pMR-1 for E. coli and Rhizobia, and pMR-2 for P. protegens Pf-5 using yeast recombineering. For the nif cluster of Paenibacillus sp. WLY78, the DNA sequence information were gleaned from contig ALJV01 and the DNA of the nif cluster was synthesized by GeneArt gene synthesis (Thermo Fisher Scientific, MA) into four fragments that were used as templates for PCR amplification and assembly. Amplified fragments from two to eight (Table 7) were assembled with a linearized vector into a single large plasmid by one-pot yeast assembly procedure (Shanks, R. M. et al. Saccharomyces cerevisiae-based molecular tool kit for manipulation of genes from gram-negative bacteria. 72, 5027-5036 (2006)). Once assembled, the nif cluster-plasmids were isolated from yeast using Zymoprep Yeast Miniprep kit (Zymo Research Cat #D2004) and transformed into E. coli. The purified plasmid was isolated from E. coli and sequenced to verify the correct assembly and sequence (MGH CCIB DNA Core facility, Cambridge, MA). E. coli containing a mutation-free plasmid were stored for further experiments. Plasmids containing nif clusters are provided in Table 9.

Construction of refactored nif v3.2. The six transcriptional units (nifHDKTY, nifENX, nifJ. nifBQ, nifF, nifUSVWZM) were amplified from the plasmid pMR-3 that harbors the native Klebsiella nif cluster. Each unit was divided onto six level-1 module plasmids where the nif genes are preceded by a terminator. T7 promoter wild-type or T7 promoter variant PT7.P2 was placed between a terminator and the first gene of the transcriptional unit. Assembly linkers (˜45 bp) were placed at both ends of the units. The level-1 plasmids (pMR32-37) were provided in Table 9 and 10. Each of the six plasmids was linearized by digestion with restriction enzymes and assembled with a linearized pMR-1 or pMR-2 vector into a single large plasmid by one-pot yeast assembly procedure, yielding pMR38 and pMR39.

Transformation. Electroporation was used to transfer plasmids into P. protegens Pf-5. A single colony was inoculated in 4 mL of LB and grown for 16 h at 30° C. with shaking at 250 rpm. The cell pellets were washed twice with 2 mL of 300 mM sucrose and dissolved in 100 ul of 300 mM sucrose at RT. A total of 50-100 ng DNA was electroporated and recovered in 1 mL of LB media for 1 h before plating on selective LB plates. Triparental mating was used to transfer DNA from E. coli to Rhizobia. An aliquot of 40 μl of late-log phase (OD₆₀₀˜0.6) donor cells and 40 μl of late-log phage helper cells containing pRK7013 were mixed with 200 μl of late-log phase (OD₆₀₀˜0.8) recipient Rhizobia cells and washed in 200 μl of TY medium. Mating was initiated by spotting 20 μl of the mixed cells on TY plates and incubated at 30° C. for 6 h. The mating mixtures were plated on TY medium supplemented with nitrofurantoin to isolate Rhizobia transconjugants.

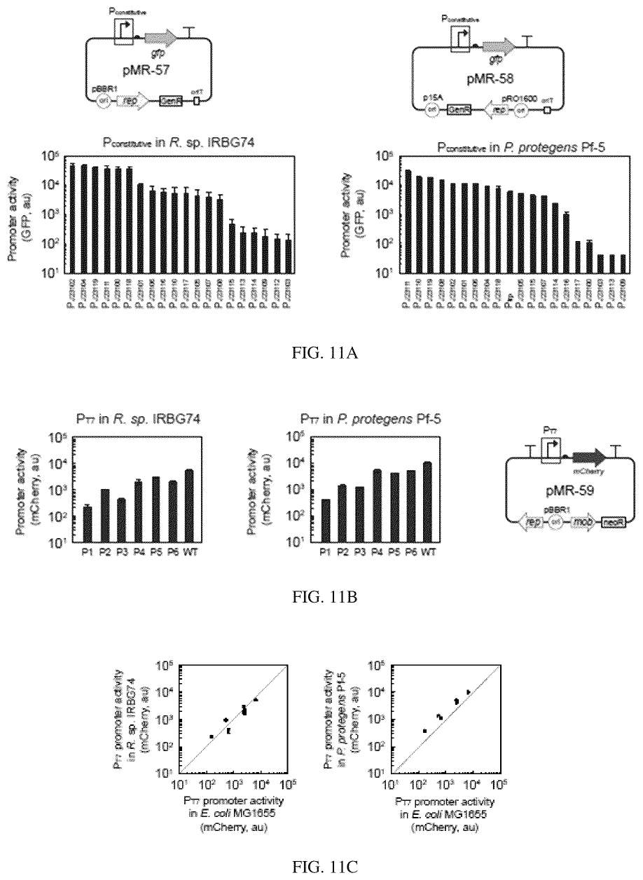

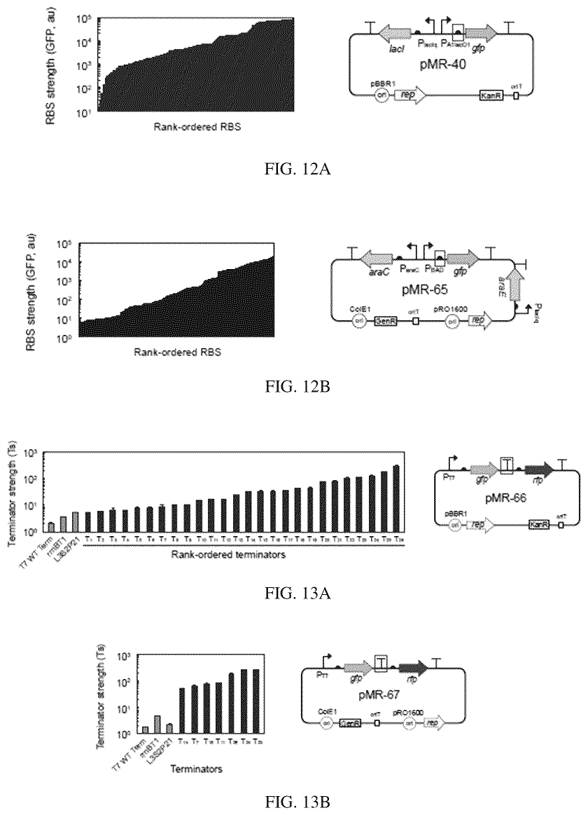

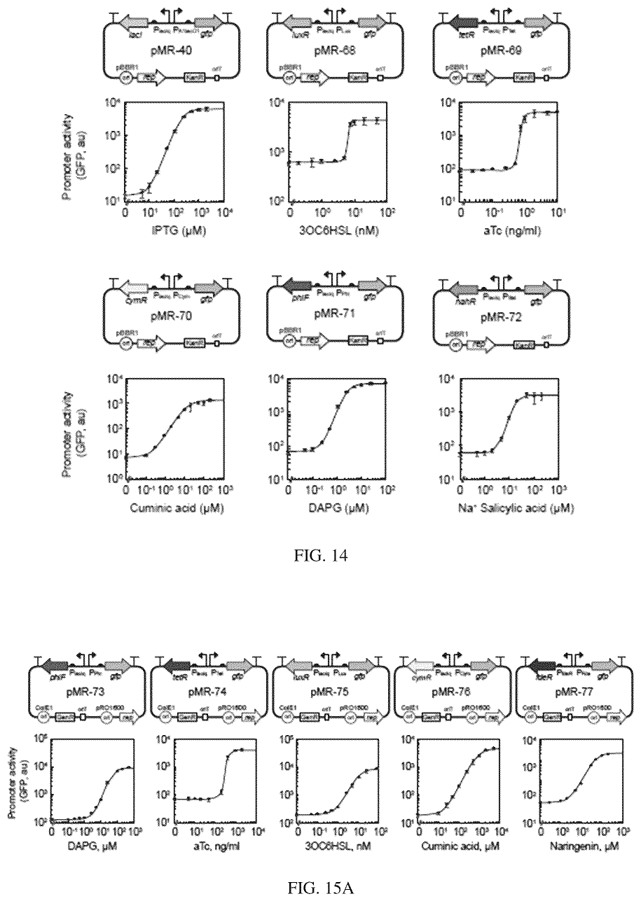

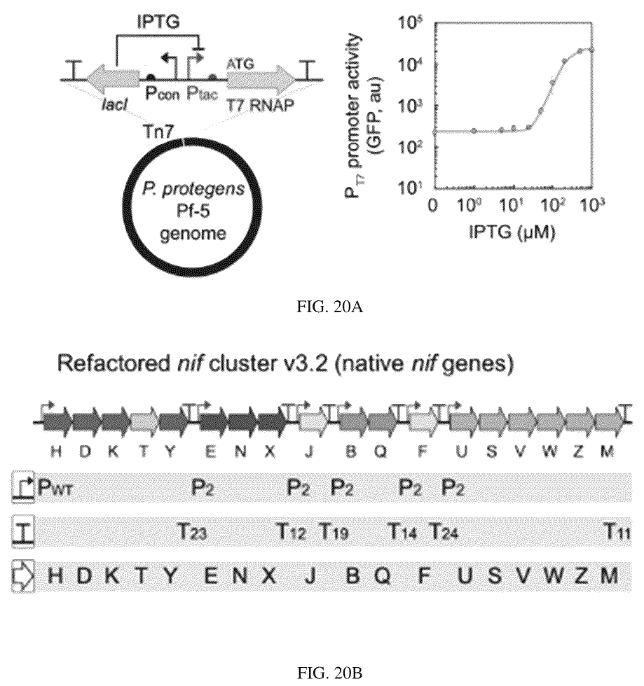

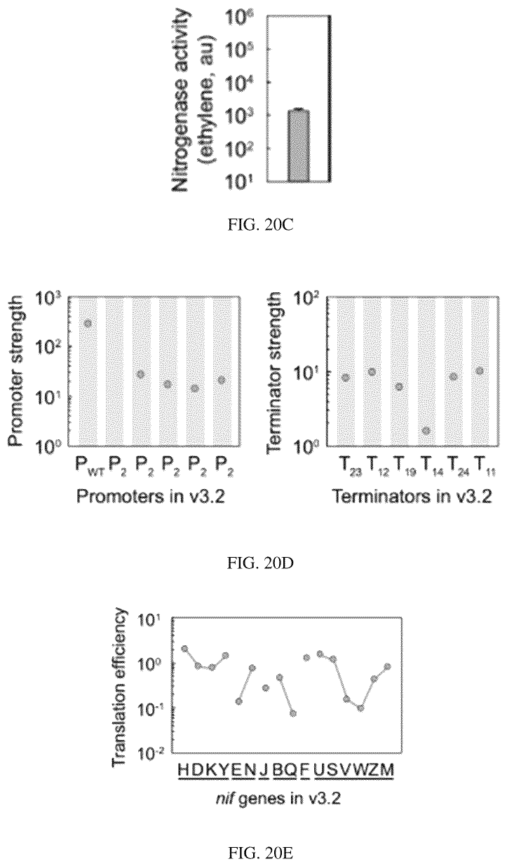

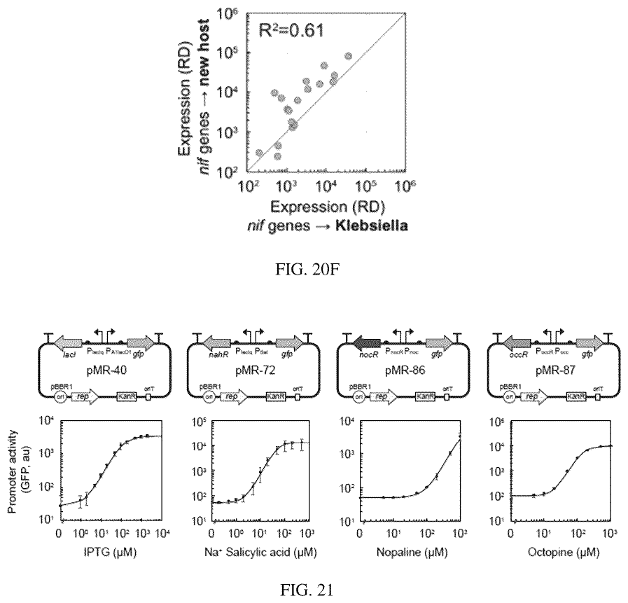

Construction and characterization genetic parts for Rhizobia. Genetic part libraries were built on a pBBR1-ori plasmid pMR-1 using Gibson assembly (New England Biolabs, Cat #E2611). The fluorescence proteins, GFPmut3b and mRFP1 were used as reporters. The Anderson promoter library (Anderson, J. et al. BglBricks: A flexible standard for biological part assembly. 4, 1 (2010)) on the BioBricks Registry were utilized for the characterization of constitutive promoters (FIGS. 11A-11C). To characterize inducible promoters, a regulator protein is constitutively expressed by the PlacIq promoter, and GFP expression is driven by a cognate inducible promoter from the opposite direction, facilitating replacement of the reporter with gene of interest (e.g., T7 RNAP and nifA) and transfer of the controller unit across different plasmid backbones for diverse microbes. The following combinations of cognate regulators and inducible promoters were characterized. IPTG inducible LacI-A1lacO1, DAPG inducible PhlF-PPhl, aTc inducible TetR-PTet, 3OC6HSL inducible LuxR-P_{Lux}, salicylic acid inducible NahR-P_{Sal}, and cuminic acid inducible CymR-P_{Cym} systems were optimized for R. sp. IRBG74 (FIG. 14). Opine inducible OccR-P_{occ}, and nopaline inducible NocR-Pnoc systems were optimized for A. caulinodans (FIGS. 20A-20F and Tables 9 and 10). For RBS characterization, an IPTG-inducible GFP expression plasmid pMR-40 was used and GFP was expressed to the highest levels with 1 mM IPTG (FIGS. 12A-12B). RBS library for GFP was designed using the RBS library calculator at the highest-resolution mode, and the 3′ end of the 16S rRNA sequences were adjusted according to the species (3′-ACCTCCTTC-5′ for R. sp. IRBG74). Terminators for T7 RNAP were characterized by placing a terminator between two fluorescence reporters expressed from a single T7 wild-type promoter located upstream of the first fluorescence protein GFP. The expression of the two fluorescence proteins is enabled by the controller strain MR18 encoding the IPTG-inducible T7 RNAP system by 1 mM IPTG (FIGS. 13A-13B). The terminator strength (Ts) was determined by normalizing fluorescence levels of a terminator construct by a reference construct pMR-66 where a 40 bp spacer was placed between the reporters. All genetic parts for Rhizobia were characterized as follows. Single colonies were inoculated into 0.5 ml TY supplemented with antibiotics in 96-deepwell plates (USA Scientific, Cat #18962110) and grown overnight at 30° C., 900 rpm in a Multitron incubator (INFORS HT, MD). 1.5 μl of overnight cultures was diluted into 200 μl of TY with antibiotics and appropriate inducers in 96-well plates (Thermo Scientific, Cat #12565215) and incubated for 7 h at 30° C., 1,000 rpm in an ELMI DTS-4 shaker (ELMI, CA). After growth, 8 μl of culture sample was diluted into 150 μl PBS with 2 mg/mL kanamycin for flow cytometry analysis. Plasmids and genetic parts are listed in Table 9 and 10.

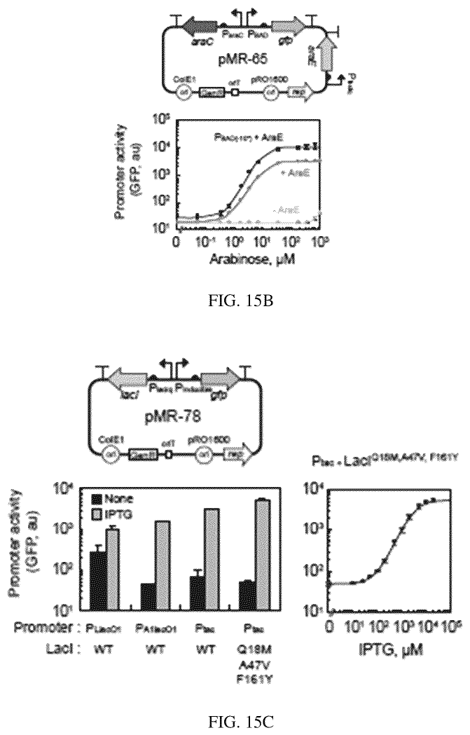

Construction and characterization genetic parts for P. protegens. Genetic part libraries were built on a pRO1600-ori plasmid pMR-2 using Gibson assembly (New England Biolabs, Cat #E2611). The fluorescence proteins, GFPmut3b and mRFP1 were used as reporters. The Anderson promoter library on the BioBricks Registry were utilized for the characterization of constitutive promoters (FIGS. 11A-11C). The following combinations of cognate regulators and inducible promoters were characterized. IPTG inducible LacI-P_{tac}, DAPG inducible PhIF-P_{Phl}, aTc inducible TetR-P_{Tet}, 3OC6HSL inducible LuxR-P_{Lux}, arabinose inducible AraC-P_{BAD}, cuminic acid inducible CymR-P_{Cym}, and naringenin inducible FdeR-P_{Fde} were optimized (FIGS. 15A-15C). For RBS characterization, an arabinose-inducible GFP expression plasmid pMR-65 was used and GFP was expressed with 1 mM IPTG (FIGS. 12A-12B). RBS library for GFP was designed using the RBS library calculator at the highest-resolution mode, and the 3′ end of the 16S IRNA sequences were adjusted according to the species (3′-ACCTCCTTA-5′ for P. protegens Pf-5). Terminators for T7 RNAP were characterized by placing a terminator between two fluorescence reporters expressed from a single T7 wild-type promoter located upstream of the first fluorescence protein GFP. The expression of the two fluorescence proteins is enabled by an IPTG-inducible T7 RNAP expression system of the controller strain MR7 (FIGS. 13A-13B). All genetic parts for P. protegens Pf-5 were characterized as follows. Single colonies were inoculated into 1 ml LB supplemented with antibiotics in 96-deepwell plates (USA Scientific, Cat #18962110) and grown overnight at 30° C., 900 rpm in a Multitron incubator (INFORS HT, MD). 0.5 μl of overnight cultures was diluted into 200 μl of LB with antibiotics and appropriate inducers in 96-well plates (Thermo Scientific, Cat #12565215) and incubated for 7 h at 30° C., 1,000 rpm in an ELMI DTS-4 shaker (ELMI, CA). After growth, 10 μl of culture sample was diluted into 150 μl PBS with 2 mg/mL kanamycin for flow cytometry analysis. Plasmids and genetic parts are listed in Tables 9 and 10.

Genomic integration and characterization of controllers. The mini-Tn7 insertion system was used to introduce a controller into the genome of P. protegens Pf-5. The IPTG-inducible T7 RNAP expression system and a tetracycline resistant marker tetA was placed between two Tn7 ends (Tn7L and Tn7R). The controller plasmid pMR-85 was introduced into P. protegens Pf-5 by double transformation with pTNS3 encoding the TasABCD transposase. A genomically-integrated controller located 25 bp downstream of the stop codon of glmS was confirmed by PCR and sequencing. A markerless insertion method using homologous recombination was employed in R. sp. IRBG74. A controller encoding inducible T7 RNAP system flanked by two homology fragments that enables the replacement of recA was cloned into a suicide plasmid. These controller plasmids (IPTG-inducible, pMR82-84; DAPG-inducible, pMR85) in E. coli was mobilized into R. sp. IRBG74 MR 18 (AhsdR. Δnif) by triparental mating, generating the controller strains (MR 19, 20, 21 and 22, respectively). The controller integration in the genome was confirmed by gentamicin sensitivity and diagnostic PCR. All controllers were characterized in a manner identical to that described in genetic part characterization.

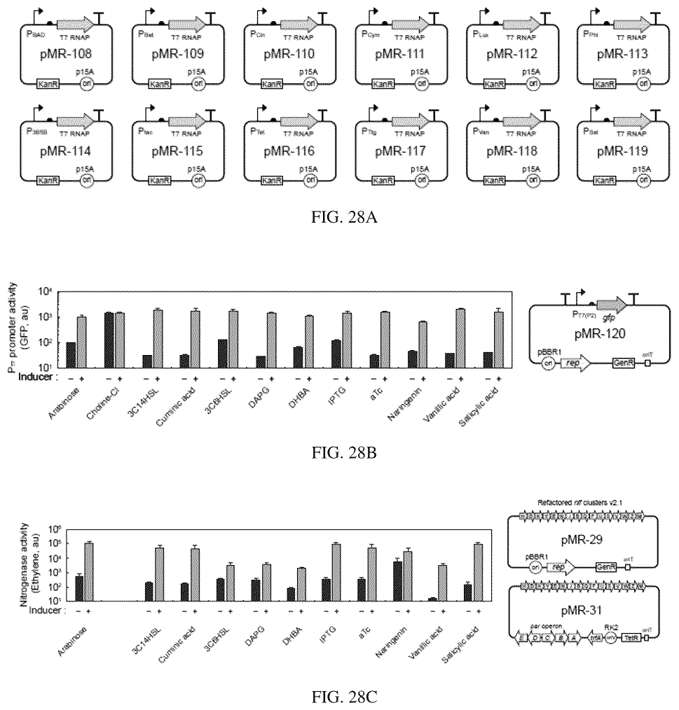

Construction and characterization of Marionette-based controllers. To regulate nitrogenase expression in the E. coli Marionette MG1655, the yfp in the 12 reporter plasmids was replaced with T7 RNAP while keeping other genetic parts (e.g., promoters and RBSs) unchanged (FIGS. 28A-28C). The reporter plasmid pMR-120 in which gfpmut3b is fused to the PT7 (P2) promoter (FIGS. 28A-28C) was co-transformed to analyze the response functions of each of the 12 T7 RNAP controller plasmids. To characterize controllers, single colonies were inoculated into 1 ml LB supplemented with antibiotics in 96-deepwell plates (USA Scientific, Cat #18962110) and grown overnight at 30° C., 900 rpm in a Multitron incubator (INFORS HT, MD). 0.5 μl of overnight cultures was diluted into 200 μl of LB with antibiotics and appropriate inducers in 96-well plates (Thermo Scientific, Cat #12565215) and incubated for 6 h at 30° C., 1,000 rpm in an ELMI DTS-4 shaker (ELMI, CA). After growth, 4 μl of culture sample was diluted into 150 μl PBS with 2 mg/mL kanamycin for flow cytometry analysis.

Flow cytometry. Cultures with fluorescence proteins were analyzed by flow cytometry using a BD Biosciences LSRII Forterssa analyzer with a 488 nm laser and 510/20-nm band pass filter for GFP and a 561 nm laser and 610/20 nm band pass filter for mCherry and mRFP1. Cells were diluted into 96-well plates containing phosphate buffered saline solution (PBS) supplemented with 2 mg/mL kanamycin after incubation. Cells were collected over 20,000 events which were gated using forward and side scatter to remove background events using FlowJo (TreeStar Inc., Ashland, OR). The median fluorescence from cytometry histograms was calculated for all samples. The median autofluorescence was subtracted from the median fluorescence and reported as the fluorescence value in arbitrary unit (au).

Nitrogenase assay (E. coli and K. oxytoca). Cultures were initiated by inoculating a single colony into 1 mL of LB supplemented with appropriate antibiotics in 96-deepwell plates (USA Scientific, Cat #18962110) and grown overnight at 30° C., 900 rpm in a Multitron incubator. 5 μl of overnight cultures was diluted into 500 μl of BB medium with 17.1 mM NH4CH3CO2 and appropriate antibiotics in 96-deepwell and incubated for 24 h at 30° C., 900 rpm in a Multitron incubator. Cultures were diluted to an OD600 of 0.4 into 2 mL of BB medium supplemented with appropriate antibiotics, 1.43 mM serine to facilitate nitrogenase depression, and an inducer (if necessary) in 10 mL glass vials with PTFE-silicone septa screw caps (Supelco Analytical, Cat #SU860103). Headspace in the vials was replaced with 100% argon gas using a vacuum manifold. Acetylene freshly generated from CaC₂ in a Burris bottle was injected to 10% (vol/vol) into each culture vial to begin the reaction. The acetylene reduction was carried out for 20 h at 30° C. with shaking at 250 rpm in an Innova 44 shaking incubator (New Brunswick) to prevent cell aggregations, followed by quenching via the addition of 0.5 mL of 4 M NaOH to each vial.

Nitrogenase assay (P. protegens Pf-5). Cultures were initiated by inoculating a single colony into 1 mL of LB supplemented with appropriate antibiotics in 96-deepwell plates (USA Scientific, Cat #18962110) and grown overnight at 30° C., 900 rpm in a Multitron incubator. 5 μl of overnight cultures was diluted into 500 μl of BB medium with 17.1 mM NH₄CH₃CO₂ and appropriate antibiotics in 96-deepwell and incubated for 24 h at 30° C., 900 rpm in a Multitron incubator. Cultures were diluted to an OD₆₀₀ of 0.4 into 2 mL of BB medium supplemented with appropriate antibiotics, 1.43 mM serine and an inducer (if necessary) in 10 mL. glass vials with PTFE-silicone septa screw caps. Headspace in the vials was replaced with 99% argon and 1% oxygen gas (Airgas, MA USA) using a vacuum manifold. Acetylene was injected to 10% (vol/vol) into each culture vial to begin the reaction. The acetylene reduction was carried out for 20 h at 30° C. with shaking at 250 rpm, followed by quenching via the addition of 0.5 mL of 4 M NaOH to each vial.

Nitrogenase assays (Rhizobia strains). Cultures were initiated by inoculating a single colony into 0.5 mL of TY medium supplemented with appropriate antibiotics in 96-deepwell plates (USA Scientific, Cat #18962110) and grown overnight at 30° C., 900 rpm in a Multitron incubator. 5 μl of overnight cultures was diluted into 500 μl of UMS medium with 30 mM succinate, 10 mM sucrose, and 10 mM NH₄Cl and appropriate antibiotics in 96-deepwell and incubated for 24 h at 30° C., 900 rpm in a Multitron incubator. Cultures were diluted to an OD₆₀₀ of 0.4 into 2 mL of UMS medium plus 30 mM succinate and 10 mM sucrose supplemented with appropriate antibiotics, 1.43 mM serine and an inducer (if necessary) in 10 mL glass vials with PTFE-silicone septa screw caps. Headspace in the vials was replaced with 99% argon and 1% oxygen gas using a vacuum manifold. Acetylene was injected to 10% (vol/vol) into each culture vial to begin the reaction. The acetylene reduction was carried out for 20 h at 30° C. with shaking at 250 rpm, followed by quenching via the addition of 0.5 mL of 4 M NaOH to each vial.

Nitrogenase assays (A. caulinodans and P. stutzeri). Cultures were initiated by inoculating a single colony into 0.2 mL of TY medium supplemented with appropriate antibiotics in 96-deepwell plates and grown overnight at 37° C. and 30° C. for A. caulinodans and P. stutzeri, respectively. 900 rpm in a Multitron incubator. 5 μl of overnight cultures was diluted into 500 μl of UMS medium with 30 mM lactate and 10 mM NH₄Cl and appropriate antibiotics in 96-deepwell and incubated for 24 h at 37° C. and 30° C. for A. caulinodans and P. stutzeri, respectively, 900 rpm in a Multitron incubator. Cultures were diluted to an OD₆₀₀ of 0.4 into 2 mL of UMS medium plus 30 mM lactate supplemented with appropriate antibiotics and an inducer (if necessary) in 10 mL glass vials with PTFE-silicone septa screw caps. Headspace in the vials was replaced with 99% argon plus 1% oxygen gas using a vacuum manifold. Acetylene was injected to 10% (vol/vol) into each culture vial to begin the reaction. The acetylene reduction was carried out for 20 h at 30° C. with shaking at 250 rpm, followed by quenching via the addition of 0.5 mL of 4 M NaOH to each vial.

Nitrogenase assays (A. vinelandii). Cultures were initiated by inoculating a single colony into 0.5 mL of Burk medium supplemented with appropriate antibiotics in 96-deepwell plates (USA Scientific, Cat #18962110) and grown overnight at 30° C., 900 rpm in a Multitron incubator. 5 μl of overnight cultures was diluted into 500 μl of Burk medium with 17.1 mM NH4CH3CO2 and appropriate antibiotics in 96-deepwell and incubated for 24 h at 30° C., 900 rpm in a Multitron incubator. Headspace in the vials was replaced with 97% argon and 3% oxygen gas (Airgas, MA USA) using a vacuum manifold. Acetylene was injected to 10% (vol/vol) into each culture vial to begin the reaction. The acetylene reduction was carried out for 20 h at 30° C. with shaking at 250 rpm, followed by quenching via the addition of 0.5 mL of 4 M NaOH to each vial.

Nitrogenase activity assay in the presence of ammonium. Following overnight incubation in minimal medium with a nitrogen source (described above), cultures were diluted to an OD₆₀₀ of 0.4 in 2 mL of nitrogen-free minimal medium, 1.43 mM serine (for E. coli and P. protegens Pf-5) and an inducer (for inducible systems) in 10 mL glass vials with PTFE-silicone septa screw caps. Ammonium (17.1 mM NH₄CH₃CO₂ for E. coli and P. protegens Pf-5 and 10 mM NH4Cl for Rhizobia) was added to a nitrogen-free minimal medium when testing ammonium tolerance of nitrogenase activity. Headspace in the vials was replaced with either 100% argon gas for E. coli, 99% argon plus 1% oxygen for Pseudomonas and Rhizobia using a vacuum manifold. Acetylene was injected to 10% (vol/vol) into each culture vial to begin the reaction. The acetylene reduction was carried out for 20 h at 30° C. with shaking at 250 rpm followed by quenching via the addition of 0.5 mL of 4 M NaOH to each vial.

Nitrogenase activity assay at varying oxygen levels. Following overnight incubation in minimal medium with a nitrogen source (described above), cultures were diluted to an OD₆₀₀ of 0.4 in 2 mL of minimal medium, 1.43 mM serine (for E. coli and P. protegens Pf-5), and an inducer (for inducible systems) in 10 mL glass vials with PTFE-silicone septa screw caps. The vial headspace was replaced with either 100% nitrogen gas for E. coli or 99% nitrogen plus 1% oxygen for P. protegens Pf-5 and A. caulinodans using a vacuum manifold. Cultures were incubated with shaking at 250 rpm at 30° C. for 6 h and 9 h for P. protegens Pf-5 and A. caulinodans, respectively, after which oxygen concentrations in the headspace were recorded with the optical oxygen meter FireStingO2 equipped with a needle-type sensor OXF500PT (Pyro Science, Germany). After the induction period, no oxygen remained in the headspace for all species as confirmed by the oxygen meter. The initial oxygen levels in the headspace were adjusted by injecting pure oxygen via syringe into the headspace of the vials and stabilized with shaking at 250 rpm at 30° C. for 15 m followed by the injection of acetylene to 10% (vol/vol) into each culture vial to begin the reaction and initial oxygen concentrations in the headspace were recorded concomitantly. The oxygen levels in the headspace were maintained around the setting points (<±0.25% (2) while incubating at 250 rpm and 30° C. by injecting oxygen every hour for 3 h with oxygen monitoring before and after oxygen spiking (FIGS. 26A-26B). The reactions were quenched after 3 h of incubation by the injection of 0.5 mL of 4 M NaOH to each vial using a syringe.

Ethylene quantification. Ethylene production was analyzed by gas chromatography using an Agilent 7890A GC system (Agilent Technologies, Inc., CA USA) equipped with a PAL headspace autosampler and flame ionization detector as follows. An aliquot of 0.5 mL headspace preincubated to 35° C. for 30 s was injected and separated for 4 min on a GS-CarbonPLOT column (0.32 mm×30 m, 3 microns; Agilent) at 60° C. and a He flow rate of 1.8 mL/min. Detection occurred in a FID heated to 300° C. with a gas flow of 35 mL/min H₂ and 400 mL/min air. Acetylene and ethylene were detected at 3.0 min and 3.7 min after injection, respectively. Ethylene production was quantified by integrating the 3.7 min peak using Agilent GC/MSD ChemStation Software.

Sample preparation for RNA-seq and Ribosome profiling. Cultures of K. oxytoca, E. coli, P. protegens Pf-5 or R. sp. IRBG74 were grown following the same protocol as used for nitrogenase activity assay (described above) with a few changes. Following overnight incubation in minimal medium with a nitrogen source, cultures were diluted to an OD₆₀₀=0.4 in 25 mL of minimal medium (with an inducer, if needed) and antibiotics in 125 mL Wheaton serum vials (DWK Life Sciences, Cat #223748) with septum stoppers (Fisher Scientific, Cat #FB57873). The vial headspace was replaced with either 100% nitrogen gas for E. coli and K. oxytoca or 99% nitrogen plus 1% oxygen for P. protegens Pf-5 and R. sp. IRBG74 using a vacuum manifold. Cultures grown 6 h at 30° C., 250 rpm were filtered onto a nitrocellulose filter 0.45 μM pore size (Fisher Scientific, Cat #GVS1215305). Cell pellets were combined from three vials using a stainless-steel scoopula, followed by flash-frozen in liquid nitrogen. The frozen pellets were added to 650 μl of frozen droplets of lysis buffer (20 mM Tris (pH 8.0), 100 mM NH₄Cl, 10 mM MgCl₂, 0.4% Triton X-100, 0.1% NP-40, 1 mM chloramphenicol and 100 U/mL DNase I) in prechilled 25 mL canister (Retsch, Germany, Cat #014620213) in liquid nitrogen and pulverized using TissueLyser II (Qiagen USA) with a setting at 15 Hz for 3 min for 5 times with intermittent cooling between cycles. The pellet was removed by centrifugation at 20,000 rcf at 4° C. for 10 min and the lysate was recovered in the supernatant.

RNA-seq experiments. RNA-seq and Ribosome-footprint profiling was carried out according to the method described earlier with a few modifications (Li, G.-W., Oh, E. & Weissman, J. S. J. N. The anti-Shine-Dalgarno sequence drives translational pausing and codon choice in bacteria. 484, 538 (2012); Li, G.-W., Burkhardt, D., Gross, C. & Weissman, J. S. Quantifying absolute protein synthesis rates reveals principles underlying allocation of cellular resources. Cell 157, 624-635 (2014)). The total RNA was isolated using the hot phenol-SDS extraction method. The rRNA fractions were determined and subtracted from the total using the MICROBExpress kit (Thermo Fisher Scientific, Cat #AM1905). The remaining mRNAs and tRNAs were fragmented by RNA fragmentation reagents (Thermo Fisher Scientific, Cat #AM8740) at 95° C. for 1 m 45 s. RNA fragments (10-45 bp) were isolated from a 15% TBE-Urea polyacrylamide gel (Thermo Fisher Scientific, Cat #EC6885). The 3′ ends of the RNA fragments were dephosphorylated using T4 polynucleotide kinase (1 U/μl, New England Biolabs, Cat #M0201S) in a 20 μl reaction volume supplemented with 1 μl of 20 U SUPERase. In at 37° C. for 1 h, after which the denatured fragments (5 pmoles) were incubated at 80° C. for 2 min and ligated to 1 μg of the oligo (/5rApp/CTGTAGGCACCATCAAT/3ddc/, Integrated DNA technologies) (SEQ ID NO: 1) in a 20 μl reaction volume supplemented with 8 μl of 50% PEG 8000, 2 μl of 10× T4 RNA ligase 2 buffer, 1 μl of 200 U/μl truncated K277Q T4 ligase 2 (New England Biolabs, Cat #M0351) and 1 μl of 20 U/μl of SUPERase. In at 25° C. for 3 h. The ligated fragments (35-65 bp) were isolated from a 10% TBE-Urea polyacrylamide gel (Invitrogen, Cat #EC6875). cDNA libraries from the purified mRNA products were reverse-transcribed using Superscript III

(Thermo Fisher Scientific, Cat #18080044) with oCJ485 primer (/5Phos/AGATCGGAAGAGCGTCGTGTAGGGAAAGAGTGT/iSp18/CAAGCAGAAGA CGGCATACGAGATATTGATGGTGCCTACAG (SEQ ID NO: 2, SEQ ID NO: 3)) at 50° C. for 30 min and RNA products subsequently were hydrolyzed by the addition of NaOH at a final concentration of 0.1 M, followed by incubation at 95° C. for 15 min. The cDNA libraries (125-150 bp) were isolated from on a 10% TBE-Urea polyacrylamide gel (Invitrogen, Cat #EC6875). The cDNA products were circularized in a 20 μl reaction volume supplemented with 2 μl of 10× CircLigase buffer, 1 μl of 1 mM ATP, 1 μl of 50 mM MnC12 and 1 μl of CircLigase (Epicenter, Cat #CL4115K) at 60° C. for 2 h and heat-inactivated at 80° C. for 10 min. 5 μl of circularized DNA was amplified using Phusion HF DNA polymerase (New England Biolabs, Cat #M0530) with o231 primer (CAAGCAGAAGACGGCATACGA (SEQ ID NO: 4)) and indexing primers (AATGATACGGCGACCACCGAGATCTACACGATCGGAAGAGCACACGTCTGAACT CCAGTCACNNNNNNACACTCTTTCCCTACAC (SEQ ID NO: 5)) for 7 to 10 cycles. The amplified products (125-150 bp) were recovered from an 8% TBE-Urea polyacrylamide gel (Invitrogen, Cat #EC62152). The purified products were analyzed by BioAnalyzer (Agilent, CA USA) and sequenced with a sequencing primer (CGACAGGTTCAGAGTTCTACAGTCCGACGATC (SEQ ID NO: 6)) using an Illumina HiSeq 2500 with a rapid run mode. To generate the RNA-seq read profile for each nif cluster, the raw trace profiles are multiplied by 107 and normalized by respective total reads from coding sequences of each species (K. oxytoca M5a1, CP020657.1; E. coli MG1655, NC_000913.3; P. protegens Pf-5, CP000076; R. sp. IRBG74 HG518322, HG518323, HG518324 and an appropriate plasmid carrying a nif cluster). The mRNA expression level of each gene was estimated using total sequencing reads mapped onto the gene, representing fragments per kilobase of transcript per million fragments mapped units (FPKM).

Ribo-seq experiments. 0.5 mg of RNA was diluted into 195 μl of the lysis buffer including 0.5 U RNase inhibitor SUPERase. In (Invitrogen, Cat #AM2694), 5 mM CaCl2 and were treated with 5 μl of 750 U of micrococcal nuclease (Sigma Aldrich, Cat #10107921001) at 25° C. for 1 h to obtain ribosome-protected monosomes. The digestions were quenched by the addition of EGTA to a final concentration of 6 mM and then kept on ice before the isolation of monosomes. Subsequently, the monosome fraction was collected by sucrose density gradient (10-55% w/v) ultracentrifugation at 35,000 rpm for 3 h, followed by a hot phenol-SDS extraction to isolate ribosome-protected mRNA fragments. The mRNA fragments (15-45 bp) were isolated from a 15% TBE-Urea polyacrylamide gel. The 3′ ends of the purified fragments were dephosphorylated and ligated to the modified oligo. cDNA libraries generated by Superscript III were circularized by CircLigase as described above. rRNA products were depleted by a respective biotinylated oligo mix for E. coli and P. protegens Pf-5. 5 μl of circularized DNA was amplified using Phusion HF DNA polymerase with 0231 primer and indexing primers for 7 to 10 cycles. The amplified products (125-150 bp) were recovered from an 8% TBE-Urea polyacrylamide gel. The purified products were analyzed by BioAnalyzer and sequenced with a sequencing primer (CGACAGGTTCAGAGTTCTACAGTCCGACGATC (SEQ ID NO: 7)) using an Illumina HiSeq 2500 with a rapid run mode. Sequences were aligned to reference sequences using Bowtie 1.1.2 with the parameters-k1-m2-v1. A center-weighting approach was used to map the aligned footprint reads ranging from 22 to 42 nucleotides in length. To map P-site of ribosome from footprint reads, 11 nucleotides from the both ends were trimmed, and the remaining nucleotide were given the same score, normalized by the length of the center region. Aligned reads (10-45 nucleotides) were mapped to the reference with equal weight of each nucleotide. A Python 3.4 script was used to perform the mapping. To generate the Ribo-seq read profile for each nif cluster, the raw trace profiles are multiplied by 10⁸ and normalized by respective total reads from coding sequences of each species. To calculate the ribosome density of each gene, read densities were first normalized in the following ways: (i) The first and last 5 codons of the gene are excluded for the calculation to remove the effects of translation initiation and termination. (ii) A genome-wide read density profile was fitted to an exponential function and the density at each nucleotide on a given gene was corrected using this function. (iii) If the average read density on a gene is higher than 1, a 90% winsorization was applied to reduce the effect of outliers. The sum of normalized reads on a gene was normalized by the gene length and the total read densities on coding sequences to yield the ribosome density.

Calculation of genetic part strengths based on-seq data. The activity of a promoter is defined as the change in RNAP flux 8/around a transcription start site Xiss (Gorochowski, T. E. et al. Genetic circuit characterization and debugging using RNA-seq. 13, 952 (2017)). The promoter strength is calculated by

$$δ \text{?} = \frac{\text{?}}{\text{?}} [ \text{?} m ( i ) - \text{?} m ( i ) ] \qquad ( 1 )$$

$$\text{?} \text{indicates text missing or illegible when filed}$$

where m (i) is the number of transcripts at each position I from FPKM-normalized transcriptomic profiles, y=0.0067 s″ is the degradation rate of mRNA, n is the window length before and after xₜₛₛ. The window length is set to 10. The terminator strength Tₛ is defined as the fold-decrease in transcription before and after a terminator, which can be quantified from FPKM-normalized transcriptomic profiles as

$$\text{?} = \frac{\text{?} m ( i )}{\text{?} m ( i )} \qquad ( 2 )$$

$$\text{?} \text{indicates text missing or illegible when filed}$$

where x₀ and x₁ are the beginning and end positions of the terminator part, respectively. Translation efficiency was calculated by dividing the ribosome density by the FPKM.

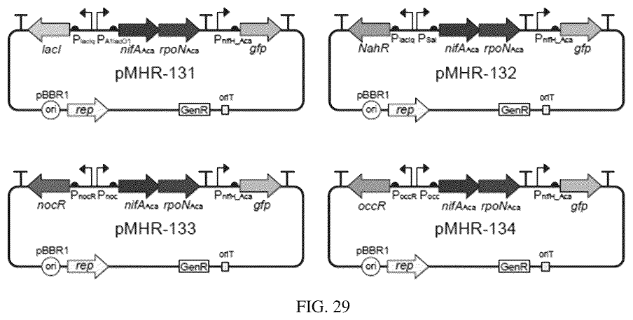

nifH expression analysis. Complementation of NifA was tested using plasmid pMR-128 to 130 that contains the sfgfp fused to the nifH promoter in the A. caulinodans ΔnifA mutant. The inducible NifA/RpoN expression was provided by the plasmid pMR-121 into which sfgfp driven by the nifH promoter was added to analyze nifH promoter activity, yielding pMR-131 (FIG. 29). The IPTG-inducible system in the plasmid pMR-124 was substituted with other inducible systems including the salicylic acid-inducible, nopaline-inducible and octopine-inducible systems, yielding pMR-125, 126, and 127, respectively. Each of the plasmids was mobilized into the A. caulinodans ΔnifA mutant, which was grown following the same protocol as used for nitrogenase activity (described herein). Following overnight incubation in minimal medium with a nitrogen source, cultures were diluted to an OD₆₀₀=0.4 in 2 mL. of UMS medium plus 30 mM lactate, antibiotics and an inducer (for inducible systems) in 10 mL glass vials with PTFE-silicone septa screw caps. Headspace in the vials was replaced with 99% argon plus 1% oxygen using a vacuum manifold. The vials were incubated with shaking at 250 rpm at 30° C. for 9 h, after which 10 μl of cultures was diluted into 150 μl PBS with 2 mg/mL kanamycin for flow cytometry analysis. To test activation of the nifH promoters by diverse NifA proteins, the plasmids pMR-51, 53, 88, 89 and 90 were introduced into E. coli MG1655 and the plasmids pMR-91, 92, 93, 94 and 95 to P. protegens Pf-5. The plasmid pMR-101 was used to provide inducible NifA expression by IPTG in E. coli. The controller encoding the IPTG-inducible NifA was inserted into the genome of P. protegens Pf-5 using the plasmids pMR-96, 97 and 98. The IPTG-inducible system of the NifA controller plasmid pMR-96 was replaced with the arabinose-inducible and the naringenin-inducible system, yielding pMR-99 and 100, respectively. The inducibility of nifH expression was assessed by the reporter plasmids pMR-105 to 107 and pMR102 to 104 for E. coli and P. protegens Pf-5, respectively. The controller plasmids were transformed into E. coli or P. protegens Pf-5 with the reporter plasmids. Following overnight incubation in minimal medium with a nitrogen source, cultures were diluted to an OD_{600=0.4} in 2 mL of BB medium, antibiotics and an inducer (for inducible systems) in 10 mL glass vials with PTFE-silicone septa screw caps. Headspace in the vials was replaced with either 100% argon for E. coli or 99% argon plus 1% oxygen for P. protegens Pf-5 using a vacuum manifold. The vials were incubated with shaking at 250 rpm at 30° C. for 9 h, after which 10 μl of cultures was diluted into 150 μl PBS with 2 mg/mL kanamycin for flow cytometry analysis.

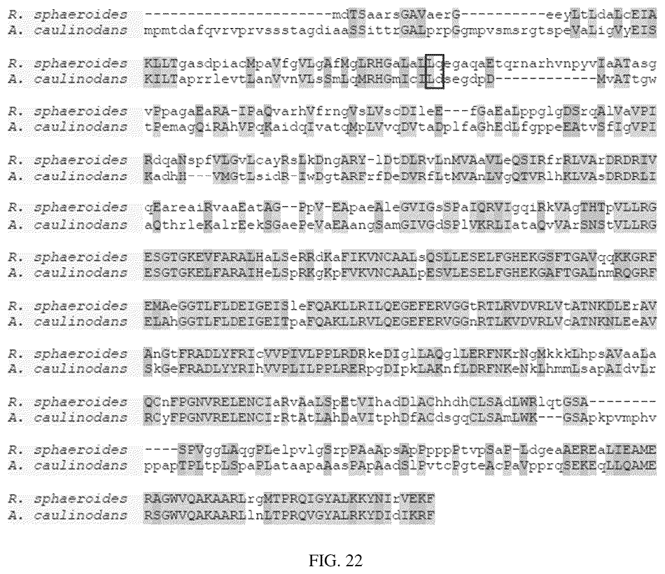

Sequence alignment. NifA sequences of R. sphaeroides 2.4.1 (RSP_0547) and A. caulinodans ORS571 (AZC_1049) were obtained from NCBI. NifA protein sequences were aligned with MUSCLE (https://www.ebi.ac.uk/Tools/msa/muscle/) with a default settings (FIG. 22).

### Results

Performance of Native Nif Clusters in E. coli, P. Protegens Pf-5, and Symbiotic Rhizobia

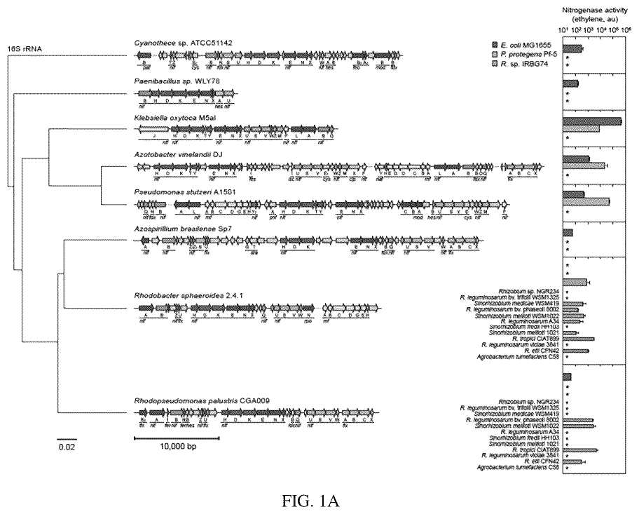

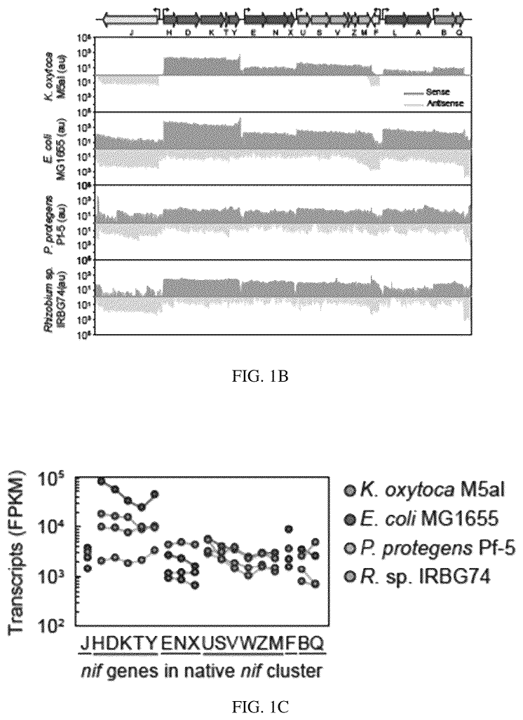

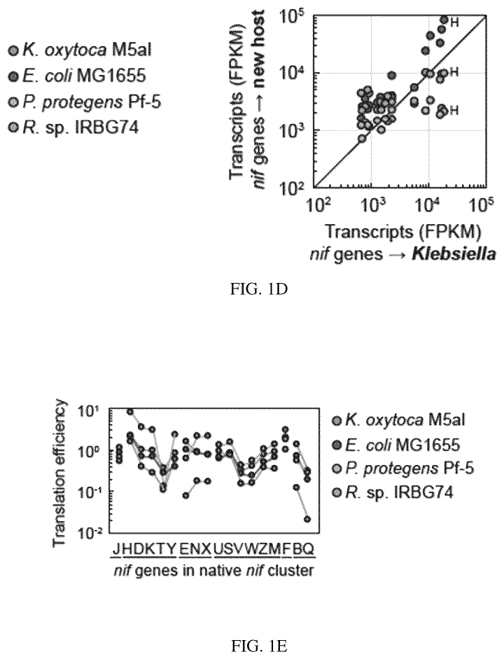

A set of diverse native nif clusters were cloned in order to determine their relative performance in different strains and the associated species barriers (FIG. 1A). Previously-defined boundaries for the well-studied nif cluster from K. oxytoca (Arnold, W., Rump, A., Klipp, W., Priefer, U. B. & Pühler, A. J. J. o. m. b. Nucleotide sequence of a 24.206-base-pair DNA fragment carrying the entire nitrogen fixation gene cluster of Klebsiella pneumoniae. 203, 715-738 (1988)) and the small (10 kb) cluster from Paenibacillus polymyxa WLY7870 were used. Similarly, the published boundaries (43.7 kb) of the P. stutzeri A1501 (Yan, Y. et al. Nitrogen fixation island and rhizosphere competence traits in the genome of root-associated Pseudomonas stutzeri A1501. Proceedings of the National Academy of Sciences (2008).) and A. vinelandii DJ clusters were used (Hamilton, T. L. et al. Transcriptional profiling of nitrogen fixation in Azotobacter vinelandii. J Bacteriol 193, 4477-4486, doi:10.1128/JB.05099-11 (2011)). A region of the P. stutzeri A1501 nif cluster (Pst1307-Pst1312) was excluded as these genes are predicted to have no effect on nitrogenase. A. vinelandii DJ contains three putative electron transport systems (the Rnf1 and Rnf2 complexes and the Fix complex) located in other regions of the genome. RNA-seq data shows that Rnf2 is not co-expressed with the nif genes, so only the Rnf1 and Fix complexes were included by fusing their DNA to create a single 46.9 kb construct. The nif cluster (40.1 kb) from Azospirillum brasilense Sp7 was selected because this species is a cereal endophyte and fixes nitrogen in free-living conditions. Several less-studied gene clusters were also cloned in order to probe species barriers. As a representative of cyanobacteria, the gene cluster from Cyanothece sp. ATCC51142 was cloned following published boundaries. Its transcriptional activator PatB occurs outside of the nif cluster, which was cloned along with its native promoter and fused to nif cluster to form a single construct (31.7 kb). Several gene clusters were selected from photosynthetic purple bacteria (Rhodopseudomonas palustris CGA009 (Oda, Y. et al. Functional genomic analysis of three nitrogenase isozymes in the photosynthetic bacterium Rhodopseudomonas palustris. 187, 7784-7794 (2005)) and Rhodobacter sphaeroides 2.4.1 (Haselkorn, R. & Kapatral, V. in Genomes and genomics of nitrogen-fixing organisms, 71-82 (Springer, 2005))) as these are members of the same alphaproteobacteria class as Rhizobia. The rnf cluster, encoded on a separate chromosome of R. sphaeroides 2.4.1, was added to the nif cluster to provide electrons to nitrogenase. Finally. the gene clusters from the sugarcane and rice endosymbiant Gluconacetobacter diazotrophicus PA15 (28.9 kb) as well as the three nif clusters from A. caulinodans ORS571 (64 kb)³⁷ were cloned together with an upstream regulator fixLJK, but these were found to be inactive in all species tested, so they are not shown in FIGS. 1A-1F. The precise genomic locations for all the nif clusters are provided in Table 7 and the plasmids containing nif clusters are provided in Table 8.

Each cluster was amplified from genomic DNA as multiple fragments by PCR and assembled with the plasmid backbone using yeast assembly (see Methods and Materials Section). The P. polymyxa WLY78 cluster was de novo synthesized based on the DNA sequence on contig ALJV01 (Shanks, R. M. et al. Saccharomyces cerevisiae-based molecular tool kit for manipulation of genes from gram-negative bacteria. 72, 5027-5036 (2006)). The clusters were cloned into different plasmid systems to facilitate transfer. For transfer to E. coli and R. sp. IRBG74, the broad-host range plasmid based on a pBBR1 origin was used (a second compatible RK2-origin plasmid was used for the nif cluster from A. caulinodans ORS571). These plasmids contain the RK2 oriT to enable the conjugative transfer of large DNA (see Materials and Methods). For transfer to P. protegens Pf-5, this plasmid system was found to be unstable and produce a mixed population. To transfer into this strain, the Pseudomonas-specific plasmid pRO1600 with the oriT was used. After construction, all of the plasmids were verified using next-generation sequencing (see Methods and Materials Section).

The set of 10 nif clusters were transferred into E. coli MG1655, the cereal epiphyte P. protegens Pf-5, and the cereal endophyte R. sp. IRBG74 to create 30 strains (FIG. 1A). E. coli was selected as a control as most of the published successful transfers have been to this recipient. Native P. protegens Pf-5 does not fix nitrogen. R. sp. IRBG74 contains two nif clusters in different genomic locations, which were left intact, but does not have nitrogenase activity under free living conditions. The genomic cluster does not have the required NifV enzyme as it obtains homocitrate from the plant. All of the clusters in the set have nifV, except the one from P. polymyxa WLY78. A test was run to determine whether the expression of recombinant nifV from A. caulinodans ORS571 in R. sp. IRBG74 would result in active nitrogenase, but no activity was detected.

The bacteria were grown in appropriate media, including antibiotics, and then evaluated for nitrogenase activity using an acetylene reduction assay (see Methods and Materials Section). E. coli and Pseudomonas were grown at 30° C. in BB minimal media, as described previously⁷¹. However, no growth was observed for R. sp. IRBG74 under these conditions. Different media and carbon sources were tested and it was found that UMS media with dicarboxylic acids (malate or succinate), the major carbon source from plants¹⁴⁷, with 10 mM sucrose yielded the highest growth rates (FIG. 6). After overnight growth, cells were transferred to stoppered test tubes in ammonium-free minimal media to a final OD₆₀₀ of 0.4. For E. coli, the headspace air is completely replaced with argon gas. For P. protegens Pf-5 and R. sp. IRBG74, the initial headspace concentration of oxygen was maintained at 1% because these bacteria require oxygen for their metabolism. The cells are incubated at 30° for 20 hours in the presence of excess acetylene and the conversion to ethylene was quantified by GC-MS (see Methods and Materials Section). There was no significant growth for any of the strains under these conditions, so the nitrogenase activities reported correspond to the same cell densities.

A surprising 6 out of 10 clusters were functional in E. coli MG1655, with the K. oxytoca cluster producing the highest activity (FIG. 1A). The K. oxytoca cluster is also functional in P. protegens Pf-5, albeit with 60-fold less activity as compared to that in E. coli MG1655. Interestingly, the clusters from P. stutzeri and A. vinelandii—both obligate aerobes—are able to achieve high activities in P. protegens Pf-5. The resulting nitrogenase activities are 3- to 7-fold higher than that achieved from K. oxytoca, which only fixes nitrogen under strict anaerobic conditions. These clusters have common organizational features and similar electron transport chains, such as the Rnf complex.

A single gene cluster, from R. sphaeroides, yielded nitrogenase activity in R. sp. IRBG74 (FIG. 1A). Notably, both Rhizobium and Rhodobacter are alphaproteobacter and their nif clusters may contain interchangeable genes. When the native nif clusters are knocked out of R. sp. IRBG74, introducing the R. sphaeroides cluster alone does not yield active nitrogenase. These data point to a complex complementation between the endogenous and introduced gene clusters. To determine whether this approach could be generalized to other symbiotic Rhizobia, the Rhodobacter and Rhodopseudomonas gene clusters were transferred to a panel of 12 species isolated from diverse legumes (FIG. 1A). Remarkably, the transfer of these clusters was able to produce detectable nitrogenase activity in 7 of the strains.

Hereafter, studies were conducted to better characterize the extent to which changes in transcription and translation impacted the differences in activity observed when a native cluster is transferred between species. Differences in promoter activity, ribosome binding sites, and codon usage could change the expression levels of nif genes in detrimental ways. To quantify this effect, RNA-seq and ribosome profiling experiments were performed to evaluate the expression K. oxytoca nif cluster in K. oxytoca as well as E. coli MG1655, P. protegens Pf-5, and R. sp. IRBG74. RNA-seq experiments provide mRNA levels of genes (calculated as FPKM) and can be used to measure the performance of promoters and terminators. Ribosome profiling can be used to quantify protein synthesis rates, ribosome binding site (RBS) strength and ribosome pausing internal to genes. The ribosome density (RD) has been shown to correlate with protein expression rates. The translation efficiency is calculated by normalizing the RD by the number of transcripts (FPKM from Ribo-seq). Ribosome profiling has been applied to determine the relative levels of proteins expressed in multi-subunit complexes.

The RNA-seq profiles in both the sense and antisense direction are very close when compared between K. oxytoca and E. coli (FIGS. 1B-1C) and the ratios between mRNAs is preserved (R²=0.89) (FIG. 1D). This is consistent with the observation that this cluster yields a similar activity in both hosts. In contrast, the RNA-seq profiles differ more significantly for P. protegens Pf-5 and R. sp. IRBG74 (FIGS. 1B-1C), and there was no correlation between mRNA transcripts (FIG. 1D).

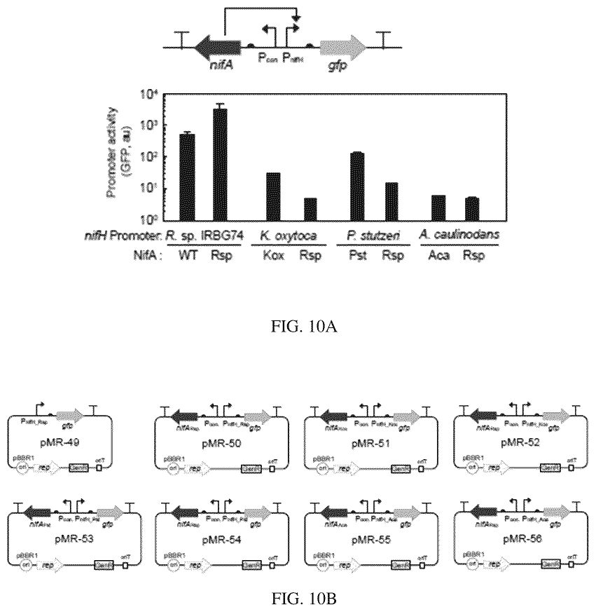

The ratios between protein expression rates were measured using ribosome profiling (FIG. 1E and FIG. 9). It is noteworthy that the ratios measured in K. oxytoca almost perfectly correlate with immunoblotting assays of A. vinelandii and the stoichiometry of H:D:K reflects the known 2:1:1 ratio. Interestingly, unlike mRNA levels, the ratios in expression rates are strongly correlated when the cluster is transferred between species: E. coli (R²=0.94), P. protegens Pf-5 (R²=0.61), and R. sp. IRBG74 (R²=0.71) (FIGS. 1E-1F). The production of NifH is significantly lower in R. sp. IRBG as compared to other strains. In an attempt to increase the induction of the cluster in this host, NifA was overexpressed, but this proved unsuccessful in producing high levels of active nitrogenase (FIGS. 10A-10B).

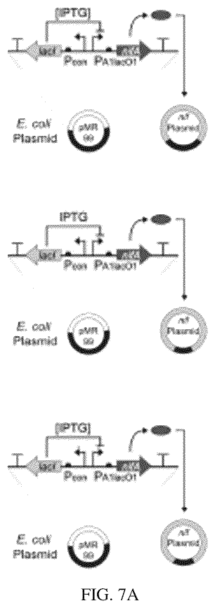

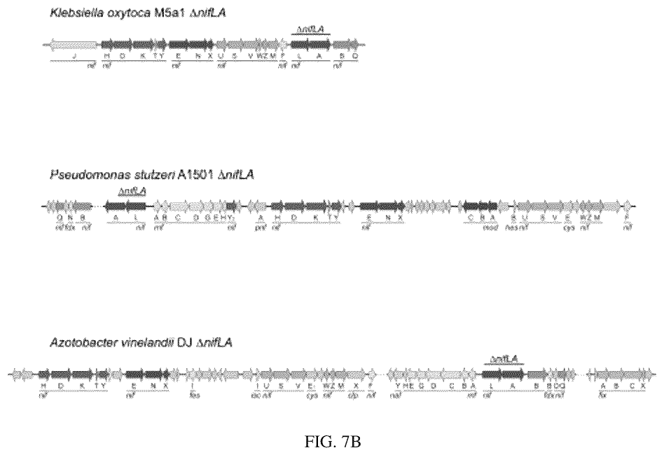

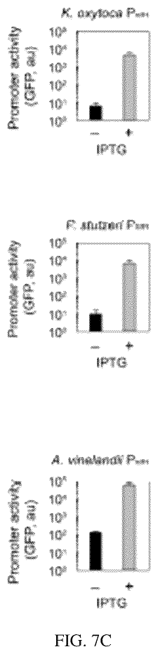

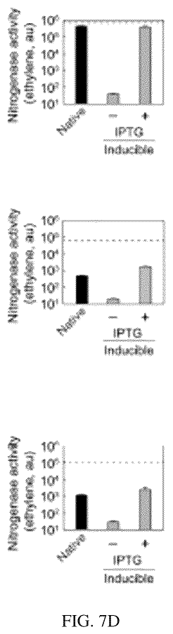

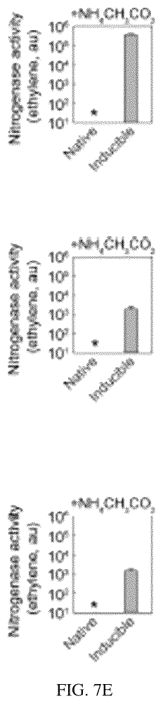

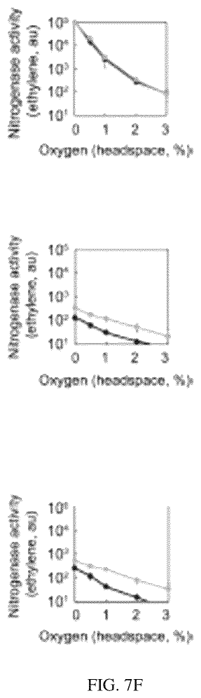

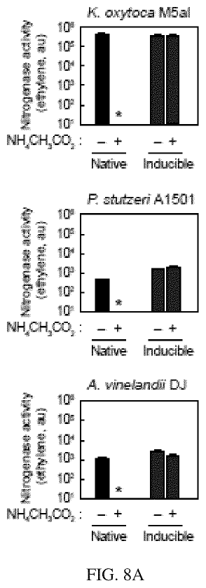

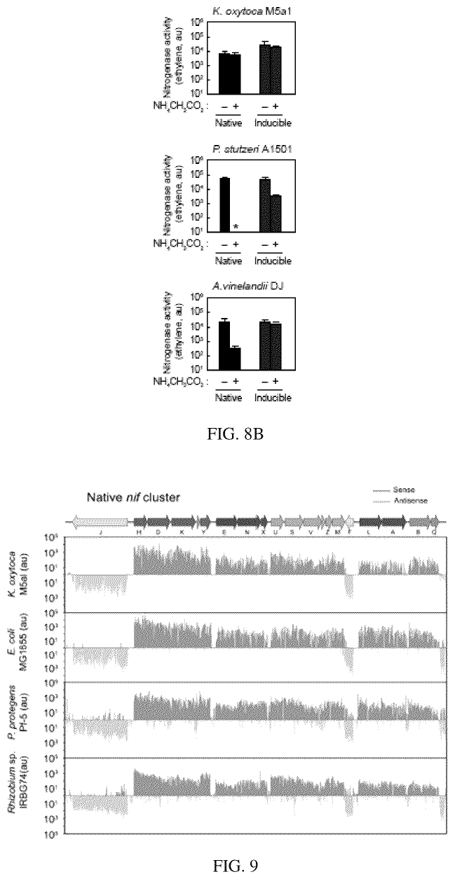

The following summarizes the results of the transfer of native nif clusters to new species. The most successful recipient is E. coli. However, this is not a viable agricultural strain and activity is eliminated in the presence of 17.1 mM ammonium, consistent with previous results (FIGS. 7A-7E, and FIGS. 8A-8B). Moderately high activity can be obtained in P. protegens Pf-5, but this yields a constitutively-on response (the K. oxytoca cluster) or is strongly repressed by ammonium (the A. vinelandii cluster). It was also found that the P. stutzeri cluster in P. protegens Pf-5 is inactive in the presence of ammonium, in disagreement with previously published results (Setten, L. et al. Engineering Pseudomonas protegens Pf-5 for nitrogen fixation and its application to improve plant growth under nitrogen-deficient conditions. PLOS One 8, e63666 (2013)). In previous studies, the published strain is not made available by the authors nor is its sequence, thus it is impossible to replicate the strain perfectly and differences in the cluster boundary or mutations to the regulation during construction could explain the discrepancy in results. Only low levels of activity could be obtained by transferring clusters to Rhizobia. To address these issues, different approaches were applied to engineer the clusters to generate higher activity, exhibit less repression by ammonium, and be inducible.

Transfer of Refactored Klebsiella Nif Clusters to R. Sp. IRBG74

The process of refactoring a gene cluster involves the complete reconstruction of the genetic system from the bottom-up, using only well-characterized genetic parts. An exhaustive approach is to recode the genes (to eliminate internal regulation), reorganize into operons, control expression with synthetic ribosome binding sites (RBSs), and use T7 RNAP promoters and terminators. A separate “controller,” carried in a genetically distinct location, links synthetic sensors and circuits to the expression of T7 RNAP. For various applications, this approach has proven useful for transferring multi-gene systems between species, simplifies optimization through part replacement and enzyme mining, and enables the replacement of environmental signals that naturally control the cluster with the stimuli that induce the synthetic sensors (Smanski, M. J. et al. Synthetic biology to access and expand nature's chemical diversity. Nature Reviews Microbiology 14, 135 (2016); Song, M. et al. Control of type III protein secretion using a minimal genetic system. 8, 14737 (2017); Guo, C.-J. et al. Discovery of reactive microbiota-derived metabolites that inhibit host proteases. 168, 517-526. e518 (2017); Ren, H., Hu, P., Zhao, H. J. B. & bioengineering. A plug-and-play pathway refactoring workflow for natural product research in Escherichia coli and Saccharomyces cerevisiae. 114, 1847-1854 (2017)). In previous studies, the Klebsiella nif cluster was refactored, which was subsequently used as a platform to optimize activity by changing the genetic organization and the parts controlling expression. The top variant (v2.1) fully recovered activity in a K. oxytoca nif knockout and is functional in E. coli. An interesting observation during optimization is that the genetic organization of the native cluster, including the existence of operons, was not correlated with activity.

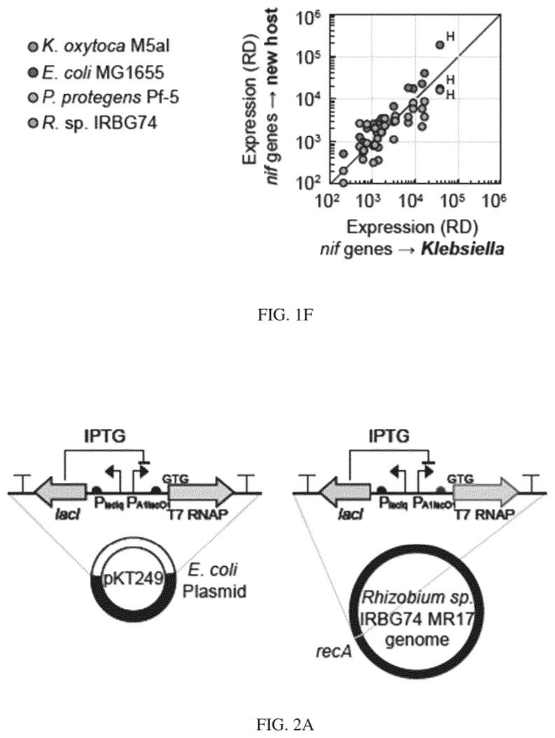

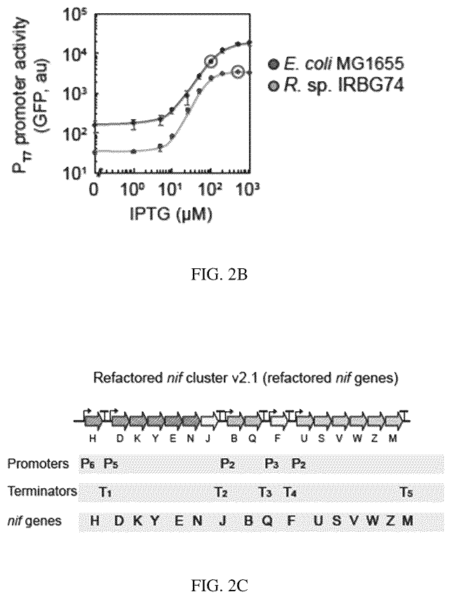

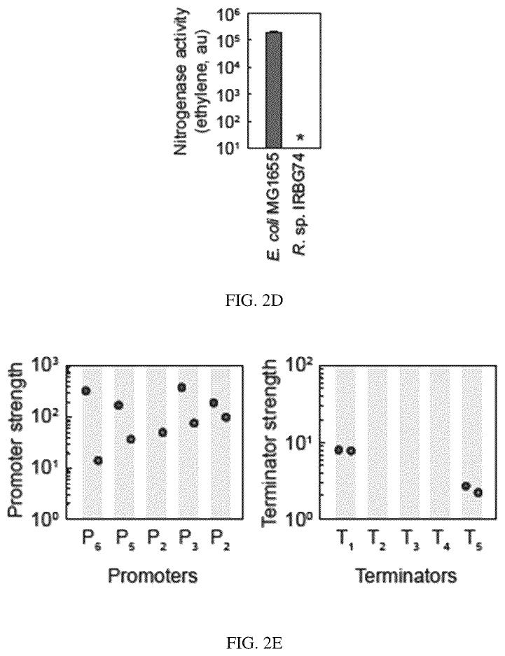

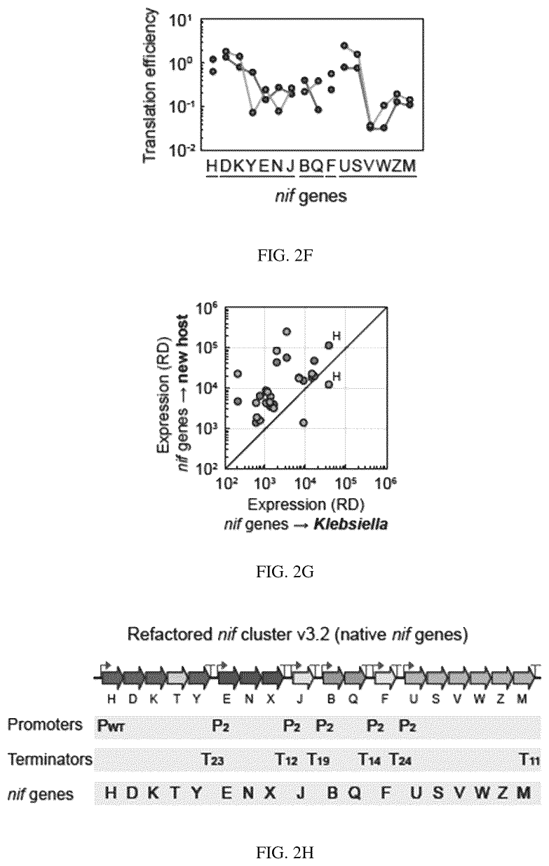

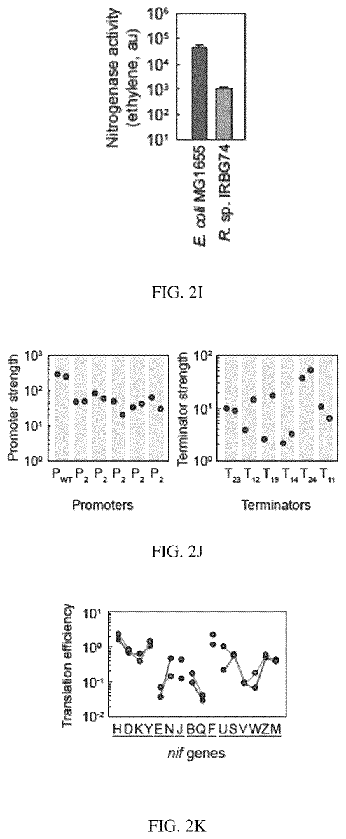

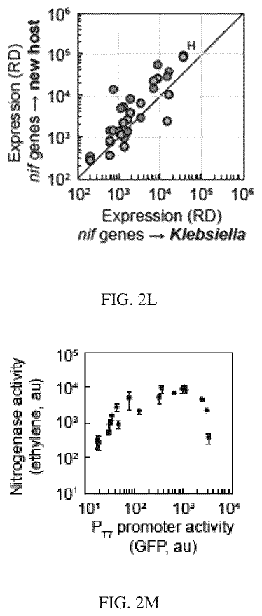

The present disclosure sought to study the performance of the refactored v2.1 cluster in R. sp. IRBG74. An advantage of using T7 RNAP is that it is functional in essentially all prokaryotes, so the refactored cluster can be transferred as-is and transcription induced by expressing T7 RNAP in the new host. However, a new controller needs to be built for each host based on regulation and regulatory parts that work in that species. Previously, a controller for E. coli was designed based on the IPTG-inducible T7 RNAP carried on a plasmid (pKT249) (FIG. 2A). To transfer the refactored cluster to R. sp. IRBG74, first a controller was constructed that functions in this species and produces an equivalent range of T7 RNAP expression.

While a handful of inducible systems and sets of genetic parts have been previously described for Rhizobia, a new part collection needed to be built and characterized in order to have those needed to create a controller with sufficient dynamic range. First, a set of 20 constitutive promoters (Anderson, J. et al. BglBricks: A flexible standard for biological part assembly. 4, 1 (2010)) and seven T7 RNAP-dependent promoters (emme, K., Zhao, D. & Voigt, C. A. Refactoring the nitrogen fixation gene cluster from Klebsiella oxytoca. Proceedings of the National Academy of Sciences 109, 7085-7090 (2012) that were found to span a range of 382-fold and 23-fold expression, respectively, were characterized (FIGS. 11A-11C). Second, a library of 285 ribosome binding sites (RBSs) were screened using the RBS Library Calculator, representing an expression range of 5,600-fold (FIGS. 12A-12B). Finally, a set of 29 terminators was characterized, of which 17 were found to have a terminator strength >10 (FIGS. 13A-13B). Using these part libraries, six inducible systems for R. sp. IRBG74 were then constructed that respond to IPTG, the quorum signal 3OC6HSL, aTe, cuminic acid, DAPG, and salicylic acid (FIG. 14). After optimization, these systems generate between 7- to 400-fold induction.

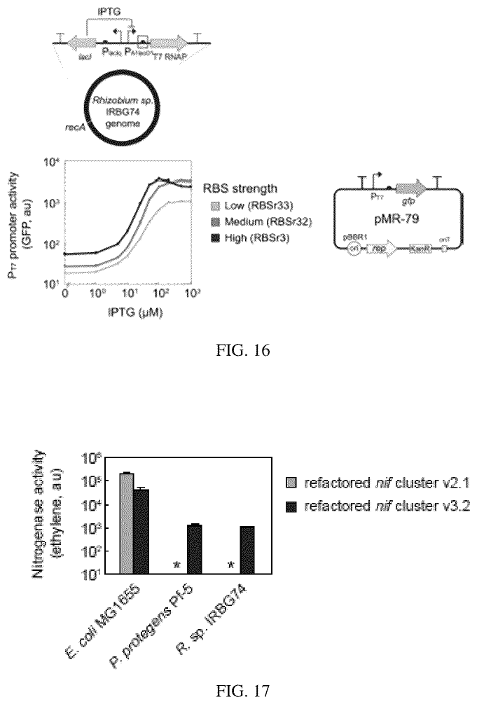

A controller was then constructed by using the optimized IPTG-inducible system to drive the expression of a variant of T7 RNAP (R6232S, N-terminal lon tag, GTG start codon) (FIG. 2A). RBS variants controlling T7 RNAP expression were tested and an intermediate strength was selected to maximize induction while limiting toxicity (FIG. 16). The controller was carried on the genome by replacing recA (see Methods and Materials). The response function of the final controller is compared to that obtained for pKT249 in E. coli, showing that they sweep through the same range of expression at intermediate levels of induction (FIG. 2B). To achieve the same level of induction in the two species, 0.1 mM IPTG is selected for E. coli and 0.5 mM for R. sp. IRBG74 (circled points in FIG. 2B).

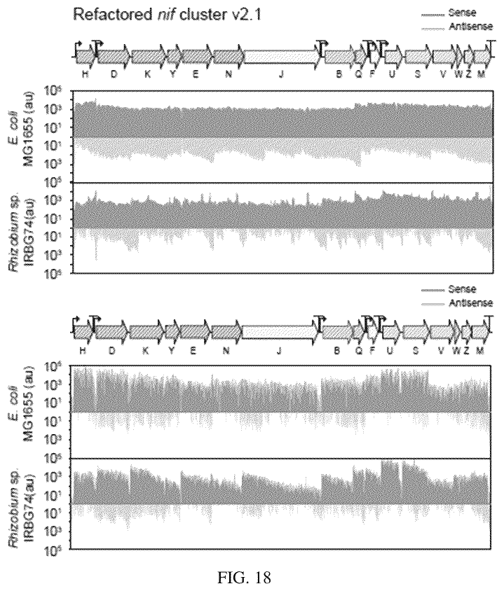

The refactored v2.1 cluster was then transferred to R. sp. IRBG74, but no activity was observed (FIGS. 2C-2D). Activity was also not observed when the v2. 1 cluster was transferred to P. protegens Pf-5 (FIG. 17). To determine if the genetic parts that make up the refactored cluster were functioning as designed, RNA-seq and ribosome profiling experiments were performed (FIG. 18). From these data, the strengths of promoters/terminators and the transcription level and translation rates of genes could be calculated (see Methods and Materials). The performance of the promoters in R. sp. IRBG74 was systematically lower than E. coli, particularly the first promoter controlling nifH (FIG. 2E). The terminators were functioning the same in the two species, albeit weakly, and no termination could be detected from the three terminators in the center of the cluster (FIG. 2E). The translation of the genes differed significantly between organisms (FIG. 2F). When the expression rates of the nif genes from the refactored cluster are compared with their levels in their native context in K. oxytoca, there is almost no correlation (FIG. 2F). Importantly, there is 9-fold less NifH expressed from the refactored cluster in R. sp. IRBG74 as compared to the same cluster in E. coli. Thus, the refactored cluster produces wildly different expression levels of the component genes when transferred between organisms, even when transcription is matched between them using different controllers.

Based on these results, a new refactored cluster (v3.2) (FIG. 2G) was designed. A very strong promoter was chosen for nifH. The transcription was broken up by adding promoters to divide nifENX and nif and selecting stronger terminators. Noting that the expression ratios between nif genes are better preserved when the native cluster is transferred to a new host (FIG. 1D) but not the refactored cluster (FIG. 2F), it was hypothesized that this could be due to the disruption of the operon structures and the associated translational coupling between genes. The K. oxytoca operons were cloned intact, including native RBSs and replaced these regions of the refactored cluster (FIG. 2G). Note that this also preserves nifT and nifX, which were not included in first versions because they were either inessential (Simon, H. M., Homer, M. J. & Roberts, G. P. J. J. o. b. Perturbation of nifT expression in Klebsiella pneumoniae has limited effect on nitrogen fixation. 178, 2975-2977 (1996)) or inhibitory (Gosink, M. M., Franklin, N. M. & Roberts, G. P. J. J. o. b. The product of the Klebsiella pneumoniae nifX gene is a negative regulator of the nitrogen fixation (nif) regulon. 172. 1441-1447 (1990)).

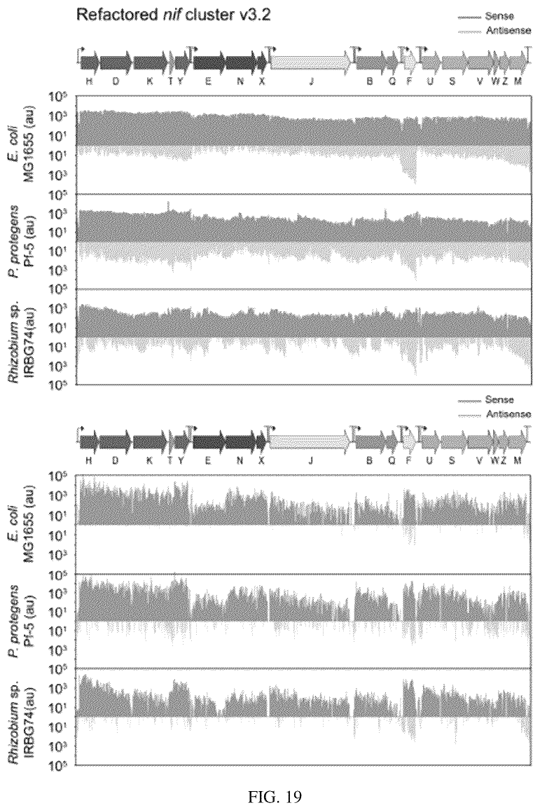

Compared to v2.1, the v3.2 cluster is less active in E. coli but is active in R. sp. IRBG74 (FIG. 2H) and P. protegens Pf-5 (FIG. 17). This experiment was performed in the double nif knockout strain in R. sp. IRBG74, thus indicating that the refactored cluster is self-contained in producing nitrogenase activity. RNA-seq and ribosome profiling was applied to evaluate the performance of v3.2 in all three species (FIG. 2I, FIG. 19, and FIGS. 20A-20F). The promoters perform similarly in the different hosts, but there was significant diversity in terminator function. Despite this, the translation rates (RD) of the genes were remarkably consistent and NifH expression is nearly identical (FIG. 2J). The higher expression of NifH and the preserved ratios between proteins is the likely reason that the refactored cluster is functional in R. sp. IRBG74. The next attempt was to increase expression level of the nif genes in R. sp. IRBG74 by increasing the concentration of inducer used, but a clear optimum beyond which increased expression caused a rapid decline in activity was found (FIG. 2M). This indicates a potential upper limit in obtaining activity in R. sp. IRBG74 under free living conditions using only the genes from K. oxytoca.

Replacement of A. caulinodans Nif Regulation with Synthetic Control

The A. caulinodans nif genes are distributed across three clusters in different genomic locations. The regulatory signals converge on the NifA activator that, in concert with the RpoN sigma factor, turns on transcription of the genomic nif clusters. Numerous and not fully characterized environmental signals are integrated upstream of this node, including NtrBC (Kaminski, P. A. & Elmerich, C. J. M. m. The control of Azorhizobium caulinodans nifA expression by oxygen, ammonia and by the HF-I-like protein, NrfA. 28, 603-613 (1998)), NtrXY (Pawlowski, K., Klosse, U., De Bruijn, F. J. M. & MGG, G. G. Characterization of a novel Azorhizobium caulinodans ORS571 two-component regulatory system, NtrY/NtrX, involved in nitrogen fixation and metabolism. 231, 124-138 (1991), FixLJK (Kaminski, P. & Elmerich, C. J. M. m. Involvement of fixLJ in the regulation of nitrogen fixation in Azorhizobium caulinodans. 5, 665-673 (1991); Kaminski, P., Mandon, K., Arigoni, F., Desnoues, N. & Elmerich, C. J. M. m. Regulation of nitrogen fixation in Azorhizobium caulinodans: identification of a fixK-like gene, a positive regulator of nifA. 5, 1983-1991 (1991)), NrfA (Kaminski, P. A. & Elmerich, C. J. M. m. The control of Azorhizobium caulinodans nifA expression by oxygen, ammonia and by the HF-I-like protein, NrfA. 28, 603-613 (1998)), and PII proteins (e.g., GlnB and GlnK (Michel-Reydellet, N. & Kaminski. P. A. J. J. o. b. Azorhizobium caulinodans Plland GlnK proteins control nitrogen fixation and ammonia assimilation. 181, 2655-2658 (1999))). Tthe clusters (64 kb total, containing 76 genes) were cloned into the plasmid systems described above and transferred into R. sp. IRBG74 and P. protegens Pf-5, but no activity was found in either strain. Overexpression of A. caulinodans NifA and RpoN did not lead to activity and, upon further investigation, these regulators were found to be inactive in these strains. The size of the clusters and the lack of genetic and gene function information would complicate fully refactoring the system. For these reasons, it was decided to modify the regulation controlling nif such that it can be placed under the control of synthetic sensors.

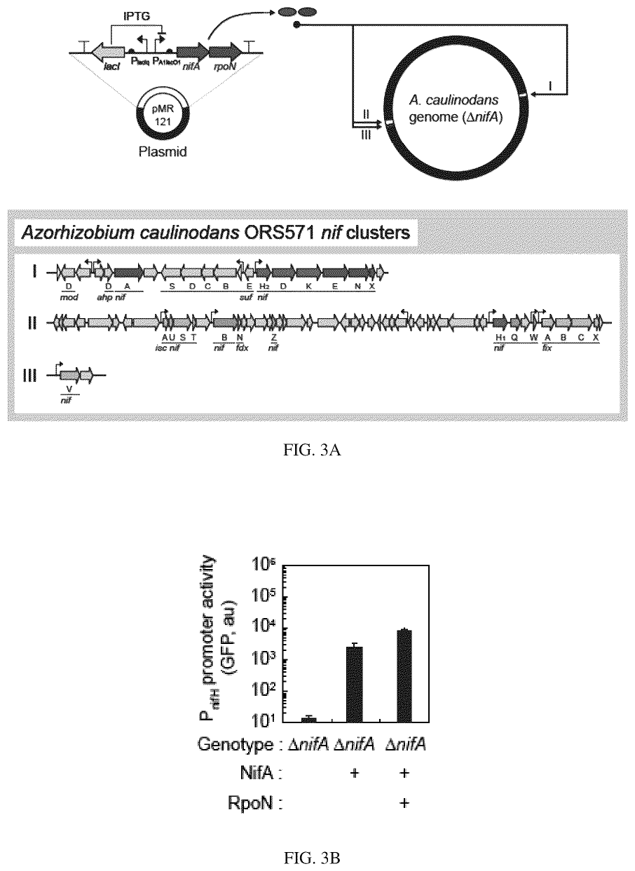

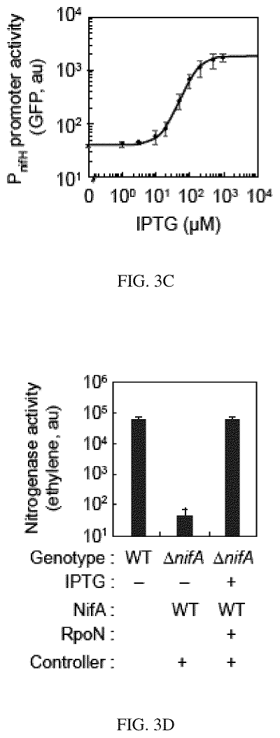

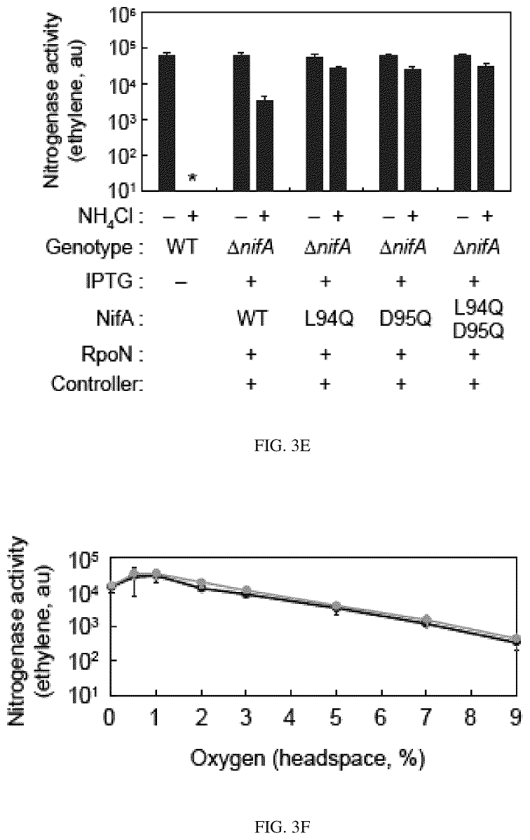

The primary goal herein was to eliminate ammonium repression of nitrogenase activity, which converges on the regulation of NifA. The native nifA gene was knocked out of the genome using the sacB markerless deletion method (see Methods and Materials), with the intent of placing NifA under inducible control (FIG. 3A). There is only basal activity from the nifH promoter in the ΔnifA strain (FIG. 3B). When NifA is overexpressed, the promoter turns on and its activity is further enhanced by the co-expression of RpoN in an operon (note that the genomic rpoN gene is left intact for these experiments). The IPTG-inducible system designed for Rhizobium (previous section) was tested in A. caulinodans carried on a pBBR1-ori plasmid. Using GFP, this was found to induce expression over several orders of magnitude (FIG. 21). Then, the A. caulinodans nifA and rpoN gene was placed under IPTG control and the fluorescent reporter fused to the A. caulinodans nifH promoter (encompassing 281 nt upstream of the ATG), carried on the same plasmid (see Materials and Methods). The response function from the nifH promoter was analyzed at the condition used for nitrogen fixation, exhibiting a wide dynamic range to 45-fold (FIG. 3C).

The controller was designed to co-express NifA and RpoN and tested for its ability to induce nitrogenase (FIG. 3D). When fully induced, there is a complete recovery of activity as compared to the wild-type strain. The repression of nitrogenase activity by ammonium was then evaluated. The presence of 10 mM ammonium chloride leads to no detectible activity by the wild-type strain (FIG. 3E). Even when both NifA and RpoN are under inducible control, there is strong repression with only 5% of the nitrogenase activity of the wild-type. This suggests that the post-transcriptional control of NifA activity by ammonium remains intact.

In related alphaproteobacteria, mutations have been identified in NifA that abrogate ammonium repression (Paschen, A., Drepper, T., Masepohl, B. & Klipp, W. Rhodobacter capsulatus nifA mutants mediating nif gene expression in the presence of ammonium. FEMS microbiology letters 200, 207-213 (2001); Rey, F. E., Heiniger, E. K. & Harwood, C. S. Redirection of metabolism for biological hydrogen production. Applied and environmental microbiology 73, 1665-1671 (2007)). These mutations occur in the N-terminal GAF domain. Using a multiple sequence alignment, two equivalent residues were identified to mutate in A. caulinodans (L94Q and D95Q) (FIG. 22). These mutations were made and then tested individually and in combination (FIG. 3D). When the double mutant of NifA is co-expressed with RpoN, the presence of ammonium only results in a slight decrease in activity.

Oxygen irreversibly inhibits nitrogenase and represses nif clusters. The inducible nif clusters were tested for oxygen sensitivity, noting that A. caulinodans is an obligate aerobe and fixes nitrogen under micro-aerobic conditions. The tolerance of nitrogenase to oxygen was then assessed as a function of the concentration of oxygen in the headspace, held constant by injecting oxygen while monitoring its level (Methods and FIG. 26A). The native and inducible gene clusters responded nearly identically to oxygen (FIG. 3F). The optimum activity occurs between 0.5% to 1% with a wide tolerance (30% activity at 3% oxygen).

Introduction of Controllable Nif Activity in P. protegens Pf-5

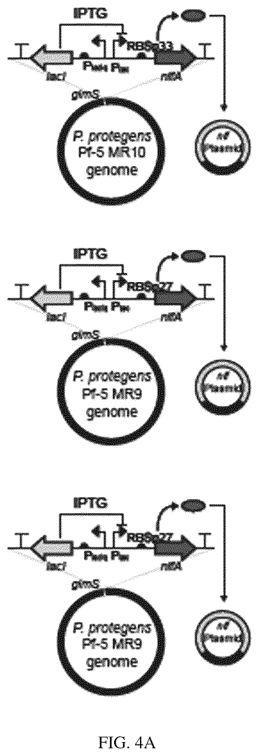

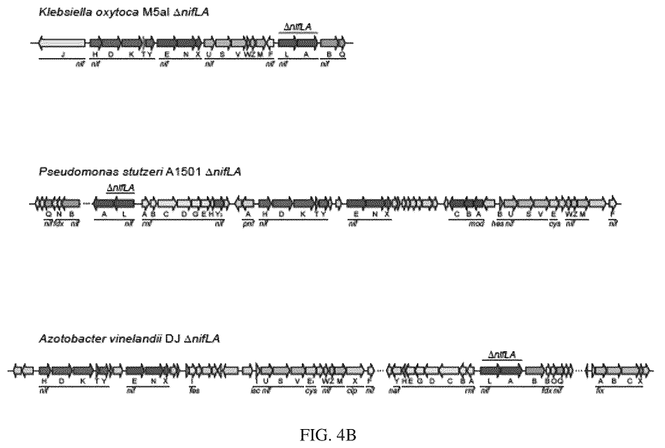

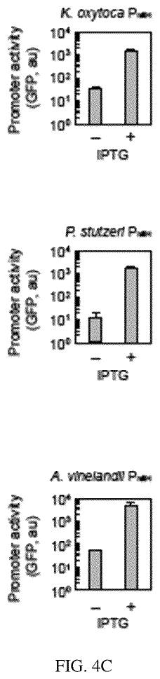

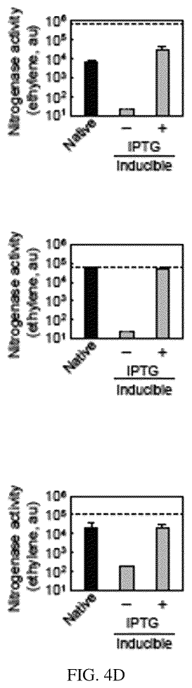

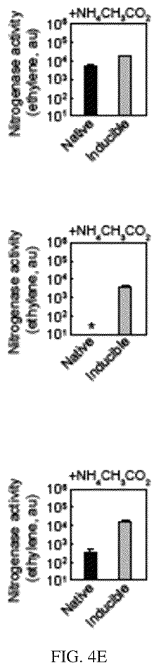

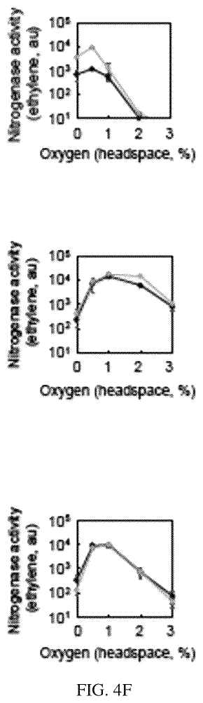

The native K. oxytoca, P. stutzeri, and A. vinelandii nif clusters are all functional in P. protegens Pf-5 (FIG. 1A). However, when the native P. stutzeri and A. vinelandii clusters are transferred, nitrogenase is strongly repressed. In contrast, transferring the native K. oxytoca cluster produces uncontrolled (constitutively on) nitrogenase activity (FIG. 4E). For these three clusters in P. protegens Pf-5, it was sought to gain regulatory control by removing the nifA master regulators from the clusters and expressing them from a controller (FIG. A).

As with Rhizobia, it was found that first, part libraries for P. protegens Pf-5 had to be built before building controllers with sufficient dynamic range. A range of 20 constitutive promoters and seven T7 promoters that span a range of 778-fold and 24-fold expression, respectively, was characterized (FIGS. 11A-11C). A library of 192 RBSs was screened, representing an expression range of 4,079-fold (FIGS. 12A-12B). A set of seven terminators that share no sequence homology between each other and have a terminator strength >10 in R. sp. IRBG74 was selected and characterized together with the three well-used terminators (e.g., T7 terminator, rrnBT1, and L3S2P21). These seven terminators showed a terminator strength >50 (FIGS. 13A-13B).

The inducible systems designed for Rhizobium were transferred as-is to a Pseudomas-specific pRO1600 plasmid (see Methods and Materials). The 3OC6HSL-, aTe-, cuminic acid-, and DAPG-inducible systems were all found to be functional (FIG. 15A). In addition, a naringenin-inducible system based on the P_{fde} promoter was constructed and found to be functional. The strength of arabinose inducible system was increased by substituting the −10 box in P_{BAD} promoter and arabinose import was improved by constitutive expression of the arabinose transporter AraE (FIG. 15B). Finally, the IPTG-inducible system was optimized for P. protegens Pf-5 by replacing the P_{A1lacO1} promoter with the P_{tac} promoter and making three amino acid substitutions to LacI (Meyer, A. J., Segall-Shapiro, T. H., Glassey, E., Zhang, J. & Voigt. C. A. J. N. c. b. Escherichia coli “Marionette” strains with 12 highly optimized small-molecule sensors. 1 (2018).). This effort resulted in seven new inducible systems that produce 41- to 554-fold induction in P. protegens Pf-5 (FIG. 15C).

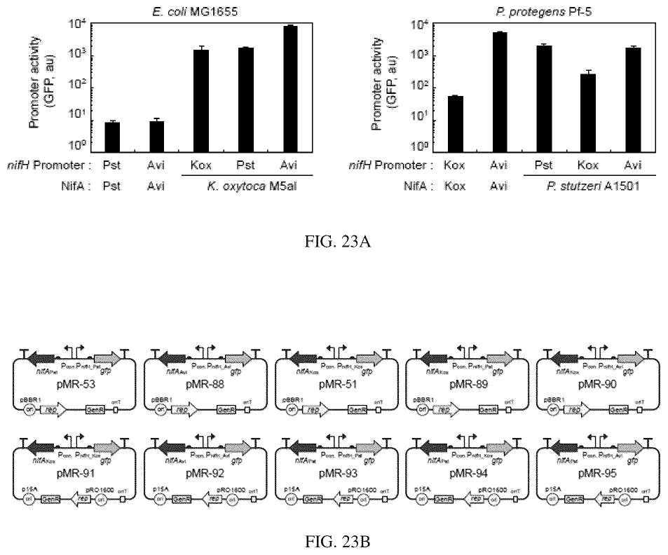

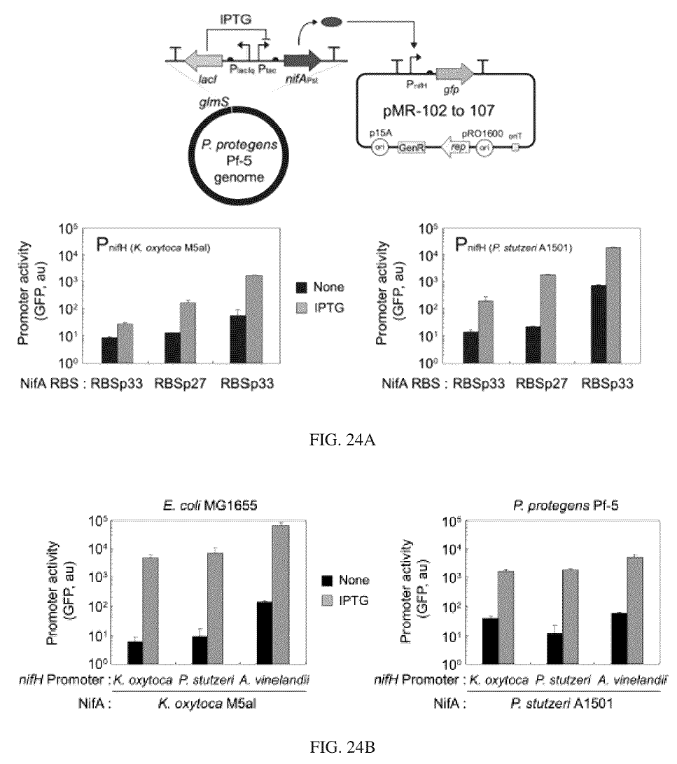

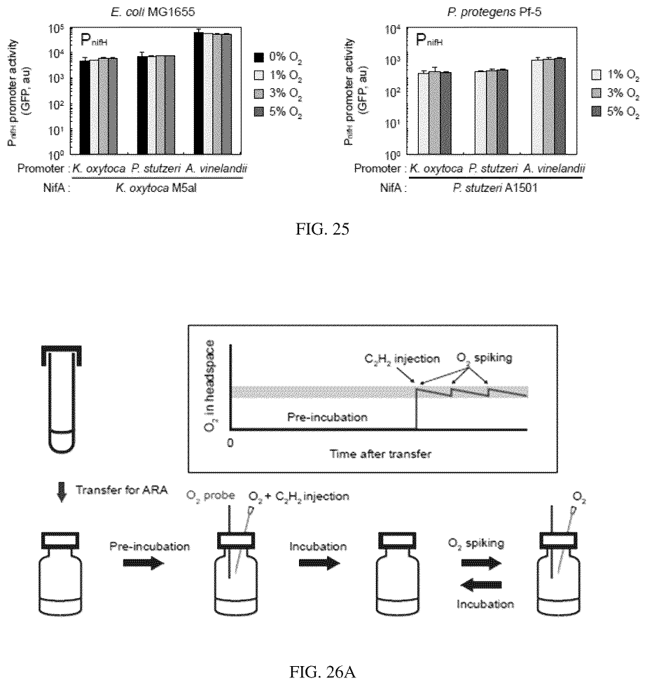

To simplify the comparison between clusters, it was sought to build a single, universal controller that could induce all three. Each has a different NifA sequence, so the ability to cross induce the gene clusters was tested. To do this, the nifH promoters from each nif cluster were cloned and fused to gfp to build plasmid-based reporters (see Methods and Materials). The ability of the various NifA homologues to activate the nifH promoters was evaluated in E. coli and P. protegens Pf-5 (FIG. 23A-23B). The results suggest that it is more important to express a NifA variant from a similar species as the host, as opposed to expressing the NifA variant that is cognate to the transferred cluster. This may be due to the need for NifA to recruit host transcriptional machinery, whereas the NifA binding sites in the promoters are well conserved across species. Based on these data, the controller was constructed using the P. stutzeri NifA, placed under the control of the optimized IPTG-inducible system, described above. The RBSs of NifA were synthetically designed to span a wide range of expression of nif genes (FIG. 24A). The controller was inserted into the genome 25 bp downstream of the stop codon of glmS using the mini-Tn7 system. The ability for this controller to induce the nifH promoter from each cluster using a fluorescent reporter is shown in FIG. 4C and FIG. 24B.

The nitrogenase activity for each of the gene clusters in P. protegens Pf-5 was then assessed (FIG. 4D). The three P. protegens Pf-5 strains containing the transferred clusters were modified to insert the controller and delete the native nifLA genes from each cluster (FIG. 4B). All three are inducible, with nitrogenase activity showing dynamic ranges of 1,200-fold, 2,300-fold, and 130-fold for the K. oxytoca, P. stutzeri, and A. vinelandii nif clusters, respectively. When induced, these systems all produce similar or even higher nitrogenase activities than can be achieved by the transfer of the unmodified native clusters (FIG. 4D). For reference, the nitrogenase activities produced by K. oxytoca, P. stutzeri, and A. vinelandii are shown as dashed lines in FIG. 4D (top to bottom) (see Methods and Materials). All three inducible clusters produce similar levels of activity that approach those measured from wild-type P. stutzeri and A. vinelandii.

The native P. stutzeri and A. vinelandii clusters are strongly repressed by ammonium: the presence of 17.1 mM eliminates activity or reduces it 7-fold, respectively (FIG. 4E and FIGS. 8A-8B). The inducible clusters show little reduction in activity and the inducible A. vinelandii cluster exhibits almost no ammonia repression. While the native K. oxytoca cluster in P. protegens Pf-5 generates a constitutive response, there is still some repression, which is reduced by the inducible version.

The inducible nif clusters were tested for oxygen sensitivity. Note that wild-type A. vinelandii is able to fix nitrogen under ambient conditions due to genetic factors internal and external to the cluster. First, it was established that the controller in P. protegens Pf-5 could induce transcription from the three nifH promoters in the presence of oxygen (FIGS. 26A-26B). The tolerance of nitrogenase to oxygen was then assessed as a function of the concentration of oxygen in the headspace, as described for A. caulinodans (previous section). The native and inducible clusters exhibited the same oxygen response (FIG. 4F). The nif cluster from K. oxytoca was the most sensitive, generating the highest activity under anaerobic conditions, but this is quickly abolished in the presence of 02. In contrast, the nif clusters from P. stutzeri and A. vinelandii showed wider tolerance with optima at 1% and 0.5%, respectively. However, both clusters lose activity at lower oxygen concentrations than A. caulinodans.

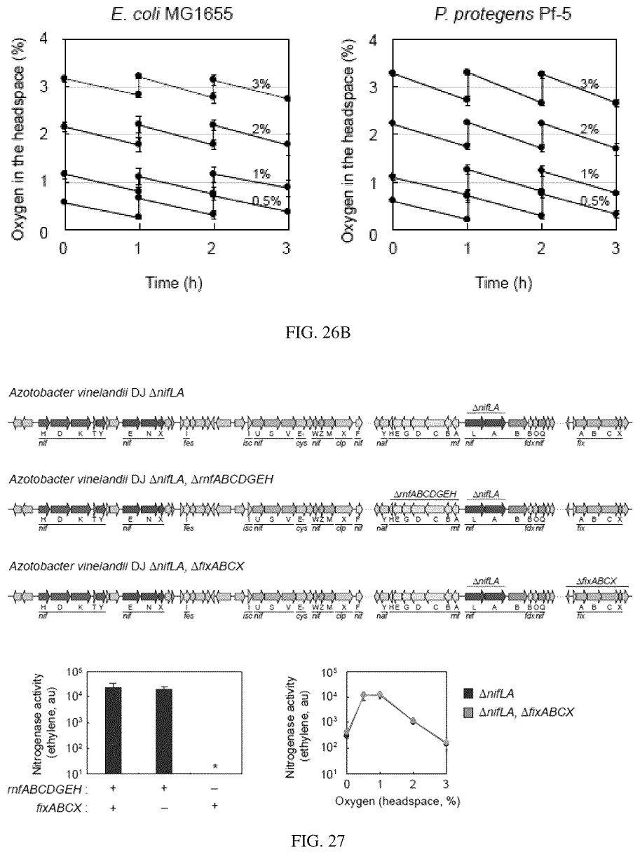

To explore the impact of the electron transport chains, several mutants to the A. vinelandii cluster were made (FIG. 27). The A. vinelandii cluster contains two potential electron transport systems to nitrogenase and the redundant system may help maintain redox status for nitrogenase at various oxygen levels. The dependence of nitrogenase activity on the oxygen concentration in various mutant backgrounds was re-measured. No effect was seen by adding the rnf2 operon or deleting the fix operon, however deleting rnf1 eliminated activity. This suggests that the rfl operon is the sole source of electrons in P. protegens Pf-5 under these conditions and the Fix complex cannot compensate the Rnf complex unlike the case of A. vinelandii.

Control of Nitrogen Fixation with Agriculturally-Relevant Sensors

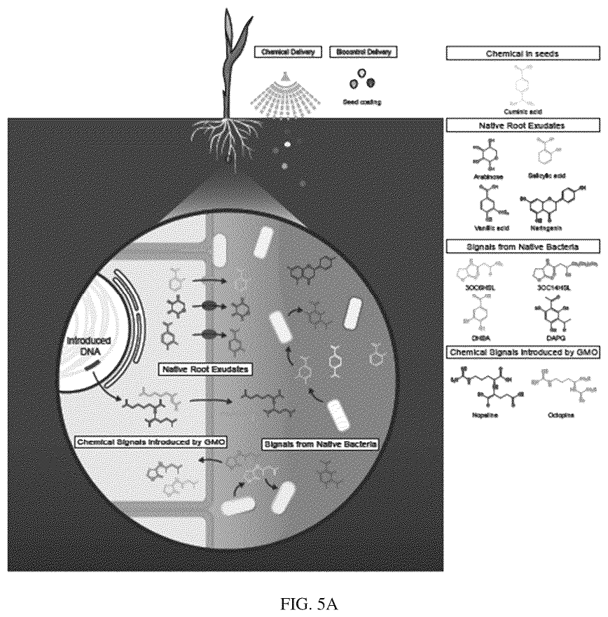

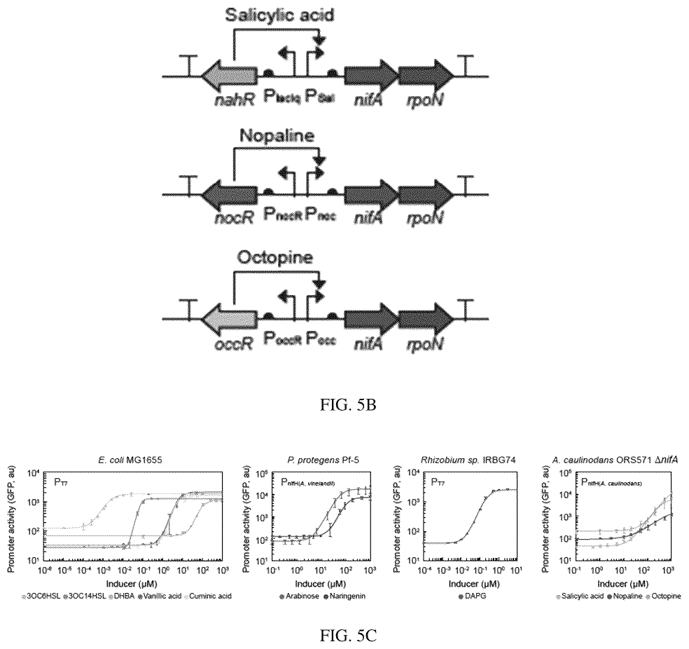

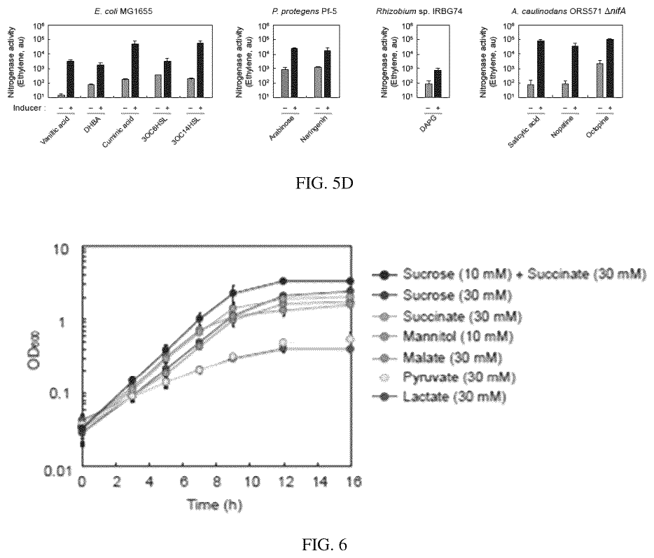

The careful design and characterization of the controller has the benefit of simplifying the process by which different synthetic sensors are used to induce nitrogenase expression. By knowing the dynamic range required to go from inactive to active nitrogenase, one can quantitatively select sensors that have the produce a compatible response. This allows different environmental signals—or combinations of signals using genetic logic circuits—to be used to control expression. To demonstrate this, 11 synthetic sensors were selected that respond to a variety of chemical signals of relevance to the rhizosphere and demonstrate that these can be used to create inducible nitrogenase in our engineered strains of E. coli (carrying the refactored v2.1 nif), R. sp. IRBG74 (carrying the refactored v3.2 nif), P protegens Pf-5 (carrying the inducible A. vinelandii nif), and A. caulinodans (inducible nifA/rpoN) (FIGS. 5A-5D).

The roles of the chemical signals in the rhizosphere are shown in FIG. 5A. Cuminic acid is present in plant seeds and functions as a fungicide. Natural root exudates may include sugars, amino acids, organic acids, phenolic compounds, phytohormones, and flavonoids. These represent potential signals to control nitrogenase production close to the root surface. Cereals have been shown to release arabinose, vanillic acid, and salicylic acid. In addition, salicylic acid regulates the plant innate immune response and the impact of its exogenous addition to cereals has been studied. Naringenin is a common precursor for many flavonoids and improves endophytic root colonization when applied to rice and wheat. Genistein, a product from naringenin catalyzed by the isoflavone synthase, is released from maize roots. A quorum sensing mimic released by rice can regulate the 3OC6HSL receptor protein LuxR, which has been visualized using E. coli biosensor strains.

Bacteria either native to the rhizome or added as biocontrol agents introduced as a spray inoculant or seed coating produce chemical signatures. Inoculation of cereals with root colonizing Pseudomonas strains that produce DAPG elicits protection against fungal pathogens. Many bacteria produce quorum molecules, such as N-acyl homoserine lactones, as a means of communication and plants can respond to these signals2. The bacterium Sinorhizobium meliloti produces 3OC14HSL, which enhances Medicago nodulation and has been shown to induce systemic resistance in cereals. DHBA can be produced by root colonizing bacteria to increase iron solubility and play a role as a chemoattractant for Agrobacterium and Rhizobium.

Sensors for these chemicals were constructed based on the controllers for each species. For E. coli MG1655, a strain that contains 12 optimized sensors, carried in the genome, that respond to various small molecules (“Marionette”) had been previously constructed (Meyer, A. J., Segall-Shapiro, T. H., Glassey, E., Zhang, J. & Voigt, C. A. J. N. c. b. Escherichia coli “Marionette” strains with 12 highly optimized small-molecule sensors. 1 (2018).). The response functions of these sensors were characterized in standard units, making it simple to identify those that can be connected to nitrogenase expression without further tuning. Marionette contains sensors for vanillic acid, DHBA, cuminic acid, 3OC6HSL, and 3OC14HSL. For each sensor, the output promoter was transcriptionally fused to T7 RNAP and the response of the responsive promoter (PT7) was measured as a function of inducer concentration (FIG. 5B and FIG. 28B). Then, the v2.1 refactored nif cluster was introduced and nitrogenase activity was measured in the presence and absence of inducer (FIG. 5C and FIG. 28C). The inducible systems constructed for P. protegens Pf-5 that respond to arabinose and naringenin were used to drive NifA expression for the control of the A. vinelandii nif cluster (FIG. 4A). The induction of the nifH promoter by these sensors was first confirmed using a reporter (FIG. 5B). When this is replaced with the nif gene cluster, it results in an inducible response of nitrogenase activity (FIG. 5C). The best nitrogenase activity in R. sp. IRBG74 is low; however, herein it was demonstrated that it could be placed under inducible control. The DAPG-inducible system developed for R. sp. IRBG74 was connected to the control of T7 RNAP and this produces a strong response from PT7 (FIG. 5B). However, when used to drive the expression of the v3.2 refactored pathway, only a 9-fold induction is observed, consistent with the low nitrogenase activity observed in this strain (FIG. 5C). Finally, the salicylic acid sensor designed for Rhizobium was used to control NifA (L94Q/D95Q)/RpoN expression in A. caulinodans (FIG. 3A and FIG. 5B). This yielded a 1000-fold dynamic range of nitrogenase activity (FIG. 5C).

Plants could be engineered to release an orthogonal chemical signal that could then be sensed by a corresponding engineered bacterium. This would have the benefit of only inducing nitrogenase in the presence of the engineered crop. Further, if the molecule is metabolizable by the engineered bacterium, it could serve as a mechanism around which a synthetic symbiosis could be designed, where the plant provides the carbon and the bacterium fixed nitrogen in an engineered relationship. To this end, legumes and Arabidopsis have been engineered to produce opines, including nopaline and octopine. Sensors were constructed for these two opines for A. caulinodans based on the LysR-type transcriptional activators OccR (octopine) and NocR (nopaline) and their corresponding P_{occ} and P_{noc} promoters (FIG. 5D and FIG. 21). These sensors were connected to the expression of NifA (L94Q/D95Q)/RpoN and the response from P_{nifH} was measured using a fluorescent reporter. Both response functions had a large dynamic range (FIG. 5B) and produced highly-inducible nitrogenase activity (FIG. 5C). The nopaline sensor yielded a 412-fold dynamic range and the octopine sensor led to 40% higher nitrogenase activity than the wild-type.

### DISCUSSION

Towards designing a bacterium that can deliver fixed nitrogen to a cereal crop, this work provides a side-by-side comparison of diverse species, natural nif clusters, and engineering strategies. The goal was to obtain inducible nitrogenase activity in a strain that can associate with cereals as an endophyte or epiphyte. To this end, ˜100 strains involving the transfer of 10 natural nif clusters ranging in size from 10 kb to 64 kb to 16 diverse species of Rhizobia, Azorhizobium, Pseudomas, and E. coli were constructed. Different approaches were taken to make these nif clusters inducible, from bioinformatics and protein engineering to complete genetic reconstruction from the ground-up (refactoring). In addition to the highest activity, it is important that nitrogen fixation be robust to the addition of nitrogenous fertilizer (ammonia) and microaerobic environments. Two lead candidates have emerged from this effort. The most promising endophyte is a variant of Azorhizobium where nifA is knocked out of the genome and a nifA mutant and rpoN are complemented on a plasmid. For the epiphyte P. protegens Pf-5, the most versatile strain is based on the transfer of the A. vinelandii nif cluster and placement of nifA of P. sturzeri under inducible control. In both cases, nitrogenase activities were obtained that are nearly identical to wild-type A. caulinodans and P. stutzeri, respectively. Neither showed significant repression by ammonia and optimal activity was obtained in 1% oxygen. Based on these strains, it was demonstrated that nitrogenase can be placed under inducible control in response to cereal root exudates (arabinose, salicylic acid), phytohormones (naringenin) and putitive signaling molecules that could be released by genetically modified plants (nopaline and octopine).

Because R. sp. IRBG74 can fix nitrogen in a legume nodule and also associates with rice, significant effort was directed to engineering this strain to fix nitrogen when cereal-associated. The first attempt was simply complementing nifV, as this is absent in R. sp. IRBG74 and produces a metabolite provided by the plant, but this attempt was unsuccessful. Then, it was found that all of the initial nif clusters transferred, some of which have high activity in P. protegens Pf-5 and E. coli, are non-functional in R. sp. IRBG74, which led to trying clusters from alphaproteobacteria, one of which produced a very low level of activity that was dependent on the nif genes native to R. sp. IRBG74. The previously-published refactored gene clusters based on Klebsiella nif were attempted in R. sp. IRBG74 but these showed no activity. It was only after the construction of a new refactored cluster (v3.2) that activity was obtained under free-living conditions that was not dependent on the native nif genes. This allowed an increase in the expression levels, and an optimum was discovered beyond which activity was lost. This is the first time that nif activity has been engineered in a Rhizobium under free-living conditions that could otherwise not perform this function. This sets the foundation for further development and optimization of this strain.

The present disclosure encompasses different degrees of nif pathway re-engineering to promote heterologous transfer. The most ambitious is the complete refactoring of all the nif genes and regulation, where all regulatory genetic parts are replaced, genes are recoded, operons are reorganized, and transcription is performed by the orthogonal T7 RNAP. When this project was initiated, DNA synthesis was a novelty and a lack of DNA assembly methods made it difficult to make alternative designs. Further, the evaluation of performance relied on the overall nitrogenase activity, rather than an understanding of the underlying parts. As such. the first refactored pathway performed poorly. In subsequent studies, better part libraries and DNA assembly and automation platforms enabled the synthesis of many variants. Further, as the cost of RNA-seq declined, it was used to evaluate the performance of internal parts, such as promoters and terminators. This revealed that the first designs were effectively large single operons with little differential control over the transcription levels of individual genes. With these techniques allowed the optimization of the function of the refactored nif pathway and the discovery that many of the underlying genetic structure were not needed to achieve high activities.

In the present disclosure, ribosome profiling, a new technique that enables the measurement of translational parts (e.g., ribosome binding sites), was applied and expression levels were inferred. Further, nitrogenase activity and the function of underlying parts were assessed as the clusters were moved between species. Interestingly, the native Klebsiella nif cluster could be transferred and it performed similarly but the refactored cluster yielded widely varying expression levels in the different hosts, sometimes leading to a total loss in activity. This could be recovered by maintaining the native operon structure in the refactored cluster, implying that it was not due to the synthetic sensors, T7 RNAP, or promoters/terminators. This is one of the hypothesized functions of operons. Achieving this required maintenance of the codon usage and translational coupling of the native cluster. However, this does not mean that it will not be possible to also encode this function synthetically. There have been computational advances that enable the calculation of RBSs internal to upstream genes when encoded on an operon. If coupled with codon optimization algorithms, this would allow the design of de novo genetic parts that achieve a desired degree of translational coupling and expression level.

This work herein is the first step of a larger effort to build strains that can efficiently deliver fixed nitrogen to cereals. The present disclosure demonstrates the deregulation of nif clusters in A. caulinodans and P. protegens Pf-5, enabling them to be placed under the control of cereal root exudates. This derepresses the pathway in the presence of exogenous nitrogenous fertilizer—critical for the use of the bacterium as part of an integrated agricultural solution. Further, these organisms retain the ability to fix nitrogen in microaerobic environments, thus avoiding the need for a root nodule that enforces strict anaerobiosis. The complete deregulation of the nif pathway makes the bacterium non-competitive in the soil and lost quickly, thus limiting its impact to particular phases of the growth cycle. Thus, it is demonstrated that nitrogenase can be placed under the control of chemical root exudates. Fully realizing the goal of engineering microbial delivery to a cereal will require significant additional genetic engineering to maximize their ability to catabolize carbon sources from the plant and increase the flux of fixed nitrogen delivery by redirecting metabolism, introducing transporters, and the optimization of electron transfer. An intriguing possibility is to also genetically engineer the plant to produce orthogonal carbon sources, such as opines or less common sugars, and then placing the corresponding catabolism pathways into the bacterium.

### EMBODIMENTS

1. A rhizobium that can fix nitrogen under aerobic free-living conditions, comprising a symbiotic rhizobium having an exogenous nif cluster, wherein the exogenous nif cluster confers nitrogen fixation capability on the symbiotic rhizobium under aerobic free-living conditions, and wherein the rhizobium is not Azorhizobium caulinodans.

2. The rhizobium of paragraph 1, wherein the exogenous nif cluster is from a free-living diazotroph.

3. The rhizobium of paragraph 1, wherein the exogenous nif cluster is from a symbiotic diazotroph.

4. The rhizobium of paragraph 1, wherein the exogenous nif cluster is from a photosynthetic Alphaproteobacteria.

5. The rhizobium of paragraph 1, wherein the exogenous nif cluster is from a Gammaproteobacteria.

6. The rhizobium of paragraph 1, wherein the exogenous nif cluster is from a cyanobacteria.

7. The rhizobium of paragraph 1, wherein the exogenous nif cluster is from a firmicutes.

8. The rhizobium of paragraph 1, wherein the exogenous nif cluster is from Rhodobacter sphaeroides.

9. The rhizobium of paragraph 1, wherein the exogenous nif cluster is from Rhodopseudomonas palustris.

10. The rhizobium of paragraph 1, wherein the exogenous nif cluster is an inducible refactored nif cluster.

11. The rhizobium of paragraph 10, wherein the inducible refactored nif cluster is an inducible refactored Klebsiella nif cluster.

12. The rhizobium of any one of the preceding paragraphs, wherein the rhizobium is IRBG74.

13. The rhizobium of any one of the preceding paragraphs, wherein the exogenous nif cluster comprises 6 nif genes.

14. The rhizobium of paragraph 13, wherein the 6 nif genes are nifHDK(T)Y, nifEN(X), nifJ, nifBQ, nifF, and nifUSVWZM.

15. The rhizobium of paragraphs 13 or 14, wherein each nif gene of the exogenous nif cluster is preceded by a T7 promoter.

16. The rhizobium of paragraph 15, wherein the T7 promoter is a wild-type promoter.

17. The rhizobium of any one of the preceding paragraphs, further comprising an endogenous nif cluster.

18. The rhizobium of any one of the preceding paragraphs, wherein the nif cluster has a nifV gene.

19. The rhizobium of paragraph 18, wherein the ni/V gene is endogenous.

20. The rhizobium of any one of the preceding paragraphs, wherein the exogenous nif cluster further comprises a terminator.

21. The rhizobium of any one of paragraphs 15-20, wherein the T7 promoter has a terminator and wherein the terminator is downstream from the T7 promoter.

22. The rhizobium of paragraph 12, wherein the exogenous nif cluster is a refactored rhizobium IRBG74 nif cluster.

23. A plant growth promoting bacterium that can fix nitrogen under aerobic free-living conditions, comprising a bacterium having an exogenous nif cluster having at least one inducible promoter, wherein the exogenous nif cluster confers nitrogen fixation capability on the bacterium, under aerobic free-living conditions, and wherein the bacterium is not Azorhizobium caulinodans.

24. The plant growth promoting bacterium of paragraph 23, wherein the bacterium is a symbiotic bacterium.

25. The plant growth promoting bacterium of paragraph 23, wherein the bacterium is an endophyte.

26. The plant growth promoting bacterium of paragraph 25, wherein the endophyte is rhizobium IRBG74.

27. The plant growth promoting bacterium of paragraph 23, wherein the bacterium is an epiphyte.

28. The plant growth promoting bacterium of paragraph 27, wherein the epiphyte is pseudomonas protogens PF-5.

29. The plant growth promoting bacterium of any one of paragraphs 23-28, wherein the plant growth promoting bacterium is associated with a genetically modified cereal plant.

30. The plant growth promoting bacterium of paragraph 29, wherein the genetically modified cereal plant includes an exogenous gene encoding a chemical signal.

31. The plant growth promoting bacterium of paragraph 29, wherein the nitrogen fixation is under the control of the chemical signal.

32. The plant growth promoting bacterium of paragraphs 30 or 31, wherein the chemical signal is opine, phlorogluconol or rhizopene.

33. The rhizobium of any one of paragraphs 23-32, wherein the exogenous nif cluster comprises 6 nif genes.

34. The rhizobium of paragraph 33, wherein the 6 nif genes are nifHDK(T)Y, nifEN(X), nifJ, nifBQ, nifF, and nifUSVWZM.

35. The rhizobium of any one of paragraphs 23-34, wherein the inducible promoter is a T7 promoter.

36. The rhizobium of any one of paragraphs 23-34, wherein the inducible promoter is P_{A1lacO1} promoter.

37. The rhizobium of any one of paragraphs 23-36, wherein the inducible promoter is activated by an agent selected from a group that includes IPTG, sodium salicylate, octapine, nopaline, the quorum signal 3OC6HSL, aTe, cuminic acid, DAPG, and salicylic acid.

38. The rhizobium of any one of paragraphs 23-37, wherein the exogenous nif cluster further comprises a terminator.

39. The rhizobium of any one of paragraphs 23-37, wherein the inducible promoter has a terminator and wherein the terminator is downstream from the inducible promoter.

40. An Azorhizobium caulinodans capable of inducible ammonium-independent nitrogen fixation in a cereal crop, comprising:
(i) a modified nif cluster, wherein an endogenous nifA gene is deleted or altered; and
(ii) at least one operon comprising nifA and RNA polymerase sigma factor (RpoN), wherein the operon comprises a regulatory element including an inducible promoter.

41. The Azorhizobium caulinodans of claim 40, wherein the inducible promoter is P_{A1lacO1} promotor.

42. The Azorhizobium caulinodans of paragraphs 40 or 41, wherein the inducible promoter is activated by an agent selected from IPTG, sodium salicylate, octapine, nopaline, the quorum signal 3OC6HSL, aTc, cuminic acid, DAPG, and salicylic acid.

43. The Azorhizobium caulinodans of any one of paragraphs 40-42, wherein the endogenous nifA gene is altered with at least one of the following substitutions:
(i) L94Q;
(ii) D95Q; and
(iii) both L94Q and D95Q.

44. A method of engineering a rhizobium that can fix nitrogen under aerobic free-living conditions, comprising transferring an exogenous nif cluster to a symbiotic rhizobium, wherein the exogenous nif cluster confers nitrogen fixation capability on the symbiotic rhizobium, under aerobic free-living conditions, and wherein the rhizobium is not Azorhizobium caulinodans.

45. The method of paragraph 44, wherein the exogenous nif cluster comprises 6 nif genes.

46. The method of paragraph 45, wherein the 6 nif genes are nifHDK(T)Y, nifEN(X), nifJ, nifBQ, nifF and nifUSVWZM.

47. The method of paragraph 45 or 46, wherein each of the nif genes is preceded by a wild-type T7 promoter.

48. The method of any one of paragraphs 44-47, wherein the exogenous nif cluster is transferred to the rhizobium in a plasmid.

49. The method of any one of paragraphs 44-48, wherein the exogenous nif cluster further comprises a terminator.

50. The method of any one of paragraphs 47-49, wherein the wild-type T7 promoter has a terminator, and wherein the terminator is downstream from the wild-type T7 promoter.

51. The method of any one of paragraphs 44-50, wherein the endogenous NifL gene is deleted.

52. A method of producing nitrogen for consumption by a cereal plant, comprising providing a plant growth promoting bacterium that can fix nitrogen under aerobic free-living conditions in proximity of the cereal plant, wherein the plant growth promoting bacterium is a symbiotic bacterium having an exogenous nif cluster, wherein the exogenous nif cluster confers nitrogen fixation capability on the symbiotic bacterium, enabling nitrogen fixation under aerobic free-living conditions.

53. The method of paragraph 52, wherein the plant growth promoting bacterium is a rhizobium.

54. The method of paragraph 52, wherein the plant growth bacterium is the bacterium of any one of paragraphs 1-22 and 23-39.

55. The method of any one of paragraphs 52-54, wherein the cereal plant is a genetically modified cereal plant.

56. The method of paragraph 55, wherein the genetically modified cereal plant includes an exogenous gene encoding a chemical signal.

57. The method of paragraph 56, wherein the nitrogen fixation is under the control of the chemical signal.

58. The method of paragraph 56 or 57, wherein the chemical signal is opine, phlorogluconol or rhizopene.

59. The method of any one of paragraphs 52-55, wherein the nitrogen fixation is under the control of a chemical signal.

60. The method of paragraph 57 or 59, wherein the chemical signal is a root exudate, biocontrol agent or phytohormone.

61. The method of paragraph 60, wherein the root exudate is selected from the group consisting of sugars, hormones, flavonoids, and antimicrobials.

62. The method of paragraph 57 or 59, wherein the chemical signal is vanillate.

63. The method of paragraph 57 or 59, wherein the chemical signal is IPTG, aTc, cuminic acid, DAPG, and salicylic acid, 3,4-dihydroxybenzoic acid, 3OC6HSL or 3OC14HSL.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”. when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

### Additional Tables

TABLE 7
Primers used for nif cluster cloning.

| | Forward primer | Reverse Primer | | GenBank |
|---|---|---|---|---|
| Nif cluster | (SEQ ID NOs: 8-64) | (SEQ ID NOs: 65-121) | Genomic location | accession No. |
| Klebsiella | CGTAGGGCGCATTAATGCAGCTGGCACGACAGGT | GTGACGCTCGCGTATCAGGTTTG | 3,897,443-3,909,294 | CP020657.1 |
| oxytoca | GAATTCTAGACTGCTGGATACGCTGCTTAAGGTC | | | |
| M5al | TACGCTGTTTGAGCTGGCAAACCT | ATCAGGCGCATATTTGAATGTAT | 3,909,255-3,920,878 | CP020657.1 |
| | | TTACTGCAGCGGCCGCTTCTAG | | |
| | | AGTGACCAAAAGCTTCCGCAACCC | | |
| Pseudomonas | GCCCGGAGAGCAAGCCCGTAGGGCGCATTAATGCAGCTGG | ACTACGCATCACTAGCAGGGCACG | 1,410,207-1,414,229 | NC_009434 |
| stutzeri A501 | CACGACAGGTGTTAGGTTGGCCTGAATTCGGTGT | CACCGCGGACGAAATCGAAGTGAG | | |
| | GGCTCACTTCGATTTCGTCCGCGGTGCGTGCCCTGCTAGT | TTGTCGACTCCCGGGGTCTGAC | 1,419,757-1,424,637 | NC_009434 |
| | GATGCGTA | | | |
| | CGCCTGATTTCGCCTGATGAACAGG | GGCTTTAACGGCATGTTCCGGGT | 1,424,588-1,429,971 | NC_009434 |
| | TGACGCTGTTGACCACCGCC | GTAGTCGTCGTTGTGGCCGAACTC | 1,429,922-1,434,417 | NC_009434 |
| | ATGGAAGTGGTCGGCACCGGCTA | AAAGCATCATCTCGGGTCGGGC | 1,434,370-1,438,503 | NC_009434 |
| | CGCAACGGTTGGGGTAGGTTGG | CGTCGAGCGACAACGCCTCGA | 1,438,454-1,442,613 | NC_009434 |
| | GACGTCCATCGCTTCGGCTTCGA | CTATGAGCTGGACTGAACCGCGATG | 1,442,565-1,448,340 | NC_009434 |
| | CTGCGATCGACGCTGTCGAGCATCATCGCGGTTCA | GAAAATACCGCATCAGGCGCATATTG | 1,448,291-1,459,252 | NC_009434 |
| | | TGAATGTATTTACTGCAGC | | |
| | | GCCGCTGGCGAATCTCCTTCCTCGGTTCG | | |

| | | | | |
|---|---|---|---|---|
| Azotobacter | ATCCATTCTCAGGCTGTCTCGTCTCGTCTCTACGTACGCG | GCCTTCGAACATGTTGTCCCAG | 134,732-144,115 | NC_012560 |
| vinelandii DJ | GATCCCAGGCAACGTCTTCGTACTGCGGTACCGGGTTGCG | | | |
| | GGGGCAGCCAGTGGAAAAAGG | | | |
| | CTACGGCACGCCCTGGTTCGA | TCGAGTTCGAGCAGTTTCTCCAGC | 144.076-148,534 | NC_012560 |
| | GCTCGGAAAGTGCTGGAGAAAC | AGCGAACAATACCTGTGGCC | 148,500-152,895 | NC_012560 |
| | AAATCAGACATTCATGGCCACAGG | TGGCGCTTGCCCTTGTTCCAA | 152,861-157,152 | NC_012560 |
| | TCTACCATGGCGTGACTCTCGG | GCGCGGTGGTAGAGTTCCGGGAGT | 157,101-162,181 | NC_012560 |
| | | TTAAACGGACAGAAGACGAGTCGTGCGGGC | | |
| | ACTCGTCTTCTGTCCGTTTAAACTCCCGGAACTCTACCAC | TTGCTCAGGGTCGGGTTGGC | 5,161,399-5,168,611 | NC_012560 |
| | CGC | | | |
| | CTTGGATAGACGAGGCACAGC | CATCATCCTCGGCCCCTTCAGGTTGCA | 5,168,561-5,175,635 | NC_012560 |
| | | GGAGCCGGCTTG | | |
| | GCCGGCTCCTGCAACCTGAAGGGGCCGAGGATGATG | GCAAGCCACTCCACTGACGAA | 995,860-1,000,698 | NC_012560 |
| Paenibacillus | GAATTGAGGATAAATGTCAGGGATTTCATG | ACAGGTTCCGCAGTTCACAAGC | 23,686-26,413 | ALJV01.1 |
| polymyxa WLY78 | | | | contig00089 |
| | CCAAGCATTTTGAGATCGCGGATG | GCTGATTGTGATCGACAATATTCGG | 26,364-27,763 | ALJV01.1 |
| | | | | contig00089 |
| | CGGAGGTGCCGGTATGAGCGA | GAAAGCCTACACGAAGCAAAGG | 27,714-29,113 | ALJV01.1 |
| | | | | contig00089 |
| | GAAGTTTGCAGCGAAAGAGGCG | CTTGAGAATCTGCCGGGCGCCT | 29.064-30,463 | ALJV01.1 |
| | | | | contig00089 |
| | GGGATGATGCAGAATACATCCCG | ATCCACAAATCAACACCCTGCG | 30,414-31,813 | ALJV01.1 |
| | | | | contig00089 |
| | GGTGACCTGGATGATGCAGAGGAGAG | AAAGCGTTCCAGTCACGGTCAC | 31,764-34,402 | ALJV01.1 |
| | | | | contig00089 |
| Cyanothece | GGCCCGCGTTAGGTTGGCCTGAATTCGGTGTGTATCCCCC | GAGACTTTCCCCACCTTATTAT | 1,931,343-1,929,132 | NC_010546.1 |
| ATCC51142 | GGAGATACGTACCCGCCCTGTCAGGGGCG | GCATGCAGATGTTATGGGAATTA | | |
| | GGGTTTTTTTTTGATAAGTCAAGCTATCAGAACCGATC | ACG | | |
| | TAATTCCCATAACATCTGCATGCATAATAAGGTGGGGAAA | ACCTTGACAATCATTACACAGCG | 555,364-562,941 | NC_010546.1 |
| | GTCTCAGC | | | |
| | AATGTATTTCTGATCGATGCGACG | CAAATATAATGATCGACATTTTCACCAC | 562,897-570,603 | NC_010546.1 |
| | GTTATCTGGCTGATGTTTGTGGTG | CGTTAACTTTGTCGCAAAACTTCG | 570,558-577,494 | NC_010546.1 |
| | GTCAAACTGTCTTGTTTAAAGCCG | ACCAAGGCGAATCTCCTTCCTCGGTTCG | 577,449-584,687 | NC_010546.1 |
| | | CGATCACGCTACTCCGC | | |
| | | CAATAAAAAAGCCCCCGGAATGATCTTC | | |
| | | CGGGGGCCAGATTCAGGTAACTGCTCAAG | | |
| Azospirillum | TTAAGGTCATGCAGCAGGAGAACTAAAGGCCCGCGTTAGG | TGCGTCTTCTTCGGGCATCGTCA | 1.043,795-1,035,568 | CP012914 |
| brasilense Sp7 | TTGGTAATAAAAAAGCCCCCGGAATGATCTTCCGGGGGCC | | | |
| | CTGCGCAAATACAACATCGAGATC | | | |
| | GACGACTGAATAAGGATCGCGGAATG | AGAAAATTGATTGCGGACGAGCG | 1,035,614-1,027,483 | CP012914 |
| | TATGTCACAGGCCCGACAAAGCG | TTCAATAAGTTAAGCAGATCGGCCTCG | 1,027,533-1,019,166 | CP012914 |
| | GATTGTCGGGTATCGCACACGAG | CGGTGTTACGAATAAATATTTCTACGAATAGAC | 1,019,211-1,010,628 | CP012914 |
| | CGAAGGAGTTCGCCCCAGTCTATTC | GCTCCAAAAGGAGCCTTTAATTGTATCGGTTTA | 1,010,677-1,003,838 | CP012914 |
| | | TCAGCTTGCTTT | | |
| | | GTTCCGCGGGTCTCGATACAACG | | |
| Rhodopseudomonas | AATACGATCGCATGTCCTAGGTAATACGAC | GGTGTTGCGGATCATCACTTTC | 5,215,514-5,207,699 | NC_005296.1 |
| palustris CGA009 | GAGAGGTAATCAGTGGTGGATTTGATGT | | | |
| | CCAAGCAAAGGACCACCCTC | GACGGTCAGGTGGTCCGAAC | 5,207,743-5,201,639 | NC_005296.1 |
| | AGCTTCGATATCATCCGCTGAT | TCAGCTTGCTTTG | 5,201,687-5,196,113 | NC_005296.1 |
| | TTGTTCATGTCGGACCTAACCGA | TCACTATAGGGGTCTTGCGGATCATCACTTTC | 5,196,162-5,187,847 | NC_005296.1 |
| | | ACGACAAGTGGAGAAGGGATAG | | |
| Rhodobacter | CAATACGATCGCATGTCCTAGGTAATACGACTCACTATAG | TCCCATGGTCATGTCCTTTGCG | 2,285,634-2,279,216 | NC_007493 |
| shaeroides | GGAGATGCATTTCACGCTTCGCGATTC | | | |
| 2,4,1. | CCGCCTTCACCAGAGACACC | GTGCGCTTTTCCACGAGGAGC | 2,279,260-2,271,404 | NC_007493 |
| | ATCGAGAAGTTCTACGATGCCGT | AATTGCCCCGCCCTGTCAGGGGCGGGGTTTTTTTT | 2,271,450-2,264,419 | NC_007493 |
| | | TGCAGCGCCCATTCCGTCTTC | | |
| | GCAAAAAAAAACCCCGCCCCTGACAGGGCGGGGTTTTTTT | GCTCCAAAAGGAGCCTTTAATTGTATCGGTTTATC | 245,956-252,936 | NC007494 |
| | TTTCAATTGGACCTGGATGGGCAGCAAG | AGCTTGCTTTGGAGAAAGCCTGCGCGGCTAG | | |
| Azorhizobium | CTCGCATCCATTCTCAGGCTGTCTCGTCTCGTCTCTCTAG | GCCCCCGGAAGGTGATCTTCCGGGGGCTTTCTCAT | 5,290,244-5,293,483 | NC_009937 |
| caulinodans | AGTCGGAGCTCTTGGGGCCTCTAAACGGGTCTTGAGGGGT | GCGTTGA | | |
| ORS571 | TTTTTGTTGTCTTCGACGCGAAGCTC | CAGCCTTGAGATAGATCAAGTGC | | |
| | ATAGGCAATACGATCGCATGTCCGTTTAAACTGATAAGGA | CTGATCCAGGCCTTCATCGG | 1,183,854-1,175,614 | NC_009937 |
| | CGGCACTGGCTGG | | | |
| | CGATGCCGTCCAGCACCTC | GACATGTCTGGTCTCCTTGGAAC | 1,175,653-1,170,712 | NC_009937 |
| | CTGCCACGGTTCCCAAGGTTC | TTCTGGAATTTGGTACCGAGTCAGTAACGTGCCACA | 1,179,751-1,162,529 | NC_009937 |
| | | GCCTCG | | |
| | TAAAAAAGCGGCTAACCACGCCGCTTTTTTTACGTCTGCA | ATCAGGCGCATATTTGAATGTATTTACTGCAG | 3,922,323-3,919,341 | NC_009937 |
| | GTGTTGTCGAAGCTTGATGCGC | CGGCCGCTACGTACTTGTGGGGT | | |
| | | CAGTTCCGGCTGGGGGTTCAGCAGCCACC | | |
| | | TGCAGTTAATTAAGGCGCTCCTTTCCTGATTCG | | |
| | CGCTGCTTAAGGTCATGCAGCAGGAGAACTAAAGGCCCGC | GCTGCTGTGTGGAGAGATCG | 3,930,607-3,934,260 | NC_009937 |
| | TCTGCGAAAGGAATAGCGTC | | | |
| | CTATCGCCGCCACCTGACC | GTCGGTGAGATTGATCATGGCC | 3,934,220-3,937,923 | NC_009937 |
| | CGTCAGAACGGCTCTGACGCATCAGGGAGA | TGCATGTCCGTTCCTCGCTG | 3,937,871-3,941,205 | NC_009937 |
| | AGTAATATTGCGGATCGGCCAGCAGCGAGGAA | ACATGTCTTGAATTCCTTCGAACC | 3,941,164-3,959,444 | NC_009937 |
| | GGTGGTCATTGGCAACGGTTCGAAG | TGCATTGCGTTCGCTCCC | 3,959,405-3,962,598 | NC_009937 |
| | TCCCCAAGAGCCCAACCGTTCCGGGAGCGAA | TGTCAGGGCAGGCAGGGCC | 3,962,559-3,966,562 | NC_009937 |
| Gluconacetobacter | TTAAGGTCATGCAGCAGGAGAACTAAAGGCCCGCGTTAGG | TCACCAGCCGTATCCGGAATATGTCAGGATCAT | 1,759,465-1,754,718 | CP001189 |
| diazotrophicus | TTGGTAATAAAAAAGCCCCCGGAATGATCTTCCGGGGGCC | GACATCCC | | |
| PA1 5 | GATCGAGGAAATCGACGTG | | | |
| | ATATTCCGGATACGGCTGGTGAGGTGGA | ACGATTTCCATGCCCAGGTC | 1,754,739-1,746,565 | CP001189 |
| | CGCCACGTCGTCAATGCCTATAAC | CCTCCAGCACCTCTTCGATG | 1,746,608-1,738,322 | CP001189 |
| | TGACCACCGTGCAGAAGATCC | GCTCCAAAAGGAGCCTTTAATTGTATCGGTTT | 1,738,366-1,730,601 | CP001189 |
| | | ATCAGCTTGC | | |
| | | TTTGGGCAATACCTGAGACGTTTCA | | |

TABLE 8
Strains used in this study

| Name | Strain | Source | Description |
|---|---|---|---|
| MR1 | E. coli DH10-beta | NEB | Cat# C3019 |
| MR2 | E. coli K-12 MG1655 | Voigt lab | |
| MR3 | Klebsiella oxytoca M5al | Voigt lab | |
| MR4 | Pseudomonas stutzeri A1501 | Poole lab | |
| MR5 | Azotobacter vinelandii DJ | Peters lab | |
| MR6 | Pseudomonas protegens Pf-5 | ATCC | BAA-477 |
| MR7 | P. protegens Pf-5 controller (P_{tac}-T7RNAP) | This study | generated by pMR86 |
| MR8 | P. protegens Pf-5 controller v1 (P_{tac}-nifA) | This study | generated by pMR97 |
| MR9 | P. protegens Pf-5 controller v2 (P_{tac}-nifA v2) | This study | generated by pMR98 |
| MR10 | P. protegens Pf-5 controller v3 (P_{tac}-nifA v3) | This study | generated by pMR99 |
| MR11 | P. protegens Pf-5 controller v4 (P_{BAD.10}-nifA) | This study | generated by pMR100 |
| MR12 | P. protegens Pf-5 controller v5 (P_{Fde}-nifA) | This study | generated by pMR101 |
| MR13 | Rhizobium sp. IRBG74 | Ané lab | |
| MR14 | R. sp. IRBG74 ΔhsdR | This study | generated by pMR44 |
| MR15 | R. sp. IRBG74 ΔrecA | This study | generated by pMR47 |
| MR16 | R. sp. IRBG74 Δnif | This study | generated by pMR45-46. Two nif clusters |
| | | | (227,127-219,579 and 234,635-234,802) were removed. |
| MR17 | R. sp. IRBG74 ΔhsdR, recA | This study | |
| MR18 | R. sp. IRBG74 ΔhsdR, Δnif | This study | |
| MR19 | R. sp. IRBG74 ΔhsdR, recA Δnif | This study | |
| MR20 | R. sp. IRBG74 ΔhsdR Δnif ΔrecA::P_{A1lacO1}-T7RNAP v1 | This study | generated by pMR82 |
| MR21 | R. sp. IRBG74 ΔhsdR Δnif ΔrecA::P_{A1lacO1}-T7RNAP v2 | This study | generated by pMR83 |
| MR22 | R. sp. IRBG74 ΔhsdR Δnif ΔrecA::P_{A1lacO1}-T7RNAP v3 | This study | generated by pMR84 |
| MR23 | R. sp. IRBG74 ΔhsdR Δnif ΔrecA::P_{Phl}-T7RNAP | This study | generated by pMR85 |
| MR24 | Azorhizobium caulinodans ORS571 | Poole lab | |
| MR25 | Azorhizobium caulinodans ORS571 ΔnifA | This study | generated by pMR48 |
| MR26 | R. spp NGR234 | Poole lab | |
| MR27 | R. leguminosarum bv. Trifolii WSM1325 | Poole lab | |
| MR28 | Sinorhizobium medicae WSM419 | Poole lab | |
| MR29 | R. leguminosarum 8002 | Poole lab | |
| MR30 | Sinorhizobium meliloti WSM1022 | Poole lab | |
| MR31 | R. leguminosarum A34 | Poole lab | |
| MR32 | Sinorhizobium fredii HH103 | Poole lab | |
| MR33 | Sinorhizobium meliloti 1021 | Poole lab | |
| MR34 | R. tropici CIAT899 | Poole lab | |
| MR35 | R. leguminosarum viciae 3841 | Poole lab | |
| MR36 | R. etli CFN42 | Poole lab | |
| MR37 | Agrobacterium tumefaciens C58 | Poole lab | |

TABLE 9
Plasmids used in this study

| | Origin of | | |
|---|---|---|---|
| Name | replication | Marker | Description |
| pMR1 | pBBR1 | Kanamycin | Plasmid for nif cluster cloning |
| pMR2 | pRO1600, | Gentamicin | Plasmid for nif cluster cloning |
| | p15A | | |
| pMR3 | pBBR1 | Kanamycin | Native nif cluster of K. oxytoca M5al |
| pMR4 | pRO1600, | Gentamicin | Native nif cluster of K. oxytoca M5al |
| | p15A | | |
| pMR5 | pBBR1 | Kanamycin | Native nif cluster of P. stutzeri A1501 |
| pMR6 | pRO1600, | Gentamicin | Native nif cluster of P. stutzeri A1501 |
| | p15A | | |
| pMR7 | pBBR1 | Kanamycin | Native nif cluster of A. vinelandii DJ |
| pMR8 | pRO1600, | Gentamicin | Native nif cluster of A. vinelandii DJ |
| | p15A | | |
| pMR9 | pBBR1 | Gentamicin | Native nif cluster of Cyanothece ATCC51142 |
| pMR10 | pRO1600, | Gentamicin | Native nif cluster of Cyanothece ATCC51142 |
| | p15A | | |
| pMR11 | pBBR1 | Kanamycin | Native nif cluster of P. polymyxa WLY78 |
| pMR12 | pRO1600, | Gentamicin | Native nif cluster of P. polymyxa WLY78 |
| | ColE1 | | |
| pMR13 | pBBR1 | Kanamycin | Native nif cluster of A. brasilense Sp7 |
| pMR14 | pRO1600, | Gentamicin | Native nif cluster of A. brasilense Sp7 |
| | ColE1 | | |
| pMR15 | pBBR1 | Kanamycin | Native nif cluster of R. sphaeroides 2.4.1 |
| pMR16 | pRO1600, | Gentamicin | Native nif cluster of R. sphaeroides 2.4.1 |
| | ColE1 | | |
| pMR17 | pBBR1 | Kanamycin | Native nif cluster of R. palustris CGA009 |
| pMR18 | pRO1600, | Gentamicin | Native nif cluster of R. palustris CGA009 |
| | ColE1 | | |
| pMR19 | pBBR1 | Kanamycin | Native nif cluster of A. caulinodans ORS571 (Part1 of 2) |
| pMR20 | RK2 | Tetracycline | Native nif cluster of A. caulinodans ORS571 (Part2 of 2) |
| pMR21 | pBBR1 | Kanamycin | Native nif cluster of G. diazotrophicus PA1 5 |
| pMR22 | pRO1600, | Gentamicin | Native nif cluster of G. diazotrophicus PA1 5 |
| | ColE1 | | |
| pMR23 | pRO1600, | Gentamicin | nifLA (3,915,521-3,918,529) deletion in the nif cluster of K. oxytoca M5al |
| | p15A | | |
| pMR24 | pRO1600, | Gentamicin | nifLA (1,420,874-1,423,084) deletion in the nif cluster of P. stutzeri A1501 |
| | p15A | | |
| pMR25 | pRO1600, | Gentamicin | nifLA (5,168,709-5,171,731) deletion in the nif cluster of A. vinelandii DJ |
| | p15A | | |
| pMR26 | pRO1600, | Gentamicin | Native nif cluster of A. vinelandii DJ with the rnf2 operon |
| | p15A | | |
| pMR27 | pRO1600, | Gentamicin | rnf1 (5,168,156-5,162,716) operon deletion in the nif cluster of A. vinelandii DJ |
| | p15A | | |
| pMR28 | pRO1600, | Gentamicin | fix operon (995,860-1,000,698) deletion in the nif cluster of A. vinelandii DJ |
| | p15A | | |
| pMR29 | pBBR1 | Kanamycin | Refactored nif cluster v2.1 |
| pMR30 | pRO1600, | Gentamicin | Refactored nif cluster v2.1 |
| | p15A | | |
| pMR31 | RK2 | Tetracycline | Refactored nif cluster v2.1 |
| pMR32 | ColE1 | Gentamicin | P_{WT}-nifHDKTY |
| pMR33 | ColE1 | Gentamicin | P₂-nifENX |
| pMR34 | ColE1 | Gentamicin | P₂-nifJ |
| pMR35 | ColE1 | Gentamicin | P₂-nifBQ |
| pMR36 | ColE1 | Gentamicin | P₂-nifF |
| pMR37 | ColE1 | Gentamicin | P₂-nifUSVWZM |
| pMR38 | pBBR1 | Kanamycin | Refactored nif cluster v3.2 |
| pMR39 | pRO1600, | Gentamicin | Refactored nif cluster v3.2 |
| | p15A | | |
| pMR40 | pBBR1 | Kanamycin | LacI, P_{A1lacO1}-gfpmut3b |
| pMR41 | RSF1010 | Gentamicin | Lacl, P_{A1lacO1}-gfpmut3b |
| pMR42 | RK2 | Tetracycline | LacI, P_{tac}-gfpmut3b |
| pMR43 | pRO1600, | Gentamicin | LacI, P_{A1lacO1}-gfpmut3b |
| | ColE1 | | |
| pMR44 | p15A | Gentamicin | Suicide plasmid for hsdR deletion in R. sp. IRBG74 |
| pMR45 | p15A | Gentamicin | Suicide plasmid for the nif cluster I (219,579-227,127) deletion in R. sp. IRBG74 |
| pMR46 | p15A | Gentamicin | Suicide plasmid for the nif cluster II (234,635-234,802) deletion in R. sp. IRBG74 |
| pMR47 | p15A | Gentamicin | Suicide plasmid for recA deletion in R. sp. IRBG74 |
| pMR48 | p15A | Gentamicin | Suicide plasmid for nifA deletion in A. caulinodans ORS571 |
| pMR49 | pBBR1 | Gentamicin | LacI, P_{A1lacO1}-nifV (A. caulinodans ORS571) |
| pMR50 | pBBR1 | Gentamicin | P_{nifH}(R. sp. IRBG7A)-sfgfp |
| pMR51 | pBBR1 | Gentamicin | NifA(R. sp. IRBG74), P_{nifH}(R. sp. IRBG74)-sfgfp |
| pMR52 | pBBR1 | Gentamicin | NifA(K. oxytoca), P_{nifH}(K. oxytoca)-sfgfp |
| pMR53 | pBBR1 | Gentamicin | NifA(R. sp. IRBG74), P_{nifH}(K. oxytoca)-sfgfp |
| pMR54 | pBBR1 | Gentamicin | NifA(P. stutzeri), P_{nifH}(P. stutzeri)-sfgfp |
| pMR55 | pBBR1 | Gentamicin | NifA(R. sp. IRBG74), P_{nifH}(P. stutzeri)-sfgfp |
| pMR56 | pBBR1 | Gentamicin | NifA(A. caulinodans), P_{nifH}(A. caulinodans)-sfgfp |
| pMR57 | pBBR1 | Gentamicin | NifA(R. sp. IRBG74), P_{nifH}(A. caulinodans)-sfgfp |
| pMR58 | pBBR1 | Kanamycin | Plasmid for consituitive promoter characterization. P_{constitutive}-gfpmut3b |
| pMR59 | pRO1600, | Gentamicin | Plasmid for consituitive promoter characterization. P_{constitutive}-gfpmut3b |
| | p15A | | |
| pMR60 | pBBR1 | Kanamycin | PT7(WT)-mCherry |
| pMR61 | pBBR1 | Kanamycin | PT7(P1)-mCherry |
| pMR62 | pBBR1 | Kanamycin | PT7(P2)-mCherry |
| pMR63 | pBBR1 | Kanamycin | PT7(P3)-mCherry |
| pMR64 | pBBR1 | Kanamycin | PT7(P4)-mCherry |
| pMR65 | pBBR1 | Kanamycin | PT7(P5)-mCherry |
| pMR66 | pRO1600, | Gentamicin | AraE, AraC, P_{BAD.10}-gfpmut3b |
| | ColE1 | | |
| pMR67 | pBBR1 | Kanamycin | Plasmid for terminator characterization. P_{T7}-gfpmut3b-mrfp1 |
| pMR68 | pRO1600, | Gentamicin | Plasmid for terminator characterization. P_{T7}-gfpmut3b-mrfp1 |
| | ColE1 | | |
| pMR69 | pBBR1 | Kanamycin | LuxR, P_{Lux}-gfpmut3b |
| pMR70 | pBBR1 | Kanamycin | TetR, P_{Tet}-gfpmut3b |
| pMR71 | pBBR1 | Kanamycin | CymR, P_{Cym}-gfpmut3b |
| pMR72 | pBBR1 | Kanamycin | PhlF, P_{Phl}-gfpmut3b |
| pMR73 | pBBR1 | Kanamycin | NahR, P_{Sal}-gfpmut3b |
| pMR74 | pRO1600, | Gentamicin | PhlF, P_{Phl}-gfpmut3b |
| | ColE1 | | |
| pMR75 | pRO1600, | Gentamicin | TetR, P_{Tet}-gfpmut3b |
| | ColE1 | | |
| pMR76 | pRO1600, | Gentamicin | LuxR, P_{Lux}-gfpmut3b |
| | ColE1 | | |
| pMR77 | pRO1600, | Gentamicin | CymR, P_{Cym}-gfpmut3b |
| | ColE1 | | |
| pMR78 | pRO1600, | Gentamicin | FdeR, P_{Fde}-gfpmut3b |
| | ColE1 | | |
| pMR79 | pRO1600, | Gentamicin | LacI(Q18M/A47V/F161Y), P_{tac}-gfpmut3b |
| | ColE1 | | |
| pMR80 | pBBR1 | Kanamycin | P_{T7}-gfpmut3b |
| pMR81 | pRO1600, | Gentamicin | P_{T7}-gfpmut3b |
| | p15A | | |
| pMR82 | p15A | Gentamicin | Controller for R. sp. IRBG74, LacI, P_{A1lacO1}-T7RNAP (RBSr33 for T7RNAP) |
| pMR83 | p15A | Gentamicin | Controller for R. sp. IRBG74, Lacl, P_{A1lacO1}-T7RNAP (RBSr32 for T7RNAP) |
| pMR84 | p15A | Gentamicin | Controller for R. sp. IRBG74, Lacl, P_{A1lacO1}-T7RNAP (RBSr3 forT7RNAP) |
| pMR85 | p15A | Gentamicin | Controller for R. sp. IRBG74, PhlF, P_{PhlF}-T7RNAP (RBSr33 for T7RNAP) |
| pMR86 | ColE1 | Tetracycline | Controller for P. protegens Pf-5, LacI(Q18M/A47V/F161Y), P_{tac}-T7RNAP |
| pMR87 | pBBR1 | Kanamycin | NocR, P_{noc}-gfpmut3b |
| pMR88 | pBBR1 | Kanamycin | OccR, P_{occ}-gfpmut3b |
| pMR89 | pBBR1 | Gentamicin | NifA(A. vinelandii), P_{nifH}(A. vinelandii)-sfgfp |
| pMR90 | pBBR1 | Gentamicin | NifA(K. oxytoca), P_{nifH}(P. stutzeri)-sfgfp |
| pMR91 | pBBR1 | Gentamicin | NifA(K. oxytoca), P_{nifH}(A. vinelandii)-sfgfp |
| pMR92 | pRO1600, | Gentamicin | NifA(K. oxytoca), P_{nif}H(K. oxytoca)-sfgfp |
| | p15A | | |
| pMR93 | pRO1600, | Gentamicin | NifA(A. vinelandii), P_{nifH}(A. vinelandii)-sfgfp |
| | p15A | | |
| pMR94 | pRO1600, | Gentamicin | NifA(P. stutzeri), P_{nifH}(P. stutzeri)-sfgfp |
| | p15A | | |
| pMR95 | pRO1600, | Gentamicin | NifA(P. stutzeri), P_{nifH}(K. oxytoca)-sfgfp |
| | p15A | | |
| pMR96 | pRO1600, | Gentamicin | NifA(P. stutzeri), P_{nifH}(A. vinelandii)-sfgfp |
| | p15A | | |
| pMR97 | ColE1 | Tetracycline | NifA controller for P. protegens Pf-5, LacI(Q18M/A47V/F161Y), P_{tac}-nifA(P. stutzeri) |
| | | | (RBSp32 for NifA) |
| pMR98 | ColE1 | Tetracycline | NifA controller for P. protegens Pf-5, LacI(Q18M/A47V/F161Y), P_{tac}-nifA(P. stutzeri) |
| | | | (RBSp27 RBS for NifA) |
| pMR99 | ColE1 | Tetracycline | NifA controller for P. protegens Pf-5, LacI(Q18M/A47V/F161Y), P_{tac}-nifA(P. stutzeri) |
| | | | (RBSp33 for NifA) |
| pMR100 | ColE1 | Tetracycline | NifA controller for P. protegens Pf-5, AraE, AraC, P_{BAD.10}-nifA |
| pMR101 | ColE1 | Tetracycline | NifA controller for P. protegens Pf-5, FdeR, P_{Fde}-nifA |
| pMR102 | IncW | Spectinomycin | NifA controller plasmid for E. coli, LacI, P_{A1lacO1}-nifA(K. oxytoca) |
| pMR103 | pRO1600, | Gentamicin | P_{nifH}(K. oxytoca)-sfgfp |
| | p15A | | |
| pMR104 | pRO1600, | Gentamicin | P_{nifH}(P. stutzeri)-sfgfp |
| | p15A | | |
| pMR105 | pRO1600, | Gentamicin | P_{nifH}(A. vinelandii)-sfgfp |
| | p15A | | |
| pMR106 | pBBR1 | Gentamicin | P_{nifH}(K. oxytoca)-sfgfp |
| pMR107 | pBBR1 | Gentamicin | P_{nifH}(P. stutzeri)-sfgfp |
| pMR108 | pBBR1 | Gentamicin | P_{nifH}(A. vinelandii)-sfgfp |
| pMR109 | p15A | Kanamycin | P_{BAD}-T7RNAP |
| pMR110 | p15A | Kanamycin | P_{Bet}-T7RNAP |
| pMR111 | p15A | Kanamycin | P_{Cin}-T7RNAP |
| pMR112 | p15A | Kanamycin | P_{cym}-T7RNAP |
| pMR113 | p15A | Kanamycin | P_{Lux}-T7RNAP |
| pMR114 | p15A | Kanamycin | P_{Phl}-T7RNAP |
| pMR115 | p15A | Kanamycin | P_{3B5B}-T7RNAP |
| pMR116 | p15A | Kanamycin | P_{tac}-T7RNAP |
| pMR117 | p15A | Kanamycin | P_{Tet}-T7RNAP |
| pMR118 | p15A | Kanamycin | P_{Ttg}-T7RNAP |
| pMR119 | p15A | Kanamycin | P_{Van}-T7RNAP |
| pMR120 | p15A | Kanamycin | P_{Sal}-T7RNAP |
| pMR121 | pBBR1 | Gentamicin | P_{T7}(P2)-gfpmut3b |
| pMR122 | pBBR1 | Gentamicin | NifA controller for A. caulinodans, LacI, P_{A1lacO1}-nifA-rpoN |
| pMR123 | pBBR1 | Gentamicin | NifA controller for A. caulinodans, LacI, P_{A1lacO1}-nifA(L94Q)-rpoN |
| pMR124 | pBBR1 | Gentamicin | NifA controller for A. caulinodans, LacI, P_{A1lacO1}-nifA(D95Q)-rpoN(A. caulinodans) |
| pMR125 | pBBR1 | Gentamicin | NifA controller for A. caulinodans, LacI, P_{A1lacO1}-nifA(L94Q/D95Q)-rpoN |
| pMR126 | pBBR1 | Gentamicin | NifA controller for A. caulinodans, NahR, P_{Sal}-nifA(L94Q/D95Q)-rpoN |
| pMR127 | pBBR1 | Gentamicin | NifA controller for A. caulinodans, NocR, P_{noc}-nifA(L94Q/D95Q)-rpoN |
| pMR128 | pBBR1 | Gentamicin | NifA controller for A. caulinodans, OccR, P_{occ}-nifA(L94Q/D95Q)-rpoN |
| pMR129 | pBBR1 | Gentamicin | P_{nifH}(A. caulinodans)-sfgfp |
| pMR130 | pBBR1 | Gentamicin | NifA, P_{nifH}(A. caulinodans)-sfgfp |
| pMR131 | pBBR1 | Gentamicin | NifA, RpoN, P_{nifH}(A. caulinodans)-sfgfp |
| pMR132 | pBBR1 | Gentamicin | LacI, P_{A1lacO1}-nifA(L94Q/D95Q)-rpoN, P_{nifH}-sfgfp |
| pMR133 | pBBR1 | Gentamicin | NahR, PSal-nifA(L94Q/D95Q)-rpoN, P_{nifH}-sfgfp |
| pMR134 | pBBR1 | Gentamicin | NocR, Pnoc-nifA(L94Q/D95Q)-rpoN, P_{nifH}-sfgfp |
| pMR135 | pBBR1 | Gentamicin | OccR, Pocc-nifA(L94Q/D95Q)-rpoN, P_{nifH}-sfgfp |
| pMR136 | pBBR1 | Gentamicin | Refactored nif cluster v2.1 |

TABLE 10
Genetic part sequences used in this study

| Name | Genetic part | DNA sequence (SEQ ID Nos: 122-225) |
|---|---|---|
| P_{A1lac01} | Promoter⁶ | AGAGTGTTGACTTGTGAGCGGATAACAATGATACTTAGATTCAATTGTGAGCGGATAACAATTTCACACA |
| T7 RNAP | Gene | ATGAACACGATTAACATCGCTAAGAACGACTTCTCTGACATCGAACTGGCTGCTATCCCGTTCAACACTCTGGCTGACCATTACGGTGAGCG |
| | | TTTAGCTCGCGAACAGTTGGCCCTTGAGCATGAGTCTTACGAGATGGGTGAAGCACGCTTCCGCAAGATGTTTGAGCGTCAACTTAAAGCTG |
| | | GTGAGGTTGCGGATAACGCTGCCGCCAAGCCTCTCATCACTACCCTACTCCCTAAGATGATTGCACGCATCAACGACTGGTTTGAGGAAGTG |
| | | AAAGCTAAGCGCGGCAAGCGCCCGACAGCCTTCCAGTTCCTGCAAGAAATCAAGCCGGAAGCCGTAGCGTACATCACCATTAAGACCACTCT |
| | | GGCTTGCCTAACCAGTGCTGACAATACAACCGTTCAGGCTGTAGCAAGCGCAATCGGTCGGGCCATTGAGGACGAGGCTCGCTTCGGTCGTA |
| | | TCCGTGACCTTGAAGCTAAGCACTTCAAGAAAAACGTTGAGGAACAACTCAACAAGCGCGTAGGGCACGTCTACAAGAAAGCATTTATGCAA |
| | | GTTGTCGAGGCTGACATGCTCTCTAAGGGTCTACTTGGTGGCGAGGCGTGGTCTTCGTGGCATAAGGAAGACTCTATTCATGTAGGAGTACG |
| | | CTGCATCGAGATGCTCATTGAGTCAACCGGAATGGTTAGCTTACACCGCCAAAATGCTGGCGTAGTAGGTCAAGACTCTGAGACTATCGAAC |
| | | TCGCACCTGAATACGCTGAGGCTATCGCAACCCGTGCAGGTGCGCTGGCTGGCATCTCTCCGATGTTCCAACCTTGCGTAGTTCCTCCTAAG |
| | | CCGTGGACTGGCATTACTGGTGGTGGCTATTGGGCTAACGGTCGTCGTCCTCTGGCGCTGGTGCGTACTCACAGTAAGAAAGCACTGATGCG |
| | | CTACGAAGACGTTTACATGCCTGAGGTGTACAAAGCGATTAACATTGCGCAAAACACCGCATGGAAAATCAACAAGAAAGTCCTAGCGGTCG |
| | | CCAACGTAATCACCAAGTGGAAGCATTGTCCGGTCGAGGACATCCCTGCGATTGAGCGTGAAGAACTCCCGATGAAACCGGAAGACATCGAC |
| | | ATGAATCCTGAGGCTCTCACCGCGTGGAAACGTGCTGCCGCTGCTGTGTACCGCAAGGACAAGGCTCGCAAGTCTCGCCGTATCAGCCTTGA |
| | | GTTCATGCTTGAGCAAGCCAATAAGTTTGCTAACCATAAGGCCATCTGGTTCCCTTACAACATGGACTGGCGCGGTCGTGTTTACGCTGTGT |
| | | CAATGTTCAACCCGCAAGGTAACGATATGACCAAAGGACTGCTTACGCTGGCGAAAGGTAAACCAATCGGTAAGGAAGGTTACTACTGGCTG |
| | | AAAATCCACGGTGCAAACTGTGCGGGTGTCGACAAGGTTCCGTTCCCTGAGCGCATCAAGTTCATTGAGGAAAACCACGAGAACATCATGGC |
| | | TTGCGCTAAGTCTCCACTGGAGAACACTTGGTGGGCTGAGCAAGATTCTCCGTTCTGCTTCCTTGCGTTCTGCTTTGAGTACGCTGGGGTAC |
| | | AGCACCACGGCCTGAGCTATAACTGCTCCCTTCCGCTGGCGTTTGACGGGTCTTGCTCTGGCATCCAGCACTTCTCCGCGATGCTCCGAGAT |
| | | GAGGTAGGTGGTCGCGCGGTTAACTTGCTTCCTAGTGAAACCGTTCAGGACATCTACGGGATTGTTGCTAAGAAAGTCAACGAGATTCTACA |
| | | AGCAGACGCAATCAATGGGACCGATAACGAAGTAGTTACCGTGACCGATGAGAACACTGGTGAAATCTCTGAGAAAGTCAAGCTGGGCACTA |
| | | AGGCACTGGCTGGTCAATGGCTGGCTTACGGTGTTACTCGCAGTGTGACTAAGCGTTCAGTCATGACGCTGGCTTACGGGTCCAAAGAGTTC |
| | | GGCTTCCGTCAACAAGTGCTGGAAGATACCATTCAGCCAGCTATTGATTCCGGCAAGGGTCTGATGTTCACTCAGCCGAATCAGGCTGCTGG |
| | | ATACATGGCTAAGCTGATTTGGGAATCTGTGAGCGTGACGGTGGTAGCTGCGGTTGAAGCAATGAACTGGCTTAAGTCTGCTGCTAAGCTGC |
| | | TGGCTGCTGAGGTCAAAGATAAGAAGACTGGAGAGATTCTTCGCAAGCGTTGCGCTGTGCATTGGGTAACTCCTGATGGTTTCCCTGTGTGG |
| | | CAGGAATACAAGAAGCCTATTCAGACGCGCTTGAACCTGATGTTCCTCGGTCAGTTCCGCTTACAGCCTACCATTAACACCAACAAAGATAG |
| | | CGAGATTGATGCACACAAACAGGAGTCTGGTATCGCTCCTAACTTTGTACACAGCCAAGACGGTAGCCACCTTCGTAAGACTGTAGTGTGGG |
| | | CACACGAGAAGTACGGAATCGAATCTTTTGCACTGATTCACGACTCCTTCGGTACGATTCCGGCTGACGCTGCGAACCTGTTCAAAGCAGTG |
| | | CGCGAAACTATGGTTGACACATATGAGTCTTGTGATGTACTGGCTGATTTCTACGACCAGTTCGCTGACCAGTTGCACGAGTCTCAATTGGA |
| | | CAAAATGCCAGCACTTCCGGCTAAAGGTAACTTGAACCTCCGTGACATCTTAGAGTCGGACTTCGCGTTCGCGTAA |
| P_{laci}_{_{q}} | Promoter⁷ | CGAATGGTGCAAAACCTTTCGCGGTATGGCATGATAGCGCCCGGAAGAGAG |
| rpoN of A. | Gene | ATGGCGATGAGCCCAAAGATGGAGTTCCGCCAGAGCCAGTCTCTGGTGATGACGCCGCAGCTGATGCAGGCCATCAAGCTGCTGCAGCTCTC |
| caulinodans | | CAATCTCGAACTGGTCGCCTATGTGGAGGCCGAGCTCGAACGCAATCCGCTGCTGGAGCGGGCGAGCGAGCCGGAAAGCCCCGAGCACGATC |
| | | CGCCGAACCCGCAGGAAGAGGCACCCACCCCGCCTGACAGTGGCGCGCCGGTGTCCGGCGACTGGATGGAAAGCGACATGGGCTCGAGCCGC |
| | | GAGGCCATCGAGACCCGGCTGGACACCGACCTCGGCAATGTCTTTCCCGATGATGCGCCGGCCGAGCGCATCGGCGCGGGCAGCGGCAGCGG |
| | | CTCGTCCATCGAATGGGGCTCGGGCGGCGACCGGGGCGAGGACTACAATCCGGAAGCCTTCCTCGCTGCCGAGACGACGCTGGCCGACCATC |
| | | TGGAAGCCCAGCTCTCCGTGGCGGAGCCCGATCCGGCGCGCCGCCTCATCGGCCTCAACCTCATCGGCCTCATCGACGAGACGGGTTATTTC |
| | | TCCGGCGACCTCGATGCGGTGGCCGAGCAACTGGGCGCCACCCACGATCAGGTGGCCGACGTGCTGCGCGTCATCCAGAGCTTCGAGCCGTC |
| | | CGGCGTCGGCGCACGGTCGCTCAGCGAATGCCTGGCCCTGCAATTGCGCGACAAGGATCGCTGCGATCCCGCCATGCAGGCGCTGCTCGACA |
| | | ATCTGGAACTCCTCGCCCGCCACGACCGCAACGCGCTGAAGCGCATCTGCGGGGTGGACGCGGAAGACCTCGCGGACATGATCGGCGAGATC |
| | | CGCCGCCTCGATCCGAAGCCCGGCCTCGCCTATGGCGGCGGCGTCGTCCACCCGCTGGTGCCGGACGTGTTCGTGCGCGAGGGCTCCGACGG |
| | | CAGCTGGATCGTGGAACTGAATTCCGAGACGCTGCCGCGCGTGCTGGTGAACCAGACCTATCACGCGACGGTGGCCAAGGCGGCGCGCTCGG |
| | | CCGAGGAAAAGACCTTCCTCGCCGACTGCCTCCAGAGCGCCTCCTGGCTTACCCGCTCGCTCGACCAGCGGGCTCGCACCATCCTCAAGGTG |
| | | GCGAGCGAGATCGTGCGCCAGCAGGACGCCTTCCTCGTGCACGGCGTGCGGCACCTGCGCCCCCTGAACCTGCGCACGGTGGCGGATGCCAT |
| | | CGGCATGCACGAATCCACCGTCTCGCGGGTGACCTCGAACAAGTACATCTCCACCCCGCGCGGGGTGCTGGAGATGAAGTTCTTCTTCTCCT |
| | | CCTCCATCGCTTCCTCGGGTGGTGGCGAGGCCCATGCGGCGGAGGCGGTGCGCCACCGCATCAAGAGCCTCATCGAGGCCGAGAGTGCGGAC |
| | | GACGTGCTGTCCGACGACACGCTGGTGCAGAAGCTGAAGGACGACGGCATCGATATCGCCCGCCGAACGGTCGCGAAATATCGCGAGAGCAT |
| | | GAACATCCCGTCCTCGGTCCAGCGCCGCCGCGAAAAGCAGGCCCTGCGCAGCGACGCCGCCGCCGCCGGCTGA |
| lacl | Gene | GTGAAACCAGTAACGTTATACGATGTCGCAGAGTATGCCGGTGTCTCTTATCAGACCGTTTCCCGCGTGGTGAACCAGGCCAGCCACGTTTC |
| | | TGCGAAAACGCGGGAAAAAGTGGAAGCGGCGATGGCGGAGCTGAATTACATTCCCAACCGCGTGGCACAACAACTGGCGGGCAAACAGTCGT |
| | | TGCTGATTGGCGTTGCCACCTCCAGTCTGGCCCTGCACGCGCCGTCGCAAATTGTCGCGGCGATTAAATCTCGCGCCGATCAACTGGGTGCC |
| | | AGCGTGGTGGTGTCGATGGTAGAACGAAGCGGCGTCGAAGCCTGTAAAGCGGCGGTGCACAATCTTCTCGCGCAACGCGTCAGTGGGCTGAT |
| | | CATTAACTATCCGCTGGATGACCAGGATGCCATTGCTGTGGAAGCTGCCTGCACTAATGTTCCGGCGTTATTTCTTGATGTCTCTGACCAGA |
| | | CACCCATCAACAGTATTATTTTCTCCCATGAAGACGGTACGCGACTGGGCGTGGAGCATCTGGTCGCATTGGGTCACCAGCAAATCGCGCTG |
| | | TTAGCGGGCCCATTAAGTTCTGTCTCGGCGCGTCTGCGTCTGGCTGGCTGGCATAAATATCTCACTCGCAATCAAATTCAGCCGATAGCGGA |
| | | ACGGGAAGGCGACTGGAGTGCCATGTCCGGTTTTCAACAAACCATGCAAATGCTGAATGAGGGCATCGTTCCCACTGCGATGCTGGTTGCCA |
| | | ACGATCAGATGGCGCTGGGCGCAATGCGCGCCATTACCGAGTCCGGGCTGCGCGTTGGTGCGGATATCTCGGTAGTGGGATACGACGATACC |
| | | GAAGACAGCTCATGTTATATCCCGCCGTTAACCACCATCAAACAGGATTTTCGCCTGCTGGGGCAAACCAGCGTGGACCGCTTGCTGCAACT |
| | | CTCTCAGGGCCAGGCGGTGAAGGGCAATCAGCTGTTGCCCGTCTCACTGGTGAAAAGAAAAACCACCCTGGCGCCCAATACGCAAACCGCCT |
| | | CTCCCCGCGCGTTGGCCGATTCATTAATGCAGCTGGCACGACAGGTTTCCCGACTGGAAAGCGGGCAGTGA |
| gfprnut3b | Gene | ATGAGTAAAGGAGAAGAACTTTTCACTGGAGTTGTCCCAATTCTTGTTGAATTAGATGGTGATGTTAATGGGCACAAATTTTCTGTTAGTGG |
| | | AGAGGGTGAAGGTGATGCAACATACGGAAAACTTACCCTTAAATTTATTTGCACTACTGGAAAACTACCTGTTCCATGGCCAACACTTGTCA |
| | | CTACTTTCGGTTATGGTGTTCAATGCTTTGCGAGATACCCAGATCATATGAAACAGCATGACTTTTTCAAGAGTGCCATGCCCGAAGGTTAT |
| | | GTACAGGAAAGAACTATATTTTTCAAAGATGACGGGAACTACAAGACACGTGCTGAAGTCAAGTTTGAAGGTGATACCCTTGTTAATAGAAT |
| | | CGAGTTAAAAGGTATTGATTTTAAAGAAGATGGAAACATTCTTGGACACAAATTGGAATACAACTATAACTCACACAATGTATACATCATGG |
| | | CAGACAAACAAAAGAATGGAATCAAAGTTAACTTCAAAATTAGACACAACATTGAAGATGGAAGCGTTCAACTAGCAGACCATTATCAACAA |
| | | AATACTCCAATTGGCGATGGCCCTGTCCTTTTACCAGACAACCATTACCTGTCCACACAATCTGCCCTTTCGAAAGATCCCAACGAAAAGAG |
| | | AGACCACATGGTCCTTCTTGAGTTTGTAACAGCTGCTGGGATTACACATGGCATGGATGAACTATACAAATAG |
| sfgfp | Gene | ATGCGTAAAGGCGAAGAGCTGTTCACTGGTGTCGTCCCTATTCTGGTGGAACTGGATGGTGATGTCAACGGTCATAAGTTTTCCGTGCGTGG |
| | | CGAGGGTGAAGGTGACGCAACTAATGGTAAACTGACGCTGAAGTTCATCTGTACTACTGGTAAACTGCCGGTACCTTGGCCGACTCTGGTAA |
| | | CGACGCTGACTTACAGTGCTTTGCTCGTTATCCGGACCATATGAAGCAGCATGACCTTCAAGTCCGCCATGCCGGAAGGGAGCCTCTGGTAT |
| | | GTGCAGGAACGCACGATTTCCTTTAAGGATGACGGCACGTACAAAACGCGTGCGGAAGTGAAATTTGAAGGCGATACCCTGGTAAACCGCAT |
| | | TGAGCTGAAAGGCATTGACTTTAAAGAAGACGGCAATATCCTGGGCCATAAGCTGGAATACAATTTTAACAGCCACAATGTTTACATCACCG |
| | | CCGATAAACAAAAAAATGGCATTAAAGCGAATTTTAAAATTCGCCACAACGTGGAGGATGGCAGCGTGCAGCTGGCTGATCACTACCAGCAA |
| | | AACACTCCAATCGGTGATGGTCCTGTTCTGCTGCCAGACAATCACTATCTGAGCACGCAAAGCGTTCTGTCTAAAGATCCGAACGAGAAACG |
| | | CGATCATATGGTTGGTCCTGTTCGCTGCCAGACAATCACTATCTTCTGCTGGAGTTAATTGTACAAATGA |
| mrfp1 | Gene | ATGGCTTCCTCCGAAGACGTTATCAAAGAGTTCATGCGTTTCAAAGTTCGTATGGAAGGTTCCGTTAACGGTCACGAGTTCGAAATCGAAGG |
| | | TGAAGGTGAAGGTCGTCCGTACGAAGGTACCCAGACCGCTAAACTGAAAGTTACCAAAGGTGGTCCGCTGCCGTTCGCTTGGGACATCCTGT |
| | | CCCCGCAGTTCCAGTACGGTTCCAAAGCTTACGTTAAACACCCGGCTGACATCCCGGACTACCTGAAACTGTCCTTCCCGGAAGGTTTCAAA |
| | | TGGGAACGTGTTATGAACTTCGAAGACGGTGGTGTTGTTACCGTTACCCAGGACTCCTCCCTGCAAGACGGTGAGTTCATCTACAAAGTTAA |
| | | ATTGCGTGGTACCAACTTCCCGTCCGACGTCCTGTTTATGCAGAAAAAAACCATGGGTTGGGAAGCTTCCACCGAACGTATGTACCCGGAAG |
| | | ACGGTGCTCTGAAAGGTGAAATCAAAATGCGTCTGAAACTGAAAGACGGTGGTATAACTGCGGCTGAAGTTAAAACCACCTACATGGCTAAA |
| | | AAACCGGTTCAGCTGCCGGGTGCTTACAAAACCGACATGCGTCAAACTGGACATCACCTCCCACAACGAAGACTACACCATCGTTAAATAGA |
| | | ACGTGCTGAAGGTCGTTCACTCCCACCGGTGCTTAA |
| mCherry | Gene | ATGGTTTCGAAGGGCGAGGAGGATAACATGGCCATCATCAAGGAGTTCATGCGCTTCAAGGTGCACATGGAGGGCTCCGTGAACGGCCACGA |
| | | GTTCGAGATCGAGGGCGAGGGCGAGGGCCGCCCCTACGAGGGCACCCAGACCGCCAAGCTGAAGGTGACCAAGGGTGGCCCCCTGCCCTTCG |
| | | CCTGGGACATCCTGTCCCCTCAGTTCATGTACGGCTCCAAGGCCTACGTGAAGCACCCCGCCGACATCCCCGACTACTTGAAGCTGTCCTTC |
| | | CCCGAGGGCTTCAAGTGGGAGCGCGTGATGAACTTCGAGGACGGCGGCGTGGTGACCGTGACCCAGGACTCCTCCTTGCAGGACGGCGAGTT |
| | | CATCTACAAGGTGAAGCTGCGCGGCACCAACTTCCCCTCCGACGGCCCCGTAATGCAGAAGAAGACGATGGGCTGGGAGGCCTCCTCCGAGC |
| | | GGATGTACCCCGAGGACGGCGCCCTGAAGGGCGAGATCAAGCAGAGGCTGAAGCTGAAGGACGGCGGCCACTACGACGCTGAGGTCAAGACC |
| | | ACCTACAAGGCCAAGAAGCCCGTGCAGCTGCCCGGCGCCTACAACGTCAACATCAAGTTGGACATCACCTCCCACAACGAGGACTACACCAT |
| | | CGTGGAACAGTACGAACGCGCCGAGGGCCGCCACTCCACCGGCGGCATGGACGAGCTGTACAAGTAA |
| P_{WT} | Promoter⁸ | TAATACGACTCACTATAGGGAGA |
| P₁ | Promoter⁸ | TAATACGACTCACTACAGGCAGA |
| P₂ | Promoter⁸ | TAATACGACTCACTAGAGAGAGA |
| P₃ | Promoter⁸ | TAATACGACTCACTAATGGGAGA |
| P₄ | Promoter⁸ | TAATACGACTCACTAAAGGGAGA |
| P₅ | Promoter⁸ | TAATACGACTCACTATAGGTAGA |
| P₆ | Promoter⁸ | TAATACGACTCACTATAGGTAGA |
| nifV of A. | Gene | GTGTTCCGTGGGAGGCCTGCCATGCTCGCCAAGACACCCGCAAACCCCGCGCCGCTTCAGCGGACGGCGTTCCTGAACGACACCACGCTGCG |
| caulinodans | | CGACGGCGAGCAGGCGCCGGGTGTCGCCTTCACCCGCAAGGAGAAGATCGAGATCGCCGCCGCCCTTGCCGCCGCCGGTGTCCCGGAGATCG |
| | | AGGCGGGAACGCCCGCCATGGGCGACGAAGAGGTGGAAACCATCCGCTCCATCGTCTCGCTGAACCTCCCGACGCGCGTCATGGCCTGGTGC |
| | | CGCATGAGCGAGGACGACCTGATGGCCGCCGTCGCGGCGGGCGTGAAGATCGTCAATGTCTCCATTCCCACCTCCGACCGGCAACTGGCCGG |
| | | CAAGCTCGGCAAGGATCGCGCCTGGGCGCTCGGCCGTGTGGCGGAGGTGGTGACACTGGCGCGTCGGCTCGGCTTTGAGGTGGCGGTAGGGG |
| | | GCGAGGATTCCTCGCGGGCCGATCCCGATTTTCTCTGCCGTCTCGCGGAGACGGCGAAGGCGGCGGGCGCCTTTCGCCTGCGGCTGGCCGAC |
| | | ACGCTTGGCGTGCTTGACCCCTTCGGCACCTATGCATTGGTGCGCCGGGTGGCCGCCACCACCGACATCGAGCTTGAGTTCCACGCCCATGA |
| | | CGATCTCGGCCTTGCCACCGCCAATACGCTGGCGGCGGTGATGGGCGGAGCGCGTCACGCCAGCGTCACCGTCGCCGGGCTCGGCGAGCGCG |
| | | CGGGCAATGCCGCGCTGGAGGAAGTGGCCATCGCCCTGCGCCAGACGGCGCGGGCGGAGACCGGCATCGCTCCGGCCGCGCTGAAGCCGCTG |
| | | GCCGAACTAGTGTGCGGCGCCGCCGCCCGTCCGGTGCCGCGCGGCAAGGCCATCGTCGGCGCGGATGTGTTCACCCACGAGTCGGGCATCCA |
| | | TGTCTCCGGCCTGCTCAAGGACCGGGCCACCTATGAAGCTCTGAATCCGGAACTGTTCGGGCGTGGCCACACGGTGGTGCTCGGAAAGCATT |
| | | CCGGTCTTGCGGCGGTGGAGAAGGCGCTGGCCGACGAGGGCATCACCGTGGATGCGGTGCGCGGGCGCGCCATTCTCGACCGGGTGCGGGCT |
| | | TTTGCTGTCCGCACCAAGGAGAATGTTTCCCGCGAGACGCTGCTGCGCTTCTATCAGGACAGCTrCACCGAGTCCGCGCTGCGTCTGCGGCG |
| | | GGCCGCCGTGGAAGGCGCAATCTGA |
| P_{nifH} of K. | Promoter | TGTTGCCTCAAGCACAGCCTGTGCCAGCTCGCGGATGACAGAAGAGTTAGCGCGAATTCAACGCGTTATGAAGAGAGTCGCCGCGCAGCGCG |
| oxytoca | | CCAAGAGATTGCGTGGAATAAGACACAGGGGGCGACAAGCTGTTGAACAGGCGACAAAGCGCCACCATGGCCCCGGCAGGCGCAATTGTTCT |
| | | GTTTCCCACATTTGGTCGCCTTATTGTGCCGTTTTGTTTTACGTCCTGCGCGGCGACAAATAACTAACTTCATAAAAATCATAAGAATACAT |
| | | AAACAGGCACGGCTGGTATGTTCCCTATTCTCTGCTGGCAAACA |
| P_{nifH} of P. | Promoter | TGTCATGTTCGCAACAGTTGCCGAAAGTGTGGAAAACCGGCGCTTGGCCCGGCCGATCTTTTTGTCGCCATTGCAACAGTCAGGCCTGTCGG |
| stutzeri | | TTGTTAACTATCGAACCGCCGAAGGATGTTGCTAGTAATTAAATTATTCTAATTAAAACAAGTGCTTAGATTATTTTAGAAACGCTGGCACA |
| | | AAGGCTGCTATTGCCCTGTTGCGCAGGCTTGTTCGTGCCTATAGCCCAC |
| P_{nifH} of A. | Promoter | TGTCATGTTTTAAACAGGGGGCCGGACCAGGATGGTGGACGCTCGATGGGGATGTCGGGCCATTGTTCGGTTGTAGCAATTACAACAGTCGG |
| vinelandii | | AGTAGGGGGATTGTAGGGGGATTGTTGTGTATCAGACCGCCCTGCAGCTCCCGTCGATGGATAATTAATCATTTAAAATCAATGCTTTATTT |
| | | ATGTGTTGCGGGTGCTGGCACAGACGCTGCATTACCTTTGGTGCGCGGAGTTGTTCGGGCTTACGGCCGAAC |
| P_{nifH} of | Promoter | TTGACAAAGCCTCCGAGAAGAGCGCCCCCTAACCCCTCCTCAGCCCTGATCGGCAGTATCATCTTGTCGAATCCTAACGTCTGATAGGCAAC |
| R. sp. | | GCTATACGACAAACGCTGGTTACAATTGTCGGTTCCGCGACAAGAATTTGCTTTGTCTGGCGGGTGCTCTATTTTGAGCTAAGTAGCTGAGA |
| IRBG74 | | AATCAGGAAAACAAAACTCTATTCGGTCTACCCGACGAGTTGGCACGGGTCTTGTAACCATCCTTGCGCAGGCGGCGAAAGCCACCGGCGAT |
| | | ATTCATGTTGCGGGCAAC |
| P_{nifH} Of A. | Promoter | TGTCGCGTTTGAAACACGGGGCTTTTGGAACCGTTCGATTCTGCAATGCACTGATTTTACTTGATTAATTCGACCACACGACCACTGGCACA |
| caulinodans | | CCCGTTGCAAAACCCCTTGGTGCAGGCGACGGGTTGCCGGTCTGGTTCGCGGATCTCCTCGATCCCCGGCTACCGACCCGCCTCCGAAAAGT |
| | | CCGGTCCCGATCCAGTTCGGCGGGGCCACAC |
| nifA of K. | Gene | ATGATCCATAAATCCGATTCGGACACCACCGTCAGACGTTTCGATCTCTCCCAGCAGTTTACCGCCATGCAGCGGATAAGCGTGGTCCTGAG |
| oxytoca | | TCGCGCCACCGAAGCGAGCAAAACCCTGCAGGAGGTTCTGAGCGTGCTACATAACGATGCCTTTATGCAGCACGGGATGATTTGCCTGTACG |
| | | ACAGCCAGCAGGAGATCCTGAGCATCGAAGCGCTGCAGCAAACGGAAGATCAGACGCTGCCCGGCAGTACGCAAATTCGCTACCGGCCGGGG |
| | | GAAGGATTAGTCGGTACCGTGCTGGCGCAGGGCCAGTCGCTGGTGCTGCCGCGCGTCGCCGACGACCAGCGTTTTCTCGATCGTCTGAGCCT |
| | | GTACGACTATGACCTGCCGTTTATCGCCGTTCCGCTGATGGGCCCCCACTCCCGGCCCATCGGCGTACTGGCGGCGCAGCCGATGGCGCGTC |
| | | AGGAAGAGCGGCTGCCCGCCTGCACGCGCTTTCTCGAAACCGTCGCCAATCTGATCGCCCAGACGATTCGCCTGATGATCCTGCCAACCTCC |
| | | GCCGCGCAGGCGCCGCAGCAGAGCCCCAGAATAGAGCGCCCGCGCGCCTGTACCCCTTCGCGCGGTTTCGGCCTGGAAAATATGGTCGGTAA |
| | | AAGCCCGGCGATGCGGCAGATTATGGATATTATTCGTCAGGTTTCCCGCTGGGATACCACGGTGCTGGTACGCGGCGAGAGCGGCACCGGGA |
| | | AAGAGCTCATCGCCAACGCCATCCACCATAATTCTCCGCGCGCCGCCGCGGCGTTCGTCAAATTTAACTGCGCGGCGCTGCCGGACAACCTG |
| | | CTGGAGAGCGAGCTGTTTGGTCATGAGAAAGGCGCGTTTACCGGCGCGGTGCGCCAGCGGAAAGGCCGCTTTGAGCTGGCGGACGGCGGCAC |
| | | CTTATTCCTCGATGAGATCGGCGAAAGCAGCGCCTCGTTTCAGGCTAAGCTACTGCGTATTCTGCAAGAGGGGGAGATGGAGCGCGTCGGCG |
| | | GCGACGAAACCCTGCGGGTCAACGTGCGCATTATCGCGGCGACCAACCGCCATCTGGAAGAGGAGGTGCGGCTGGGTCATTTCCGCGAGGAT |
| | | CTATACTACCGCCTGAACGTAATGCCTATCGCGCTGCCGCCGCTGCGCGAGCGCCAGGAGGATATCGCCGAGCTGGCGCACTTTCTGGTGCG |
| | | AAAAATCGCCCACAGCCAGGGGCGAACGCTGCGCATCAGCGATGGGGCGATTCGCCTGCTGATGGAGTACAGCTGGCCGGGAAACGTGCGCG |
| | | AACTGGAAAACTGTCTCGAACGTTCGGCGGTGCTGTCGGAAAGCGGCCTGATAGACCGGGACGTGATTCTGTTCAACCATCGCGATAACCCG |
| | | CCGAAAGCGCTCGCCAGCAGCGGCCCGGCGGAGGACGGCTGGCTCGATAACAGCCTCGACGAGCGCCAGCGGCTGATCGCCGCCCTGGAAAA |
| | | AGCGGGCTGGGTGCAGGCCAAAGCGGCGCGGCTGCTCGGCATGACCCCGCGCCAGGTGGCGTATCGCATTCAGATTATGGATATCACCATGC |
| | | CGCGACTGTGA |
| nifA of P. | Gene | ATGAACGCCACATTCGCCGAACGCCCCAGCGCGCCAACCCGCAACGAACTGCTGGATGCCCAACTGCAGGCGCTGGCGCAGATCGCCCGCAT |
| stutzeri | | CCTTAACCGCGGCCGGCCCATCGAGGAACTGCTGGCCGAGATCCTCGCCGTGCTGCACGAAGACCTCGGCCTGCTGCACGGGCTGGTCTCCA |
| | | TCTGCAACCCGAAGGACGGCAGCCTGCAGGTGGGCGCCGTGCACAGCGACTCCGAAACCGTGGTACGGGCCTGCGAAAGCACCCGCTACCGC |
| | | ATCGGCGAAGGCGTGTTCGGCAACATCCTCAAGCATGGCAACAGCGTGGTGCTCGGGCGTATCGACGCCGAACCGCGCTTTCTCGACCGACT |
| | | GGCGCTGTACGACATGGACCTGCCCTTCATCGCCGTGCCGATCAAGGCCGTCGACGGCACCACCATCGGCGTGCTGGCTGCCCAGCCCGACC |
| | | GCCGCGCCGACGAGCTGATGCCCGAACGCACCCGTTTGATGGAAATCGTCGCCCGCCTACTGGCGCAGACCGTGCGCCTGGTGGTGAACCTC |
| | | GAGGACGGCCAGGAAGTGGTCGACGAGCGCGACGAGCTACGCCGCGAAGTCCGCGCCAAGTACGGCTTCGAGAACATGGTGGTGGGCCACAC |
| | | CGCCTCCATGCGCCGGGTTTTCGACCAGGTTCGACGGGTCGCCAAGTGGAACAGCACCGTGCTGATCCTCGGCGAATCCGGCACCGGCAAGG |
| | | AGCTGATCGCCAGCGCCATCCACTACAACTCACCGCGCGCTCACCAGCCGCTGGTACGCCTGAACTGCGCCGCGCTACCGGAAACCCTGCTC |
| | | GAATCGGAACTGTTCGGTCACGAGAAAGGCGCCTTCACCGGCGCCGTGAAGCAGCGCAAGGGACGTTTCGAACAGGCCGACGGCGGCACCCT |
| | | GTTCCTCGACGAGATCGGCGAGATCTCGCCGATGTTCCAGGCCAAGCTGCTGCGCGTGCTGCAGGAAGGCGAGCTGGAGCGCGTCGGCGGCA |
| | | GCCAGACGGTGAAGGTCAACGTGCGCATCGTCGCCGCCACCAACCGCGACCTGGAGCACGAGGTGGAGCAAGGCAAGTTCCGCGAAGACCTC |
| | | TATATAATATATAATAATTATTTTAAA |
| | | GATCGCCCGCCAGCAGGGTCGCAAACTCAAGCTGACCGACAGCGCCCTGCGTCTGCTGATGAGCCACCGCTGGCCGGGCAACGTGCGCGAAC |
| | | TGGAAAACTGCCTGGAACGCTCGGCCATCATGAGCGAGGATGGCACCATCAGCCGCGACGTGGTCTCCCTCACCGGCCTCGACCACGACGCC |
| | | ACGCCGCTGGCGCCGGTCCCCGAAGTCGACCTCGCCGACGACAGCCTCGACGACCGCGAGCGCGTCATCGCCGCGCTGGAACAGGCCGGCTG |
| | | GGTCCAGGCCAAGGCCGCCCGCCTGCTCGGCATGACGCCCCGGCAGATCGCCTACCGAGTGCAGACGCTGAACATTCATATGCGCAAGATCT |
| | | GA |
| nifA of A. | Gene | ATGAATGCAACCATCCCTCAGCGCTCGGCCAAACAGAACCCGGTCGAACTCTATGACCTGCAATTGCAGGCCCTGGCGAGCATCGCCCGCAC |
| vinelandii | | GCTCAGCCGCGAACAACAGATCGACGAACTGCTCGAACAGGTCCTGGCCGTACTGCACAATGACCTCGGCCTGCTGCATGGCCTGGTGACCA |
| | | TTTCCGACCCGGAACACGGCGCCCTGCAGATCGGCGCCATCCACACCGACTCGGAAGCGGTGGCCCAGGCCTGCGAAGGCGTGCGCTACAGA |
| | | AGCGGCGAAGGCGTGATCGGCAACGTGCTCAAGCACGGCAACAGCGTGGTGCTCGGGCGCATCTCCGCCGACCCGCGCTTTCTCGACCGCCT |
| | | GGCGCTGTACGACCTGGAAATGCCGTTCATCGCCGTGCCGATCAAGAACCCCGAGGGCAACACCATCGGCGTGCTGGCGGCCCAGCCGGACT |
| | | GCCGCGCCGACGAGCACATGCCCGCGCGCACGCGCCTTCTGGAGATCGTCGCCAACCTGCTGGCGCAGACCGTGCGCCTGGTGGTGAACATC |
| | | GAGGACGGCCGCGAGGCGGCCGACGAGCGCGACGAACTGCGTCGCGAGGTGCGCGGCAAGTACGGCTTCGAGAACATGGTGGTGGGCCACAC |
| | | CCCCACCATGCGCCGGGTGTTCGATCAGATCCGCCGGGTCGCCAAGTGGAACAGCACCGTACTGGTCCTCGGCGAGTCCGGTACCGGCAAGG |
| | | AACTGATCGCCAGCGCCATCCACTACAACTCGCCGCGCGCGCACCGCCCCTTCGTGCGCCTGAACTGCGCCGCGCTGCCGGAAACCCTGCTC |
| | | GAGTCCGAACTCTTCGGCCACGAGAAGGGCGCCTTCACCGGCGCGGTGAAGCAGCGCAAGGGGCGTTTCGAGCAGGCCGACGGCGGCACCCT |
| | | GTTCCTCGACGAGATCGGCGAGATCTCGCCGATGTTCCAGGCCAAGCTGCTGCGCGTGCTGCAGGAAGGCGAGTTCGAGCGGGTCGGCGGCA |
| | | ACCAGACGGTGCGGGTCAACGTGCGCATCGTCGCCGCCACCAACCGCGACCTGGAAAGCGAGGTGGAAAAGGGCAAGTTCCGCGAGGACCTC |
| | | TACTACCGCCTGAACGTCATGGCCATCCGCATTCCGCCGCTGCGCGAGCGTACCGCCGACATTCCCGAACTGGCGGAATTCCTGCTCGGCAA |
| | | GATCGGCCGCCAGCAGGGCCGCCCGCTGACCGTCACCGACAGCGCCATCCGCCTGCTGATGAGCCACCGCTGGCCGGGCAACGTGCGCGAAC |
| | | TGGAGAACTGCCTGGAGCGCTCGGCGATCATGAGCGAGGACGGCACCATCACCCGCGACGTGGTCTCGCTGACCGGGGTCGACAACGAGAGC |
| | | CCGCCGCTCGCCGCGCCGCTGCCCGAGGTCAACCTGGCCGACGAGACCCTGGACGACCGCGAACGGGTGATCGCCGCCCTCGAACAGGCCGG |
| | | CTGGGTGCAGGCCAAGGCCGCGCGGCTGCTGGGCATGACGCCGCGGCAGATCGCCTACCGCATCCAGACCCTCAACATCCACATGCGCAAGA |
| | | TCTGA |
| nifA of | Gene | ATGCTGCACAATGGGCTCAATGAGGGTATGACTGAACGATCCGCTCAAACCATCCACAAACCGGATTTCTGGGGCAGCGGTATCTATCGGAT |
| R. sp. | | ATCGAAAGTTTTGATTGGTCCAGACAGTCTCGAGACGAAGCTTGCCAATGTCATTAACGCCCTCTCAGTAATTCTCCCAATGCGGCGCGGCG |
| IRBG74 | | CAATCGTCGTTCTAAATGTTAAAGGAGAGCCCGAGATGGTTGCAATGCTGGGCCTAGAGCAAGCATCTCAAGGCGCCCGCTCCATTCCGGCG |
| | | GAGGCTGCGATAGATAGAATCGTCGCCAAAGGCGCGCCGCTGGTCGTACCGGACATTTGCAAGTCGGACCTGTTCCAGGCGGAGCTCCAAAC |
| | | CAACTCGAACGCCACAGGCCCAGCCACGTTCGTTGGCGTCCCGATGAAGGTCGAAAAAGAAACGCTTGGAACACTATGGATCGACCGCGCCA |
| | | AAGATGGCAGCACTAGGATCCAATTTGAGGAAGAGGTGCGCTTCCTCTCCATGGTCGCCAACCTTTCGGCCCGGGCCATTTGGCTGGATCGC |
| | | CACCAGAGCCGCGATGGTCAGCCAATCGTGGGCGAGGAAGGAACTCGCAAGACTAGTTCAGGCGACAAGGAACTGCCCGAATCTGCCCGACA |
| | | AAGGCCCACAAAAATCGATTGGATTGTCGGGGAAAGCCCTGCCCTCAAGCAGGTGGTTGAAAGCGTCAAAGTCGTTGCAACAACCAATTCTG |
| | | CGGTGCTTCTCAGGGGCGAAAGCGGCACGGGCAAGGAGTTCTTTGCAAAGGCCATCCACGAGCTTTCATACCGGAAAAAGAAGCCCTTCGTG |
| | | AAGTTGAACTGCGCCGCGCTGTCTGCAGGCGTTTTGGAATCGGAATTGTTTGGACATGAAAAGGGCGCCTTCACGGGGGCCATCTCTCAGCG |
| | | CGCAGGCCGCTTCGAACTCGCAGACGGCGGAACGCTGCTGCTCGATGAGATCGGCGACATTTCGCCGGGCTTCCAAGCGAAACTGTTGCGCG |
| | | TCTTGCAGGAAGGTGAGCTTGAGCGAGTCGGCGGCACAAAAACACTCAAAGTGGACGTTCGACTCATATGCGCCACGAACAAAGACCTAGAA |
| | | GCGGCAGTCGCGGATGGGGAGTTCAGGGCCGACCTTTATTACCGGATCAATGTGGTGCCCCTATTTCTGCCGCCTCTCCGGGAGCGAAATGG |
| | | GGATATTCCACGCCTTGCGAGAGTTTTCCTCGGCCGATTCAACAGGGAAAACAATCGCGATCTCGCGTTCGCGCCGGCTGCGCTCGAGCTCT |
| | | TGTCAAAATGCAACTTTCCCGGCAACGTCCGAGAGCTTGAAAACTGCGTCCGCAGGACCGCCACTCTCGCGCGTTCGGAGACGATCGTTCCA |
| | | TCAGATTTCTCCTGCCTGAAGAACCAGTGCTTTTCTTCAATGCTCTGGAAAACCGGTGACCGTCCACTTGGGGATACGCTCAATGGGTTGGC |
| | | CATGCGTAAGAGTTTGTCGGTCGAATCGCCGATCAGCCTCGGTTACTCCAATGGACCGGCCGGCTTAACGGTGGCACCACATCTAACGGACC |
| | | GCGAGCTGCTAATCAGTGCGATGGAGAAGGCCGGTTGGGTTCAGGCAAAGGCAGCTCGGATCCTCGGCCTCACACCGCGACAGGTCGGCTAT |
| | | GCTTTACGTAGGCATCGTATACAGGTGAAGAAAATCTAA |
| nifA of A. | Gene | ATGCCAATGACCGACGCCTTCCAGGTCCGCGTACCTCGGGTTTCGTCGAGCACCGCCGGAGACATCGCCGCGTCATCCATCACCACGCGGGG |
| caulinodans | | CGCGCTGCCGCGCCCGGGAGGGATGCCTGTGTCCATGTCGCGGGGGACCTCGCCCGAGGTGGCACTCATCGGGGTCTATGAGATATCGAAGA |
| | | TCCTGACGGCGCCCCGGCGCCTCGAAGTCACGCTCGCCAATGTGGTGAACGTGCTCTCCTCCATGCTGCAGATGCGGCATGGCATGATCTGC |
| | | ATCCTCGACAGCGAGGGCGATCCCGACATGGTGGCCACCACCGGCTGGACGCCTGAGATGGCGGGCCAGATCCGCGCGCATGTGCCCCAGAA |
| | | GGCCATCGACCAGATCGTCGCCACGCAGATGCCGCTGGTGGTGCAGGACGTGACGGCCGATCCGCTCTTCGCCGGTCACGAGGATCTGTTCG |
| | | GCCCGCCTGAGGAGGCCACCGTCTCCTTCATCGGCGTGCCGATCAAGGCCGACCACCATGTGATGGGCACCCTCTCCATCGACCGCATCTGG |
| | | GACGGCACCGCCCGTTTCCGCTTCGACGAGGACGTGCGCTTCCTCACCATGGTGGCCAATCTCGTCGGCCAGACCGTGCGCCTGCACAAGCT |
| | | GGTGGCGAGCGACCGCGACCGGCTGATCGCCCAGACGCACCGCCTCGAAAAGGCGCTGCGGGAAGAAAAATCCGGGGCCGAGCCGGAGGTGG |
| | | CCGAGGCCGCCAACGGATCCGCCATGGGCATCGTGGGCGATAGCCCGCTGGTGAAACGCCTGATCGCGACCGCGCAAGTGGTCGCCCGCTCA |
| | | AACTCCACCGTGCTGCTGCGCGGGGAGAGCGGCACCGGCAAGGAGTTGTTCGCCCGTGCCATCCACGAACTGTCGCCCCGCAAGGGCAAGCC |
| | | CTTCGTGAAGGTGAACTGCGCCGCCCTCCCGGAATCGGTGCTGGAATCGGAACTGTTCGGCCATGAGAAGGGCGCCTTCACCGGTGCGCTGA |
| | | ACATGCGCCAGGGCCGCTTCGAGCTGGCGCACGGCGGCACGCTCTTCCTTGACGAGATCGGCGAGATCACCCCCGCTTTCCAGGCCAAGCTG |
| | | CTGCGCGTGCTGCAGGAAGGCGAGTTCGAGCGGGTCGGCGGCAATCGCACGCTGAAGGTGGATGTGCGGCTCGTGTGCGCCACCAACAAGAA |
| | | TCTGGAAGAGGCGGTCTCCAAGGGCGAGTTCCGGGCCGATCTCTACTACCGCATCCATGTGGTGCCGCTGATCCTGCCGCCGCTGCGCGAAC |
| | | GGCCGGGCGACATTCCCAAGCTCGCGAAGAACTTCCTCGACCGCTTCAACAAGGAAAACAAGCTCCACATGATGCTCTCGGCGCCGGCCATC |
| | | GACGTGCTGCGGCGCTGCTATTTCCCGGGCAACGTGCGCGAGCTGGAGAACTGTATCCGGCGGACGGCAACGCTCGCCCACGATGCCGTCAT |
| | | CACCCCCCATGACTTCGCCTGCGACAGCGGCCAGTGCCTCTCGGCCATGCTCTGGAAGGGCTCGGCCCCGAAGCCTGTGATGCCGCACGTGC |
| | | CGCCGGCGCCCACGCCGCTGACTCCGCTCTCCCCTGCTCCGCTCGCGACCGCAGCGCCCGCTGCGGCGAGCCCGGCGCCGGCGGCCGACAGC |
| | | CTGCCGGTCACTTGCCCCGGCACCGAGGCCTGTCCCGCGGTGCCCCCCCGCCAGAGCGAAAAGGAGCAGTTGCTCCAGGCCATGGAGCGCTC |
| | | CGGCTGGGTGCAGGCGAAGGCCGCGCGCCTCCTCAACCTCACGCCGCGCCAGGTGGGTTATGCGCTGCGCAAATATGACATCGACATCAAGC |
| | | GCTTCTGA |
| P_{J23100} | Promoter⁹ | TAGGTGTTGACGGCTAGCTCAGTCCTAGGTACAGTGCTAGCTCTAGA |
| P_{J23101} | Promoter⁹ | TAGGTGTTTACAGCTAGCTCAGTCCTAGGTATTATGCTAGCTCTAGA |
| P_{J23102} | Promoter⁹ | TAGGTGTTGACAGCTAGCTCAGTCCTAGGTACTGTGCTAGCTCTAGA |
| P_{J23103} | Promoter⁹ | TAGGTGCTGATAGCTAGCTCAGTCCTAGGGATTATGCTAGCTCTAGA |
| P_{J23104} | Promoter⁹ | TAGGTGTTGACAGCTAGCTCAGTCCTAGGTATTGTGCTAGCTCTAGA |
| P_{J23105} | Promoter⁹ | TAGGTGTTTACGGCTAGCTCAGTCCTAGGTACTATGCTAGCTCTAGA |
| P_{J23106} | Promoter⁹ | TAGGTGTTTACGGCTAGCTCAGTCCTAGGTATAGTGCTAGCTCTAGA |
| P_{J23107} | Promoter⁹ | TAGGTGTTTACGGCTAGCTCAGCCCTAGGTATTATGCTAGGTCTAGA |
| P_{J23108} | Promoter⁹ | TAGGTGCTGACAGCTAGCTCAGTCCTAGGTATAATGCTAGGTCTAGA |
| P_{J23109} | Promoter⁹ | TAGGTGTTTACAGCTAGGTCAGTCCTAGGGACTGTGCTAGGTCTAGA |
| P_{J23110} | Promoter⁹ | TAGGTGTTTACGGCTAGGTCAGTCCTAGGTACAATGCTAGGTCTAGA |
| P_{J23111} | Promoter⁹ | TAGGTGTTGACGGCTAGGTCAGTCCTAGGTATAGTGCTAGGTCTAGA |
| P_{J23112} | Promoter⁹ | TAGGTGCTGATAGCTAGGTCAGTCCTAGGGATTATGCTAGGTCTAGA |
| P_{J23113} | Promoter⁹ | TAGGTGCTGATGGCTAGGTCAGTCCTAGGGATTATGCTAGGTCTAGA |
| P_{J23114} | Promoter⁹ | TAGGTGTTTATGGCTAGGTCAGTCCTAGGTACAATGCTAGGTCTAGA |
| P_{J23115} | Promoter⁹ | TAGGTGTTTATAGCTAGCTCAGCCCTTGGTACAATGCTAGCTCTAGA |
| P_{J23116} | Promoter⁹ | TAGGTGTTGACAGCTAGGTCAGTCCTAGGGACTATGCTAGGTCTAGA |
| P_{J23117} | Promoter⁹ | TAGGTGTTGACAGCTAGGTCAGTCCTAGGGATTGTGCTAGGTCTAGA |
| P_{J23118} | Promoter⁹ | TAGGTGTTGACGGCTAGGTCAGTCCTAGGTATTGTGCTAGCTCTAGA |
| P_{J23119} | Promoter⁹ | TAGGTGTTGACAGCTAGCTCAGTCCTAGGTATAATGCTAGCTCTAGA |
| Pₐₚ | Promoter¹⁰ | TAGGTGTTGACATTATTCCATCGAACTAGTTAACTAGTACGAAAGTT |
| T_{T7} | Terminator¹¹ | TAGCATAACCCCTTGGGGCCTCTAAACGGGTCTTGAGGGGTTTTTTGT |
| T_{T7.2} | Terminator¹¹ | TACTCGAACCCCTAGCCCGCTCTTATCGGGCGGCTAGGGGTTTTTTGT |
| T_{F7.3} | Terminator¹¹ | TACATATCGGGGGGGTAGGGGTTTTTTGT |
| T_{rmBT1} | Terminator¹² | CCAGGCATCAAATAAAACGAAAGGCTCAGTCGAAAGACTGGGCCTTTCGTTTTATCTGTTGTTTGTCGGTGAACGCTCTC |
| T_{L3S2P21} | Terminator¹³ | CTCGGTACCAAATTCCAGAAAAGAGGCCTCCCGAAAGGGGGGCCTTTTTTCGTTTTGGTCC |
| T₁ | Terminator¹³ | CTCGGTACCAAATTCCAGAAAAGACACCCGAAAGGGTGTTTTTTCGTTTTGGTCCTCCTTGGCCCTCCATCCTTAGATAGCAGATAAAAAAA |
| | | ATCCTTAGCTTTCGCTAAGGATGATTTCTTCATAGGCAATACGATCGCATGTCC |
| T₂ | Terminator¹⁴ | CCAGGCATCAAATAAAACGAAAGGCTCAGTCGAAAGACTGGGCCTTTCGTTTTATCTGTTGTTTGTCGGTGAACGCTCTCTACTAGAGTCAC |
| | | ACTGGCTCACCTTCGGGTGGGCCTTTCTGCGTTTATA |
| T₃ | Terminator¹³ | CCAGGCATCAAATAAAACGAAAGGCTCAGTCGAAAGACTGGGCCTTTCGTTTTATCTGTTGTTTGTCGGTGAACGCTCTCTACTAGAGTCAC |
| | | ACTGGCTCACCTTCGGGTGGGCCTTTCTGCGTTTATA |
| T₄ | Terminator¹³ | GGTCTTGTCCACTACCTTGCAGTAATGCGGTGGACAGGATCGGCGGTTTTCTTTTCTCTTCTCAATGACTGAATAGAAAAGACGAACATTAA |
| | | CGCATGAGAAAGCCCCCGGAAGATCACCTTCCGGGGGCTTTTTTATTGCGCTACAAATGAAAGTACATAGAAATTA |
| T₅ | Terminator¹³ | CAGATAAAAAAAATCCTTAGCTTTCGCTAAGGATGATTTCTTCCTTGGCCCTCCATCCTTAGATAGCTCGGTACCAAATTCCAGAAAAGACA |
| | | CCCGAAAGGGTGTTTTTTCGTTTTGGTCCTCATAGGCAATACGATCGCATGTCC |
| T₆ | Terminator¹³ | CCAGGCATCAAATAAAACGAAAGGCTCAGTCGAAAGACTGGGCCTTTCGTTTTATCTGTTGTTTGTCGGTGAACGCTCTCCTAGCATAACCC |
| | | CTTGGGGCCTCTAAACGGGTCTTGAGGGGTTTTTTG |
| T₇ | Terminator¹³ | CTCGGTACCAAATTCCAGAAAAGAGACGCTTTCGAGCGTCTTTTTTCGTTTTGGTCCTCCTTGGCCCTCCATCCTTAGATAGAGTTAACCAA |
| | | AAAGGGGGGATTTTATCTCCCCTTTAATTTTTCCTTCATAGGCAATACGATCGCATGTCC |
| T₈ | Terminator¹³ | CGCAGATAGCAAAAAAGCGCCTTTAGGGCGCTTTTTTACATTGGTGGTCCTTGGCCCTCCATCCTTAGATAGAGGCGACTGACGAAACCTCG |
| | | CTCCGGCGGGGTTTTTTGTTATCTGCATCATAGGCAATACGATCGCATGTCC |
| T₉ | Terminator¹⁴ | TCGGTCAGTTTCACCTGATTTACGTAAAAACCCGCTTCGGCGGGTTTTTGCTTTTGGAGGGGCAGAAAGATGAATGACTGTC |
| T₁₀ | Terminator¹⁴ | GCCCCCGGAAGATCACCTTCCGGGGGCTTTTTTATTGGCGGCCGGCTGATTGATCAGGCGGCCGGCTGATTGGCGCGTTACCTGGTAGCGCG |
| | | CCATTTTGTTT |
| T₁₁ | Terminator¹⁴ | GTAATCGTTAATCCGCAAATAACGTAAAAACCCGCTTCGGCGGGTTTTTTTATGGGGGGAGTTTAGGGAAAGAGCATTTGTCA |
| T₁₂ | Terminator¹⁴ | AAAAAAAAACCCCGCCCCTGACAGGGCGGGGTTTTTTTTTT |
| T₁₃ | Terminator¹³ | TCCGGCAATTAAAAAAGCGGCTAACCACGCCGCTTTTTTTACGTCTGCATGACTGAATAGAAAAGACGAACATTAACGCATGAGAAAGCCCC |
| | | CGGAAGATCACCTTCCGGGGGCTTTTTTATTGCGCTCCTTGGCCCTCCATCCTTAGATAG |
| T₁₄ | Terminator¹³ | GGAAGACCATACTGGAAACACAGAAAAAAGCCCGCACCTGACAGTGCGGGCTTTTTTTTTCGACCAAAGGTGACTGAATAGAAAAGACGAAC |
| | | ATTCGCAGATAGCAAAAAAGCGCCTTTAGGGCGCTTTTTTACATTGGTGGTCATAGGCAATACGATCGCATGTCC |
| T₁₅ | Terminator¹³ | TCCGGCAATTAAAAAAGCGGCTAACCACGCCGCTTTTTTTACGTCTGCATCCTTGGCCCTCCATCCTTAGATAGCTCGGTACCAAATTCCAG |
| | | AAAAGAGGCCTCCCGAAAGGGGGGCCTTTTTTCGTTTTGGTCCTCATAGGCAATACGATCGCATGTCC |
| T₁₆ | Terminator¹³ | TTCAGCCAAAAAACTTAAGACCGCCGGTCTTGTCCACTACCTTGCAGTAATGCGGTGGACAGGATCGGCGGTTTTCTTTTCTCTTCTCAATA |
| | | CATGAAAGTACATAGAAATTACTCGGTACCAAATTCCAGAAAAGAGGCCTCCCGAAAGGGGGGCCTTTTTTCGTTTTGGTCCTCATAGGCAA |
| | | TACGATCGCATGTCC |
| T₁₇ | Terminator¹³ | TTCAGCCAAAAAACTTAAGACCGCCGGTCTTGTCCACTACCTTGCAGTAATGCGGTGGACAGGATCGGCGGTTTTCTTTTCTCTTCTCAATC |
| | | CTTGGCCCTCCATCCTTAGATAGTCCGGCAATTAAAAAAGCGGCTAACCACGCCGCTTTTTTTACGTCTGCATCATAGGCAATACGATCGCA |
| | | TGTCC |
| T₁₈ | Terminator¹³ | CTCGGTACCAAATTCCAGAAAAGAGGCCTCCCGAAAGGGGGGCCTTTTTTCGTTTTGGTCCTGACTGAATAGAAAAGACGAACATTAACGCA |
| | | TGAGAAAGCCCCCGGAAGATCACCTTCCGGGGGCTTTTTTATTGCGCTCCTTGGCCCTCCATCCTTAGATAG |
| T₁₉ | Terminator¹³ | CTCGGTACCAAATTCCAGAAAAGAGGCCTCCCGAAAGGGGGGCCTTTTTTCGTTTTGGTCCTCCTTGGCCCTCCATCCTTAGATGTCCGGCA |
| | | ATTAAAAAAGCGGCTAACCACGCCGCTTTTTTTACGTCTGCATCATAGGCAATACGATCGCATGTCC |
| T₂₀ | Terminator¹³ | CTCGGTACCAAAGACGAACAATAAGACGCTGAAAAGCGTCTTTTTTCGTTTTGGTCCTACAAATGAAAGTACATAGAAATTATTCAGCCAAA |
| | | AAACTTAAGACCGCCGGTCTTGTCCACTACCTTGCAGTAATGCGGTGGACAGGATCGGCGGTTTTCTTTTCTCTTCTCAATCCTTGGCCCTC |
| | | CATCCTTAGATAG |
| T₂₁ | Terminator¹⁴ | GGGAACTGCCAGACATCAAATAAAACAAAAGGCTCAGTCGGAAGACTGGGCCTTTTGTTTTATCTGTTGTTTGTCGGTGAACACTCTCCCGA |
| | | CTAGTAGCGGCCGCTGCAGAAAGAGGAGA |
| T₂₂ | Terminator¹³ | AACGCATGAGAAAGCCCCCGGAAGATCACCTTCCGGGGGCTTTTTTATTGCGCTCATAGGCAATACGATCGCATGTCCTCCGGCAATTAAAA |
| | | AAGCGGCTAACCACGCCGCTTTTTTTACGTCTGCATCCTTGGCCCTCCATCCTTAGATAG |
| T₂₃ | Terminator¹⁴ | GGGAACTGCCAGACATCAAATAAAACAAAAGGCTCAGTCGGAAGACTGGGCCTTTTGTTTTATCTGTTGTTTGTCGGTGAACACTCTCCCG |
| T₂₄ | Terminator¹⁴ | AAAGCAAGCTGATAAACCGATACAATTAAAGGCTCCTTTTGGAGCCTTTTTTTTTGGAGATTTTCAACATGAAAAAATTATTATTTGATGAT |
| | | CAGATAGCGGCGGGGAACTGCCAGACATCAAATAAAACAAAAGGCTCAGTCGGAAGACTGGGCCTTTTGTTTTATCTGTTGTTTGTCGGTGA |
| | | ACACTCTCCCG |
| T₂₅ | Terminator¹³ | AAATAAAAAAATATTATATTAAZATT |
| | | CCAGAAAAGAGGCCTCCCGAAAGGGGGGCCTTTTTTCGTTTTGGTCCTCATAGGCAATACGATCGCATGTCC |
| T₂₆ | Terminator¹⁴ | AAATAAAAAAATATTATATTAAAZ1AA |
| | | TTAAGACCGCCGGTCTTGTCCACTACCTTGCAGTAATGCGGTGGACAGGATCGGCGGTTTTCTTTTCTCTTCTCAATCATAGGCAATACGAT |
| | | CGCATGTCC |
| P_{Lux} | Promoter¹⁵ | CCTAGGACCTGTAGGATCGTACAGGTTTACGCAAGAAAATGGTTTGTTACTTTCGAATAAATCTAGA |
| P_{Tet} | Promoter¹⁶ | CGGTGGAATCCCTATCAGTGATAGAGATTGACATCCCTATCAGTGATAGATATAATGAGCACTCTAGA |
| P_{Cym} | Promoter⁵ | AACAAACAGACAATCTGGTCTGTTTGTATTATGGAAAATTTTTCTGTATAATAGATTCAACAAACAGACAATCTGGTCTGTTTGTATTAT |
| P_{Phl} | Promoter¹⁷ | AAAAAGAGTTTGACATGATACGAAACGTACCGTATCGTTAAGGTTACTAGAGTCTAGA |
| P_{Sal} | Promoter⁵ | GGGGCCTCGCTTGGGTTATTGCTGGTGCCCGGCCGGGCGCAATATTCATGTTGATGATTTATTATATATCGAGTGGTGTATTTATTTATATT |
| | | GTTTGCTCCGTTACCGTTATTAAC |
| luxR | Gene | ATGAAAAACATAAATGCCGACGACACATACAGAATAATTAATAAAATTAAAGCTTGTAGAAGCAATAATGATATTAATCAATGCTTATCTGA |
| | | TATGACTAAAATGGTACATTGTGAATATTATTTACTCGCGATCATTTATCCTCATTCTATGGTTAAATCTGATATTTCAATCCTAGATAATT |
| | | ACCCTAAAAAATGGAGGCAATATTATGATGACGCTAATTTAATAAAATATGATCCTATAGTAGATTATTCTAACTCCAATCATTCACCAATT |
| | | AATTGGAATATATTTGAAAACAATGCTGTAAATAAAAAATCTCCAAATGTAATTAAAGAAGCGAAAACATCAGGTCTTATCACTGGGTTTAG |
| | | TTTCCCTATTCATACGGCTAACAATGGCTTCGGAATGCTTAGTTTTGCACATTCAGAAAAAGACAACTATATAGATAGTTTATTTTTACATG |
| | | CGTGTATGAACATACCATTAATTGTTCCTTCTCTAGTTGATAATTATCGAAAAATAAATATAGCAAATAATAAATCAAACAACGATTTAACC |
| | | AAAAGAGAAAAAGAATGTTTAGCGTGGGCATGCGAAGGAAAAAGCTCTTGGGATATTTCAAAAATATTAGGTTGCAGTGAGCGTACTGTCAC |
| | | TTTCCATTTAACCAATGCGCAAATGAAACTCAATACAACAAACCGCTGCCAAAGTATTTCTAAAGCAATTTTAACAGGAGCAATTGATTGCC |
| | | CATACTTTAAAAATTAA |
| tetR | Gene | ATGTCCAGATTAGATAAAAGTAAAGTGATTAACAGCGCATTAGAGCTGCTTAATGAGGTCGGAATCGAAGGTTTAACAACCCGTAAACTCGC |
| | | CCAGAAGCTAGGTGTAGAGCAGCCTACATTGTATTGGCATGTAAAAAATAAGCGGGCTTTGCTCGACGCCTTAGCCATTGAGATGTTAGATA |
| | | GGCACCATACTCACTTTTGCCCTTTAGAAGGGGAAAGCTGGCAAGATTTTTTACGTAATAACGCTAAAAGTTTTAGATGTGCTTTACTAAGT |
| | | CATCGCGATGGAGCAAAAGTACATTTAGGTACACGGCCTACAGAAAAACAGTATGAAACTCTCGAAAATCAATTAGCCTTTTTATGCCAACA |
| | | AGGTTTTTCACTAGAGAATGCATTATATGCACTCAGCGCTGTGGGGCATTTTACTTTAGGTTGCGTATTGGAAGATCAAGAGCATCAAGTCG |
| | | CTAAAGAAGAAAGGGAAACACCTACTACTGATAGTATGCCGCCATTATTACGACAAGCTATCGAATTATTTGATCACCAAGGTGCAGAGCCA |
| | | GCCTTCTTATTCGGCCTTGAATTGATCATATGCGGATTAGAAAAACAACTTAAATGTGAAAGTGGGTCCTAA |
| cymR | Gene | ATGAGCCCGAAACGTCGTACCCAGGCAGAACGTGCAATGGAAACCCAGGGTAAACTGATTGCAGCAGCACTGGGTGTTCTGCGTGAAAAAGG |
| | | TTATGCAGGTTTTCGTATTGCAGATGTTCCGGGTGCAGCCGGTGTTAGCCGTGGTGCACAGAGCCATCATTTTCCGACCAAACTGGAACTGC |
| | | TGCTGGCAACCTTTGAATGGCTGTATGAGCAGATTACCGAACGTAGCCGTGCACGTCTGGCAAAACTGAAACCGGAAGATGATGTTATTCAG |
| | | CAGATGCTGGATGATGCAGCAGAATTTTTTCTGGATGATGATTTTAGCATCAGCCTGGATCTGATTGTTGCAGCAGATCGTGATCCGGCACT |
| | | GCGTGAAGGTATTCAGCGTACCGTTGAACGTAATCGTTTTGTTGTTGAAGATATGTGGCTGGGTGTGCTGGTGAGCCGTGGTCTGAGCCGTG |
| | | ATGATGCCGAAGATATTCTGTGGCTGATTTTTAACAGCGTTCGTGGTCTGGCAGTTCGTAGCCTGTGGCAGAAAGATAAAGAACGTTTTGAA |
| | | CGTGTGCGTAATAGCACCCTGGAAATTGCACGTGAACGTTATGCAAAATTCAAACGTTGA |
| phlF | Gene | ATGGCACGTACCCCGAGCCGTAGCAGCATTGGTAGCCTGCGTAGTCCGCATACCCATAAAGCAATTCTGACCAGCACCATTGAAATCCTGAA |
| | | AGAATGTGGTTATAGCGGTCTGAGCATTGAAAGCGTTGCACGTCGTGCCGGTGCAAGCAAACCGACCATTTATCGTTGGTGGACCAATAAAG |
| | | AATATTAATTATATTATAAAAAATTAZATTTATTTATTTAZAATTATTTTT |
| | | CTGCGTAATCTGTGGAAAGTTTGGCGTGAAACCATTTGTGGTGAAGCATTTCGTTGTGTTATTGCAGAAGCACAGCTGGACCCTGCAACCCT |
| | | GACCCAGCTGAAAGATCAGTTTATGGAACGTCGTCGTGAGATGCCGAAAAAACTGGTTGAAAATGCCATTAGCAATGGTGAACTGCCGAAAG |
| | | ATACCAATCGTGAACTGCTGCTGGATATGATTTTTGGTTTTTGTTGGTATCGCCTGCTGACCGAACAGCTGACCGTTGAACAGGATATTGAA |
| | | GAATTTACCTTCCTGCTGATTAATGGTGTTTGTCCGGGTACACAGCGTTAA |
| nahR | Gene | ATGGAACTGCGTGACCTGGATTTAAACCTGCTGGTGGTGTTCAACCAGTTGCTGGTCGACAGACGCGTCTCTGTCACTGCGGAGAACCTGGG |
| | | CCTGACCCAGCCTGCCGTGAGCAATGCGCTGAAACGCCTGCGCACCTCGCTACAGGACCCACTCTTCGTGCGCACACATCAGGGAATGGAAC |
| | | CCACACCCTATGCCGCGCATCTGGCCGAGCACGTCACTTCGGCCATGCACGCACTGCGCAACGCCCTACAGCACCATGAAAGCTTCGATCCG |
| | | CTGACCAGCGAGCGTACCTTCACCCTGGCCATGACCGACATTGGCGAGATCTACTTCATGCCGCGGCTGATGGATGCGCTGGCTCACCAGGC |
| | | CCCCAATTGCGTGATCAGTACGGTGCGCGACAGTTCGATGAGCCTGATGCAGGCCTTGCAGAACGGAACCGTGGACTTGGCCGTGGGCCTGC |
| | | TTCCCAATCTGCAAACTGGCTTCTTTCAGCGCCGGCTGCTCCAGAATCACTACGTGTGCCTATGTCGCAAGGACCATCCAGTCACCCGCGAA |
| | | CCCCTGACTCTGGAGCGCTTCTGTTCCTACGGCCACGTGCGTGTCATCGCCGCTGGCACCGGCCACGGCGAGGTGGACACGTACATGACACG |
| | | TATAATTTAATATTTTAATTAAATTTATT |
| | | CGATATGTTTAGCCGACTGCTGCGTAGAGCCCTTCGGCCTAAGCGCCTTGCCGCACCCAGTCGTCTTGCCTGAAATAGCCATCAACATGTTC |
| | | TATAATAAAAATAAATATTTTTAATATTTTATTTTAATTATAA |
| P_{Fde} | Promoter¹⁸ | TCAATGTATTGATGCCGTCCATATCATGAATCAAAACAATCCATTTGATCAATATCAAGCTCACTCTTAAGCTTCACTCATCCGCTGCAT |
| fdeR | Gene | ATGCGTTTCAACAAGCTCGACCTCAATCTTCTGGTCGCCCTGGATGCACTGCTCACGGAGATGAGCATCAGCCGCGCCGCCGAAAAGATCCA |
| | | TCTGAGCCAGTCGGCCATGAGCAATGCCCTGGCGCGGCTGCGCGAGTATTTCGATGATGAATTGCTGATCCAGGTGGGCCGGCGCATGGAGC |
| | | CCACGCCGCGCGCCGAGGTGCTCAAGGATGCGGTGCATGATGTGCTGCGGCGTATCGATGGCTCCATCGCGGCGCTGCCGGCCTTCGTGCCG |
| | | GCCGAGTCCACGCGCGAGTTTCGCATCTCGGTTTCGGACTTTACGCTCTCCGTCCTCATCCCCCGGGTGCTGGCGCGCGCGCACGCCGAGGG |
| | | CAAGCACATCCGCTTTGCCCTGATGCCGCAGGTGCAAGACCCGACCCGCTCGCTG3ATCGGCCCGAGGTGGACCTGCT3GTCTTGCCGCAGG |
| | | AATTCTGCACGCCCGATCATCCTGCCGAAGAGGTCTTCCGCGAACGGCATGTCTGCGTGGTCTGGCGCGACAGTGCGCTGGCGCAAGGCGAG |
| | | CTGACGCTGGAACGCTAATGGCCTCAGGCCATGTGGTGATGGTGCCGCCTGTGGGCCAATGCGTCGTCGGTGGAGGCGTGGATGGCCAGGAA |
| | | GCTGGGCTTTGCGCGCCGGGTGGAAGTGACCAGCTTAGCTTCGCTTCTGCGCTGGCGCTGGTACAGGGAGACGGACCGCATCGCCACGGTGC |
| | | ATGCCCGGCTGGCGCAGCTGCTGGCTCCGCAATGGCCGGTGGTGATCAAGGAGAGTCCGCTGTCGCTGGGCGAGATGCGGCAGATGATGCAG |
| | | TGGCATCGCTACCGCAGCAATATTTGGCATCCTAGTGGCTGCGTCGGGTGTTTCTGGAGAGTGACGCAGGAGATGGATGCGGCGCTGCCAGG |
| | | CATTGCTGA |
| P_{BAD.10} | Promoter | CAGACATTGCCGTCACTGCGTCTTTTACTGGCTCTTCTCGCTAACCAAACCGGTAACCCCGCTTATTAAAAGCATTCTGTAACAAAGCGGGA |
| | | CCAAAGCCATGACAAAAACGCGTAACAAAAGTGTCTATAATCACGGCAGAAAAGTCCACATTGATTATTTGCACGGCGTCACACTTTGCTAT |
| | | GCCATAGCATTTTTATCCATAAGATTAGCGGATCCTACCTGACGCTTTTTATCGCAACTCTCTATATTTTCTCCATACCCGTTTTTTTGGGC |
| | | TAGCGAATTC |
| araC | Gene | ATGCAATATGGACAATTGGTTTCTTCTCTGAATGGCGGGAGTATGAAAAGTATGGCTGAAGCGCAAAATGATCCCCTGCTGCCGGGATACTC |
| | | GTTTAATGCCCATCTGGTGGCGGGTTTAACGCCGATTGAGGCCAACGGTTATCTCGATTTTTTTATCGACCGACCGCTGGGAATGAAAGGTT |
| | | ATATTCTCAATCTCACCATTCGCGGTCAGGGGGTGGTGAAAAATCAGGGACGAGAATTTGTTTGCCGACCGGGTGATATTTTGCTGTTCCCG |
| | | CCAGGAGAGATTCATCACTACGGTCGTCATCCGGAGGCTCGCGAATGGTATCACCAGTGGGTTTACTTTCGTCCGCGCGCCTACTGGCATGA |
| | | ATGGCTTAACTGGCCGTCAATATTTGCCAATACGGGGTTCTTTCGCCCGGATGAAGCGCACCAGCCGCATTTCAGCGACCTGTTTGGGCAAA |
| | | TCATTAACGCCGGGCAAGGGGAAGGGCGCTATTCGGAGCTGCTGGCGATAAATCTGCTTGAGCAATTGTTACTGCGGCGCATGGAAGCGATT |
| | | AACGAGTCGCTCCATCCACCGATGGATAATCGGGTACGCGAGGCTTGTCAGTACATCAGCGATCACCTGGCAGACAGCAATTTTGATATCGC |
| | | CAGCGTCGCACAGCATGTTTGCTTGTCGCCGTCGCGTCTGTCACATCTTTTCCGCCAGCAGTTAGGGATTAGCGTCTTAAGCTGGCGCGAGG |
| | | ACCAACGTATCAGCCAGGCGAAGCTGCTTTTGAGCACCACCCGGATGCCTATCGCCACCGTCGGTCGCAATGTTGGTTTTGACGATCAACTC |
| | | TATTTCTCGCGGGTATTTAAAAAATGCACCGGGGCCAGCCCGAGCGAGTTCCGTGCCGGTTGTGAAGAAAAAGTGAATGATGTAGCCGTCAA |
| | | GTTGTCATAA |
| araE | Gene | ATGGTTACTATCAATACGGAATCTGCTTTAACGCCACGTTCTTTGCGGGATACGCGGCGTATGAATATGTTTGTTTCGGTAGCTGCTGCGGT |
| | | CGCAGGATTGTTATTTGGTCTTGATATCGGCGTAATCGCCGGAGCGTTGCCGTTCATTACCGATCACTTTGTGCTGACCAGTCGTTTGCAGG |
| | | AATGGGTGGTTAGTAGCATGATGCTCGGTGCAGCAATTGGTGCGCTGTTTAATGGTTGGCTGTCGTTCCGCCTGGGGCGTAAATACAGCCTG |
| | | ATGGCGGGGGCCATCCTGTTTGTACTCGGTTCTATAGGGTCCGCTTTTGCGACCAGCGTAGAGATGTTAATCGCCGCTCGTGTGGTGCTGGG |
| | | GATTGCTGTCGGGATCGCGTCTTACACCGCTCCTCTGTATCTTTCTGAAATGGCAAGTGAAAACGTTCGCGGTAAGATGATCAGTATGTACC |
| | | AGTTGATGGTCACACTCGGCATCGTGCTGGCGTTTTTATCCGATACAGCGTTCAGTTATAGCGGTAACTGGCGCGCAATGTTGGGGGTTCTT |
| | | GCTTTACCAGCAGTTCTGCTGATTATTCTGGTAGTCTTCCTGCCAAATAGCCCGCGCTGGCTGGCGGAAAAGGGGCGTCATATTGAGGCGGA |
| | | AGAAGTATTGCGTATGCTGCGCGATACGTCGGAAAAAGCGCGAGAAGAACTCAACGAAATTCGTGAAAGCCTGAAGTTAAAACAGGGCGGTT |
| | | GGGCACTGTTTAAGATCAACCGTAACGTCCGTCGTGCTGTGTTTCTCGGTATGTTGTTGCAGGCGATGCAGCAGTTTACCGGTATGAACATC |
| | | ATCATGTACTACGCGCCGCGTATCTTCAAAATGGCGGGCTTTACGACCACAGAACAACAGATGATTGCGACTCTGGTCGTAGGGCTGACCTT |
| | | TATGTTCGCCACCTTTATTGCGGTGTTTACGGTAGATAAAGCAGGGCGTAAACCGGCTCTGAAAATTGGTTTCAGCGTGATGGCGTTAGGCA |
| | | CTCTGGTGCTGGGCTATTGCCTGATGCAGTTTGATAACGGTACGGCTTCCAGTGGC+32TCCTGGCTCTCTGTTGGCATGACGATGATGTGT |
| | | ATTGCCGGTTATGCGATGAGCGCCGCGCCAGTGGTGTGGATCCTGTGCTCTGAAATTCAGCCGCTGAAATGCCGCGATTTCGGTATTACCTG |
| | | TTCGACCACCACGAACTGGGTGTCGAATATGATTATCGGCGCGACCTTCCTGACACTGCTTCATAGCATTGGCGCTGCCGGTACGTTCTGGC |
| | | TCTACACTGCGCTGAACATTGCGTTTGTGGGCATTACTTTCTGGCTCATTCCGGAAACCAAAAATGTCACGCTGGAACATATCGAACGCAAA |
| | | CTGATGGCAGGCGAGAAGTTGAGAAATATCGGCGTCTGA |
| P_{tac} | Promoter¹⁰ | CTCGAGTGTTGACAATTAATCATCGGCTCGTATAATGTGTGGAATTGTGAGCGCTCACAATTTCACACATCTAGA |
| P_{noc} | Promoter | AACAAATACACATGGGCGCATGCCTATTACTGCCCTTGCGATATGGAAGGCAAGCTTTTAGTAACAATAGAAAACTGGGTCCTACTCTCGAA |
| | | GAATGCACTGCGGCGGTCACGTCAACACGTGCTGCACCGTTGAGAATGTATGCTGGGCAGATTGCCAGCGGCGTGATTTTCGGCTGTCCCGT |
| | | CCTCACGGTTTTGCGCTGCATCGCAAGAGATTGGGAA |
| nocR | Gene | ATGACGTCAGCAGCGAATCTGGTGAGGATCACGCAGCCCGCGATCAGCCGGCTGATCAGGGATCTCGAAGAGGAAATTGGGATCAGCCTCTT |
| | | CGAAAGAACGGGCAACCGGTTACGTCCTACGCGGGAGGCCGGTATTCTGTTCAAGGAAGTGTCGCGACATTTCAACGGGATTCAGCACATCG |
| | | ACAAAGTCGCGGCTGAACTGAAGAAGTCTCATATGGGGTCCCTAAGGGTCGCCTGTTATACAGCGCCAGCTCTGAGTTTTATGTCCGGCGTC |
| | | ATTCAGACGTTCATCGCCGATCGGCCCGACGTGTCGGTCTACCTCGACACAGTTCCTTCCCAGACGGTCCTCGAATTGGTCTCGCTCCAGCA |
| | | CTACGATTCGGAATATCGATATTGGCTGGCGACTATCCTGGTCTCACCACCGAACCTGTCCC+32CCTTTCGTGCGGTCTGCCTGCTGCCGC |
| | | CGGGGCATCGTCTCGAAGACAAGGAAACTGTTCATGCGACGGACCTTGAAGGAGAGTCATTGATTTGCCTCTCTCCAGTGAGCCTTCTACGG |
| | | ATGCAAACGGACGCCGCACTGGACAGCTGCGGCGTCCACTGTAATCGCAGGATAGAAAGTAGTCTGGCGCTGAATCTCTGCGATCTGGTAAG |
| | | CAGCGGAATGGGGGTTGGTATCGTCGACCCCTTCACTGCCGACTACTACAGTGCAAATCCGGTTATTCAGCGCTCCTTTGATCCGGTTGTCC |
| | | CCTACCATTTTGCTATAGTTCTTCCGACCGACAGCCCACCGCCGCGCTTGGTTAGCGAGTTCCGGGCAGCGTTGCTTGATGCTTTGAAAGCC |
| | | TTGCCCTATGAAACCATTTGA |
| P_{occ} | Promoter | AAACGCACCATAACATCTGCTTATTCTTGCCCGGTCATTATGAATTTGACCGAATGCATATCGAATGTAAAGCTCACCCTATAAATCACAAC |
| | | TCTTATACAGACGT |
| occR | Gene | ATGAATCTCAGGCAGGTCGAGGCGTTCCGGGCAGTCATGCTGACGGGGCAAATGACGGCGGCGGCTGAACTAATGCTGGTGACTCAGCCGGC |
| | | CATCAGTCGCCTAATCAAGGACTTTGAACAGGCGACAAAACTGCAGCTCTTCGAGAGGCGTGGGAACCATATTATCCCGACACAGGAGGCAA |
| | | AGACGCTGTGGAAAGAGGTCGATCGGGCGTTCGTCGGGCTTAATCATATAGGCAACCTGGCTGCCGACATCGGCAGGCAGGCAGCGGGGACG |
| | | CTCCGCATTGCTGCAATGCCTGCTCTGGCAAACGGCCTCTTGCCGCGGTTTCTTGCTCAGTTCATCCGTGACAGACCAAATCTCCAGGTCTC |
| | | CCTAATGGGACTGCCCTCAAGCATGGTCATGGAAGCCGTTGCGTCCGGCAGGGCCGACATCGGTTATGCCGATGGCCCACAGGAGCGCCAAG |
| | | GTTTTCTAATCGAAACCCGGTCGCTTCCCGCTGTTGTCGCTGTCCCGATGGGACATCGACTTGCTGGCCTTGACCGTGTCACGCCACAGGAC |
| | | CTTGCCGGTGAGCGTATTATAAAACAGGAGACTGGCACTCTCTTCGCCATGCGGGTAGAGGTGGCGATTGGTGGTATTCAACGCCGGCCGTC |
| | | AATTGAAGTGAGCCTGTCGCATACTGCGCTAAGTCTCGTCCGCGAAGGCGCCGGGATCGCAATTATCGATCCAGCCGCGGCGATCGAGTTCA |
| | | CGGACAGGATCGTACTGCGACCGTTCTCGATCTTCATTGACGCCGGATTCCTCGAAGTCCGGTCAGCAATTGGCGCTCCCTCAACCATCGTC |
| | | GATCGTTTCACAACCGAATTCTGGAGGTTTCATATGATTATGAAGCAGAACGGCCTAATGGAGTAA |
| P_{Bet} | Promoter¹⁷ | AGCGCGGGTGAGAGGGATTCGTTACCAATAGACAATTGATTGGACGTTCAATATAATGCTAGC |
| P_{Cln} | Promoter | CCCTTTGTGCGTCCAAACGGACGCACGGCGCTCTAAAGCGGGTCGCGATCTTTCAGATTCGCTCCTCGCGCTTTCAGTCTTTGTTTTGGCGC |
| | | ATGTCGTTATCGCAAAACCGCTGCACACTTTTGCGCGACATGCTCTGATCCCCCTCATCTGGGGGGGCCTATCTGAGGGAATTTCCGATCCG |
| | | GCTCGCCTGAACCATTCTGCTTTCCACGAACTTGAAAACGCT |
| P_{3B5B} | Promoter⁵ | TTTTGTTCGATTATCGAACAAATTATTGAAATATCGAACAAAACCTCTAAACTACTGTGGCACTGAATCAAAAAATTATAAACCCTGATCAG |
| | | A |
| P_{TTg} | Promoter¹⁹ | CACCCAGCAGTATTTACAAACAACCATGAATGTAAGTATATTCCTTAGCAA |
| P_{Van} | Promoter²⁰ | ATTGGATCCAATTGACAGCTAGCTCAGTCCTAGGTACCATTGGATCCAAT |

TABLE 11
RBS sequences used in this study

| | | | Strength |
|---|---|---|---|
| Name | Strain | RBS sequence^{a} (SEQ ID NOs: 226-291) | (GFP, au) |
| RBSr1 | R. sp. IRBG74 | ATTTCACACATCTAGAGCTAATCATCTCGTACTAAAGAGGAGAAATTAACCATG | 8242 |
| RBSr2 | R. sp. IRBG74 | ATTTCACACATCTAGAGCTAATCATCGCGTACTCAGGAGGCAAGTAATG | 7181.5 |
| RBSr3 | R. sp. IRBG74 | ATTTCACACATCTAGAATTAAAGAGGAGAAATTAACCATG | 6238.5 |
| RBSr4 | R. sp. IRBG74 | TAACAATTTCACACATCTAGAGCTAATCATCTCGTACTAAAGAGGCAAGTAATG | 3618 |
| RBSr5 | R. sp. IRBG74 | TAACAATTTCACACATCTAGAGCTAATCATCGCGTACTAAGGAGGCAAGTAATG | 3560 |
| RBSr6 | R. sp. IRBG74 | TAACAATTTCACACATCTAGAGCTAATCATCGCGTACTCAAGAGGCAAGTAATG | 2614.5 |
| RBSr7 | R. sp. IRBG74 | TAACAATTTCACACATCTAGAGCTAATCTTCGCGTACTAAAGAGGCAAGTAATG | 2418.5 |
| RBSr8 | R. sp. IRBG74 | TAACAATTTCACACATCTAGAGCTAATCATCTCGTACTCAGGAGGCAAGTAATG | 1882.5 |
| RBSr9 | R. sp. IRBG74 | TAACAATTTCACACATCTAGAGCTAATCATCTCGTACTAATGAGGCAAGTAATG | 1593.5 |
| RBSr10 | R. sp. IRBG74 | TAACAATTTCACACATCTAGAGCTAATCATCGCGTACTAATGAGGCAAGTAATG | 1590 |
| RBSr11 | R. sp. IRBG74 | TAACAATTTCACACATCTAGAGCTAATCATCGCGTACTCACGAGGCAAGTAATG | 1554 |
| RBSr12 | R. sp. IRBG74 | TAACAATTTCACACATCTAGAGCTAATCATCGCGTACTAAAAAGGCAAGTAATG | 1138 |
| RBSr13 | R. sp. IRBG74 | TAACAATTTCACACATCTAGAGCTAATCTTCGCGTACTAAAAAGGCAAGTAATG | 895.5 |
| RBSr14 | R. sp. IRBG74 | TAACAATTTCACACATCTAGAGCTAATCTTCGCGTACTAAGAAGGCAAGTAATG | 632.5 |
| RBSr15 | R. sp. IRBG74 | TAACAATTTCACACATCTAGAGCTAATCATCTCGTACTAAATAGGCAAGTAATG | 648.5 |
| RBSr16 | R. sp. IRBG74 | TAACAATTTCACACATCTAGAGCTAATCATCTCGTACTAATAAGGCAAGTAATG | 532 |
| RBSr17 | R. sp. IRBG74 | TAACAATTTCACACATCTAGAGCTAATCTTCTCGTACTAAAGAGGCAAGTAATG | 488 |
| RBSr18 | R. sp. IRBG74 | TAACAATTTCACACATCTAGAGCTAATCATCGCGTACTCAATAGGCCAGTAATG | 305.5 |
| RBSr19 | R. sp. IRBG74 | TAACAATTTCACACATCTAGAGCTAATCATCGCGTACTAAGTAGGCAAGTAATG | 242 |
| RBSr20 | R. sp. IRBG74 | TAACAATTTCACACATCTAGAGCTAATCATCTCGTACTAACGAGGCAAGTAATG | 248 |
| RBSr21 | R. sp. IRBG74 | TAACAATTTCACACATCTAGAGCTAATCATCGCGTACTCAGCAGGCAAGTAATG | 183 |
| RBSr22 | R. sp. IRBG74 | TAACAATTTCACACATCTAGAGCTAATCTTCGCGTACTAAGTAGGCAAGTAATG | 130 |
| RBSr23 | R. sp. IRBG74 | TAACAATTTCACACATCTAGAGCTAATCTTCGCGTACTAATTAGGCAAGTAATG | 84.4 |
| RBSr24 | R. sp. IRBG74 | TAACAATTTCACACATCTAGAGCTAATCTTCTCGTACTAACAAGGCAAGTAATG | 75.15 |
| RBSr25 | R. sp. IRBG74 | TAACAATTTCACACATCTAGAGCTAATCATCTCGTACTCAATAGGCAAGTAATG | 45.45 |
| RBSr26 | R. sp. IRBG74 | TAACAATTTCACACATCTAGAGCTAATCATCTCGTACTAAGCACGCAAGTAATG | 36 |
| RBSr27 | R. sp. IRBG74 | TAACAATTTCACACATCTAGAGCTAATCATCGCGTACTAACTACGCAAGTAATG | 12.2 |
| RBSr28 | R. sp. IRBG74 | TAACAATTTCACACATCTAGAGCTAATCTTCGCGTACTAAGAACGCAAGTAATG | 13 |
| RBSr29 | R. sp. IRBG74 | TAACAATTTCACACATCTAGAGCTAATCTTCGCGTACTAAAAACGCAAGTAATG | 4.6 |
| RBSr30 | R. sp. IRBG74 | TAACAATTTCACACATCTAGAGCTAATCTTCGCGTACTAACAACGCAAGTAATG | 2.95 |
| RBSr31 | R. sp. IRBG74 | TAACAATTTCACACATCTAGAGCTAATCTTCTCGTACTCATGACGCAAGTAATG | 1.45 |
| RBSr32 | R. sp. IRBG74 | ATTTCACACATCTAGAATTAAAGAGAAGAAATTAACCATG | N/A^{b} |
| RBSr33 | R. sp. IRBG74 | CTAGTGCGAACTAGCTCATACCGCAGATG | N/A^{b} |
| RBSp1 | P. protegens Pf-5 | CTAGCGCAGGTCCAACGTTTTTCTAAGCAAGGAGGTCATATG | 25090 |
| RBSp2 | P. protegens Pf-5 | CTAGCGAAGGTCCAACGTTTTTCTAAGCAAGGAGGTCATATG | 21590 |
| RBSp3 | P. protegens Pf-5 | CTAGCGAAGGTCCAACGTTTTTCTAAGCCAGGAGGTCATATG | 19690 |
| RBSp4 | P. protegens Pf-5 | CTAGCGCAGGTCCAACGTTTTTCTAAGCCAGGAGGTCATATG | 19490 |
| RBSp5 | P. protegens Pf-5 | CTAGCGAAGCTCCAACGTTTTTCTAAGCAAGGAGGTCATATG | 17990 |
| RBSp6 | P. protegens Pf-5 | GAATTCTACACTAACGGACAGGAGGGTCCGATG | 14490 |
| RBSp7 | P. protegens Pf-5 | GAATTCTAAACTAACGGACAGGAGGGTCCGATG | 13390 |
| RBSp8 | P. protegens Pf-5 | GAATTCTAAGCTAACGGACAGGAGGGTCCGATG | 12790 |
| RBSp9 | P. protegens Pf-5 | GAATTCTTAACTAACGGACAGGAGGGTCCGATG | 11490 |
| RBSp10 | P. protegens Pf-5 | GAATTCTACACTAACGGACAGGAGGGTCGGATG | 11090 |
| RBSp11 | P. protegens Pf-5 | GAATTCTACGCTAACGGACAGGAGGGTCCGATG | 10390 |
| RBSp12 | P. protegens Pf-5 | GAATTCTCAACTAACGGACAGGAGGGTCCGATG | 9590 |
| RBSp13 | P. protegens Pf-5 | GAATTCTAAGCTAACGGACAGGAGGGTCGGATG | 8918 |
| RBSp14 | P. protegens Pf-5 | GAATTCTCAGCTAACGGACAGGAGGGTCCGATG | 8766 |
| RBSp15 | P. protegens Pf-5 | GAATTCTCAACTAACGGACAGGAGGGTCCGATG | 7596 |
| RBSp16 | P. protegens Pf-5 | GAATTCTACGCTAACGGACAGGAGGGTCGGATG | 6055 |
| RBSp17 | P. protegens Pf-5 | GAATTCTCAACTAACGGACAGGAGATATACATATG | 5939 |
| RBSp18 | P. protegens Pf-5 | GAATTCTCAGCTAACGGACAGGAGGGTCGGATG | 5915 |
| RBSp19 | P. protegens Pf-5 | GAATTCTAAACTAACGGACAGGAGGGTCGGATG | 4867 |
| RBSp20 | P. protegens Pf-5 | GAATTCTCAGCTCACGGACAGGAGGGTCGGATG | 4426 |
| RBSp21 | P. protegens Pf-5 | GAATTCTCAACTAACGGACAGGAGGGTCGGGATG | 4110 |
| RBSp22 | P. protegens Pf-5 | GAATTCTACACTCACGGACAGGAGGGTCGGATG | 3977 |
| RBSp23 | P. protegens Pf-5 | GAATTCTAAGCTCACGGACAGGAGGGTCGGATG | 3829 |
| RBSp24 | P. protegens Pf-5 | GAATTCTCAACTCACGGACAGGAGGGTCGGATG | 3661 |
| RBSp25 | P. protegens Pf-5 | GAATTCTACACTAACGGACAGCAGGGTCGGATG | 3542 |
| RBSp26 | P. protegens Pf-5 | CTAGCGCAGGTCCAACCTT+32CTAAGCAAGTAGGTCATATG | 2139 |
| RBSp27 | P. protegens Pf-5 | GAATTCTCAGCTAACGGACAGCAGGGTCGGATG | 1265 |
| RBSp28 | P. protegens Pf-5 | CTAGCGCAGGTCCAACCTTTTTCTAAGCAACTAGGTCATATG | 389 |
| RBSp29 | P. protegens Pf-5 | CTAGCGAAGGTCCAACCTTTTTCTAAGCCAGTAGGTCATATG | 377 |
| RBSp30 | P. protegens Pf-5 | GAATTCTACGCTCACGGACAGCAGGGTCGGATG | 221 |
| RBSp31 | P. protegens Pf-5 | GAATTCTCCGCTCACGGACAGGAGGGTCCGATG | 23.3 |
| RBSp32 | P. protegens Pf-5 | CTTCTCGGCCAGCTGACAGGGGAAGCTCGCATG | N/A^{b} |
| RBSp33 | P. protegens Pf-5 | CTTCTCGGCCAGCTGACAGGAGGAAGCTCGCATG | N/A^{b} |

^{a}The start codon is underlined.
^{b}RBSs are rationally designed for the controllers by the RBS Calculator²

TABLE 12
Chemicals used in this study

| Chemicals | Source | Identifier |
|---|---|---|
| Tryptone | Fisher Scientific | Cat# BP1421 |
| Yeast extract | BD Bacto | Cat# DF0127 |
| NaCl | Fisher Scientific | Cat# S271 |
| CaCl₂•2H₂O | Sigma-Aldrich | Cat# C3306 |
| MgSO₄•7H₂O | Fisher Scientific | Cat# M80 |
| FeCl₃ | Alfa Aesar | Cat# AA1235709 |
| Na₂MoO₄•2H₂O | Sigma-Aldrich | Cat# 331058 |
| NH₄CH₃CO₂ | Sigma-Aldrich | Cat# A1542 |
| Na₂HPO₄ | Fisher Scientific | Cat# S375 |
| KH₂PO₄ | Sigma-Aldrich | Cat# P9791 |
| EDTA—Na2 | Sigma-Aldrich | Cat# E5134 |
| ZnSO₄•7H₂O | ACROS Organics | Cat# AC424605000 |
| H₃BO₃ | Fisher Scientific | Cat# A73 |
| MnSO₄•H₂O | MP Biomedicals | Cat# ICN225099 |
| CuSO₄•5H₂O | Aldon Corp | Cat# CC0535 |
| CoCl₂•6H₂O | Sigma-Aldrich | Cat# C8661 |
| FeSO₄•7H₂O | Sigma-Aldrich | Cat# 215422 |
| Thiamine hydrochloride | ACROS Organics | Cat#148990100 |
| D-pantothenic acid hemicalcium salt | Sigma-Aldrich | Cat# P5155 |
| Biotin | Sigma-Aldrich | Cat# B4501 |
| Nicotinic acid | Sigma-Aldrich | Cat# 72309 |
| MOPS | Fisher Scientific | Cat# BP308 |
| Isopropyl-beta-D-thiogalactoside (IPTG) | GoldBio | Cat# 12481 |
| L-arabinose | Sigma | Cat# A3256 |
| Anhydrotetracycline hydrochloride (aTc) | Sigma | Cat# 37919 |
| N-(3-Oxohexanoyl)-L-homoserine lactone (3OC6HSL) | Sigma | Cat# K3007 |
| N-(3-Hydroxytetradecanoyl)-DL-homoserine lactone | Sigma | Cat# 51481 |
| (3OC14HSL) | | |
| Naringenin | Sigma | Cat# N5893 |
| 2,4-Diacetylphloroglucinol (DAPG) | Santa Cruz | Cat# sc-206518 |
| Salicylic acid sodium salt | Sigma | Cat# S3007 |
| 3,4-Dihydroxybenzoic acid (DHBA) | Sigma | Cat# 37580 |
| Vanillic acid | Sigma | Cat# 94770 |
| Cuminic acid | Sigma | Cat# 268402 |
| Nopaline | Toronto Research Chemicals | Cat# N650600 |
| Octopine | Toronto Research Chemicals | Cat# 0239850 |
| Choline chloride | Sigma | Cat# C7017 |
| Tris (1M), pH 8.0 | Invitrogen | Cat# AM9855 |
| Triton X-100 | Sigma-Aldrich | Cat# T8787 |
| Tergitol solution | Sigma-Aldrich | Cat# NP40S |
| DNase I | Sigma-Aldrich | Cat# 4716728001 |
| RNA Fragmentation Reagents | Invitrogen | Cat# AM8740 |
| T4 Polynucleotide kinase | New England Biolabs | Cat# M0201 |
| SUPERase•In | Invitrogen | Cat# AM2694 |
| PEG 8000 | Sigma-Aldrich | Cat# 1546605 |
| T4 RNA ligase 2, truncated K277Q | New England Biolabs | Cat# M0351 |
| Superscript III reverse transcriptase | Invitrogen | Cat# 18080044 |
| CircLigase ssDNA ligase | Epicentre | Cat# CL4115K |
| Phusion High-Fidelity DNA polymerase | New England Biolabs | Cat# M0530 |
| Micrococcal nuclease | Roche | 10107921001 |

What is claimed is:

1. A rhizobium that can fix nitrogen under aerobic free-living conditions, comprising a symbiotic rhizobium having an exogenous nif cluster, wherein the exogenous nif cluster confers nitrogen fixation capability on the symbiotic rhizobium under aerobic free-living conditions, and wherein the rhizobium is not Azorhizobium caulinodans.

2. The rhizobium of claim 1, wherein the exogenous nif cluster is selected from a group consisting of a free-living diazotroph, a symbiotic diazotroph, a photosynthetic Alphaproteobacteria, a Gammaproteobacteria, a cyanobacteria, a firmicutes, a Rhodobacter sphaeroides, and a Rhodopseudomonas palustris.

3. The rhizobium of claim 1, wherein the exogenous nif cluster is an inducible refactored nif cluster.

4. The rhizobium of claim 3, wherein the inducible refactored nif cluster is an inducible refactored Klebsiella nif cluster.

5. The rhizobium of any one of claims 1-4, wherein the rhizobium is IRBG74.

6. The rhizobium of any one of claims 1-5, wherein the exogenous nif cluster comprises 6 nif genes.

7. The rhizobium of claim 6, wherein the 6 nif genes are nifHDK(T)Y, nifEN(X), nifJ, nifBQ, nifF, and nifUSVWZM.

8. The rhizobium of claim 6 or 7, wherein each nif gene of the exogenous nif cluster is preceded by a T7 promoter.

9. The rhizobium of any one of claims 1-8, further comprising an endogenous nif cluster.

10. The rhizobium of any one of claims 1-9, wherein the exogenous nif cluster further comprises a terminator.

11. The rhizobium of any one of claims 8-10, wherein the T7 promoter has a terminator and wherein the terminator is downstream from the T7 promoter.

12. The rhizobium of claim 12, wherein the exogenous nif cluster is a refactored rhizobium IRBG74 nif cluster.

13. A plant growth promoting bacterium that can fix nitrogen under aerobic free-living conditions, comprising a bacterium having an exogenous nif cluster having at least one inducible promoter, wherein the exogenous nif cluster confers nitrogen fixation capability on the bacterium, under aerobic free-living conditions, and wherein the bacterium is not Azorhizobium caulinodans.

14. The plant growth promoting bacterium of claim 13, wherein the bacterium is a symbiotic bacterium.

15. The plant growth promoting bacterium of claim 13, wherein the bacterium is an endophyte.

16. The plant growth promoting bacterium of claim 15, wherein the endophyte is rhizobium IRBG74.

17. The plant growth promoting bacterium of claim 13, wherein the bacterium is an epiphyte.

18. The plant growth promoting bacterium of claim 17, wherein the epiphyte is pseudomonas protogens PF-5.

19. The plant growth promoting bacterium of any one of claims 13-18, wherein the plant growth promoting bacterium is associated with a genetically modified cereal plant.

20. The plant growth promoting bacterium of claim 19, wherein the genetically modified cereal plant includes an exogenous gene encoding a chemical signal.

21. The plant growth promoting bacterium of claim 19, wherein the nitrogen fixation is under the control of the chemical signal.

22. The plant growth promoting bacterium of claim 20 or 21, wherein the chemical signal is opine, phlorogluconol or rhizopene.

23. The rhizobium of any one of claims 13-22, wherein the inducible promoter is a T7 promoter, and optionally wherein the inducible promoter is P_{A1lacO1} promoter.

24. The rhizobium of any one of claims 13-23, wherein the inducible promoter is activated by an agent selected from a group that includes IPTG, sodium salicylate, octapine, nopaline, the quorum signal 3OC6HSL, aTe, cuminic acid, DAPG, and salicylic acid.

25. The rhizobium of any one of claims 13-24, wherein the inducible promoter has a terminator and wherein the terminator is downstream from the inducible promoter.

26. An Azorhizobium caulinodans capable of inducible ammonium-independent nitrogen fixation in a cereal crop, comprising:
(i) a modified nif cluster, wherein an endogenous nifA gene is deleted or altered; and
(ii) at least one operon comprising nifA and RNA polymerase sigma factor (RpoN), wherein the operon comprises a regulatory element including an inducible promoter.

27. The Azorhizobium caulinodans of claim 26, wherein the endogenous nifA gene is altered with at least one of the following substitutions:
(i) L94Q;
(ii) D95Q; and
(iii) both L94Q and D95Q.

28. A method of engineering a rhizobium that can fix nitrogen under aerobic free-living conditions, comprising transferring an exogenous nif cluster to a symbiotic rhizobium, wherein the exogenous nif cluster confers nitrogen fixation capability on the symbiotic rhizobium, under aerobic free-living conditions, and wherein the rhizobium is not Azorhizobium caulinodans.

29. The method of any one of claims 26-28, wherein the exogenous nif cluster is transferred to the rhizobium in a plasmid.

30. The method of any one of claim 28 or 29, wherein the endogenous NifL gene is deleted.

31. A method of producing nitrogen for consumption by a cereal plant, comprising providing a plant growth promoting bacterium that can fix nitrogen under aerobic free-living conditions in proximity of the cereal plant, wherein the plant growth promoting bacterium is a symbiotic bacterium having an exogenous nif cluster, wherein the exogenous nif cluster confers nitrogen fixation capability on the symbiotic bacterium, enabling nitrogen fixation under aerobic free-living conditions.

32. The method of claim 31, wherein the plant growth bacterium is the bacterium of any one of claims 1-19 and 23-39.

33. The method of any one of claim 31 or 32, wherein the cereal plant is a genetically modified cercal plant.


SEQUENCE LISTING (293 sequences)

SEQ ID NO: 1 - DNA, 17 bp, synthetic construct
  misc_feature 1..17: note = Synthetic polynucleotide
  misc_feature 1: note = linked to /5rApp/
  misc_feature 17: note = linked to /3ddc/
  source 1..17: mol_type = other DNA
ctgtaggcaccatcaat

SEQ ID NO: 2 - DNA, 33 bp, synthetic construct
  misc_feature 1..33: note = Synthetic polynucleotide
  misc_feature 1: note = linked to /5Phos/
  misc_feature 33: note = linked to /iSp18/
  source 1..33: mol_type = other DNA
agatcggaagagcgtcgtgtagggaaagagtgt

SEQ ID NO: 3 - DNA, 41 bp, synthetic construct
  misc_feature 1..41: note = Synthetic polynucleotide
  misc_feature 1: note = linked to /iSp18/
  source 1..41: mol_type = other DNA
caagcagaagacggcatacgagatattgatggtgcctacag

SEQ ID NO: 4 - DNA, 21 bp, synthetic construct
  misc_feature 1..21: note = Synthetic polynucleotide
  source 1..21: mol_type = other DNA
caagcagaagacggcatacga

SEQ ID NO: 5 - DNA, 85 bp, synthetic construct
  misc_feature 1..85: note = Synthetic polynucleotide
  source 1..85: mol_type = other DNA
aatgatacggcgaccaccgagatctacacgatcggaagagcacacgtctgaactccagtcacnnnnnnacactctttccctacac

SEQ ID NO: 6 - DNA, 32 bp, synthetic construct
  misc_feature 1..32: note = Synthetic polynucleotide
  source 1..32: mol_type = other DNA
cgacaggttcagagttctacagtccgacgatc

SEQ ID NO: 7 - DNA, 32 bp, synthetic construct; identical to SEQ ID NO: 6
  misc_feature 1..32: note = Synthetic polynucleotide
  source 1..32: mol_type = other DNA

SEQ ID NO: 8 - DNA, 68 bp, Klebsiella oxytoca
  source 1..68: mol_type = genomic DNA
cgtagggcgcattaatgcagctggcacgacaggtgaattctagactgctggatacgctgcttaaggtc

SEQ ID NO: 9 - DNA, 24 bp, Klebsiella oxytoca
  source 1..24: mol_type = genomic DNA
tacgctgtttgagctggcaaacct

SEQ ID NO: 10 - DNA, 74 bp, Pseudomonas stutzeri
  source 1..74: mol_type = genomic DNA
gcccggagagcaagcccgtagggcgcattaatgcagctggcacgacaggtgttaggttggcctgaattcggtgt

SEQ ID NO: 11 - DNA, 48 bp, Pseudomonas stutzeri
  source 1..48: mol_type = genomic DNA
ggctcacttcgatttcgtccgcggtgcgtgccctgctagtgatgcgta

SEQ ID NO: 12 - DNA, 25 bp, Pseudomonas stutzeri
  source 1..25: mol_type = genomic DNA
cgcctgatttcgcctgatgaacagg

SEQ ID NO: 13 - DNA, 20 bp, Pseudomonas stutzeri
  source 1..20: mol_type = genomic DNA
tgacgctgttgaccaccgcc

SEQ ID NO: 14 - DNA, 23 bp, Pseudomonas stutzeri
  source 1..23: mol_type = genomic DNA
atggaagtggtcggcaccggcta

SEQ ID NO: 15 - DNA, 22 bp, Pseudomonas stutzeri
  source 1..22: mol_type = genomic DNA
cgcaacggttggggtaggttgg

SEQ ID NO: 16 - DNA, 23 bp, Pseudomonas stutzeri
  source 1..23: mol_type = genomic DNA
gacgtccatcgcttcggcttcga

SEQ ID NO: 17 - DNA, 37 bp, Pseudomonas stutzeri
  source 1..37: mol_type = genomic DNA
ctgcgaaatcgacgctgtcgagcatcatcgcggttca

SEQ ID NO: 18 - DNA, 101 bp, Azotobacter vinelandii
  source 1..101: mol_type = genomic DNA
atccattctcaggctgtctcgtctcgtctctacgtacgcggatcccaggcaacgtcttcgtactgcggtaccgggttgcgggggcagccagtggaaaaagg

SEQ ID NO: 19 - DNA, 21 bp, Azotobacter vinelandii
  source 1..21: mol_type = genomic DNA
ctacggcacgccctggttcga

SEQ ID NO: 20 - DNA, 22 bp, Azotobacter vinelandii
  source 1..22: mol_type = genomic DNA
gctcggaaagtgctggagaaac

SEQ ID NO: 21 - DNA, 24 bp, Azotobacter vinelandii
  source 1..24: mol_type = genomic DNA
aaatcagacattcatggccacagg

SEQ ID NO: 22 - DNA, 22 bp, Azotobacter vinelandii
  source 1..22: mol_type = genomic DNA
tctaccatggcgtgactctcgg

SEQ ID NO: 23 - DNA, 43 bp, Azotobacter vinelandii
  source 1..43: mol_type = genomic DNA
actcgtcttctgtccgtttaaactcccggaactctaccaccgc

SEQ ID NO: 24 - DNA, 21 bp, Azotobacter vinelandii
  source 1..21: mol_type = genomic DNA
cttggatagacgaggcacagc

SEQ ID NO: 25 - DNA, 36 bp, Azotobacter vinelandii
  source 1..36: mol_type = genomic DNA
gccggctcctgcaacctgaaggggccgaggatgatg

SEQ ID NO: 26 - DNA, 30 bp, Paenibacillus polymyxa
  source 1..30: mol_type = genomic DNA
gaattgaggataaatgtcagggatttcatg

SEQ ID NO: 27 - DNA, 24 bp, Paenibacillus polymyxa
  source 1..24: mol_type = genomic DNA
ccaagcattttgagatcgcggatg

SEQ ID NO: 28 - DNA, 21 bp, Paenibacillus polymyxa
  source 1..21: mol_type = genomic DNA
cggaggtgccggtatgagcga

SEQ ID NO: 29 - DNA, 22 bp, Paenibacillus polymyxa
  source 1..22: mol_type = genomic DNA
gaagtttgcagcgaaagaggcg

SEQ ID NO: 30 - DNA, 23 bp, Paenibacillus polymyxa
  source 1..23: mol_type = genomic DNA
gggatgatgcagaatacatcccg

SEQ ID NO: 31 - DNA, 26 bp, Paenibacillus polymyxa
  source 1..26: mol_type = genomic DNA
ggtgacctggatgatgcagaggagag

SEQ ID NO: 32 - DNA, 118 bp, unidentified
  misc_feature 1..118: note = Cyanothece
  source 1..118: mol_type = genomic DNA
ggcccgcgttaggttggcctgaattcggtgtgtatcccccggagatacgtaaaaaaaaaaaccccgccctgtcaggggcggggtttttttttgataagtcaagctatcagaaccgatc

SEQ ID NO: 33 - DNA, 48 bp, unidentified
  misc_feature 1..48: note = Cyanothece
  source 1..48: mol_type = genomic DNA
taattcccataacatctgcatgcataataaggtggggaaagtctcagc

SEQ ID NO: 34 - DNA, 24 bp, unidentified
  misc_feature 1..24: note = Cyanothece
  source 1..24: mol_type = genomic DNA
aatgtatttctgatcgatgcgacg

SEQ ID NO: 35 - DNA, 24 bp, unidentified
  misc_feature 1..24: note = Cyanothece
  source 1..24: mol_type = genomic DNA
gttatctggctgatgtttgtggtg

SEQ ID NO: 36 - DNA, 24 bp, unidentified
  misc_feature 1..24: note = Cyanothece
  source 1..24: mol_type = genomic DNA
gtcaaactgtcttgtttaaagccg

SEQ ID NO: 37 - DNA, 104 bp, Azospirillum brasilense
  source 1..104: mol_type = genomic DNA
ttaaggtcatgcagcaggagaactaaaggcccgcgttaggttggtaataaaaaagcccccggaatgatcttccgggggccctgcgcaaatacaacatcgagatc

SEQ ID NO: 38 - DNA, 26 bp, Azospirillum brasilense
  source 1..26: mol_type = genomic DNA
gacgactgaataaggatcgcggaatg

SEQ ID NO: 39 - DNA, 23 bp, Azospirillum brasilense
  source 1..23: mol_type = genomic DNA
tatgtcacaggcccgacaaagcg

SEQ ID NO: 40 - DNA, 23 bp, Azospirillum brasilense
  source 1..23: mol_type = genomic DNA
gattgtcgggtatcgcacacgag

SEQ ID NO: 41 - DNA, 25 bp, Azospirillum brasilense
  source 1..25: mol_type = genomic DNA
cgaaggagttcgccccagtctattc

SEQ ID NO: 42 - DNA, 68 bp, Rhodopseudomonas palustris
  source 1..68: mol_type = genomic DNA
aatacgatcgcatgtcctaggtaatacgactcactatagggagaggtaatcagtggtggatttgatgt

SEQ ID NO: 43 - DNA, 20 bp, Rhodopseudomonas palustris
  source 1..20: mol_type = genomic DNA
ccaagcaaaggaccaccctc

SEQ ID NO: 44 - DNA, 22 bp, Rhodopseudomonas palustris
  source 1..22: mol_type = genomic DNA
agcttcgatatcatccgctgat

SEQ ID NO: 45 - DNA, 23 bp, Rhodopseudomonas palustris
  source 1..23: mol_type = genomic DNA
ttgttcatgtcggacctaaccga

SEQ ID NO: 46 - DNA, 67 bp, Rhodobacter sphaeroides
  source 1..67: mol_type = genomic DNA
caatacgatcgcatgtcctaggtaatacgactcactatagggagatgcatttcacgcttcgcgattc

SEQ ID NO: 47 - DNA, 20 bp, Rhodobacter sphaeroides
  source 1..20: mol_type = genomic DNA
ccgccttcaccagagacacc

SEQ ID NO: 48 - DNA, 23 bp, Rhodobacter sphaeroides
  source 1..23: mol_type = genomic DNA
atcgagaagttctacgatgccgt

SEQ ID NO: 49 - DNA, 68 bp, Rhodobacter sphaeroides
  source 1..68: mol_type = genomic DNA
gcaaaaaaaaaccccgcccctgacagggcggggttttttttttcaattggacctggatgggcagcaag

SEQ ID NO: 50 - DNA, 106 bp, Azorhizobium caulinodans
  source 1..106: mol_type = genomic DNA
ctcgcatccattctcaggctgtctcgtctcgtctctctagagtcggagctcttggggcctctaaacgggtcttgaggggttttttgttgtcttcgacgcgaagctc

SEQ ID NO: 51 - DNA, 53 bp, Azorhizobium caulinodans
  source 1..53: mol_type = genomic DNA
ataggcaatacgatcgcatgtccgtttaaactgataaggacggcactggctgg

SEQ ID NO: 52 - DNA, 19 bp, Azorhizobium caulinodans
  source 1..19: mol_type = genomic DNA
cgatgccgtccagcacctc

SEQ ID NO: 53 - DNA, 21 bp, Azorhizobium caulinodans
  source 1..21: mol_type = genomic DNA
ctgccacggttcccaaggttc

SEQ ID NO: 54 - DNA, 62 bp, Azorhizobium caulinodans
  source 1..62: mol_type = genomic DNA
taaaaaagcggctaaccacgccgctttttttacgtctgcagtgttgtcgaagcttgatgcgc

SEQ ID NO: 55 - DNA, 60 bp, Azorhizobium caulinodans
  source 1..60: mol_type = genomic DNA
cgctgcttaaggtcatgcagcaggagaactaaaggcccgctctgcgaaaggaatagcgtc

SEQ ID NO: 56 - DNA, 19 bp, Azorhizobium caulinodans
  source 1..19: mol_type = genomic DNA
ctatcgccgccacctgacc

SEQ ID NO: 57 - DNA, 30 bp, Azorhizobium caulinodans
  source 1..30: mol_type = genomic DNA
cgtcagaacggctctgacgcatcagggaga

SEQ ID NO: 58 - DNA, 32 bp, Azorhizobium caulinodans
  source 1..32: mol_type = genomic DNA
agtaatattgcggatcggccagcagcgaggaa

SEQ ID NO: 59 - DNA, 25 bp, Azorhizobium caulinodans
  source 1..25: mol_type = genomic DNA
ggtggtcattggcaacggttcgaag

SEQ ID NO: 60 - DNA, 31 bp, Azorhizobium caulinodans
  source 1..31: mol_type = genomic DNA
tccccaagagcccaaccgttccgggagcgaa

SEQ ID NO: 61 - DNA, 99 bp, Gluconacetobacter diazotrophicus
  source 1..99: mol_type = genomic DNA
ttaaggtcatgcagcaggagaactaaaggcccgcgttaggttggtaataaaaaagcccccggaatgatcttccgggggccgatcgaggaaatcgacgtg

SEQ ID NO: 62 - DNA, 28 bp, Gluconacetobacter diazotrophicus
  source 1..28: mol_type = genomic DNA
atattccggatacggctggtgaggtgga

SEQ ID NO: 63 - DNA, 24 bp, Gluconacetobacter diazotrophicus
  source 1..24: mol_type = genomic DNA
cgccacgtcgtcaatgcctataac

SEQ ID NO: 64 - DNA, 21 bp, Gluconacetobacter diazotrophicus
  source 1..21: mol_type = genomic DNA
tgaccaccgtgcagaagatcc

SEQ ID NO: 65 - DNA, 23 bp, Klebsiella oxytoca
  source 1..23: mol_type = genomic DNA
gtgacgctcgcgtatcaggtttg

SEQ ID NO: 66 - DNA, 69 bp, Klebsiella oxytoca
  source 1..69: mol_type = genomic DNA
atcaggcgcatatttgaatgtatttactgcagcggccgcttctagagtgaccaaaagcttccgcaaccc

SEQ ID NO: 67 - DNA, 48 bp, Pseudomonas stutzeri
  source 1..48: mol_type = genomic DNA
actacgcatcactagcagggcacgcaccgcggacgaaatcgaagtgag

SEQ ID NO: 68 - DNA, 22 bp, Pseudomonas stutzeri
  source 1..22: mol_type = genomic DNA
ttgtcgactcccggggtctgac

SEQ ID NO: 69 - DNA, 23 bp, Pseudomonas stutzeri
  source 1..23: mol_type = genomic DNA
ggctttaacggcatgttccgggt

SEQ ID NO: 70 - DNA, 24 bp, Pseudomonas stutzeri
  source 1..24: mol_type = genomic DNA
gtagtcgtcgttgtggccgaactc

SEQ ID NO: 71 - DNA, 22 bp, Pseudomonas stutzeri
  source 1..22: mol_type = genomic DNA
aaagcatcatctcgggtcgggc

SEQ ID NO: 72 - DNA, 21 bp, Pseudomonas stutzeri
  source 1..21: mol_type = genomic DNA
cgtcgagcgacaacgcctcga

SEQ ID NO: 73 - DNA, 25 bp, Pseudomonas stutzeri
  source 1..25: mol_type = genomic DNA
ctatgagctggactgaaccgcgatg

SEQ ID NO: 74 - DNA, 74 bp, Pseudomonas stutzeri
  source 1..74: mol_type = genomic DNA
gaaaataccgcatcaggcgcatatttgaatgtatttactgcagcggccgctggcgaatctccttcctcggttcg

SEQ ID NO: 75 - DNA, 22 bp, Azotobacter vinelandii
  source 1..22: mol_type = genomic DNA
gccttcgaacatgttgtcccag

SEQ ID NO: 76 - DNA, 24 bp, Azotobacter vinelandii
  source 1..24: mol_type = genomic DNA
tcgagttcgagcagtttctccagc

SEQ ID NO: 77 - DNA, 20 bp, Azotobacter vinelandii
  source 1..20: mol_type = genomic DNA
agcgaacaatacctgtggcc

SEQ ID NO: 78 - DNA, 21 bp, Azotobacter vinelandii
  source 1..21: mol_type = genomic DNA
tggcgcttgcccttgttccaa

SEQ ID NO: 79 - DNA, 54 bp, Azotobacter vinelandii
  source 1..54: mol_type = genomic DNA
gcgcggtggtagagttccgggagtttaaacggacagaagacgagtcgtgcgggc

SEQ ID NO: 80 - DNA, 20 bp, Azotobacter vinelandii
  source 1..20: mol_type = genomic DNA
ttgctcagggtcgggttggc

SEQ ID NO: 81 - DNA, 39 bp, Azotobacter vinelandii
  source 1..39: mol_type = genomic DNA
catcatcctcggccccttcaggttgcaggagccggcttg

SEQ ID NO: 82 - DNA, 21 bp, Azotobacter vinelandii
  source 1..21: mol_type = genomic DNA
gcaagccactccactgacgaa

SEQ ID NO: 83 - DNA, 22 bp, Paenibacillus polymyxa
  source 1..22: mol_type = genomic DNA
acaggttccgcagttcacaagc

SEQ ID NO: 84 - DNA, 25 bp, Paenibacillus polymyxa
  source 1..25: mol_type = genomic DNA
gctgattgtgatcgacaatattcgg

SEQ ID NO: 85 - DNA, 22 bp, Paenibacillus polymyxa
  source 1..22: mol_type = genomic DNA
gaaagcctacacgaagcaaagg

SEQ ID NO: 86 - DNA, 22 bp, Paenibacillus polymyxa
  source 1..22: mol_type = genomic DNA
cttgagaatctgccgggcgcct

SEQ ID NO: 87 - DNA, 22 bp, Paenibacillus polymyxa
  source 1..22: mol_type = genomic DNA
atccacaaatcaacaccctgcg

SEQ ID NO: 88 - DNA, 22 bp, Paenibacillus polymyxa
  source 1..22: mol_type = genomic DNA
aaagcgttccagtcacggtcac

SEQ ID NO: 89 - DNA, 48 bp, unidentified
  misc_feature 1..48: note = Cyanothece
  source 1..48: mol_type = genomic DNA
gagactttccccaccttattatgcatgcagatgttatgggaattaacg

SEQ ID NO: 90 - DNA, 23 bp, unidentified
  misc_feature 1..23: note = Cyanothece
  source 1..23: mol_type = genomic DNA
accttgacaatcattacacagcg

SEQ ID NO: 91 - DNA, 28 bp, unidentified
  misc_feature 1..28: note = Cyanothece
  source 1..28: mol_type = genomic DNA
caaatataatgatcgacattttcaccac

SEQ ID NO: 92 - DNA, 24 bp, unidentified
  misc_feature 1..24: note = Cyanothece
  source 1..24: mol_type = genomic DNA
cgttaactttgtcgcaaaacttcg

SEQ ID NO: 93 - DNA, 102 bp, unidentified
  misc_feature 1..102: note = Cyanothece
  source 1..102: mol_type = genomic DNA
accaaggcgaatctccttcctcggttcgcgatcacgctactccgccaataaaaaagcccccggaatgatcttccgggggccagattcaggtaactgctcaag

SEQ ID NO: 94 - DNA, 23 bp, Azospirillum brasilense
  source 1..23: mol_type = genomic DNA
tgcgtcttcttcgggcatcgtca

SEQ ID NO: 95 - DNA, 23 bp, Azospirillum brasilense
  source 1..23: mol_type = genomic DNA
agaaaattgattgcggacgagcg

SEQ ID NO: 96 - DNA, 27 bp, Azospirillum brasilense
  source 1..27: mol_type = genomic DNA
ttcaataagttaagcagatcggcctcg

SEQ ID NO: 97 - DNA, 33 bp, Azospirillum brasilense
  source 1..33: mol_type = genomic DNA
cggtgttacgaataaatatttctacgaatagac

SEQ ID NO: 98 - DNA, 68 bp, Azospirillum brasilense
  source 1..68: mol_type = genomic DNA
gctccaaaaggagcctttaattgtatcggtttatcagcttgctttgttccgcgggtctcgatacaacg

SEQ ID NO: 99 - DNA, 22 bp, Rhodopseudomonas palustris
  source 1..22: mol_type = genomic DNA
ggtcttgcggatcatcactttc

SEQ ID NO: 100 - DNA, 20 bp, Rhodopseudomonas palustris
  source 1..20: mol_type = genomic DNA
gacggtcaggtggtccgaac

SEQ ID NO: 101 - DNA, 23 bp, Rhodopseudomonas palustris
  source 1..23: mol_type = genomic DNA
ggtgagaatgatcatgatcggcc

SEQ ID NO: 102 - DNA, 67 bp, Rhodopseudomonas palustris
  source 1..67: mol_type = genomic DNA
ctccaaaaggagcctttaattgtatcggtttatcagcttgctttgacgacaagtggagaagggatag

SEQ ID NO: 103 - DNA, 22 bp, Rhodobacter sphaeroides
  source 1..22: mol_type = genomic DNA
tcccatggtcatgtcctttgcg

SEQ ID NO: 104 - DNA, 21 bp, Rhodobacter sphaeroides
  source 1..21: mol_type = genomic DNA
gtgcgcttttccacgaggagc

SEQ ID NO: 105 - DNA, 66 bp, Rhodobacter sphaeroides
  source 1..66: mol_type = genomic DNA
aattgaaaaaaaaaaccccgccctgtcaggggcggggtttttttttgcagcgcccattccgtcttc

SEQ ID NO: 106 - DNA, 66 bp, Rhodobacter sphaeroides
  source 1..66: mol_type = genomic DNA
gctccaaaaggagcctttaattgtatcggtttatcagcttgctttggagaaagcctgcgcggctag

SEQ ID NO: 107 - DNA, 65 bp, Azorhizobium caulinodans
  source 1..65: mol_type = genomic DNA
gcccccggaaggtgatcttccgggggctttctcatgcgttgacagccttgagatagatcaagtgc

SEQ ID NO: 108 - DNA, 20 bp, Azorhizobium caulinodans
  source 1..20: mol_type = genomic DNA
ctgatccaggccttcatcgg

SEQ ID NO: 109 - DNA, 23 bp, Azorhizobium caulinodans
  source 1..23: mol_type = genomic DNA
gacatgtctggtctccttggaac

SEQ ID NO: 110 - DNA, 42 bp, Azorhizobium caulinodans
  source 1..42: mol_type = genomic DNA
ttctggaatttggtaccgagtcagtaacgtgccacagcctcg

SEQ ID NO: 111 - DNA, 117 bp, Azorhizobium caulinodans
  source 1..117: mol_type = genomic DNA
atcaggcgcatatttgaatgtatttactgcagcggccgctacgtacttgtggggtcagttccggctgggggttcagcagccacctgcagttaattaaggcgctcctttcctgattcg

SEQ ID NO: 112 - DNA, 20 bp, Azorhizobium caulinodans
  source 1..20: mol_type = genomic DNA
gctgctgtgtggagagatcg

SEQ ID NO: 113 - DNA, 22 bp, Azorhizobium caulinodans
  source 1..22: mol_type = genomic DNA
gtcggtgagattgatcatggcc

SEQ ID NO: 114 - DNA, 20 bp, Azorhizobium caulinodans
  source 1..20: mol_type = genomic DNA
tgcatgtccgttcctcgctg

SEQ ID NO: 115 - DNA, 24 bp, Azorhizobium caulinodans
  source 1..24: mol_type = genomic DNA
acatgtcttgaattccttcgaacc

SEQ ID NO: 116 - DNA, 18 bp, Azorhizobium caulinodans
  source 1..18: mol_type = genomic DNA
tgcattgcgttcgctccc

SEQ ID NO: 117 - DNA, 19 bp, Azorhizobium caulinodans
  source 1..19: mol_type = genomic DNA
tgtcagggcaggcagggcc

SEQ ID NO: 118 - DNA, 41 bp, Gluconacetobacter diazotrophicus
  source 1..41: mol_type = genomic DNA
tcaccagccgtatccggaatatgtcaggatcatgacatccc

SEQ ID NO: 119 - DNA, 20 bp, Gluconacetobacter diazotrophicus
  source 1..20: mol_type = genomic DNA
acgatttccatgcccaggtc

SEQ ID NO: 120 - DNA, 20 bp, Gluconacetobacter diazotrophicus
  source 1..20: mol_type = genomic DNA
cctccagcacctcttcgatg

SEQ ID NO: 121 - DNA, 67 bp, Gluconacetobacter diazotrophicus
  source 1..67: mol_type = genomic DNA
gctccaaaaggagcctttaattgtatcggtttatcagcttgctttgggcaatacctgagacgtttca

SEQ ID NO: 122 - DNA, 70 bp, synthetic construct
  misc_feature 1..70: note = Synthetic polynucleotide
  source 1..70: mol_type = other DNA
agagtgttgacttgtgagcggataacaatgatacttagattcaattgtgagcggataacaatttcacaca

SEQ ID NO: 123 - DNA, 2652 bp, synthetic construct
  misc_feature 1..2652: note = Synthetic polynucleotide
  source 1..2652: mol_type = other DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctacttggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgacaaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtacgattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 124 - DNA, 51 bp, Azorhizobium caulinodans
  source 1..51: mol_type = genomic DNA
cgaatggtgcaaaacctttcgcggtatggcatgatagcgcccggaagagag

SEQ ID NO: 125 - DNA, 1545 bp, synthetic construct
  misc_feature 1..1545: note = Synthetic polynucleotide
  source 1..1545: mol_type = other DNA
atggcgatgagcccaaagatggagttccgccagagccagtctctggtgatgacgccgcagctgatgcaggccatcaagctgctgcagctctccaatctcgaactggtcgcctatgtggaggccgagctcgaacgcaatccgctgctggagcgggcgagcgagccggaaagccccgagcacgatccgccgaacccgcaggaagaggcacccaccccgcctgacagtggcgcgccggtgtccggcgactggatggaaagcgacatgggctcgagccgcgaggccatcgagacccggctggacaccgacctcggcaatgtctttcccgatgatgcgccggccgagcgcatcggcgcgggcagcggcagcggctcgtccatcgaatggggctcgggcggcgaccggggcgaggactacaatccggaagccttcctcgctgccgagacgacgctggccgaccatctggaagcccagctctccgtggcggagcccgatccggcgcgccgcctcatcggcctcaacctcatcggcctcatcgacgagacgggttatttctccggcgacctcgatgcggtggccgagcaactgggcgccacccacgatcaggtggccgacgtgctgcgcgtcatccagagcttcgagccgtccggcgtcggcgcacggtcgctcagcgaatgcctggccctgcaattgcgcgacaaggatcgctgcgatcccgccatgcaggcgctgctcgacaatctggaactcctcgcccgccacgaccgcaacgcgctgaagcgcatctgcggggtggacgcggaagacctcgcggacatgatcggcgagatccgccgcctcgatccgaagcccggcctcgcctatggcggcggcgtcgtccacccgctggtgccggacgtgttcgtgcgcgagggctccgacggcagctggatcgtggaactgaattccgagacgctgccgcgcgtgctggtgaaccagacctatcacgcgacggtggccaaggcggcgcgctcggccgaggaaaagaccttcctcgccgactgcctccagagcgcctcctggcttacccgctcgctcgaccagcgggctcgcaccatcctcaaggtggcgagcgagatcgtgcgccagcaggacgccttcctcgtgcacggcgtgcggcacctgcgccccctgaacctgcgcacggtggcggatgccatcggcatgcacgaatccaccgtctcgcgggtgacctcgaacaagtacatctccaccccgcgcggggtgctggagatgaagttcttcttctcctcctccatcgcttcctcgggtggtggcgaggcccatgcggcggaggcggtgcgccaccgcatcaagagcctcatcgaggccgagagtgcggacgacgtgctgtccgacgacacgctggtgcagaagctgaaggacgacggcatcgatatcgcccgccgaacggtcgcgaaatatcgcgagagcatgaacatcccgtcctcggtccagcgccgccgcgaaaagcaggccctgcgcagcgacgccgccgccgccggctga

SEQ ID NO: 126 - DNA, 1083 bp, synthetic construct
  misc_feature 1..1083: note = Synthetic polynucleotide
  source 1..1083: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 127 - DNA, 717 bp, synthetic construct
  misc_feature 1..717: note = Synthetic polynucleotide
  source 1..717: mol_type = other DNA
atgagtaaaggagaagaacttttcactggagttgtcccaattcttgttgaattagatggtgatgttaatgggcacaaattttctgttagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatag

SEQ ID NO: 128 - DNA, 717 bp, synthetic construct
  misc_feature 1..717: note = Synthetic polynucleotide
  source 1..717: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatga

SEQ ID NO: 129 - DNA, 678 bp, synthetic construct
  misc_feature 1..678: note = Synthetic polynucleotide
  source 1..678: mol_type = other DNA
atggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaa

SEQ ID NO: 130 - DNA, 711 bp, synthetic construct
  misc_feature 1..711: note = Synthetic polynucleotide
  source 1..711: mol_type = other DNA
atggtttcgaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccttgcaggacggcgagttcatctacaaggtgaagctgcgcggcaccaacttcccctccgacggccccgtaatgcagaagaagacgatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 131 - DNA, 23 bp, synthetic construct
  misc_feature 1..23: note = Synthetic polynucleotide
  source 1..23: mol_type = other DNA
taatacgactcactatagggaga

SEQ ID NO: 132 - DNA, 23 bp, synthetic construct
  misc_feature 1..23: note = Synthetic polynucleotide
  source 1..23: mol_type = other DNA
taatacgactcactacaggcaga

SEQ ID NO: 133 - DNA, 23 bp, synthetic construct
  misc_feature 1..23: note = Synthetic polynucleotide
  source 1..23: mol_type = other DNA
taatacgactcactagagagaga

SEQ ID NO: 134 - DNA, 23 bp, synthetic construct
  misc_feature 1..23: note = Synthetic polynucleotide
  source 1..23: mol_type = other DNA
taatacgactcactaatgggaga

SEQ ID NO: 135 - DNA, 23 bp, synthetic construct
  misc_feature 1..23: note = Synthetic polynucleotide
  source 1..23: mol_type = other DNA
taatacgactcactaaagggaga

SEQ ID NO: 136 - DNA, 23 bp, synthetic construct
  misc_feature 1..23: note = Synthetic polynucleotide
  source 1..23: mol_type = other DNA
taatacgactcactataggtaga

SEQ ID NO: 137 - DNA, 23 bp, Azorhizobium caulinodans
  source 1..23: mol_type = genomic DNA
taatacgactcactattgggaga

SEQ ID NO: 138 - DNA, 1221 bp, Klebsiella oxytoca
  source 1..1221: mol_type = genomic DNA
gtgttccgtgggaggcctgccatgctcgccaagacacccgcaaaccccgcgccgcttcagcggacggcgttcctgaacgacaccacgctgcgcgacggcgagcaggcgccgggtgtcgccttcacccgcaaggagaagatcgagatcgccgccgcccttgccgccgccggtgtcccggagatcgaggcgggaacgcccgccatgggcgacgaagaggtggaaaccatccgctccatcgtctcgctgaacctcccgacgcgcgtcatggcctggtgccgcatgagcgaggacgacctgatggccgccgtcgcggcgggcgtgaagatcgtcaatgtctccattcccacctccgaccggcaactggccggcaagctcggcaaggatcgcgcctgggcgctcggccgtgtggcggaggtggtgacactggcgcgtcggctcggctttgaggtggcggtagggggcgaggattcctcgcgggccgatcccgattttctctgccgtctcgcggagacggcgaaggcggcgggcgcctttcgcctgcggctggccgacacgcttggcgtgcttgaccccttcggcacctatgcattggtgcgccgggtggccgccaccaccgacatcgagcttgagttccacgcccatgacgatctcggccttgccaccgccaatacgctggcggcggtgatgggcggagcgcgtcacgccagcgtcaccgtcgccgggctcggcgagcgcgcgggcaatgccgcgctggaggaagtggccatcgccctgcgccagacggcgcgggcggagaccggcatcgctccggccgcgctgaagccgctggccgaactagtgtgcggcgccgccgcccgtccggtgccgcgcggcaaggccatcgtcggcgcggatgtgttcacccacgagtcgggcatccatgtctccggcctgctcaaggaccgggccacctatgaagctctgaatccggaactgttcgggcgtggccacacggtggtgctcggaaagcattccggtcttgcggcggtggagaaggcgctggccgacgagggcatcaccgtggatgcggtgcgcgggcgcgccattctcgaccgggtgcgggcttttgctgtccgcaccaaggagaatgtttcccgcgagacgctgctgcgcttctatcaggacagcttcaccgagtccgcgctgcgtctgcggcgggccgccgtggaaggcgcaatctga

SEQ ID NO: 139 - DNA, 323 bp, Pseudomonas stutzeri
  source 1..323: mol_type = genomic DNA
tgttgcctcaagcacagcctgtgccagctcgcggatgacagaagagttagcgcgaattcaacgcgttatgaagagagtcgccgcgcagcgcgccaagagattgcgtggaataagacacagggggcgacaagctgttgaacaggcgacaaagcgccaccatggccccggcaggcgcaattgttctgtttcccacatttggtcgccttattgtgccgttttgttttacgtcctgcgcggcgacaaataactaacttcataaaaatcataagaatacataaacaggcacggctggtatgttccctgcacttctctgctggcaaaca

SEQ ID NO: 140 - DNA, 233 bp, Azotobacter vinelandii
  source 1..233: mol_type = genomic DNA
tgtcatgttcgcaacagttgccgaaagtgtggaaaaccggcgcttggcccggccgatctttttgtcgccattgcaacagtcaggcctgtcggttgttaactatcgaaccgccgaaggatgttgctagtaattaaattattctaattaaaacaagtgcttagattattttagaaacgctggcacaaaggctgctattgccctgttgcgcaggcttgttcgtgcctatagcccac

SEQ ID NO: 141 - DNA, 256 bp, unidentified
  misc_feature 1..256: note = R. sp. IRBG74
  source 1..256: mol_type = genomic DNA
tgtcagttttgtcacagggggccggaccaggatggtggacgctcgatggggatgtcgggccattgttcggttgtagcaattacaacagtcggagtagggggattgtagggggattgttgtgtatcagaccgccctgcagctcccgtcgatggataattaatcatttaaaatcaatggtttatttatgtgttgcgggtgctggcacagacgctgcattacctttggtgcgcggagttgttcgggcttacggccgaac

SEQ ID NO: 142 - DNA, 294 bp, Azorhizobium caulinodans
  source 1..294: mol_type = genomic DNA
ttgacaaagcctccgagaagagcgccccctaacccctcctcagccctgatcggcagtatcatcttgtcgaatcctaacgtctgataggcaacgctatacgacaaacgctggttacaattgtcggttccgcgacaagaatttgctttgtctggcgggtggtctattttgagctaagtagctgagaaatcaggaaaacaaaactctattcggtctacccgacgagttggcacgggtcttgtaaccatccttgcgcaggcggcgaaagccaccggcgatattcatgttgcgggcaac

SEQ ID NO: 143 - DNA, 215 bp, Klebsiella oxytoca
  source 1..215: mol_type = genomic DNA
tgtcgcgtttgaaacacggggcttttggaaccgttcgattctgcaatgcactgattttacttgattaattcgaccacacgaccactggcacacccgttgcaaaaccccttggtgcaggcgacgggttgccggtctggttcgcggatctcctcgatccccggctaccgacccgcctccgaaaagtccggtcccgatccagttcggcggggccacac

SEQ ID NO: 144 - DNA, 1575 bp, Pseudomonas stutzeri
  source 1..1575: mol_type = genomic DNA
atgatccataaatccgattcggacaccaccgtcagacgtttcgatctctcccagcagtttaccgccatgcagcggataagcgtggtcctgagtcgcgccaccgaagcgagcaaaaccctgcaggaggttctgagcgtgctacataacgatgcctttatgcagcacgggatgatttgcctgtacgacagccagcaggagatcctgagcatcgaagcgctgcagcaaacggaagatcagacgctgcccggcagtacgcaaattcgctaccggccgggggaaggattagtcggtaccgtgctggcgcagggccagtcgctggtgctgccgcgcgtcgccgacgaccagcgttttctcgatcgtctgagcctgtacgactatgacctgccgtttatcgccgttccgctgatgggcccccactcccggcccatcggcgtactggcggcgcagccgatggcgcgtcaggaagagcggctgcccgcctgcacgcgctttctcgaaaccgtcgccaatctgatcgcccagacgattcgcctgatgatcctgccaacctccgccgcgcaggcgccgcagcagagccccagaatagagcgcccgcgcgcctgtaccccttcgcgcggtttcggcctggaaaatatggtcggtaaaagcccggcgatgcggcagattatggatattattcgtcaggtttcccgctgggataccacggtgctggtacgcggcgagagcggcaccgggaaagagctcatcgccaacgccatccaccataattctccgcgcgccgccgcggcgttcgtcaaatttaactgcgcggcgctgccggacaacctgctggagagcgagctgtttggtcatgagaaaggcgcgtttaccggcgcggtgcgccagcggaaaggccgctttgagctggcggacggcggcaccttattcctcgatgagatcggcgaaagcagcgcctcgtttcaggctaagctactgcgtattctgcaagagggggagatggagcgcgtcggcggcgacgaaaccctgcgggtcaacgtgcgcattatcgcggcgaccaaccgccatctggaagaggaggtgcggctgggtcatttccgcgaggatctatactaccgcctgaacgtaatgcctatcgcgctgccgccgctgcgcgagcgccaggaggatatcgccgagctggcgcactttctggtgcgaaaaatcgcccacagccaggggcgaacgctgcgcatcagcgatggggcgattcgcctgctgatggagtacagctggccgggaaacgtgcgcgaactggaaaactgtctcgaacgttcggcggtgctgtcggaaagcggcctgatagaccgggacgtgattctgttcaaccatcgcgataacccgccgaaagcgctcgccagcagcggcccggcggaggacggctggctcgataacagcctcgacgagcgccagcggctgatcgccgccctggaaaaagcgggctgggtgcaggccaaagcggcgcggctgctcggcatgaccccgcgccaggtggcgtatcgcattcagattatggatatcaccatgccgcgactgtga

SEQ ID NO: 145 - DNA, 1566 bp, Azotobacter vinelandii
  source 1..1566: mol_type = genomic DNA
atgaacgccacattcgccgaacgccccagcgcgccaacccgcaacgaactgctggatgcccaactgcaggcgctggcgcagatcgcccgcatccttaaccgcggccggcccatcgaggaactgctggccgagatcctcgccgtgctgcacgaagacctcggcctgctgcacgggctggtctccatctgcaacccgaaggacggcagcctgcaggtgggcgccgtgcacagcgactccgaaaccgtggtacgggcctgcgaaagcacccgctaccgcatcggcgaaggcgtgttcggcaacatcctcaagcatggcaacagcgtggtgctcgggcgtatcgacgccgaaccgcgctttctcgaccgactggcgctgtacgacatggacctgcccttcatcgccgtgccgatcaaggccgtcgacggcaccaccatcggcgtgctggctgcccagcccgaccgccgcgccgacgagctgatgcccgaacgcacccgtttgatggaaatcgtcgcccgcctactggcgcagaccgtgcgcctggtggtgaacctcgaggacggccaggaagtggtcgacgagcgcgacgagctacgccgcgaagtccgcgccaagtacggcttcgagaacatggtggtgggccacaccgcctccatgcgccgggttttcgaccaggttcgacgggtcgccaagtggaacagcaccgtgctgatcctcggcgaatccggcaccggcaaggagctgatcgccagcgccatccactacaactcaccgcgcgctcaccagccgctggtacgcctgaactgcgccgcgctaccggaaaccctgctcgaatcggaactgttcggtcacgagaaaggcgccttcaccggcgccgtgaagcagcgcaagggacgtttcgaacaggccgacggcggcaccctgttcctcgacgagatcggcgagatctcgccgatgttccaggccaagctgctgcgcgtgctgcaggaaggcgagctggagcgcgtcggcggcagccagacggtgaaggtcaacgtgcgcatcgtcgccgccaccaaccgcgacctggagcacgaggtggagcaaggcaagttccgcgaagacctctactaccgcctcaacgtcatggccatccgcgtcccgccgctgcgcgagcgcagcgccgacatcccggaactggccgaattcctcctcgacaagatcgcccgccagcagggtcgcaaactcaagctgaccgacagcgccctgcgtctgctgatgagccaccgctggccgggcaacgtgcgcgaactggaaaactgcctggaacgctcggccatcatgagcgaggatggcaccatcagccgcgacgtggtctccctcaccggcctcgaccacgacgccacgccgctggcgccggtccccgaagtcgacctcgccgacgacagcctcgacgaccgcgagcgcgtcatcgccgcgctggaacaggccggctgggtccaggccaaggccgcccgcctgctcggcatgacgccccggcagatcgcctaccgagtgcagacgctgaacattcatatgcgcaagatctga

SEQ ID NO: 146 - DNA, 1569 bp, unidentified
  misc_feature 1..1569: note = R. sp. IRBG74
  source 1..1569: mol_type = genomic DNA
atgaatgcaaccatccctcagcgctcggccaaacagaacccggtcgaactctatgacctgcaattgcaggccctggcgagcatcgcccgcacgctcagccgcgaacaacagatcgacgaactgctcgaacaggtcctggccgtactgcacaatgacctcggcctgctgcatggcctggtgaccatttccgacccggaacacggcgccctgcagatcggcgccatccacaccgactcggaagcggtggcccaggcctgcgaaggcgtgcgctacagaagcggcgaaggcgtgatcggcaacgtgctcaagcacggcaacagcgtggtgctcgggcgcatctccgccgacccgcgctttctcgaccgcctggcgctgtacgacctggaaatgccgttcatcgccgtgccgatcaagaaccccgagggcaacaccatcggcgtgctggcggcccagccggactgccgcgccgacgagcacatgcccgcgcgcacgcgccttctggagatcgtcgccaacctgctggcgcagaccgtgcgcctggtggtgaacatcgaggacggccgcgaggcggccgacgagcgcgacgaactgcgtcgcgaggtgcgcggcaagtacggcttcgagaacatggtggtgggccacacccccaccatgcgccgggtgttcgatcagatccgccgggtcgccaagtggaacagcaccgtactggtcctcggcgagtccggtaccggcaaggaactgatcgccagcgccatccactacaactcgccgcgcgcgcaccgccccttcgtgcgcctgaactgcgccgcgctgccggaaaccctgctcgagtccgaactcttcggccacgagaagggcgccttcaccggcgcggtgaagcagcgcaaggggcgtttcgagcaggccgacggcggcaccctgttcctcgacgagatcggcgagatctcgccgatgttccaggccaagctgctgcgcgtgctgcaggaaggcgagttcgagcgggtcggcggcaaccagacggtgcgggtcaacgtgcgcatcgtcgccgccaccaaccgcgacctggaaagcgaggtggaaaagggcaagttccgcgaggacctctactaccgcctgaacgtcatggccatccgcattccgccgctgcgcgagcgtaccgccgacattcccgaactggcggaattcctgctcggcaagatcggccgccagcagggccgcccgctgaccgtcaccgacagcgccatccgcctgctgatgagccaccgctggccgggcaacgtgcgcgaactggagaactgcctggagcgctcggcgatcatgagcgaggacggcaccatcacccgcgacgtggtctcgctgaccggggtcgacaacgagagcccgccgctcgccgcgccgctgcccgaggtcaacctggccgacgagaccctggacgaccgcgaacgggtgatcgccgccctcgaacaggccggctgggtgcaggccaaggccgcgcggctgctgggcatgacgccgcggcagatcgcctaccgcatccagaccctcaacatccacatgcgcaagatctga

SEQ ID NO: 147 - DNA, 1695 bp, Azorhizobium caulinodans
  source 1..1695: mol_type = genomic DNA
atgctgcacaatgggctcaatgagggtatgactgaacgatccgctcaaaccatccacaaaccggatttctggggcagcggtatctatcggatatcgaaagttttgattggtccagacagtctcgagacgaagcttgccaatgtcattaacgccctctcagtaattctcccaatgcggcgcggcgcaatcgtcgttctaaatgttaaaggagagcccgagatggttgcaatgctgggcctagagcaagcatctcaaggcgcccgctccattccggcggaggctgcgatagatagaatcgtcgccaaaggcgcgccgctggtcgtaccggacatttgcaagtcggacctgttccaggcggagctccaaaccaactcgaacgccacaggcccagccacgttcgttggcgtcccgatgaaggtcgaaaaagaaacgcttggaacactatggatcgaccgcgccaaagatggcagcactaggatccaatttgaggaagaggtgcgcttcctctccatggtcgccaacctttcggcccgggccatttggctggatcgccaccagagccgcgatggtcagccaatcgtgggcgaggaaggaactcgcaagactagttcaggcgacaaggaactgcccgaatctgcccgacaaaggcccacaaaaatcgattggattgtcggggaaagccctgccctcaagcaggtggttgaaagcgtcaaagtcgttgcaacaaccaattctgcggtgcttctcaggggcgaaagcggcacgggcaaggagttctttgcaaaggccatccacgagctttcataccggaaaaagaagcccttcgtgaagttgaactgcgccgcgctgtctgcaggcgttttggaatcggaattgtttggacatgaaaagggcgccttcacgggggccatctctcagcgcgcaggccgcttcgaactcgcagacggcggaacgctgctgctcgatgagatcggcgacatttcgccgggcttccaagcgaaactgttgcgcgtcttgcaggaaggtgagcttgagcgagtcggcggcacaaaaacactcaaagtggacgttcgactcatatgcgccacgaacaaagacctagaagcggcagtcgcggatggggagttcagggccgacctttattaccggatcaatgtggtgcccctatttctgccgcctctccgggagcgaaatggggatattccacgccttgcgagagttttcctcggccgattcaacagggaaaacaatcgcgatctcgcgttcgcgccggctgcgctcgagctcttgtcaaaatgcaactttcccggcaacgtccgagagcttgaaaactgcgtccgcaggaccgccactctcgcgcgttcggagacgatcgttccatcagatttctcctgcctgaagaaccagtgcttttcttcaatgctctggaaaaccggtgaccgtccacttggggatacgctcaatgggttggccatgcgtaagagtttgtcggtcgaatcgccgatcagcctcggttactccaatggaccggccggcttaacggtggcaccacatctaacggaccgcgagctgctaatcagtgcgatggagaaggccggttgggttcaggcaaaggcagctcggatcctcggcctcacaccgcgacaggtcggctatgctttacgtaggcatcgtatacaggtgaagaaaatctaa

SEQ ID NO: 148 - DNA, 1848 bp, synthetic construct
  misc_feature 1..1848: note = Synthetic polynucleotide
  source 1..1848: mol_type = other DNA
atgccaatgaccgacgccttccaggtccgcgtacctcgggtttcgtcgagcaccgccggagacatcgccgcgtcatccatcaccacgcggggcgcgctgccgcgcccgggagggatgcctgtgtccatgtcgcgggggacctcgcccgaggtggcactcatcggggtctatgagatatcgaagatcctgacggcgccccggcgcctcgaagtcacgctcgccaatgtggtgaacgtgctctcctccatgctgcagatgcggcatggcatgatctgcatcctcgacagcgagggcgatcccgacatggtggccaccaccggctggacgcctgagatggcgggccagatccgcgcgcatgtgccccagaaggccatcgaccagatcgtcgccacgcagatgccgctggtggtgcaggacgtgacggccgatccgctcttcgccggtcacgaggatctgttcggcccgcctgaggaggccaccgtctccttcatcggcgtgccgatcaaggccgaccaccatgtgatgggcaccctctccatcgaccgcatctgggacggcaccgcccgtttccgcttcgacgaggacgtgcgcttcctcaccatggtggccaatctcgtcggccagaccgtgcgcctgcacaagctggtggcgagcgaccgcgaccggctgatcgcccagacgcaccgcctcgaaaaggcgctgcgggaagaaaaatccggggccgagccggaggtggccgaggccgccaacggatccgccatgggcatcgtgggcgatagcccgctggtgaaacgcctgatcgcgaccgcgcaagtggtcgcccgctcaaactccaccgtgctgctgcgcggggagagcggcaccggcaaggagttgttcgcccgtgccatccacgaactgtcgccccgcaagggcaagcccttcgtgaaggtgaactgcgccgccctcccggaatcggtgctggaatcggaactgttcggccatgagaagggcgccttcaccggtgcgctgaacatgcgccagggccgcttcgagctggcgcacggcggcacgctcttccttgacgagatcggcgagatcacccccgctttccaggccaagctgctgcgcgtgctgcaggaaggcgagttcgagcgggtcggcggcaatcgcacgctgaaggtggatgtgcggctcgtgtgcgccaccaacaagaatctggaagaggcggtctccaagggcgagttccgggccgatctctactaccgcatccatgtggtgccgctgatcctgccgccgctgcgcgaacggccgggcgacattcccaagctcgcgaagaacttcctcgaccgcttcaacaaggaaaacaagctccacatgatgctctcggcgccggccatcgacgtgctgcggcgctgctatttcccgggcaacgtgcgcgagctggagaactgtatccggcggacggcaacgctcgcccacgatgccgtcatcaccccccatgacttcgcctgcgacagcggccagtgcctctcggccatgctctggaagggctcggccccgaagcctgtgatgccgcacgtgccgccggcgcccacgccgctgactccgctctcccctgctccgctcgcgaccgcagcgcccgctgcggcgagcccggcgccggcggccgacagcctgccggtcacttgccccggcaccgaggcctgtcccgcggtgcccccccgccagagcgaaaaggagcagttgctccaggccatggagcgctccggctgggtgcaggcgaaggccgcgcgcctcctcaacctcacgccgcgccaggtgggttatgcgctgcgcaaatatgacatcgacatcaagcgcttctga

SEQ ID NO: 149 - DNA, 47 bp, synthetic construct
  misc_feature 1..47: note = Synthetic polynucleotide
  source 1..47: mol_type = other DNA
taggtgttgacggctagctcagtcctaggtacagtgctagctctaga

SEQ ID NO: 150 - DNA, 47 bp, synthetic construct
  misc_feature 1..47: note = Synthetic polynucleotide
  source 1..47: mol_type = other DNA
taggtgtttacagctagctcagtcctaggtattatgctagctctaga

SEQ ID NO: 151 - DNA, 47 bp, synthetic construct
  misc_feature 1..47: note = Synthetic polynucleotide
  source 1..47: mol_type = other DNA
taggtgttgacagctagctcagtcctaggtactgtgctagctctaga

SEQ ID NO: 152 - DNA, 47 bp, synthetic construct
  misc_feature 1..47: note = Synthetic polynucleotide
  source 1..47: mol_type = other DNA
taggtgctgatagctagctcagtcctagggattatgctagctctaga

SEQ ID NO: 153 - DNA, 47 bp, synthetic construct
  misc_feature 1..47: note = Synthetic polynucleotide
  source 1..47: mol_type = other DNA
taggtgttgacagctagctcagtcctaggtattgtgctagctctaga

SEQ ID NO: 154 - DNA, 47 bp, synthetic construct
  misc_feature 1..47: note = Synthetic polynucleotide
  source 1..47: mol_type = other DNA
taggtgtttacggctagctcagtcctaggtactatgctagctctaga

SEQ ID NO: 155 - DNA, 47 bp, synthetic construct
  misc_feature 1..47: note = Synthetic polynucleotide
  source 1..47: mol_type = other DNA
taggtgtttacggctagctcagtcctaggtatagtgctagctctaga

SEQ ID NO: 156 - DNA, 47 bp, synthetic construct
  misc_feature 1..47: note = Synthetic polynucleotide
  source 1..47: mol_type = other DNA
taggtgtttacggctagctcagccctaggtattatgctagctctaga

SEQ ID NO: 157 - DNA, 47 bp, synthetic construct
  misc_feature 1..47: note = Synthetic polynucleotide
  source 1..47: mol_type = other DNA
taggtgctgacagctagctcagtcctaggtataatgctagctctaga

SEQ ID NO: 158 - DNA, 47 bp, synthetic construct
  misc_feature 1..47: note = Synthetic polynucleotide
  source 1..47: mol_type = other DNA
taggtgtttacagctagctcagtcctagggactgtgctagctctaga

SEQ ID NO: 159 - DNA, 47 bp, synthetic construct
  misc_feature 1..47: note = Synthetic polynucleotide
  source 1..47: mol_type = other DNA
taggtgtttacggctagctcagtcctaggtacaatgctagctctaga

SEQ ID NO: 160 - DNA, 47 bp, synthetic construct
  misc_feature 1..47: note = Synthetic polynucleotide
  source 1..47: mol_type = other DNA
taggtgttgacggctagctcagtcctaggtatagtgctagctctaga

SEQ ID NO: 161 - DNA, 47 bp, synthetic construct; identical to SEQ ID NO: 152
  misc_feature 1..47: note = Synthetic polynucleotide
  source 1..47: mol_type = other DNA

SEQ ID NO: 162 - DNA, 47 bp, synthetic construct
  misc_feature 1..47: note = Synthetic polynucleotide
  source 1..47: mol_type = other DNA
taggtgctgatggctagctcagtcctagggattatgctagctctaga

SEQ ID NO: 163 - DNA, 47 bp, synthetic construct
  misc_feature 1..47: note = Synthetic polynucleotide
  source 1..47: mol_type = other DNA
taggtgtttatggctagctcagtcctaggtacaatgctagctctaga

SEQ ID NO: 164 - DNA, 47 bp, synthetic construct
  misc_feature 1..47: note = Synthetic polynucleotide
  source 1..47: mol_type = other DNA
taggtgtttatagctagctcagcccttggtacaatgctagctctaga

SEQ ID NO: 165 - DNA, 47 bp, synthetic construct
  misc_feature 1..47: note = Synthetic polynucleotide
  source 1..47: mol_type = other DNA
taggtgttgacagctagctcagtcctagggactatgctagctctaga

SEQ ID NO: 166 - DNA, 47 bp, synthetic construct
  misc_feature 1..47: note = Synthetic polynucleotide
  source 1..47: mol_type = other DNA
taggtgttgacagctagctcagtcctagggattgtgctagctctaga

SEQ ID NO: 167 - DNA, 47 bp, synthetic construct
  misc_feature 1..47: note = Synthetic polynucleotide
  source 1..47: mol_type = other DNA
taggtgttgacggctagctcagtcctaggtattgtgctagctctaga

SEQ ID NO: 168 - DNA, 47 bp, synthetic construct
  misc_feature 1..47: note = Synthetic polynucleotide
  source 1..47: mol_type = other DNA
taggtgttgacagctagctcagtcctaggtataatgctagctctaga

SEQ ID NO: 169 - DNA, 47 bp, synthetic construct
  misc_feature 1..47: note = Synthetic polynucleotide
  source 1..47: mol_type = other DNA
taggtgttgacattattccatcgaactagttaactagtacgaaagtt

SEQ ID NO: 170 - DNA, 48 bp, synthetic construct
  misc_feature 1..48: note = Synthetic polynucleotide
  source 1..48: mol_type = other DNA
tagcataaccccttggggcctctaaacgggtcttgaggggttttttgt

SEQ ID NO: 171 - DNA, 48 bp, synthetic construct
  misc_feature 1..48: note = Synthetic polynucleotide
  source 1..48: mol_type = other DNA
tactcgaacccctagcccgctcttatcgggcggctaggggttttttgt

SEQ ID NO: 172 - DNA, 29 bp, synthetic construct
  misc_feature 1..29: note = Synthetic polynucleotide
  source 1..29: mol_type = other DNA
tacatatcgggggggtaggggttttttgt

SEQ ID NO: 173 - DNA, 80 bp, synthetic construct
  misc_feature 1..80: note = Synthetic polynucleotide
  source 1..80: mol_type = other DNA
ccaggcatcaaataaaacgaaaggctcagtcgaaagactgggcctttcgttttatctgttgtttgtcggtgaacgctctc

SEQ ID NO: 174 - DNA, 61 bp, synthetic construct
  misc_feature 1..61: note = Synthetic polynucleotide
  source 1..61: mol_type = other DNA
ctcggtaccaaattccagaaaagaggcctcccgaaaggggggccttttttcgttttggtcc

SEQ ID NO: 175 - DNA, 146 bp, synthetic construct
  misc_feature 1..146: note = Synthetic polynucleotide
  source 1..146: mol_type = other DNA
ctcggtaccaaattccagaaaagacacccgaaagggtgttttttcgttttggtcctccttggccctccatccttagatagcagataaaaaaaatccttagctttcgctaaggatgatttcttcataggcaatacgatcgcatgtcc

SEQ ID NO: 176 - DNA, 129 bp, synthetic construct
  misc_feature 1..129: note = Synthetic polynucleotide
  source 1..129: mol_type = other DNA
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 177 - DNA, 129 bp, synthetic construct; identical to SEQ ID NO: 176
  misc_feature 1..129: note = Synthetic polynucleotide
  source 1..129: mol_type = other DNA

SEQ ID NO: 178 - DNA, 168 bp, synthetic construct
  misc_feature 1..168: note = Synthetic polynucleotide
  source 1..168: mol_type = other DNA
ggtcttgtccactaccttgcagtaatgcggtggacaggatcggcggttttcttttctcttctcaatgactgaatagaaaagacgaacattaacgcatgagaaagcccccggaagatcaccttccgggggcttttttattgcgctacaaatgaaagtacatagaaatta

SEQ ID NO: 179 - DNA, 146 bp, synthetic construct
  misc_feature 1..146: note = Synthetic polynucleotide
  source 1..146: mol_type = other DNA
cagataaaaaaaatccttagctttcgctaaggatgatttcttccttggccctccatccttagatagctcggtaccaaattccagaaaagacacccgaaagggtgttttttcgttttggtcctcataggcaatacgatcgcatgtcc

SEQ ID NO: 180 - DNA, 128 bp, synthetic construct
  misc_feature 1..128: note = Synthetic polynucleotide
  source 1..128: mol_type = other DNA
ccaggcatcaaataaaacgaaaggctcagtcgaaagactgggcctttcgttttatctgttgtttgtcggtgaacgctctcctagcataaccccttggggcctctaaacgggtcttgaggggttttttg

SEQ ID NO: 181 - DNA, 152 bp, synthetic construct
  misc_feature 1..152: note = Synthetic polynucleotide
  source 1..152: mol_type = other DNA
ctcggtaccaaattccagaaaagagacgctttcgagcgtcttttttcgttttggtcctccttggccctccatccttagatagagttaaccaaaaaggggggattttatctcccctttaatttttccttcataggcaatacgatcgcatgtcc

SEQ ID NO: 182 - DNA, 144 bp, synthetic construct
  misc_feature 1..144: note = Synthetic polynucleotide
  source 1..144: mol_type = other DNA
cgcagatagcaaaaaagcgcctttagggcgcttttttacattggtggtccttggccctccatccttagatagaggcgactgacgaaacctcgctccggcggggttttttgttatctgcatcataggcaatacgatcgcatgtcc

SEQ ID NO: 183 - DNA, 82 bp, synthetic construct
  misc_feature 1..82: note = Synthetic polynucleotide
  source 1..82: mol_type = other DNA
tcggtcagtttcacctgatttacgtaaaaacccgcttcggcgggtttttgcttttggaggggcagaaagatgaatgactgtc

SEQ ID NO: 184 - DNA, 103 bp, synthetic construct
  misc_feature 1..103: note = Synthetic polynucleotide
  source 1..103: mol_type = other DNA
gcccccggaagatcaccttccgggggcttttttattggcggccggctgattgatcaggcggccggctgattggcgcgttacctggtagcgcgccattttgttt

SEQ ID NO: 185 - DNA, 83 bp, synthetic construct
  misc_feature 1..83: note = Synthetic polynucleotide
  source 1..83: mol_type = other DNA
gtaatcgttaatccgcaaataacgtaaaaacccgcttcggcgggtttttttatggggggagtttagggaaagagcatttgtca

SEQ ID NO: 186 - DNA, 41 bp, synthetic construct
  misc_feature 1..41: note = Synthetic polynucleotide
  source 1..41: mol_type = other DNA
aaaaaaaaaccccgcccctgacagggcggggtttttttttt

SEQ ID NO: 187 - DNA, 152 bp, synthetic construct
  misc_feature 1..152: note = Synthetic polynucleotide
  source 1..152: mol_type = other DNA
tccggcaattaaaaaagcggctaaccacgccgctttttttacgtctgcatgactgaatagaaaagacgaacattaacgcatgagaaagcccccggaagatcaccttccgggggcttttttattgcgctccttggccctccatccttagatag

SEQ ID NO: 188 - DNA, 167 bp, synthetic construct
  misc_feature 1..167: note = Synthetic polynucleotide
  source 1..167: mol_type = other DNA
ggaagaccatactggaaacacagaaaaaagcccgcacctgacagtgcgggctttttttttcgaccaaaggtgactgaatagaaaagacgaacattcgcagatagcaaaaaagcgcctttagggcgcttttttacattggtggtcataggcaatacgatcgcatgtcc

SEQ ID NO: 189 - DNA, 160 bp, synthetic construct
  misc_feature 1..160: note = Synthetic polynucleotide
  source 1..160: mol_type = other DNA
tccggcaattaaaaaagcggctaaccacgccgctttttttacgtctgcatccttggccctccatccttagatagctcggtaccaaattccagaaaagaggcctcccgaaaggggggccttttttcgttttggtcctcataggcaatacgatcgcatgtcc

SEQ ID NO: 190 - DNA, 199 bp, synthetic construct
  misc_feature 1..199: note = Synthetic polynucleotide
  source 1..199: mol_type = other DNA
ttcagccaaaaaacttaagaccgccggtcttgtccactaccttgcagtaatgcggtggacaggatcggcggttttcttttctcttctcaatacatgaaagtacatagaaattactcggtaccaaattccagaaaagaggcctcccgaaaggggggccttttttcgttttggtcctcataggcaatacgatcgcatgtcc

SEQ ID NO: 191 - DNA, 189 bp, synthetic construct
  misc_feature 1..189: note = Synthetic polynucleotide
  source 1..189: mol_type = other DNA
ttcagccaaaaaacttaagaccgccggtcttgtccactaccttgcagtaatgcggtggacaggatcggcggttttcttttctcttctcaatccttggccctccatccttagatagtccggcaattaaaaaagcggctaaccacgccgctttttttacgtctgcatcataggcaatacgatcgcatgtcc

SEQ ID NO: 192 - DNA, 164 bp, synthetic construct
  misc_feature 1..164: note = Synthetic polynucleotide
  source 1..164: mol_type = other DNA
ctcggtaccaaattccagaaaagaggcctcccgaaaggggggccttttttcgttttggtcctgactgaatagaaaagacgaacattaacgcatgagaaagcccccggaagatcaccttccgggggcttttttattgcgctccttggccctccatccttagatag

SEQ ID NO: 193 - DNA, 159 bp, synthetic construct
  misc_feature 1..159: note = Synthetic polynucleotide
  source 1..159: mol_type = other DNA
ctcggtaccaaattccagaaaagaggcctcccgaaaggggggccttttttcgttttggtcctccttggccctccatccttagatgtccggcaattaaaaaagcggctaaccacgccgctttttttacgtctgcatcataggcaatacgatcgcatgtcc

SEQ ID NO: 194 - DNA, 197 bp, synthetic construct
  misc_feature 1..197: note = Synthetic polynucleotide
  source 1..197: mol_type = other DNA
ctcggtaccaaagacgaacaataagacgctgaaaagcgtcttttttcgttttggtcctacaaatgaaagtacatagaaattattcagccaaaaaacttaagaccgccggtcttgtccactaccttgcagtaatgcggtggacaggatcggcggttttcttttctcttctcaatccttggccctccatccttagatag

SEQ ID NO: 195 - DNA, 121 bp, synthetic construct
  misc_feature 1..121: note = Synthetic polynucleotide
  source 1..121: mol_type = other DNA
gggaactgccagacatcaaataaaacaaaaggctcagtcggaagactgggccttttgttttatctgttgtttgtcggtgaacactctcccgactagtagcggccgctgcagaaagaggaga

SEQ ID NO: 196 - DNA, 152 bp, synthetic construct
  misc_feature 1..152: note = Synthetic polynucleotide
  source 1..152: mol_type = other DNA
aacgcatgagaaagcccccggaagatcaccttccgggggcttttttattgcgctcataggcaatacgatcgcatgtcctccggcaattaaaaaagcggctaaccacgccgctttttttacgtctgcatccttggccctccatccttagatag

SEQ ID NO: 197 - DNA, 91 bp, synthetic construct
  misc_feature 1..91: note = Synthetic polynucleotide
  source 1..91: mol_type = other DNA
gggaactgccagacatcaaataaaacaaaaggctcagtcggaagactgggccttttgttttatctgttgtttgtcggtgaacactctcccg

SEQ ID NO: 198 - DNA, 195 bp, synthetic construct
  misc_feature 1..195: note = Synthetic polynucleotide
  source 1..195: mol_type = other DNA
aaagcaagctgataaaccgatacaattaaaggctccttttggagcctttttttttggagattttcaacatgaaaaaattattatttgatgatcagatagcggcggggaactgccagacatcaaataaaacaaaaggctcagtcggaagactgggccttttgttttatctgttgtttgtcggtgaacactctcccg

SEQ ID NO: 199 - DNA, 164 bp, synthetic construct
  misc_feature 1..164: note = Synthetic polynucleotide
  source 1..164: mol_type = other DNA
aacgcatgagaaagcccccggaagatcaccttccgggggcttttttattgcgctccttggccctccatccttagatagctcggtaccaaattccagaaaagaggcctcccgaaaggggggccttttttcgttttggtcctcataggcaatacgatcgcatgtcc

SEQ ID NO: 200 - DNA, 193 bp, synthetic construct
  misc_feature 1..193: note = Synthetic polynucleotide
  source 1..193: mol_type = other DNA
aacgcatgagaaagcccccggaagatcaccttccgggggcttttttattgcgctccttggccctccatccttagatagttcagccaaaaaacttaagaccgccggtcttgtccactaccttgcagtaatgcggtggacaggatcggcggttttcttttctcttctcaatcataggcaatacgatcgcatgtcc

SEQ ID NO: 201 - DNA, 67 bp, synthetic construct
  misc_feature 1..67: note = Synthetic polynucleotide
  source 1..67: mol_type = other DNA
cctaggacctgtaggatcgtacaggtttacgcaagaaaatggtttgttactttcgaataaatctaga

SEQ ID NO: 202 - DNA, 68 bp, synthetic construct
  misc_feature 1..68: note = Synthetic polynucleotide
  source 1..68: mol_type = other DNA
cggtggaatccctatcagtgatagagattgacatccctatcagtgatagatataatgagcactctaga

SEQ ID NO: 203 - DNA, 90 bp, synthetic construct
  misc_feature 1..90: note = Synthetic polynucleotide
  source 1..90: mol_type = other DNA
aacaaacagacaatctggtctgtttgtattatggaaaatttttctgtataatagattcaacaaacagacaatctggtctgtttgtattat

SEQ ID NO: 204 - DNA, 58 bp, synthetic construct
  misc_feature 1..58: note = Synthetic polynucleotide
  source 1..58: mol_type = other DNA
aaaaagagtttgacatgatacgaaacgtaccgtatcgttaaggttactagagtctaga

SEQ ID NO: 205 - DNA, 116 bp, synthetic construct
  misc_feature 1..116: note = Synthetic polynucleotide
  source 1..116: mol_type = other DNA
ggggcctcgcttgggttattgctggtgcccggccgggcgcaatattcatgttgatgatttattatatatcgagtggtgtatttatttatattgtttgctccgttaccgttattaac

SEQ ID NO: 206 - DNA, 753 bp, synthetic construct
  misc_feature 1..753: note = Synthetic polynucleotide
  source 1..753: mol_type = other DNA
atgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggaatgcttagttttgcacattcagaaaaagacaactatatagatagtttatttttacatgcgtgtatgaacataccattaattgttccttctctagttgataattatcgaaaaataaatatagcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaa

SEQ ID NO: 207 - DNA, 624 bp, synthetic construct
  misc_feature 1..624: note = Synthetic polynucleotide
  source 1..624: mol_type = other DNA
atgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcctaa

SEQ ID NO: 208 - DNA, 612 bp, synthetic construct
  misc_feature 1..612: note = Synthetic polynucleotide
  source 1..612: mol_type = other DNA
atgagcccgaaacgtcgtacccaggcagaacgtgcaatggaaacccagggtaaactgattgcagcagcactgggtgttctgcgtgaaaaaggttatgcaggttttcgtattgcagatgttccgggtgcagccggtgttagccgtggtgcacagagccatcattttccgaccaaactggaactgctgctggcaacctttgaatggctgtatgagcagattaccgaacgtagccgtgcacgtctggcaaaactgaaaccggaagatgatgttattcagcagatgctggatgatgcagcagaattttttctggatgatgattttagcatcagcctggatctgattgttgcagcagatcgtgatccggcactgcgtgaaggtattcagcgtaccgttgaacgtaatcgttttgttgttgaagatatgtggctgggtgtgctggtgagccgtggtctgagccgtgatgatgccgaagatattctgtggctgatttttaacagcgttcgtggtctggcagttcgtagcctgtggcagaaagataaagaacgttttgaacgtgtgcgtaatagcaccctggaaattgcacgtgaacgttatgcaaaattcaaacgttga

SEQ ID NO: 209 - DNA, 603 bp, synthetic construct
  misc_feature 1..603: note = Synthetic polynucleotide
  source 1..603: mol_type = other DNA
atggcacgtaccccgagccgtagcagcattggtagcctgcgtagtccgcatacccataaagcaattctgaccagcaccattgaaatcctgaaagaatgtggttatagcggtctgagcattgaaagcgttgcacgtcgtgccggtgcaagcaaaccgaccatttatcgttggtggaccaataaagcagcactgattgccgaagtgtatgaaaatgaaagcgaacaggtgcgtaaatttccggatctgggtagctttaaagccgatctggattttctgctgcgtaatctgtggaaagtttggcgtgaaaccatttgtggtgaagcatttcgttgtgttattgcagaagcacagctggaccctgcaaccctgacccagctgaaagatcagtttatggaacgtcgtcgtgagatgccgaaaaaactggttgaaaatgccattagcaatggtgaactgccgaaagataccaatcgtgaactgctgctggatatgatttttggtttttgttggtatcgcctgctgaccgaacagctgaccgttgaacaggatattgaagaatttaccttcctgctgattaatggtgtttgtccgggtacacagcgttaa

SEQ ID NO: 210 - DNA, 906 bp, synthetic construct
  misc_feature 1..906: note = Synthetic polynucleotide
  source 1..906: mol_type = other DNA
atggaactgcgtgacctggatttaaacctgctggtggtgttcaaccagttgctggtcgacagacgcgtctctgtcactgcggagaacctgggcctgacccagcctgccgtgagcaatgcgctgaaacgcctgcgcacctcgctacaggacccactcttcgtgcgcacacatcagggaatggaacccacaccctatgccgcgcatctggccgagcacgtcacttcggccatgcacgcactgcgcaacgccctacagcaccatgaaagcttcgatccgctgaccagcgagcgtaccttcaccctggccatgaccgacattggcgagatctacttcatgccgcggctgatggatgcgctggctcaccaggcccccaattgcgtgatcagtacggtgcgcgacagttcgatgagcctgatgcaggccttgcagaacggaaccgtggacttggccgtgggcctgcttcccaatctgcaaactggcttctttcagcgccggctgctccagaatcactacgtgtgcctatgtcgcaaggaccatccagtcacccgcgaacccctgactctggagcgcttctgttcctacggccacgtgcgtgtcatcgccgctggcaccggccacggcgaggtggacacgtacatgacacgggtcggcatccggcgcgacatccgtctggaagtgccgcacttcgccgccgttggccacatcctccagcgcaccgatctgctcgccactgtgccgatatgtttagccgactgctgcgtagagcccttcggcctaagcgccttgccgcacccagtcgtcttgcctgaaatagccatcaacatgttctggcatgcgaagtaccacaaggacctagccaatatttggttgcggcaactgatgtttgacctgtttacggattgataa

SEQ ID NO: 211 - DNA, 90 bp, synthetic construct
  misc_feature 1..90: note = Synthetic polynucleotide
  source 1..90: mol_type = other DNA
tcaatgtattgatgccgtccatatcatgaatcaaaacaatccatttgatcaatatcaagctcactcttaagcttcactcatccgctgcat

SEQ ID NO: 212 - DNA, 930 bp, synthetic construct
  misc_feature 1..930: note = Synthetic polynucleotide
  source 1..930: mol_type = other DNA
atgcgtttcaacaagctcgacctcaatcttctggtcgccctggatgcactgctcacggagatgagcatcagccgcgccgccgaaaagatccatctgagccagtcggccatgagcaatgccctggcgcggctgcgcgagtatttcgatgatgaattgctgatccaggtgggccggcgcatggagcccacgccgcgcgccgaggtgctcaaggatgcggtgcatgatgtgctgcggcgtatcgatggctccatcgcggcgctgccggccttcgtgccggccgagtccacgcgcgagtttcgcatctcggtttcggactttacgctctccgtcctcatcccccgggtgctggcgcgcgcgcacgccgagggcaagcacatccgctttgccctgatgccgcaggtgcaagacccgacccgctcgctggatcgggccgaggtggacctgctggtcttgccgcaggaattctgcacgcccgatcatcctgccgaagaggtcttccgcgaacggcatgtctgcgtggtctggcgcgacagtgcgctggcgcaaggcgagctgacgctggaacgctacatggcctcaggccatgtggtgatggtgccgcctggggccaatgcgtcgtcggtggaggcgtggatggccaggaagctgggctttgcgcgccgggtggaagtgaccagcttcagcttcgcttctgcgctggcgctggtacaggggacggaccgcatcgccacggtgcatgcccggctggcgcagctgctggctccgcaatggccggtggtgatcaaggagagtccgctgtcgctgggcgagatgcggcagatgatgcagtggcatcgctaccgcagcaatgatcctggcatccagtggctgcgtcgggtgtttctggagagtgcgcaggagatggatgcggcgctgccaggcatctgctga

SEQ ID NO: 213 - DNA, 286 bp, synthetic construct
  misc_feature 1..286: note = Synthetic polynucleotide
  source 1..286: mol_type = other DNA
cagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctatattttctccatacccgtttttttgggctagcgaattc

SEQ ID NO: 214 - DNA, 930 bp, synthetic construct
  misc_feature 1..930: note = Synthetic polynucleotide
  source 1..930: mol_type = other DNA
atgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataa

SEQ ID NO: 215 - DNA, 1419 bp, synthetic construct
  misc_feature 1..1419: note = Synthetic polynucleotide
  source 1..1419: mol_type = other DNA
atggttactatcaatacggaatctgctttaacgccacgttctttgcgggatacgcggcgtatgaatatgtttgtttcggtagctgctgcggtcgcaggattgttatttggtcttgatatcggcgtaatcgccggagcgttgccgttcattaccgatcactttgtgctgaccagtcgtttgcaggaatgggtggttagtagcatgatgctcggtgcagcaattggtgcgctgtttaatggttggctgtcgttccgcctggggcgtaaatacagcctgatggcgggggccatcctgtttgtactcggttctatagggtccgcttttgcgaccagcgtagagatgttaatcgccgctcgtgtggtgctgggcattgctgtcgggatcgcgtcttacaccgctcctctgtatctttctgaaatggcaagtgaaaacgttcgcggtaagatgatcagtatgtaccagttgatggtcacactcggcatcgtgctggcgtttttatccgatacagcgttcagttatagcggtaactggcgcgcaatgttgggggttcttgctttaccagcagttctgctgattattctggtagtcttcctgccaaatagcccgcgctggctggcggaaaaggggcgtcatattgaggcggaagaagtattgcgtatgctgcgcgatacgtcggaaaaagcgcgagaagaactcaacgaaattcgtgaaagcctgaagttaaaacagggcggttgggcactgtttaagatcaaccgtaacgtccgtcgtgctgtgtttctcggtatgttgttgcaggcgatgcagcagtttaccggtatgaacatcatcatgtactacgcgccgcgtatcttcaaaatggcgggctttacgaccacagaacaacagatgattgcgactctggtcgtagggctgacctttatgttcgccacctttattgcggtgtttacggtagataaagcagggcgtaaaccggctctgaaaattggtttcagcgtgatggcgttaggcactctggtgctgggctattgcctgatgcagtttgataacggtacggcttccagtggcttgtcctggctctctgttggcatgacgatgatgtgtattgccggttatgcgatgagcgccgcgccagtggtgtggatcctgtgctctgaaattcagccgctgaaatgccgcgatttcggtattacctgttcgaccaccacgaactgggtgtcgaatatgattatcggcgcgaccttcctgacactgcttgatagcattggcgctgccggtacgttctggctctacactgcgctgaacattgcgtttgtgggcattactttctggctcattccggaaaccaaaaatgtcacgctggaacatatcgaacgcaaactgatggcaggcgagaagttgagaaatatcggcgtctga

SEQ ID NO: 216 - DNA, 75 bp, synthetic construct
  misc_feature 1..75: note = Synthetic polynucleotide
  source 1..75: mol_type = other DNA
ctcgagtgttgacaattaatcatcggctcgtataatgtgtggaattgtgagcgctcacaatttcacacatctaga

SEQ ID NO: 217 - DNA, 221 bp, synthetic construct
  misc_feature 1..221: note = Synthetic polynucleotide
  source 1..221: mol_type = other DNA
aacaaatacacatgggcgcatgcctattactgcccttgcgatatggaaggcaagcttttagtaacaatagaaaactgggtcctactctcgaagaatgcactgcggcggtcacgtcaacacgtgctgcaccgttgagaatgaatgctgggcagattgccagcggcgtcattttcggctgtcccgtcctcacggttttgcgctgcatcgcaagagattgggaa

SEQ ID NO: 218 - DNA, 849 bp, synthetic construct
  misc_feature 1..849: note = Synthetic polynucleotide
  source 1..849: mol_type = other DNA
atgacgtcagcagcgaatctggtgaggatcacgcagcccgcgatcagccggctgatcagggatctcgaagaggaaattgggatcagcctcttcgaaagaacgggcaaccggttacgtcctacgcgggaggccggtattctgttcaaggaagtgtcgcgacatttcaacgggattcagcacatcgacaaagtcgcggctgaactgaagaagtctcatatggggtccctaagggtcgcctgttatacagcgccagctctgagttttatgtccggcgtcattcagacgttcatcgccgatcggcccgacgtgtcggtctacctcgacacagttccttcccagacggtcctcgaattggtctcgctccagcactacgatctcggaatatcgatattggctggcgactatcctggtctcaccaccgaacctgtcccttcctttcgtgcggtctgcctgctgccgccggggcatcgtctcgaagacaaggaaactgttcatgcgacggaccttgaaggagagtcattgatttgcctctctccagtgagccttctacggatgcaaacggacgccgcactggacagctgcggcgtccactgtaatcgcaggatagaaagtagtctggcgctgaatctctgcgatctggtaagcaggggaatgggggttggtatcgtcgaccccttcactgccgactactacagtgcaaatccggttattcagcgctcctttgatccggttgtcccctaccattttgctatagttcttccgaccgacagcccaccgccgcgcttggttagcgagttccgggcagcgttgcttgatgctttgaaagccttgccctatgaaaccatttga

SEQ ID NO: 219 - DNA, 119 bp, synthetic construct
  misc_feature 1..119: note = Synthetic polynucleotide
  source 1..119: mol_type = other DNA
aaacgcaccataacatctgcttattcttgcccggtcattatgaatttgaccgaatgcatatcgaatgtaaagctcaccctataaatcacaactcttccgggccaaccgggatcagacgt

SEQ ID NO: 220 - DNA, 897 bp, synthetic construct
  misc_feature 1..897: note = Synthetic polynucleotide
  source 1..897: mol_type = other DNA
atgaatctcaggcaggtcgaggcgttccgggcagtcatgctgacggggcaaatgacggcggcggctgaactaatgctggtgactcagccggccatcagtcgcctaatcaaggactttgaacaggcgacaaaactgcagctcttcgagaggcgtgggaaccatattatcccgacacaggaggcaaagacgctgtggaaagaggtcgatcgggcgttcgtcgggcttaatcatataggcaacctggctgccgacatcggcaggcaggcagcggggacgctccgcattgctgcaatgcctgctctggcaaacggcctcttgccgcggtttcttgctcagttcatccgtgacagaccaaatctccaggtctccctaatgggactgccctcaagcatggtcatggaagccgttgcgtccggcagggccgacatcggttatgccgatggcccacaggagcgccaaggttttctaatcgaaacccggtcgcttcccgctgttgtcgctgtcccgatgggacatcgacttgctggccttgaccgtgtcacgccacaggaccttgccggtgagcgtattataaaacaggagactggcactctcttcgccatgcgggtagaggtggcgattggtggtattcaacgccggccgtcaattgaagtgagcctgtcgcatactgcgctaagtctcgtccgcgaaggcgccgggatcgcaattatcgatccagccgcggcgatcgagttcacggacaggatcgtactgcgaccgttctcgatcttcattgacgccggattcctcgaagtccggtcagcaattggcgctccctcaaccatcgtcgatcgtttcacaaccgaattctggaggtttcatgatgacttgatgaagcagaacggcctaatggagtaa

SEQ ID NO: 221 - DNA, 63 bp, synthetic construct
  misc_feature 1..63: note = Synthetic polynucleotide
  source 1..63: mol_type = other DNA
agcgcgggtgagagggattcgttaccaatagacaattgattggacgttcaatataatgctagc

SEQ ID NO: 222 - DNA, 226 bp, synthetic construct
  misc_feature 1..226: note = Synthetic polynucleotide
  source 1..226: mol_type = other DNA
ccctttgtgcgtccaaacggacgcacggcgctctaaagcgggtcgcgatctttcagattcgctcctcgcgctttcagtctttgttttggcgcatgtcgttatcgcaaaaccgctgcacacttttgcgcgacatgctctgatccccctcatctgggggggcctatctgagggaatttccgatccggctcgcctgaaccattctgctttccacgaacttgaaaacgct

SEQ ID NO: 223 - DNA, 93 bp, synthetic construct
  misc_feature 1..93: note = Synthetic polynucleotide
  source 1..93: mol_type = other DNA
ttttgttcgattatcgaacaaattattgaaatatcgaacaaaacctctaaactactgtggcactgaatcaaaaaattataaaccctgatcaga

SEQ ID NO: 224 - DNA, 51 bp, synthetic construct
  misc_feature 1..51: note = Synthetic polynucleotide
  source 1..51: mol_type = other DNA
cacccagcagtatttacaaacaaccatgaatgtaagtatattccttagcaa

SEQ ID NO: 225 - DNA, 50 bp, synthetic construct
  misc_feature 1..50: note = Synthetic polynucleotide
  source 1..50: mol_type = other DNA
attggatccaattgacagctagctcagtcctaggtaccattggatccaat

SEQ ID NO: 226 - DNA, 54 bp, unidentified
  misc_feature 1..54: note = R. sp. IRBG74
  source 1..54: mol_type = genomic DNA
atttcacacatctagagctaatcatctcgtactaaagaggagaaattaaccatg

SEQ ID NO: 227 - DNA, 49 bp, unidentified
  misc_feature 1..49: note = R. sp. IRBG74
  source 1..49: mol_type = genomic DNA
atttcacacatctagagctaatcatcgcgtactcaggaggcaagtaatg

SEQ ID NO: 228 - DNA, 40 bp, unidentified
  misc_feature 1..40: note = R. sp. IRBG74
  source 1..40: mol_type = genomic DNA
atttcacacatctagaattaaagaggagaaattaaccatg

SEQ ID NO: 229 - DNA, 54 bp, unidentified
  misc_feature 1..54: note = R. sp. IRBG74
  source 1..54: mol_type = genomic DNA
taacaatttcacacatctagagctaatcatctcgtactaaagaggcaagtaatg

SEQ ID NO: 230 - DNA, 54 bp, unidentified
  misc_feature 1..54: note = R. sp. IRBG74
  source 1..54: mol_type = genomic DNA
taacaatttcacacatctagagctaatcatcgcgtactaaggaggcaagtaatg

SEQ ID NO: 231 - DNA, 54 bp, unidentified
  misc_feature 1..54: note = R. sp. IRBG74
  source 1..54: mol_type = genomic DNA
taacaatttcacacatctagagctaatcatcgcgtactcaagaggcaagtaatg

SEQ ID NO: 232 - DNA, 54 bp, unidentified
  misc_feature 1..54: note = R. sp. IRBG74
  source 1..54: mol_type = genomic DNA
taacaatttcacacatctagagctaatcttcgcgtactaaagaggcaagtaatg

SEQ ID NO: 233 - DNA, 54 bp, unidentified
  misc_feature 1..54: note = R. sp. IRBG74
  source 1..54: mol_type = genomic DNA
taacaatttcacacatctagagctaatcatctcgtactcaggaggcaagtaatg

SEQ ID NO: 234 - DNA, 54 bp, unidentified
  misc_feature 1..54: note = R. sp. IRBG74
  source 1..54: mol_type = genomic DNA
taacaatttcacacatctagagctaatcatctcgtactaatgaggcaagtaatg

SEQ ID NO: 235 - DNA, 54 bp, unidentified
  misc_feature 1..54: note = R. sp. IRBG74
  source 1..54: mol_type = genomic DNA
taacaatttcacacatctagagctaatcatcgcgtactaatgaggcaagtaatg

SEQ ID NO: 236 - DNA, 54 bp, unidentified
  misc_feature 1..54: note = R. sp. IRBG74
  source 1..54: mol_type = genomic DNA
taacaatttcacacatctagagctaatcatcgcgtactcacgaggcaagtaatg

SEQ ID NO: 237 - DNA, 54 bp, unidentified
  misc_feature 1..54: note = R. sp. IRBG74
  source 1..54: mol_type = genomic DNA
taacaatttcacacatctagagctaatcatcgcgtactaaaaaggcaagtaatg

SEQ ID NO: 238 - DNA, 54 bp, unidentified
  misc_feature 1..54: note = R. sp. IRBG74
  source 1..54: mol_type = genomic DNA
taacaatttcacacatctagagctaatcttcgcgtactaaaaaggcaagtaatg

SEQ ID NO: 239 - DNA, 54 bp, unidentified
  misc_feature 1..54: note = R. sp. IRBG74
  source 1..54: mol_type = genomic DNA
taacaatttcacacatctagagctaatcttcgcgtactaagaaggcaagtaatg

SEQ ID NO: 240 - DNA, 54 bp, unidentified
  misc_feature 1..54: note = R. sp. IRBG74
  source 1..54: mol_type = genomic DNA
taacaatttcacacatctagagctaatcatctcgtactaaataggcaagtaatg

SEQ ID NO: 241 - DNA, 54 bp, unidentified
  misc_feature 1..54: note = R. sp. IRBG74
  source 1..54: mol_type = genomic DNA
taacaatttcacacatctagagctaatcatctcgtactaataaggcaagtaatg

SEQ ID NO: 242 - DNA, 54 bp, unidentified
  misc_feature 1..54: note = R. sp. IRBG74
  source 1..54: mol_type = genomic DNA
taacaatttcacacatctagagctaatcttctcgtactaaagaggcaagtaatg

SEQ ID NO: 243 - DNA, 54 bp, unidentified
  misc_feature 1..54: note = R. sp. IRBG74
  source 1..54: mol_type = genomic DNA
taacaatttcacacatctagagctaatcatcgcgtactcaataggccagtaatg

SEQ ID NO: 244 - DNA, 54 bp, unidentified
  misc_feature 1..54: note = R. sp. IRBG74
  source 1..54: mol_type = genomic DNA
taacaatttcacacatctagagctaatcatcgcgtactaagtaggcaagtaatg

SEQ ID NO: 245 - DNA, 54 bp, unidentified
  misc_feature 1..54: note = R. sp. IRBG74
  source 1..54: mol_type = genomic DNA
taacaatttcacacatctagagctaatcatctcgtactaacgaggcaagtaatg

SEQ ID NO: 246 - DNA, 54 bp, unidentified
  misc_feature 1..54: note = R. sp. IRBG74
  source 1..54: mol_type = genomic DNA
taacaatttcacacatctagagctaatcatcgcgtactcagcaggcaagtaatg

SEQ ID NO: 247 - DNA, 54 bp, unidentified
  misc_feature 1..54: note = R. sp. IRBG74
  source 1..54: mol_type = genomic DNA
taacaatttcacacatctagagctaatcttcgcgtactaagtaggcaagtaatg

SEQ ID NO: 248 - DNA, 54 bp, unidentified
  misc_feature 1..54: note = R. sp. IRBG74
  source 1..54: mol_type = genomic DNA
taacaatttcacacatctagagctaatcttcgcgtactaattaggcaagtaatg

SEQ ID NO: 249 - DNA, 54 bp, unidentified
  misc_feature 1..54: note = R. sp. IRBG74
  source 1..54: mol_type = genomic DNA
taacaatttcacacatctagagctaatcttctcgtactaacaaggcaagtaatg

SEQ ID NO: 250 - DNA, 54 bp, unidentified
  misc_feature 1..54: note = R. sp. IRBG74
  source 1..54: mol_type = genomic DNA
taacaatttcacacatctagagctaatcatctcgtactcaataggcaagtaatg

SEQ ID NO: 251 - DNA, 54 bp, unidentified
  misc_feature 1..54: note = R. sp. IRBG74
  source 1..54: mol_type = genomic DNA
taacaatttcacacatctagagctaatcatctcgtactaagcacgcaagtaatg

SEQ ID NO: 252 - DNA, 54 bp, unidentified
  misc_feature 1..54: note = R. sp. IRBG74
  source 1..54: mol_type = genomic DNA
taacaatttcacacatctagagctaatcatcgcgtactaactacgcaagtaatg

SEQ ID NO: 253 - DNA, 54 bp, unidentified
  misc_feature 1..54: note = R. sp. IRBG74
  source 1..54: mol_type = genomic DNA
taacaatttcacacatctagagctaatcttcgcgtactaagaacgcaagtaatg

SEQ ID NO: 254 - DNA, 54 bp, unidentified
  misc_feature 1..54: note = R. sp. IRBG74
  source 1..54: mol_type = genomic DNA
taacaatttcacacatctagagctaatcttcgcgtactaaaaacgcaagtaatg

SEQ ID NO: 255 - DNA, 54 bp, unidentified
  misc_feature 1..54: note = R. sp. IRBG74
  source 1..54: mol_type = genomic DNA
taacaatttcacacatctagagctaatcttcgcgtactaacaacgcaagtaatg

SEQ ID NO: 256 - DNA, 54 bp, unidentified
  misc_feature 1..54: note = R. sp. IRBG74
  source 1..54: mol_type = genomic DNA
taacaatttcacacatctagagctaatcttctcgtactcatgacgcaagtaatg

SEQ ID NO: 257 - DNA, 40 bp, unidentified
  misc_feature 1..40: note = R. sp. IRBG74
  source 1..40: mol_type = genomic DNA
atttcacacatctagaattaaagagaagaaattaaccatg

SEQ ID NO: 258 - DNA, 29 bp, unidentified
  misc_feature 1..29: note = R. sp. IRBG74
  source 1..29: mol_type = genomic DNA
ctagtgcgaactagctcataccgcagatg

SEQ ID NO: 259 - DNA, 42 bp, Pseudomonas protegens
  source 1..42: mol_type = genomic DNA
ctagcgcaggtccaacgtttttctaagcaaggaggtcatatg

SEQ ID NO: 260 - DNA, 42 bp, Pseudomonas protegens
  source 1..42: mol_type = genomic DNA
ctagcgaaggtccaacgtttttctaagcaaggaggtcatatg

SEQ ID NO: 261 - DNA, 42 bp, Pseudomonas protegens
  source 1..42: mol_type = genomic DNA
ctagcgaaggtccaacgtttttctaagccaggaggtcatatg

SEQ ID NO: 262 - DNA, 42 bp, Pseudomonas protegens
  source 1..42: mol_type = genomic DNA
ctagcgcaggtccaacgtttttctaagccaggaggtcatatg

SEQ ID NO: 263 - DNA, 42 bp, Pseudomonas protegens
  source 1..42: mol_type = genomic DNA
ctagcgaagctccaacgtttttctaagcaaggaggtcatatg

SEQ ID NO: 264 - DNA, 33 bp, Pseudomonas protegens
  source 1..33: mol_type = genomic DNA
gaattctacactaacggacaggagggtccgatg

SEQ ID NO: 265 - DNA, 33 bp, Pseudomonas protegens
  source 1..33: mol_type = genomic DNA
gaattctaaactaacggacaggagggtccgatg

SEQ ID NO: 266 - DNA, 33 bp, Pseudomonas protegens
  source 1..33: mol_type = genomic DNA
gaattctaagctaacggacaggagggtccgatg

SEQ ID NO: 267 - DNA, 33 bp, Pseudomonas protegens
  source 1..33: mol_type = genomic DNA
gaattcttaactaacggacaggagggtccgatg

SEQ ID NO: 268 - DNA, 33 bp, Pseudomonas protegens
  source 1..33: mol_type = genomic DNA
gaattctacactaacggacaggagggtcggatg

SEQ ID NO: 269 - DNA, 33 bp, Pseudomonas protegens
  source 1..33: mol_type = genomic DNA
gaattctacgctaacggacaggagggtccgatg

SEQ ID NO: 270 - DNA, 33 bp, Pseudomonas protegens
  source 1..33: mol_type = genomic DNA
gaattctcaactaacggacaggagggtccgatg

SEQ ID NO: 271 - DNA, 33 bp, Pseudomonas protegens
  source 1..33: mol_type = genomic DNA
gaattctaagctaacggacaggagggtcggatg

SEQ ID NO: 272 - DNA, 33 bp, Pseudomonas protegens
  source 1..33: mol_type = genomic DNA
gaattctcagctaacggacaggagggtccgatg

SEQ ID NO: 273 - DNA, 33 bp, Pseudomonas protegens; identical to SEQ ID NO: 270
  source 1..33: mol_type = genomic DNA

SEQ ID NO: 274 - DNA, 33 bp, Pseudomonas protegens
  source 1..33: mol_type = genomic DNA
gaattctacgctaacggacaggagggtcggatg

SEQ ID NO: 275 - DNA, 35 bp, Pseudomonas protegens
  source 1..35: mol_type = genomic DNA
gaattctcaactaacggacaggagatatacatatg

SEQ ID NO: 276 - DNA, 33 bp, Pseudomonas protegens
  source 1..33: mol_type = genomic DNA
gaattctcagctaacggacaggagggtcggatg

SEQ ID NO: 277 - DNA, 33 bp, Pseudomonas protegens
  source 1..33: mol_type = genomic DNA
gaattctaaactaacggacaggagggtcggatg

SEQ ID NO: 278 - DNA, 33 bp, Pseudomonas protegens
  source 1..33: mol_type = genomic DNA
gaattctcagctcacggacaggagggtcggatg

SEQ ID NO: 279 - DNA, 34 bp, Pseudomonas protegens
  source 1..34: mol_type = genomic DNA
gaattctcaactaacggacaggagggtcgggatg

SEQ ID NO: 280 - DNA, 33 bp, Pseudomonas protegens
  source 1..33: mol_type = genomic DNA
gaattctacactcacggacaggagggtcggatg

SEQ ID NO: 281 - DNA, 33 bp, Pseudomonas protegens
  source 1..33: mol_type = genomic DNA
gaattctaagctcacggacaggagggtcggatg

SEQ ID NO: 282 - DNA, 33 bp, Pseudomonas protegens
  source 1..33: mol_type = genomic DNA
gaattctcaactcacggacaggagggtcggatg

SEQ ID NO: 283 - DNA, 33 bp, Pseudomonas protegens
  source 1..33: mol_type = genomic DNA
gaattctacactaacggacagcagggtcggatg

SEQ ID NO: 284 - DNA, 42 bp, Pseudomonas protegens
  source 1..42: mol_type = genomic DNA
ctagcgcaggtccaacctttttctaagcaagtaggtcatatg

SEQ ID NO: 285 - DNA, 33 bp, Pseudomonas protegens
  source 1..33: mol_type = genomic DNA
gaattctcagctaacggacagcagggtcggatg

SEQ ID NO: 286 - DNA, 42 bp, Pseudomonas protegens
  source 1..42: mol_type = genomic DNA
ctagcgcaggtccaacctttttctaagcaactaggtcatatg

SEQ ID NO: 287 - DNA, 42 bp, Pseudomonas protegens
  source 1..42: mol_type = genomic DNA
ctagcgaaggtccaacctttttctaagccagtaggtcatatg

SEQ ID NO: 288 - DNA, 33 bp, Pseudomonas protegens
  source 1..33: mol_type = genomic DNA
gaattctacgctcacggacagcagggtcggatg

SEQ ID NO: 289 - DNA, 33 bp, Pseudomonas protegens
  source 1..33: mol_type = genomic DNA
gaattctccgctcacggacaggagggtccgatg

SEQ ID NO: 290 - DNA, 33 bp, Pseudomonas protegens
  source 1..33: mol_type = genomic DNA
cttctcggccagctgacaggggaagctcgcatg

SEQ ID NO: 291 - DNA, 34 bp, Pseudomonas protegens
  source 1..34: mol_type = genomic DNA
cttctcggccagctgacaggaggaagctcgcatg

SEQ ID NO: 292 - protein, 582 aa, Rhodobacter sphaeroides
MDTSAARSGAVAERGEEYLTLDALCEIAKLLTGASDPIACMPAVFGVLGAFMGLRHGALAILQEGAQAETQRNARHVNPYVIAATASGVPPAGAEARAIPAQVARHVFRNGVSLVSCDILEEFGAEALPPGLGDSRQALVAVPIRDQANSPFVLGVLCAYRSLKDNGARYLDTDLRVLNMVAAVLEQSIRFRRLVARDRDRIVQEAREAIRVAAEATAGPPVEAPAELALEGVIGSSPAIQRVIGQIRKVAGTHTPVLLRGESGTGKEVFARALHALSERRDKAFIKVNCAALSQSLLESELFGHEKGSFTGAVQQKKGRPEMAEGGTLFLDEIGEISLEFQAKLLRILQEGEFERVGGTRTLRVDVRLVTATNKDLERAVANGTFRADLYFRICVVPIVLPPLRDRKEDIGLLAQGLLERFNKRNGMKKKLHPSAVAALAQCNFPGNVRELENCIARVAALSPETVIHADDLACHHDHCLSADLWRLQTGSASPVGGLAQGPLELPVLGSRPPAAAPSAPPPPPPTVPSAPLDGEAAEREALIEAMERAGWVQAKAARLRGMTPRQIGYALKKYNIRVEKF

SEQ ID NO: 293 - protein, 615 aa, Azorhizobium caulinodans
MPMTDAFQVRVPRVSSSTAGDIAASSITTRGALPRPGGMPVSMSRGTSPEVALIGVYEISKILTAPRRLEVTLANVVNVLSSMLQMRHGMICILDSEGDPDMVATTGWTPEMAGQIRAHVPQKAIDQIVATQMPLVVQDVTADPLFAGHEDLFGPPEEATVSFIGVPIKADHHVMGTLSIDRIWDGTARFRFDEDVRFLTMVANLVGQTVRLHKLVASDRDRLIAQTHRLEKALREEKSGAEPEVAEAANGSAMGIVGDSPLVKRLIATAQVVARSNSTVLLRGESGTGKELFARAIHELSPRKGKPFVKVNCAALPESVLESELFGHEKGAFTGALNMRQGRFELAHGGTLFLDEIDEITPAFQAKLLRVLQEGEFERVGGNRTLKVDVRLVCATNKNLEEAVSKGEFRADLYYRIHVVPLILPPLRERPGDIPKLAKNFLDRFNKENKLHMMLSAPAIDVLRRCYFPGNVRELENCIRRTATLAHDAVITPHDFACDSGQCLSAMLWKGSAPKPVMPHVPPAPTPLTPLSPAPLATAAPAAASPAPAADSLPVTCPGTEACPAVPPRQSEKEQLLQAMERSGWVQAKAARLLNLTPRQVGYALRKYDIDIKRF